SEQUENCE LISTING (92 sequences)

SEQ ID NO: 1 - protein, 60 aa, Acinetobacter baumannii
MTQLINKGGFRERANRSRKYQQSENKQVALPSKKYQPQTKLQDNQSEMIQAKAGTAETSD

SEQ ID NO: 2 - protein, 66 aa, Acinetobacter baumannii
MKLAKTLLATTLALTAASTFAASKHDQAHNTAGEEKVVVSTQEQANTANAASDAVGSASEAAPATR

SEQ ID NO: 3 - protein, 66 aa, Acinetobacter baumannii
MIDEEKPLNFEDDDEPLDFEDEEFIDDKKEDEMYNSITKDGSSVDPADDGTRHIRPEDGDPIEIDE

SEQ ID NO: 4 - protein, 73 aa, Acinetobacter baumannii
MSTTNNQANQRNNQQQQQQQNDNRNQQQHGNQQQNDQQQQNNQQQQQNDNRGQQQGSNQKDSGQQNSNNNQQR

SEQ ID NO: 5 - protein, 108 aa, Acinetobacter baumannii
MSAKLVVTLLATSLLTVGCVAYTDDPYYRGGYGYHDHDDDRYDRNDGRRYSEWERKRWEERKRLYEQQRKDIREQQKDRREWEKRHREWEKKRLEDRDHDHRDYRHDD

SEQ ID NO: 6 - protein, 114 aa, Acinetobacter baumannii
MNKLLVALGLAATVALVGCNKDKAPETGATTGEHLENAAQQATADIKSAGDQAASDIATATDNASAKIDAAADHAADATAKAAAETEATARKATADTAQAVENAAADVKKDAQH

SEQ ID NO: 7 - protein, 126 aa, Acinetobacter baumannii
MKMTAKIALFSAAIVTMGSLAACQSTTQPPKPEHGMMQDGPRDGHHHRMKHREFTPEQKAAWEQHRAERKARFEQIQKACEGKVVGQTVNVQVGDKTLEGTCNLRFEPKRPQPPVNAPAPVATQAK

SEQ ID NO: 8 - protein, 139 aa, Acinetobacter baumannii
MKAIKILCITSSILVSSSLFAETPQPQQVNEATSKTMPYGDNPSLGRVLLYKTGKGIQNLGDSIQGASEKTSNKISEKWKDTKEFTAEKAEVVQQKADTAKVFTEQKIEQAKQNITSSRNGENIPIEQGELSKSSTTAN

SEQ ID NO: 9 - protein, 159 aa, Acinetobacter baumannii
MKKSLLAIALMSTLLVACNKHENKTETTSDASTPVQTAQSNNNEAVDTAHTAENSLDWDGKYKGTLPCADCEGIKTELELKDDKTYELTETYLGKGDANPFETHGKFTFDKDNTSVITLDDKAQNRKFFIGENTATALDMEGKKVEGSLAEHYVLKKED

SEQ ID NO: 10 - protein, 162 aa, Acinetobacter baumannii
MANKKLLICAAIAAGLLLTACVKKETPKEEEQDKVETAVSEPQPQKPAKFESLESVDTQEAQVQEQPQVEVHREETANTTTEIRRETRPARSDESSQTQVAEQPKSETPKVEPKPEKKPEPKAEPKPEKAQSKPAAKATEPANTEDDAVAAAIAAATPALKN

SEQ ID NO: 11 - protein, 166 aa, Acinetobacter baumannii
MTTENKLDELKANAADAKVQGEKALDDLKENVKEKQTAGKEAIADKVDELKTKAADAKVQGEKALEDLKENVKEKQAAAKEAVEDKASDLKGKLDDAQHSLQDKFDHLRTEAAHKLDDAKAKAAELKEEAATKFDELKTQATAKFDELKKTATEKLNKLKNHDSAE

SEQ ID NO: 12 - protein, 230 aa, Acinetobacter baumannii
MHTRRILLAFSLAASAASVAFADYQNINQSTDSDRLEQLSKTLSQGSYTHPDDLDLPASAKVSVTLREKTVELNNDSLAKKYGTTTAKNSFKTSSSNPYSWLVSHPLPDTVRVSSNFGGRTMGGRAEHHGGLDMAAPSGTPIYATGPGIVTKSGWGTGYGQYVEINHGNGYLTRYAHASRLMVRVGDQVSAGDHIANVGCTGRCTGPHLHYEVVKDGQRKNPSTYLAMLP

SEQ ID NO: 13 - protein, 238 aa, Acinetobacter baumannii
MGMTFTDIENKSAKRLIGIAAVIFLHLLVAYILMSGLANNIQKPAEKPVELQIIQDIKPPPPPKPEEPKPKEKPPEPPKMVEKVAKVPEPPKEVEKVATPVQKTTPVAQTTKVATPAPAAPSTPSPSPVAAPAPVAAAAPALKPAGVTRGVSEGSAGCEKPEYPREALMNEEQGTVRIRVLVDTSGKVIDAKVKKSSGSKTLDKAATKAYSLCTFKPAMKDGVPQQDWYEIEYPFVIE

SEQ ID NO: 14 - protein, 253 aa, Acinetobacter baumannii
MKMMKTAIVTASVLASASIFAQSAGVNAGASAQVNVQPGGLVSGVANTVKNTAHTVGNTAKHAGHVAADTTVKATKKTTGKVTELSSKAATGTKHVASEAVTGTKHFATEAATGTKNLATKAATGTKNLAVEAKADTKAHLDAVKTKVAEKQADQKEFTAEKQADAQARVDAVKARVAQNQAEQKEFVADTKADAQAKLNTAQPAHGVNAQTGVNVGVNVAGINANANVNAGAQASTQKGEKKSFIKGLFGTN

SEQ ID NO: 15 - protein, 294 aa, Acinetobacter baumannii
MQMKKHSLLFIALMSTTSLYANIPIESRGLSQNDGSASNTSSSNISVPTNLNWELMQKNQQLENDIRTLRGQLEEQANDIEQLKKDLANRYTDLDQRLELLHQKVDPDSATQDDSSNATSDNTTPASAPAPQTTESNKVAAVPATQTSEQQPSAPTTTTQPAPAAAQNQSNSLELEKAAYTVALDAYKQGGAKKAIAPMQNFIKNHPNSIYTGNAYFWLAEFHLATDPVNYNEAKKNYNVVANQYPNSSKAPRALYQLYSIAKDVDKNTVSANQYKNKLLSQYPKSEEAKFFNK

SEQ ID NO: 16 - protein, 356 aa, Acinetobacter baumannii
MSMNNKQRWMGGVVLLGGGVLLAALLLKGNEEIKQVDVQPQTSTSPKLQAKPKQSAQEGQMVQLQPLAVDVETEKRLLEEQRRSREKAVAEQEARAAEFLAMQQQAEADAARKAAAEYAAINARRAAAQESSDNIPPEVAGSENKAKGQQTDTKKSVDLAKADADKKAAEAKRLAEADKKAAEAKRQAEADKKAAEAKRQAEADKKAAEAKRQAEADKKAAEAKRQAEADKKAAEAKRQAEADKKAAEAKRKAEAEKKAEAEKARELLENGDKKWMVQVALAANQANADAVVSKLRAKGYKVTTSPTSKGIRIMVGPAKDRDTADTTRKKITSDASLNMKSAWVIDWVPLDQRKSD

SEQ ID NO: 17 - protein, 367 aa, Acinetobacter baumannii
MANTRYEDDNNSSGTSNRGFASMDPERVREIASKGGRAAHASGNAHEFTSEEAREAGRAAHASGNAHEFTSEEAREAGALSHKNDDRNGRGRSRYDDDEDDDRGRSSGRGRGRSRYDDDDEDDDRGRSGGRGRGRGRDDDDEDDDRGRSGGRGRGRSRDDDDEDDDRGRSGGRGRGRSRRDDDDEDDDRGRSGGRGRGRSRRDDDDEDDDRGRSGGRGRGRSRYDDDDEDDDRGRSGGRGRGRSRRDDDDEDDDRGRSGGRGRGRSRYDDDDEDDDRGRSGGRGRGRSRRDDDDEDDDRGRSGGRGRGRSRYDDDDEDDDRGRSGGRGRGRSRRDDDDDDDDRRGRSDGRGQNSRNQKRDAYGRFTS

SEQ ID NO: 18 - protein, 420 aa, Acinetobacter baumannii
MLYVIPFIILLVVAVILKKRENSQKQEATSPKNINRKSGKKASAKSSKSSREKIKAKVIEENIPAIPQSNPVPEALRHNIQQLIQEKQFSAAEAQVNQALKKDNTQHELYLLLLEIHIAQKDEFAIQQLISHIRSLGLNEIAAQAETRQKEYESSSQPDAIDFPQAQTYEEPKNTDTTAQFDELTTSSSEASFDDLQKDYTPVKQEPAIEIEPLEFNFSFEQNSATENTNQPAQQPELSSTQETNELADLEFSFDLAPLHETEEKSQAVEVKADQENSINALDFNFDLNPSSSETKSVQQAPSLDEIKLIEQAPLEATSIAPLEFSLDEPALVPAPELETQNHIDVVNEAATQTQIEDPLLEAFPELKQINENELDLKLAEQYIKFGANQAARNLLQGDEQKFNTEQQQHAKNLLNRIAS

SEQ ID NO: 19 - protein, 445 aa, Acinetobacter baumannii
MPKIKPIKLVIIVVCIAIIAVLAWKFLKPKQQQPQYITAEVTRGDIENNVLATGTLDATKLISVGAQVSGQVKKMYVQLGDQVKQGQLIAQIDSTTQENSLKTSDANIKNLEAQRLQQIASLNEKQLEYRRQQQMYAQDATPRADLESAEAAYKTAQAQVKALDAQIESAKITRSTAQTNIGYTRIVAPTDGTVVAIVTEEGQTVNANQSAPTIVKIAKLQNMTIKAQVSEADIMKVEKGQQVYFTTLGDETKRYATLRQIEPAPDSISSESNSTTSSTTSSAVYYNALFDVPNTDGKLRIDMTAQVYIVLNSAKNALLVPSSALSSKQFSGQRKQGQSADKASSTPSAERKHQGNGVRLERLNLTPEQKQLIEQGKATLSVVRVLQADGTTKPTQILVGINNRVNAQVLAGLKQGDQVVIADSSENSAASANSGNNRRRGPMGM

SEQ ID NO: 20 - protein, 711 aa, Acinetobacter baumannii
MNIPPRPFKLSVIACAICYANLTYAQDAQVQALQTIQVKASNAEQSSEQTKAYNVKNSSSATKLNIEAKETPQTINVVTRQQIEDFGLTSTRDVLRNTPGVTVSNQETERTTYMARGFEISNILTDGVGFPLSGYNYNNTNPDTYFYDRVEVVKGADSLTNAFGDPSATINNIRKRPTQEFQASGGVSYGSWDTQRYEADVSGSILPSGKVRGRIMGYEQTGDSYLDRYSAEKNGFAGIVEADLTDSTLLTAGYSQEQNKPNANNWGALPLLDANGKQISYDRSYNPNPDWAHWDNETQNAFVELKQKLNDQWNAKLTYNYLDTKHNSRLLYYYGYPKADGSGVSLTPWGGQEHQEKHAVDFNLEGTYKLFNREHEATLGYSYVRNHQQDKQSTGTINDSNVIKSTTTDWASWTPQSITWSDFTEAANYKQNINSIYAATRLHLNEDLKLLLGANYVQAESKGESYSSPMSYSESKVSPYVGLTYNFTPEYTGYMSYTSIFRPQTGIDKDTNQALKPIEGKSYEMGVKSSWLDDRLTGTLSVFKTEQNNYPLRNSDGNPLNRKVPTSDLESQGVEVGLSGQITDNVNLSFGYAQFSIKDTKNGGEARTYNPNQTLNLLTTYTPPVLPKLKVGAGLQWQDGIKLYDSNVNGTIKQDAYALVNLMASYEVNDHITLQANGNNIFDKKYLNSFPDGQAFYGAPANYTVAVKFKY

SEQ ID NO: 21 - protein, 727 aa, Acinetobacter baumannii
MKLQTIACAVAIATGGLFFSHTMNEARAATNTAAVSQSIQPTQEQALVARQLATLVDRQHYLNMRLDANTSNRILDMYLDSLDPDHSLFLDAEVQNYKKLYGSNFGASLKAGNLTGPFAIHQQYRERLKQFYEFMLAELKKPQNLKQPNTFIEVDREKAPYFKTSAEQQNHWRKMLVSQLINLTISREEEQAKQKALKENPSLADGQDLTGPEDLTPAQTLTKRYTRQLERISRVKSDDVLDKTLNAMLATYDPHSNYYPPIDAIELNRQTTLQLEGIGVSIRPERGNEDYTKIETIVEGGPASKSGQVKSGDRIVGVAQEGGKMIDVVGWSSSEIVGLIRGKRGTKVTLKLLGAGASMSQARNVTLVRDVIQEEDAGVRSRTVEVTRDGKKHLLGVIEIPSFYFDYRSRRAGQQYRSVSEDTANAFEALKAKKVEGIIIDLRNDPGGSLEEVARMLGQVIKSGPVVQIRDGNGNVSVFEDNDGGQQIYTGPLAVLVNLASASASEIYSAAIQDYERGIIIGSTTTGKGTAQVQLDTLAYGQATLTQRKFYRVTGGSTQNKGVVPDIKLVDIYNEEFGERKSKNALKWDTIPTAPFKREGSVQPYVAKLSQLSEQRVAVDPQFKYLNKRTAIAKVTSDQKQVVLDIDKRRAELLSLEKQTLDAENERRIATGQKPFPNWESYQASLDALAESRAKMKANQRPALPEEETFVNEAANVLMDYAKLQNR

SEQ ID NO: 22 - protein, 728 aa, Acinetobacter baumannii
MTRIIVASKEGLDVLQDGQLNKVVLNQPTIIQIGVSQKDIASMEKQGGSLVIHLKNGETIVLENFFNEATNTTEHSLVFPTEQGKFVEAQFDAQGKVIDYRGLNHVTDLAYTSTSPSAATMAVDNDPSFSMGNVLKAGLAVLAAEGLYLWAFDKDDKDDSPSTPDLIAPAAPTATLADDTVTVTGKTEANAKIYIKDAAGNTVASGVADASGNYTIKLDKPLVNGDKLNVIAQDAAGNNSKVTVVTGTKDTIAPDVPQAQLSDDGSLLTGKAEANAKITVYDATGKVLGTVFANKDGIYSLKLTPPLTSEAGGKVVAEDAAGNKSEEVKIIAGKDTIPPASPFVEVNKEGSVIHGKTEANAKVQIKDADGKVIGSGTADAQGEFQITLSPALKEAQKGTVVVEDAAGNVSKPVEITPGFDSIAPDKPTVQINTDGTSVTGTAEANAKIEIKDTTGKVIGSGTADANGKFTISISPALTDNKHASVSAIDNAGNKSEVVDIVGTKDTTPPAKPILNSVDDDVGAVKGAITAGSETDDARPKLTGSGEANATLTIYDNGVAIGVVTVTSGRSWSFTFDKDLALGKHTITLTQTDAAGLTSEASSPFTFYVVAPKAASLSETSVDILSTEGPSLADSVGLHTLKVAQNTTTETNNPQKSVPLDDLLKSSTASESDPIAKLLSSTALKTTQASEPIEVNASVGQTTSNPNHPLPDTTSSVLQNLLDQTYPVV

SEQ ID NO: 23 - protein, 754 aa, Acinetobacter baumannii
MSKRIIQSVLSVSVLASMMSMAFAAQNEQEQAEQTLEKPAEPVKLETIFVTAEEQVKQSLGVSVITKEDLEKLPVRNDISDYVRRMPGVNLTGNSATGQRGNNRQIDIRGMGPENTLILVDGKPINSRNSVRYGWKGERDTRGDSNWVPAEAIESIEVLRGPAAARYGSGAAGGVVNIITKKVTNETHGSVEFYTSQPEDSKEGSSNRVGFNVSGPLIKDVLSYRLYGNYNKTEADDVDINKSIGSTAAGREGVKNKDISGRLAWQATDQQTVLLDISSSKQGNIYSGDSQLNANAEADAILSQLIGKETNTMYRDSYALTHEGDWSWGKSKLVAQYDKTHNKRLPEGLAGSVEGKINNLDDKATSRLETLRFNGEANIPFEYYLPQVLTVGTEWVEDRFKDNVSTTQGKDSSGSGYGDQLAKGDRSKMESRIASAYIEDNLKVTDSTDVVLGLRFDDHSKSGSNWSPSLNITQKLNDNFTLKGGVAKAYKAPNMYQNAEGYLLSTNGNGCPANIESRCLLQGNGDLKPETSVNKELGIQFQRDIVNASLTWFRNDYKDKIVAGTHVVGTVDGSSTNANTGAVTNTKWNILRWENTPKALIQGFEGSLGLDFGDIRWTNNFTYMMDSKDKQTGNPLSLVPIYTINSIFDYDITDQLDVNFVFTQYGRQKSRQFAENRLESGIGSGGANSALKPSTVKSYSTAGINVGYKFSDQISTRVGVSNLFDKQILRDSNSISQTYNEPGRAYYASLKYSF

SEQ ID NO: 24 - protein, 906 aa, Acinetobacter baumannii
MPSKIKFKQSTLSHSMHLILKMQSIPKLICSSLLLSLCVTPCYAQSSAETVIPEANQTVTDSLVQQTNTNNPSDVPITDVATLVTQAQQQQDSLAILQQQEQFPNQIEEFKPITLDNLEDLPVMPVDQNMANEIYRVAEEAKNEAQNFQNGTQKQPEMVVSDASQAELHEINQAPVNIDQLMHEIQSDSKIVVEANETGKTLPELTAAVEEPPEEKGFFRRIFNKIRPPRVIPMEQIPRITAEVTGAPDDLAKNIKGKLSTFTQESFEDFNAALPQLRSLSNQAAQAVGYYNAEFRFEKLSASRVRVNVTPNEPVRINEQNIEFTGAGAKQPQFQVIRLVPDQDVGDIFNHGLYETTKSRIVDAASDNGYFDAYWRLHDVKVSQPENKADINLKYETGERYKLGKVEFRMSDPSKPLPLNMNILESMAPWKEGDDYAFWRVNVLANNLTNSRYFNYTLVDSIKPDPIEKPLELPPDLQALVDQQNVDIDESKLLPLEQQQLAKARQLASSSKEVTQNVVDEKQFAGTESVQAAPASLKAATVQHEEQESEQDRLQAQAREEKRIPVIVTLNADKLNSLETGIGYGTDTGARLRSQYRRSIVNKYGHSFDANLELSQIRQSIDGRYSIPYKHPLNDYFNIVGGYERETRDDIGPDVSLLTESAVLGGERVIKKPLGNWQHTIGVRYRLDRLTQKGNVDISELPDAFKTAASEQEALLFSYETSKTSSNTRLNPTKAFKQTYKLELGSESLLSDANMAIATAGWRFIYSLGENDDHQFVGRSDFSYIFTDEFDKVPYNLRFFTGGDQTIRGFDYKSLSPEDNGYKIGGQALAVGSLEYNYQFKEGWRAAVFSDFGNAYDKSFSNPTAYSVGVGIRWKSPIGPIRLDVASGISDDNHPIRLHFFIGPQL

SEQ ID NO: 25 - protein, 974 aa, Acinetobacter baumannii
MFIKSILSSITSIIPLPENSNTSSNLGNGSGDGLLNGISSGNGEHNYGIGNGIADDASITAPITIPLNLSGNSITLIGNSSSSSVNSSPTTTSNNVNDNDVTNNGNGSTIGSGTGNGSGDGLLNGAASGNGEHNYGIGNGIADDASITAPLSIPINLAGNSITLIGDSSSSSVNNSATNTSNTVNDNDTTYNGNGSGGGNGSGDGLLNGIGSGNGEQNYGIGNGIADDASITAPITLPINLSGNSITLIGNSSASSVNSSPTTTSNTVNDNDTTYNGNGTGDSGVSALGGSGNGSGDGAGNGIASGNGEHNYGIGNGNGDDVDITAPITGVLNISGNSFTLIGNSSSSSVNTAPTTTSNTVNDNDTIDNGNSGGTGSGSGNGSGDGLLNGAASGNGEHNYGIGNGNGDDVDITAPITGVFNFSGNSFSIIGNSSSSSINTAPTTTTNTVNDNDVTDNGNDGGGLVGGSSGNGSGDGLLNGAASGNGEHNYGIGNGNGDDADFTFPLTGVLNFSGNSLSGFGSSSSDSVNVAPTTATNTVNDNDTIDNANTGGLGDGSGNGSGDGLLNGAASGNGEHNYGIGNGNGDDADFTLPFTGGLNILGNALSGIGGSSTDSINISPTTTSNTVNDNDTTNNGNTSGGVIGSGDSGNGSGDGLLNGISSGNGEHNYGIGNGNGDDVDVVAPITTPLNVLGNSFSFIGGEGTGDILGPITGIIGGIGGDGDILSPITGIIGGIGGDGDILSPITGIIGSIGGIGGDLGDNPLTGIIQSGIDVLQNLESLKTGLINTGIDTIAGTIIGVFPDAEHPVGDFADLGKLLFETSRDSVNGTLEAISDLAGADLEGASGSITGVIDTLITNGSTASTIIQHIVGDDLVTENGGLLGSITTIIGGVDSGDGGLLGGLDGLISINYGDSDNSNSIDVEDILGNILGSVGSNQGIAVGEPDPTGGSLIHTISLNTVNQLTDQLLHALPTV

SEQ ID NO: 26 - protein, 1071 aa, Acinetobacter baumannii
MYKPTTFVWQPSAASLFKITVLSSALAALGITTGCSSTPQSAKTSKTKQVSGAGYLDASSLDSLEDLLSATDMRAVEGDRLLILKHGDVWKRMAVGFKMDLNHWDPRIEAQRSWFISRQPYLDRLSARASRYLYHTVKEAERRGLPTELALLPVIESSYDPAATSSAAAAGLWQFIPSTGRIYGLQQTGMYDGRRDVVESTRAAYEFLGSLYNQFGSWELALAAYNAGPGRIQQAINRNQAAGLPTDYWSLKLPQETMNYVPRFLAVAQIIKNPRAYGVSLPPIANRPHFREVTLSAPLSLNEIASVTGLSRAELYALNPGYRGETVDPASPMRILIPADISPSVDNKLKGMKAGGSSGWWASVTSPSKPTTTTSTSVTVRTTPSTPAQPVRPSTPAKTSSSSVTVKTTTPRGSDALAAFAASADVPSAPRIPVAVTPAANIKPVRTEPPISATEREKILAAVRAEGEKETVDQALEPQATQAEKDQVVAELKALAPQGTEIVDPYDGKIKLTAIQTSQSVAEQQGKEVSKGFAYPKTLAEDATLANSEDAQRNKDKPYIKTDTDVVVVQPKGKRSTYTVQPGDTLAVIAMKNGVNWRDVAKWNQIDPEKTLFVGTSLYLYDAKPQEAETTAKSAAKPDVYVVQANDSLTGVANQFNLSVKQLAEYNDLSVTDGLFVGQKLQLKEPKGNRAAKVEPKAIQASTRRIATKSYTVKRGEYLKLIADRYALSNQELADLTPGLSAGSNLIVGQKINVPAKEITVDEVDDSKASGKYEKLAAGPSYKTESYKVQRGDTLSSIATKSKISLAELAELNNLKANSHVQLGQTLKVPAGASVPDQYVVQSGDSLNAIAAKYNLQTSYLADLNGLSRTAGLRAGQRLKLTGEVETTSKVSAKNTKEETPETYTVKSGDSLGNIANRYHLQLDYLAALNGLSRNSNVRVGQRLKLTGDLPTVETAKTDTAKSSPKAVVAGKNTEKYTVKAGESLNAIASRAGISVRELAEMNALKANANLQRGQNIVIPKTVVEYKVKRGDTLIGLASKYGLETTLLAELNNLTPSTQLRIGDIIKVPNL

SEQ ID NO: 27 - protein, 1110 aa, Acinetobacter baumannii
MKRMLINATHAEEVRVALITGNRLYDFDLENRTREQKKSNIYKGHVTRVEPSLEAVFVEYGAGRQGFLSMREIANSYFQADPRQTSNIRELITEGTELLVQVEKEERGNKGAALSTFISLAGRYLVLMPNNPKGGGISRQISGSVREELKEILASLNVPRGMSVIVRTAGIGRTQEELQLDLQHLLDLWAQIQGTASSGPSPMLVHQEAGVVTRAIRDYLRDDVAEILIDSEQAYNEAYNFVKAVMPRQLDKLKTYTLNEPLFAHFGIESQIQTAYEREVKLPSGGSIVIDQTEALVSIDINSAKSTRGHDVEETALNTNLEAAEEIARQLRLRDIGGLVVIDFIDMTKERNQRMVEAKLREATQSDRARIQFGQLSRFGLMEMSRQRLRPSLEEATGYVCPRCHGTGMVRDLRSLSLSIMRKVEEIALRERHGEVQVEVPVEIAAFLLNEKRHSLVYLEQTSGVRVTVLPHPHLETPHYEIAYNPDGFAPSSYERTEATRSSEKELGYESSEWHLEEADHGHAHVTATASTHAAAQKKANHATQPVAQPSAQKAASPCAWLENLFVQKQAQTVDQSRSAQNAAAAIEQMVNTGAVSRGQFGQVAVPAVAEVAPVQSNNAYISQSPVKQDVREHVEKDDKSQQQRQNNKKRKHKEQREQHHQSHEQQHQVHEEVVQLSRQEQRELKRQQKRQQQQDQQHQNNDVQHTENAVPRRDRNNQQRPNRPNRHRDPSVLNENQNTLVVVDEKQIKVDVIDAPKHDVMNTALIINVDQGQSEIVALTPERRHVERVETTSTEVAQEPTPAPVVAEKAAVVETKEEAQPSQEAAQPQIKRASNDPRMRRRQQREAKHAKAATPSIAPSQIPTLAQHTIGSLIRHVYGEDCTVLIEQFGLVPTFNRALQKFAEQYASTLVVEVTAETEEKKPVTRDAELPSHKPAEEAEPAPVLPLTPPQAPAPRVANDPRERRRLAKLAAEQAFEQVKQQHSAQEEVATPAPVAEETVAAPTAETQATVEPAQQPLELNQSTEVVQPEAAPAEEKATEETVAEAPAAKEPAPSKAASKAKAAAEETVAPTEATTDAESEDVKADKDKPSRPRRPRGRPPKKANPVAE

SEQ ID NO: 28 - protein, 1228 aa, Acinetobacter baumannii
MSTLATLKALLAKRILIIDGAMGTMIQRHKLEEADYRGERFADWAHDLKGNNDLLVLTQPQIIQGIHEAYLDAGADIIETNSFNGTRVSMSDYHMEDLVPEINREAARLAKAACEKYSTPDKPRFVAGVLGPTSRTCSISPDVNNPAFRNISFDELKENYIEATHALIEGGADIILIETVFDTLNCKAAIFAVKEVFKQIGRELPIMISGTITDASGRTLTGQTAEAFWNSVRHGDLLSIGFNCALGADAMRPHVKTISDVADTFVSAHPNAGLPNAFGEYDETPEQTAAFLKEFAESGLINITGGCCGTTPDHIRAIANAVKDIAPRQVPETVPACRLSGLEPFNIYDDSLFVNVGERTNVTGSKKFLRLIREENFAEALEVAQQQVEAGAQIIDINMDEGMLDSQNAMVHFLNLVASEPDISRVPIMIDSSKWEIIEAGLKCVQGKPVVNSISLKEGYDEFVEKARLCRQYGAAIIVMAFDEVGQADTAERKREICKRSYDILVNEVGFPAEDIIFDPNVFAVATGIEEHNNYAVDFIEATGWIKQNLPHAMISGGVSNVSFSFRGNEPVREAIHSVFLYHAIKQGMTMGIVNAGQMAIYDDIPTELKEAVEDVILNQNQGESGQAATEKLLEVAEKYRGQGGATKEAENLEWRNESVEKRLEYALVKGITTYIDQDTEEARLKSKRPLDVIEGPLMDGMNVVGDLFGSGKMFLPQVVKSARVMKQAVAWLNPYIEAEKTEGQSKGKVLMATVKGDVHDIGKNIVGVVLGCNGYDIVDLGVMVPCEKILQTAIDEKCDIIGLSGLITPSLDEMVFVAKEMQRKGFNIPLLIGGATTSKAHTAVKIDPQYQNDAVIYVADASRAVGVATTLLSKEMRGAFIEEHRAEYAKIRERLANKQPKAAKLTYKESVENGFKIDESYVPPKPNLLGTQVLKNYPLATLVDYFDWTPFFISWSLTGKFPKILEDEVVGEAATDLYNQAQAMLKDIIDNNRFDARAVFGMFPAQRTDADTVSVFDEAGQNVTHTFEHLRQQSDKVTGKPNLSLADYIRADREQQDYLGGFTVSIFGAEELANEYKAKGDDYSAILVQSLADRFAEAFAEHLHERIRKEFWGYKADEQLSNEELIKEKYVGIRPAPGYPACPEHSEKAVLFDWLGSTDKIGTKLTEHFAMMPPSSVSGFYYSHPQSEYFNVGKISQDQLEDYAKRKGWTLDEAKRWLAPNLDDSIV

SEQ ID NO: 29 - protein, 1284 aa, Acinetobacter baumannii
MKLKLKNFKPNNLWYAVCSSSMIFTWLMTSSVVQASDLQIYASPTAGKKTIVMMLDTSGSMTNNSYGENRLAMLKNGMNAFLASNNPVLNDTRVGLGNFSANGDSRSGQILVAAAPLGDASTLNTVGSQRYKLKQAVANLTAGGSTPSAHAYAEAAAYLMGTTTYSETNYAIRKDSYIKRVRRSDNRTEYSYCTNYRDSQIDTANLWQPCRSNSYWSSWSTNNPGVDNATAYDTSSDWTYYYTYYYTTFNYAVANADSGIPKSKSNDTASNPNIVVDRNATNSNAVYQSPLPAVANRQSCDGQGIYFLSDGEPNNTTNTRSASVMSTALGSTFGADFNCSGGLSNTTADSGWACMGEFAKRLFDKTKNPAGVSIQTAFVGFGSDFSSLNSSDVKNACRLSSRTQSDRKGDDACSPNQSTNAVAAPGYGNGGFFPTQSSQGVTDSVIAFINNLDKVPLEPLTTGAISVPYDALNPKNLQEYGYLRAFEPNPANTYLTWRGNLKKYHVVLSGANAGAFEANSGGLVYNASGAFRTGTKDYWNSSTYTDGGKVFLGGSYANVPLPIAGQPETRDAEGNITKYYYAVQSKIRNLFTDVSAVAADGSLTKISTSGTNLLKIPAAPPEETNPFDTVANTASYVLGKFDPSTGQNILKAFPISLKLKILNYLGYSTDINATTLPSSLVTSNEPYLSMGGSIHSLPVQLTYNGTLDDNGNLTSAREQSILYGTMEGGLHIVDASSGIEQMVFVPADILNDSVASKALVVGQSDASAPAHGMDGAWVSDPAYNITTVGSGSSAVSKVTAKQMNIYGGMRMGGSSYYGLDVLSPTSPKLLFRIGADQNDYSRMGQSWSKPVLANIRYNGSIRRVLIVGGGYDQCYEKPNITLTDACFTNGKAKGNAVYIIDAKTGQRLWWTSDTGSNTDNANMKHSIVSRISTLDRDADGLVDHLYFGDLGGQIFRVDLNNNQTKTNSTYSSFGVRVVRLANLATNDSTYDGTNDYTGGNAPRFYEPPTVTIHDYGIHTFITVGIASGDRSTPLDVYPLTGREGMTPASALSGRPVNNVYGIIDRDFVKKNLMSLTDNQLETKDITRTGLRKNPQILRTGETRVAQIFFPTTGVGKGGWYRSLSSTSDGTEKANNSFRIKGGLKAFEEPMAITGNLIILVYDPQGTGIVAADPCLPRVVGETDRQTYCLPFGACLNSDGSIDQNKENHSGFETQTGTNCPVGASECNKNVIGSGIRSVTFVPTEDNPPTTNSCGKLKLSGNEQGTGQWQCTSHLVPTRWYERYR

SEQ ID NO: 30 - protein, 3047 aa, Acinetobacter baumannii
MTDAAGNTSEQAVQKVVVDTTAPQAGELTLSDLSDTGISATDQITQDKNFNLKLEGQESGSRVTYLVSTDEGKTWQETTIAQKDLTDGVYQYKAVVTDAAGNTSETAVQKVVVDTTTPQAGELTLSDLNDTGVSVTDQITQDKNFNLKLEGQETGSRVTYLVSTDEGKTWQETTIAQKDLADGVYKYKAVVTDAAGNTSETAVQKVVVDTTAPQAGKLTLSDLNDTGVSATDQITQDNSFTLKLAQPIVIGEQAALLDHYEVSKDEGKTWQETTADQKDLADGIYQYKAIVTDLAGNISESAIQKVVVDNSLNVESTTVIVKPITEDNTISLVEKDQVISIRLEIANLPTDLNSSLTSVNTTLGNVTYNFHFDEVTQEWVTEIPAEFLWSVEPQTNISIEISLTDQAGNTAIIKHTQNYNVDHTPNSPTLDSLTFNNIDGAIISGSAYKGSKVDIYNKNGDWLASTITNEEGKFTLQDLSINSNQEVYAVATYNGYSSENSSIGLVTEVPAISITRISPEGVISGYATEGSHFIVKDQNGNILQEFNSNVFDSSGITPFSVMALGEVRPFILSLDQPLEEGAQIIISIDKDNISGHPQYITADYTPAVFLETPQFDISGETLSVHVNEPNSFIRAFSGEGNLIATGFTDEQGFASLQVFQFLKEGETVSVQVVDKNQNTSETLIEVPNFAYIPHVERITQEGLISGVAEDNSTVIVRDADGNELGKVTLGDDNSWSDFSHFSLSVNRPLIDGEKISVQIIDNKGLMSPEQNIIVDLTPPPAPTELNFNDAGDLVYGHAEPFSEILVKDGQGNILNKWFWNNWTDESGSFSIELGTFLTNAETVYVTATDVNGNVSLAAQIQAPNYAFAPYVDSFTSDGVISGQAENNSTLVVKDAKGDVVAEIKVGEDNGWNGSSYFKLQLDRPLVDGEQFFLSIKDARGQVSADTVITADTVAPTPASNLVFSEDGSYLTGVAELNTTIQVFDHNGQLVNIWNNTINSDGTFTIYLGSNNLHGEAFTVTVKDQAGNVSEAISINAPLDDIAPNPIKNILLDANGQNFTAQAEANSQIEVFDSLGNQTGWGSTDSAGNVSGSFNQTYLHGEELTFVVIDRAGNRSIEFKQNALIDTIAPNPIANIIFNEDGQSFTAQAEAGSSIDVLDQTGNKIGFGYTDSSGNVSGYFQQVYLHGEELTFVVIDRAGNRSAEVKQSALNDDVVPNPIENIVLDLNGQNFTAQAEANSQIEIKNNNGDVVGYGSADSAGNVSGYLYQVHLHGEELTFIVVDRAGNRSTEVKQNALIDDIAPNPIENIVLDINGQNFTAQAEANTQIEVKNAVGEIVGLGYVDGAGNVSGYLYQVYLHGEELTFVVVDRAGNRSTEVKQNALIDDIAPNPIENIVLDINGQNFTAQAEANTQIEVKNAVGEIVGLGYVDGAGNVSGYLYQVYLHGEELTFVVVDRAGNRSTEVKQNALIDDIAPNPIENILLDANGQNFTAQAEANTQIEVKNTAGEVIGSGSTDSMGNVSGYFYQVYLHGEELTFVVVDRAGNRSTEVKQNALIDDIAPNAIENIIFNENGQNFTAQAEANSKVEVKNAAGEVVGSGYVDSVGNVSGYLNQVYLKGEELTFVVIDQAGNRSIEVKQTAFLDNTAPENATNLVFSEDGSYLSGMAEPNATIQIFDQYGQLLNQWNNNVNWDGTFNIYLNSNYMHGEVFKVVVVDHAGNLSGEVTVKAPLDDIAPVAASDLVFNEDGSSLSGVAEPNTFIQIFDQNGQQMNTWSQSVNADGTFTIFFGTYNLHGEEFTVIVKDLAGNVSEAVSVKAPLDDIAPKPIKNIVFDANGQSFTAQAEANSQIEIFDSFGSQIGWGSTDSTGSVTGYFYQVYLHGEELTFVVIDRVGNRSDEMKLNALMDTIAPKPIENIIFNENGQNFTAQAEANSFISVKNAAGEFVGYGYVDSTGNVSGHFNQVYLKGEELTFIVIDKAGNQSIEYKQNALTDDIAPNPIENIVLNKNGQNFTAQAEADSQIEVKNTAGEVVGSGYVDSIGNVSGSFNQVYLHGEELTFVVVDRAGNRSTEVKQNALIDDIAPNQIENIVFDVNGQYFTGHAEADTRIEVLDQFGNRAGWGYVDSQGNVIGYFNQVYLHGEELTFIVVDIAGNRSVEVKQNALIDNVAPPAAANITLTSDGLLFGEAEPNSTVEIIDQYGAVITTTYVWYDGTFNQWINLSQYQTQNLSIVVKDQAGNRSEVVHELVPVFTNSPIAATELKLDIDGHILTGKATVGMSVVVTSTDGQTINGGWNNAVNEDGSFAIQLNDYYLQGQTLQVRVYDQNTNQYSLISEIIAPLDNIAPVINEVVINNDGYGITGQTDSKAIIQVMDADGDLRAEFQADETGYFNASIYPPILRGEQLFITAIDLAKNISKPFNITFNADTNAPPSAEHIVVSENGFFIEGTAVAISTVHIFDVHSNHVATNVADEAGNFNIQLYPPLASGQILRIVVEYNGYQSAYTEITAPIDTVAPNAATQLLLEDGNVLSGQAEAYSIVNIFDANNNLVGQTNVGSDGAFLTHLWYEYWHGETLTVKVVDANQNVSVGTTIVAINDTVVPDVVTQLAIDEWGSLTGRVESYATVELTYHFTDQPLSVTSTTALANGMFFIYLDRNATSLDLTVIDRAGNRSETISQIISDLPTVIIDHFKGDATDNTYNIDTIDDFVQEYIVEPYAIYKDVWIDNSYMYSDWVIEGHYEQIWFVDGYYESQWATSGYSTVQNIYQNQNGITYIDNGTADSDYSRYEQQYYDFVNGQWQEGYELTYIRSEEGWVDTSHYEDVYIDTSHYEEVWVDTSHYQDIWVENSYWESQLVESGRRDVDLGGHDKIISSVNYSLVGLYQTVNDPTTVDSFLESGRYVEDLELVGSAHLNATGNALDNLLTGNSGNNVLNGREGNDTYITNEGTDTIVFQLLNSQDATGGNGHDTVLDFTLGDIRTNLQADKIDLSELLIDYSKDVSALAKFITVEQDAGNTTISLDRDGEGTMFNSVSLLTLNQVNTTLDELLNNQQIIV

SEQ ID NO: 31 - DNA, 183 bp, Acinetobacter baumannii
  source 1..183: mol_type = genomic DNA
atgacacagctcattaataaaggtggctttcgtgaaagagccaatagaagccgaaaatatcaacaatctgaaaacaagcaagtagctctgccttcaaaaaagtatcaacctcaaactaaactccaagataatcaaagcgagatgattcaagcaaaagcaggtactgctgaaacttcagattaa

SEQ ID NO: 32 - DNA, 201 bp, Acinetobacter baumannii
  source 1..201: mol_type = genomic DNA
atgaaattagctaaaactttactcgctactaccctagctttaactgctgcttcaactttcgctgcttctaagcacgaccaagcacataacactgctggtgaagaaaaagttgttgtttcaactcaagagcaagcaaacactgcaaatgctgcttctgacgctgtaggttctgcttctgaagctgctcctgcaactcgttaa

SEQ ID NO: 33 - DNA, 201 bp, Acinetobacter baumannii
  source 1..201: mol_type = genomic DNA
atgatagatgaagaaaagcctttaaattttgaagatgatgacgagcctcttgattttgaagatgaagaattcatagacgataaaaaagaggacgaaatgtataactcgattactaaggatggctctagtgtcgatcctgctgatgatgggacacgacatattcgtcctgaagacggcgatccgattgaaattgatgagtaa

SEQ ID NO: 34 - DNA, 222 bp, Acinetobacter baumannii
  source 1..222: mol_type = genomic DNA
atgagcactactaataatcaagcaaatcaacgcaataatcagcagcaacaacaacagcaaaatgacaaccgtaatcaacagcaacacggtaaccaacagcaaaacgatcaacagcaacaaaataaccagcaacaacagcaaaatgataaccgcggtcaacaacaaggatctaatcaaaaagattctggccaacaaaactcaaataacaaccaacaacgttaa

SEQ ID NO: 35 - DNA, 327 bp, Acinetobacter baumannii
  source 1..327: mol_type = genomic DNA
atgtcagcaaaactagttgttactttgttagcaaccagtcttttaactgtaggctgtgttgcttatacagatgatccttattatcgcggtggttatggttaccatgatcatgacgacgatcgttatgaccgtaacgatggacgtcgatatagtgagtgggaacgcaaacgttgggaagagcgtaaaagattatatgaacaacaacgcaaagatattcgtgaacagcaaaaggatcgtcgtgaatgggaaaaacgacaccgtgaatgggagaagaaacgcttagaagaccgagatcatgatcatagagattatcgtcatgatgactaa

SEQ ID NO: 36 - DNA, 345 bp, Acinetobacter baumannii
  source 1..345: mol_type = genomic DNA
atgaataaattacttgttgctttaggtcttgctgcgactgttgcccttgttggttgtaataaagataaagctccagaaactggtgctactacaggtgaacacttagaaaatgctgctcaacaagcaacagcagatatcaaatctgctggtgatcaagctgcgagcgatattgcaactgcaacagacaacgcttcagctaaaattgatgctgctgctgaccatgctgctgatgcgactgctaaagctgcagctgaaacagaagctactgcacgtaaagcgactgctgatacagctcaagctgttgaaaatgcagctgctgatgtgaaaaaagatgctcaacactaa

SEQ ID NO: 37 - DNA, 381 bp, Acinetobacter baumannii
  source 1..381: mol_type = genomic DNA
atgaaaatgacggctaaaattgcattattcagtgcagcaattgtaactatgggtagtttggctgcttgtcaatcaacaactcaaccacctaagccagaacatggcatgatgcaagatggcccacgtgatggtcaccaccatcgtatgaaacaccgtgaatttacgcctgaacaaaaagcagcatgggaacaacaccgtgcagagcgtaaagctcgttttgagcaaattcaaaaagcatgtgaaggtaaagttgttggacaaactgtcaatgttcaagttggagataaaacacttgaaggtacatgtaacctccgctttgagccaaaacgtcctcaaccaccagtcaatgcccctgctccagtagcgactcaagcaaaataa

SEQ ID NO: 38 - DNA, 420 bp, Acinetobacter baumannii
  source 1..420: mol_type = genomic DNA
atgaaagccataaaaattttatgtattacaagctctatcttggtttcatcatctctctttgctgaaacacctcaacctcaacaagtgaatgaagcaacttctaagacaatgccttatggtgataatccaagcttaggccgcgtactgttatacaaaacaggaaaaggcattcaaaatttaggagattctattcaaggcgcttctgaaaaaacttcaaataaaattagtgaaaaatggaaagatacgaaagaatttaccgccgaaaaagcagaagttgttcaacaaaaagcggatacagccaaagtatttactgaacagaaaatagaacaagctaagcaaaatatcaccagcagtagaaacggtgaaaatattccaatcgaacaaggtgagctaagtaaatctagtacgactgccaattaa

SEQ ID NO: 39 - DNA, 480 bp, Acinetobacter baumannii
  source 1..480: mol_type = genomic DNA
atgaaaaaatcattattagcaattgcacttatgagtactcttcttgtcgcatgtaataaacatgaaaataaaacagaaacaacctctgatgcatctactcctgtacaaactgctcaatcaaataataatgaagcggtagatacagcacatacagccgagaattctttagattgggatggtaaatataaaggtacgcttccttgtgcagactgtgaaggcatcaaaactgaattagagttaaaagatgataaaacttatgagctaacagaaacctatcttggtaaaggtgatgcaaacccatttgaaacccatggtaagtttactttcgataaagacaatacttctgttattaccttagatgataaagctcaaaaccgtaaattctttattggtgaaaatacagcgacagctttagatatggaaggtaaaaaagttgaaggttctttagctgaacattatgttttgaaaaaagaagattaa

SEQ ID NO: 40 - DNA, 489 bp, Acinetobacter baumannii
  source 1..489: mol_type = genomic DNA
atggcaaataaaaaacttttaatctgtgctgcaattgcggctggactattgttaaccgcatgcgttaaaaaagagacaccgaaagaagaagagcaagacaaggttgaaactgctgtttcagaacctcaaccacagaagcctgcaaaatttgaatctttagagagtgtagatactcaagaagctcaagttcaagagcaaccacaagttgaagttcatcgtgaagaaactgctaacacgacaactgaaattcgccgtgaaactcgtcctgcgcgttctgatgaatcttcacagacacaagttgctgaacaacctaaatctgagacaccaaaagtagaacctaaaccagaaaaaaaacctgagcctaaagctgaacctaaaccggaaaaagctcagtcaaaaccagctgctaaggcaactgagcctgcgaacacagaagatgatgccgttgctgctgctattgctgctgcaacaccagcacttaagaactaa

SEQ ID NO: 41 - DNA, 501 bp, Acinetobacter baumannii
  source 1..501: mol_type = genomic DNA
atgactactgaaaataaattagatgaattaaaggcaaacgcggctgatgcaaaagtacaaggtgaaaaagctttggacgatttaaaagaaaatgtaaaagagaaacaaactgcgggtaaagaagcaattgcagataaagtagatgagctaaagaccaaagcggccgatgcaaaagtacaaggtgaaaaagccttggaagatttaaaggaaaatgtaaaagaaaaacaagctgctgcaaaagaggcagttgaagataaagctagtgatttgaaaggcaaacttgatgatgctcagcatagcctgcaagacaagtttgatcatttacgtactgaagcagcgcataaacttgatgatgctaaagccaaagctgcagaattaaaggaagaggcggctacaaaatttgatgagttaaaaactcaagcaactgcgaaatttgatgagttgaagaaaacagctaccgaaaaacttaataaattgaaaaatcatgattctgctgaataa

SEQ ID NO: 42 - DNA, 693 bp, Acinetobacter baumannii
  source 1..693: mol_type = genomic DNA
atgcacacgcgtcgtattttattagcgttttcacttgccgcttcggcagcatcggttgcttttgcagattatcaaaatattaaccaatctactgattcagatcgattggaacagttatcaaaaacactatctcaaggctcatatactcatcctgatgatttagatcttccggcgagtgcaaaagtttcagtgactttacgtgagaaaacagttgaactcaacaatgattcactggcaaagaaatacggcacaacgacagctaaaaactcatttaaaacctcttcttcaaatccttattcttggttagtttctcatcctcttcctgatacagtacgtgtttcttctaactttggtggtcgtaccatgggtggtcgtgcagagcatcatggtggtttagatatggctgcaccaagtggtacgccaatctatgccactggtccgggtattgtgactaaatcaggctggggcacaggttatggccaatatgttgaaattaaccatggtaatggttatttaacacgttatgcgcatgcttcacgcttaatggttcgggtaggtgatcaggtttctgcgggtgaccatattgctaatgttggttgtacaggccgctgtactggtccacatttacattacgaagtagttaaagatggtcaacgtaagaatccatcaacttacttagcaatgttgccttaa

SEQ ID NO: 43 - DNA, 717 bp, Acinetobacter baumannii
  source 1..717: mol_type = genomic DNA
atgggcatgacttttacagacatagaaaataaatctgccaaacgcctaattggtattgctgcagtcatttttctgcatcttcttgttgcctatattctgatgtcaggtttagcaaacaatattcaaaaaccagcagaaaaacctgtggaattacaaattattcaggatattaagccacctcctccaccaaagccggaagagccgaaaccaaaggaaaaaccacctgagccaccaaaaatggtagaaaaagttgccaaggttcctgagccaccaaaagaagtagagaaagtagcaacaccagtacaaaaaacgacgccagtagctcaaacgactaaagtcgctactccggctcctgctgcacctagtactccttctccgagccctgttgctgcaccagctccagtggcagctgctgcaccagcacttaaacccgctggtgtaactcgtggtgtttcagaaggctctgcgggctgcgaaaaaccagaatatccacgcgaagcactcatgaacgaagagcaaggtacggtgcgtatacgtgttttagttgatacttccggcaaagtcattgatgccaaagtaaaaaaatcgagtggtagcaaaaccttagataaagcagcaactaaagcctacagcttatgtacgttcaaaccagcaatgaaagatggcgtacctcagcaagactggtatgaaattgaatatccattcgtaattgaataa

SEQ ID NO: 44 - DNA, 762 bp, Acinetobacter baumannii
  source 1..762: mol_type = genomic DNA
atgaaaatgatgaaaacagctatcgttactgcaagtgtacttgcttctgcctctatttttgcacaaagtgcgggcgttaatgcaggtgcatctgctcaagtcaacgtacaaccaggtggtcttgttagtggcgtagccaatacagtcaaaaatactgcacatactgtagggaacacagcaaaacatgctggtcacgtagctgctgacacaaccgttaaagctacaaaaaaaacaacaggtaaagtaactgaactttcttctaaagctgctactggtactaaacatgtagcaagtgaagctgtaactggtactaaacattttgctaccgaagcagcaacaggtacaaaaaatttagcgactaaggctgcaactggcactaaaaacctagcagttgaagctaaagcagatacaaaagcacatcttgatgctgtaaaaactaaggtagcagaaaaacaagctgaccaaaaagaatttactgctgaaaaacaggccgatgctcaagcacgtgtagatgctgttaaagctcgtgtagcacaaaatcaagctgagcaaaaagaattcgttgctgacactaaagctgatgctcaagcaaaattaaatacagctcaaccagctcatggagttaacgctcaaacaggtgtaaacgttggtgttaatgttgctggtattaatgcaaatgcaaacgtaaatgcaggcgctcaagcgtctacacaaaaaggcgaaaagaaatcttttattaaaggtcttttcggtactaactaa

SEQ ID NO: 45 - DNA, 885 bp, Acinetobacter baumannii
  source 1..885: mol_type = genomic DNA
atgcaaatgaagaagcattctttattatttattgccttaatgagcaccacttctctatacgcaaatatccctatcgagtctcgtggtttaagtcaaaatgatggtagtgcatctaatacttcttcaagcaatatttcagttcctacaaatttaaactgggaactcatgcaaaaaaaccaacaattagaaaatgatattcgtacattaagaggtcagcttgaagaacaagctaatgatattgagcaattaaagaaagatctagcaaaccgatataccgacttagatcaacgtttagaattattacaccaaaaagttgaccctgacagtgctacacaagatgatagttcgaatgcaacgtccgataacactactcctgctagtgcaccagcccctcaaaccacggagagtaataaagtagctgccgtaccagccacacaaacttctgagcagcagcctagcgcaccaacaaccactactcaaccagcaccagctgctgcgcaaaaccagtcaaattccctcgaattagaaaaagctgcttatactgtagctttagatgcgtataaacaaggcggtgctaaaaaagcaattgctccaatgcaaaactttataaaaaaccatcctaatagtatctatacaggtaatgcttatttttggttagccgagtttcatttagcgacagatccagtaaattataatgaagcaaagaaaaactataatgtcgtagctaaccaatacccaaattcaagtaaggcacctcgagcattgtaccaactttatagtatcgctaaagatgttgataagaatactgtgtcagcaaatcaatacaagaacaagttacttagccaatatccaaaatctgaagaagcaaaattctttaacaaataa

SEQ ID NO: 46 - DNA, 1071 bp, Acinetobacter baumannii
  source 1..1071: mol_type = genomic DNA
atgtcaatgaataacaaacaacgctggatgggtggcgttgttttattaggtggtggtgttttattggcagcattacttctaaaaggaaatgaagaaataaaacaggtagatgttcaaccccaaacttcaacatcaccgaaattacaagctaagccaaagcaatcagcacaagaagggcagatggtacagctacagccacttgcagttgatgttgaaacagaaaaacgtcttcttgaagaacagcgccgttctcgtgaaaaagctgtagcagaacaagaggctcgtgctgccgaattcttggcaatgcaacaacaggcagaagcggatgcagctcgtaaagctgctgctgaatatgccgcgattaatgctcgccgagctgctgcacaagaaagttccgataatattcctcctgaagtcgcgggtagtgagaataaggcaaaaggacagcaaactgatacaaaaaagtcagtagatctggctaaagcggatgcagataaaaaagctgctgaagcgaaacgcttagctgaggcggataagaaagcggctgaggcaaaacgtcaggcagaagcagacaagaaagcagcagaggctaaacgccaagcagaagcagataagaaagcagctgaggcaaaacgtcaggcagaagcggataagaaagcagcagaggcgaagcgccaagcagaagcggacaaaaaggctgctgaagctaaacgtcaagcagaagcagataagaaagcagctgaagcgaaacgtaaggcagaggccgagaaaaaagctgaagcagagaaagcacgtgagttgcttgaaaatggtgataaaaaatggatggtacaggttgcattggctgctaaccaagcaaatgcagatgctgtagtctctaaattacgtgcaaaaggctataaggtcacgacgagtccaaccagtaaaggtatccgtattatggttggtcctgcaaaagatagagatacggcagatactacacgtaagaaaattacttctgacgccagtttaaatatgaagtcagcttgggtgattgactgggtgcctttagatcagcgtaagtcagattaa

SEQ ID NO: 47 - DNA, 1104 bp, Acinetobacter baumannii
  source 1..1104: mol_type = genomic DNA
atggctaatactagatatgaagatgataacaatagttcaggtacttcaaatcgtggatttgcaagtatggatcctgaaagagtcagagaaatagccagtaaaggtgggcgagctgcacatgccagtggcaatgcgcatgaatttacttcagaagaagctcgtgaagctggccgtgctgcacatgccagtggtaacgcacatgaatttacctcagaagaggctcgtgaagctggtgctttaagtcataaaaacgatgatcgtaatggtcgtggtcgcagccgttatgatgacgacgaagatgatgaccgcggccgttcaagtggtcgaggccgtggccgcagtcgttatgatgatgacgacgaagatgatgatcgcggtcgctcaggcggtcgtggccgtggtcgcggtcgtgatgatgacgacgaagatgatgatcgcggtcgctcaggtggccgaggccgtggtcgcagtcgtgatgatgacgatgaagatgatgatcgcggtcgttcaggtggccgaggtcgtggtcgcagccgccgtgatgatgacgatgaagatgatgatcgcggtcgttcaggcggtcgaggtcgtggccgcagccgtcgtgatgacgacgatgaagatgatgatcgcggtcgctcaggtggccgaggtcgtggtcgcagccgttatgatgacgacgatgaagatgatgaccgtggccgttcaggcggtcgaggtcgtggccgcagccgtcgtgacgatgatgacgaagatgatgatcgtggtcgttcaggtggccgaggccgtggccgcagtcgttatgatgacgacgatgaagatgatgatcgtggccgttcaggtggccgaggtcgtggccgcagccgtcgtgatgatgacgatgaagatgatgatcgcggtcgttcaggtggtcgaggccgtggccgcagtcgttatgatgatgacgatgaagatgatgatcgtggtcgttcaggtggccgaggccgtggtcgcagccgtcgtgatgacgatgacgacgatgatgaccgccgtggccgttcagacggtcgtggtcagaactctcgtaatcaaaaacgcgatgcttatggacgctttacgtcttaa

SEQ ID NO: 48 - DNA, 1263 bp, Acinetobacter baumannii
  source 1..1263: mol_type = genomic DNA
atgttatatgtgattccgtttatcatattactcgtggtcgccgttattttaaaaaaacgcgagaatagtcaaaaacaagaggccacttccccaaaaaatataaatagaaaatccggcaaaaaagcgagtgctaaatcaagcaaaagctcgcgtgaaaaaataaaagccaaggtcattgaggaaaatattcctgctatccctcaaagtaatcctgtaccggaagctttacgacacaatattcaacagctcattcaagaaaaacagttttcagcagctgaagctcaagtcaatcaggcgctaaaaaaagataatactcaacatgaactgtatttattacttcttgaaattcatattgctcagaaagatgaatttgcaattcaacagttgattagccatattcgtagcttgggtttaaatgaaatagcagctcaagcagaaactcgacaaaaagaatatgaatcatccagccaacctgatgctattgatttccctcaagctcaaacatatgaagaaccgaaaaacacagatacaacggctcagttcgatgaattaacaacaagttcttccgaggcttcttttgatgacctacagaaagactatacccctgtaaaacaagaacctgctattgaaattgaacctttagagtttaacttttcattcgaacagaattcggctacggaaaataccaaccaaccagcacaacaacctgagttatcatccactcaagaaacaaatgagctggccgatttagagttttcttttgacttggcccctctgcatgaaactgaggaaaaatctcaagcagtagaggtaaaagcagaccaagaaaatagcatcaatgcattagattttaactttgacttaaatccttcaagttcagagacgaaatctgttcaacaagctccctcattagatgaaattaagctcatagaacaagccccattagaagcgacttctatcgcacctcttgagttttcgttagatgagcctgccttagttccagcaccagagcttgaaactcaaaaccatatagatgtagtaaatgaagcagccactcaaacccaaatagaggatccgcttttagaagcttttccagaattaaaacaaataaatgaaaatgaactcgatttaaaattggctgaacaatacattaagtttggcgcaaaccaagcagcgcgtaatttactacagggtgatgagcaaaaattcaacacagaacaacaacaacatgcgaaaaacctacttaatcgcatagcttcttag

SEQ ID NO: 49 - DNA, 1338 bp, Acinetobacter baumannii
  source 1..1338: mol_type = genomic DNA
atgccaaaaataaagccaattaaactcgtaattattgtcgtctgtatcgccattattgctgtcttagcttggaaattcttaaagcctaaacaacagcaacctcaatatattacagcagaggtaactcgtggggatattgagaataatgtacttgcaaccggtacacttgatgcaaccaagctcattagtgtgggtgctcaggtatctggtcaggttaaaaagatgtatgtgcagcttggtgatcaagtaaaacaaggtcaacttattgcacaaattgactcgaccacccaagaaaacagtttaaaaacatctgatgctaatattaaaaatttagaggcacagcgtcttcagcaaatcgcttctttaaacgagaaacaactcgaatatcgtcgtcaacaacaaatgtatgctcaagatgcaacacctcgtgcagatttagagtcggctgaagctgcttataaaacagctcaggcacaagttaaagcattagatgcacaaattgagtctgcaaaaataacgcgttcaacagcacaaaccaatattggctatacccgtattgttgcgccaactgatggtacggttgttgcgattgtgactgaagaaggtcaaacggtaaacgcaaaccaaagtgctcctactatcgtcaaaattgcaaaacttcaaaatatgacgattaaagcacaggtgagtgaagccgatattatgaaagtggaaaaaggtcagcaggtctatttcacgaccttaggtgatgaaaccaagcgctatgcaaccttacgtcaaatcgaacctgctccagattcaatctctagtgaatcaaacagcaccacaagttcaacaacaagctcagctgtttactacaacgctttatttgatgttccaaatacggacggcaaattgcgtattgatatgactgcacaagtttatatcgtattaaattcagcaaaaaatgccttattggttccatcttctgcgttaagtagcaaacaattttctggccaaagaaaacaggggcaatcggcagataaagcaagttctactccaagtgcagaacgcaagcatcaaggtaacggcgtccgtttagaacgcttaaatttaactcctgaacaaaaacaacttattgaacaaggcaaagcaactctgagtgtagttcgcgttttacaagcagatggtacgactaaaccaacacaaattttggtaggtattaataaccgtgtaaatgcacaagtacttgccggattaaaacaaggtgaccaagttgtgattgctgatagttcagaaaactctgcagcttctgcaaacagtggtaataaccgccgccgtggcccaatgggaatgtaa

SEQ ID NO: 50 - DNA, 2136 bp, Acinetobacter baumannii
  source 1..2136: mol_type = genomic DNA
atgaatatcccacctcgaccatttaagttgtctgtaatcgcctgcgcgatttgttatgccaacctcacctatgcccaagatgcacaggtacaagctttgcaaacgattcaggtcaaagcatcaaatgcagagcagtcttctgaacagaccaaggcatataatgttaaaaactcaagtagtgcaaccaagcttaatattgaggctaaggaaacaccccaaaccattaatgtcgttactcgtcagcaaattgaagattttggccttaccagtactcgtgatgttttaagaaatacccctggtgtaacagtctctaatcaagaaactgagcgaaccacttatatggctcgtggttttgagatttcaaatatcttgacggatggcgtaggctttcctctttcaggttataactataataatacgaatcctgatacttacttttatgatcgtgtagaagtagttaaaggagctgactcgttaactaatgcctttggcgatccgagtgcaaccatcaataatattcgaaaacgtccaacccaagaatttcaagcaagcggtggtgtaagttacggttcatgggatacgcagcgctatgaagctgacgtatctggctcaattctaccaagtggaaaagtacgtggccgtattatgggctatgaacaaactggtgattcttacctagaccgatattctgcggaaaaaaacggctttgctggtattgttgaagccgatttaacagatagcactttacttaccgcaggttatagccaagaacaaaataaacccaatgcaaacaactggggtgcactacctttattagatgccaacggtaaacaaatttcctatgaccgctcatataatccaaaccctgattgggcacattgggataatgagacacaaaatgcttttgttgaattaaagcaaaaacttaatgaccaatggaatgctaaactgacttacaactatcttgatacgaagcataatagccgtcttctctattactatggttacccaaaagctgatgggtccggtgtttctctaacgccttggggtggacaagaacatcaagaaaaacatgctgtagattttaatctcgaagggacctataagctatttaaccgagaacatgaagcaactctaggctacagctatgtacgtaatcatcaacaagataaacaatctacaggaacgattaacgatagtaacgttataaagtcaactacgaccgattgggcaagttggacaccgcaatctataacttggtcagatttcacagaagcggctaactataaacaaaatattaactcaatttatgccgcgacacgtttacatcttaatgaagatttaaaacttttacttggtgcaaactatgttcaagctgagagtaaaggcgaaagttatagctcaccaatgtcatatagtgaaagtaaagtttctccatatgtcggattaacctataattttacacctgaatatacgggttacatgagttatacctctattttccgtccacaaactggtattgataaagataccaatcaagctttaaaacctattgagggtaaaagctatgaaatgggtgtaaaaagctcatggctagatgaccgtttaacaggcacactttcagtatttaaaactgaacaaaacaattaccctttacgtaactcggatggaaacccacttaaccgaaaagtaccaacgagtgatctagaatcgcaaggtgtagaagtcggtctatcaggtcaaattactgataacgtaaatctttctttcggctatgctcaatttagtattaaagacactaaaaatggtggcgaagcaagaacatacaatccaaaccagacacttaacttgctaactacctatactccgccagttttacctaagcttaaagttggtgcaggtttacagtggcaagatgggataaagttatatgactcaaatgtaaacggtacgatcaaacaagatgcatatgctttagtcaatttaatggcaagctatgaagtcaatgatcatattacgcttcaagcaaatggtaataatatttttgacaagaaatatttaaatagtttcccagatgggcaggctttttatggtgccccagccaactatacagttgctgtaaagtttaaatattaa

SEQ ID NO: 51 - DNA, 2184 bp, Acinetobacter baumannii
  source 1..2184: mol_type = genomic DNA
atgaaacttcaaactatagcttgtgcagtagcaatcgcgactggcggtttattcttctctcatacgatgaacgaagcaagagcagcaaccaatactgctgctgtttctcaatcgatccagccaacgcaagagcaagccttggtggctcgtcaactggcaactttagtagaccgccaacattatttaaatatgcgtctggatgcgaacacatctaaccgtattcttgatatgtacttagacagtcttgatccggaccactcattatttttagacgctgaagttcaaaactataaaaagctctatggttcaaattttggtgcttcattaaaagcggggaatttaactgggccatttgctattcaccagcaatatcgtgagcgcttaaagcagttttatgagttcatgcttgctgagttgaagaaaccacaaaacttaaaacagccaaatacttttattgaagtagatcgcgaaaaagcaccgtattttaaaacgtcagctgaacagcaaaatcactggcgtaaaatgctggtttctcagttaattaatttaacgattagccgtgaagaagagcaggcgaaacaaaaggcgttaaaagaaaacccttcacttgctgatggtcaagacttaacaggcccagaagatttaacgccagctcagactttgactaagcgttatacgcgtcagcttgaaagaattagtcgtgtgaaaagcgacgatgtgttggataaaacattaaatgcaatgttggcaacttatgatccgcacagtaactattatccgccaattgatgcgatagaactgaaccgccaaacaaccttacagcttgaaggtattggggtatcgattcgcccagaacgtggtaatgaagattacaccaagattgaaactattgtagaaggtggtcctgcaagtaagtctggtcaagtgaaatcaggagaccgaatcgttggtgtcgctcaagagggcggcaaaatgatcgatgtggtcggctggtcgagttcggaaattgttgggttgattcgtggtaagcgcggtacaaaagtaacgttgaagcttcttggtgctggtgcatcaatgagtcaagcacgcaatgtgactttggtacgtgatgttattcaagaagaagatgccggtgttcgttcacgtacagttgaagtgacacgtgatggtaaaaagcatctattaggtgtgattgaaattccatcgttctattttgactatcgttcacgtcgtgctggtcagcaatatcgctcagtttctgaagatactgcaaatgcgtttgaggcattaaaagccaagaaagttgaaggtattattattgacttgcgtaatgacccaggtggttcattagaagaagttgcacgtatgctcggacaagtgattaagtcaggtccagttgtgcaaattcgtgatggcaatggcaacgtaagtgtatttgaagacaacgatggtggtcagcaaatctatacaggtccactcgctgtattggtgaacttggcatcagcatctgcaagtgaaatttactctgctgcaattcaagattatgagcgtggcattatcattggtagtacaactacaggtaaaggtacagctcaggttcaactcgatactttggcatatggtcaagcaaccttaactcagcgtaaattctaccgtgtaacgggtggtagtacacaaaacaaaggtgtagtgccagatattaagcttgttgatatctataacgaagagtttggtgagcgtaaatcgaaaaatgcgttgaagtgggatacgattccgactgcaccatttaagcgtgaaggttcagtgcagccatatgtagcgaagctgtctcaactttcggaacaacgtgttgctgttgatccacagtttaagtatttaaataaacgtacggcaattgcgaaggttacgagtgaccagaaacaagttgtgcttgatattgataagcgccgtgcagagcttttgagtttagaaaagcaaactttagatgctgaaaatgaacgtcgtatagcaacaggtcaaaaacctttccctaactgggaaagctatcaggcttctctagatgctctagctgaatctcgtgccaaaatgaaagctaatcaacgtcctgcgttgccagaagaagaaacgttcgtaaatgaagctgcgaatgtattgatggattatgcgaaattgcagaaccgttaa

SEQ ID NO: 52 - DNA, 2187 bp, Acinetobacter baumannii
  source 1..2187: mol_type = genomic DNA
atgacaagaataattgtagcatccaaagagggtttggacgttctgcaagatggtcagctcaataaggtggttttaaaccaacctactattattcaaattggtgtaagtcaaaaagatatcgcatcgatggaaaagcagggtggaagtctagtcatccatttaaaaaatggagaaacaattgtcttagaaaatttctttaatgaagcaacgaatacaacagagcattcactcgtttttccaactgaacaaggaaaatttgttgaagcacaatttgatgctcaaggtaaggttatagattatagaggcttaaatcatgttacggatttggcctataccagtaccagcccttcagctgcaacaatggccgttgataatgatccaagcttttcgatgggtaatgtactaaaagcaggcttagcagttttagccgctgaaggtttatatctttgggcatttgataaagatgataaagatgattcacccagtactcccgatttaatagcacctgctgctcctacagctacgcttgctgatgatactgtgacagtgactggcaaaactgaagcaaatgcgaaaatctacattaaagatgcagcaggtaatacagtggcctcaggtgttgctgatgcgagcggaaattacacgattaaattagataagccgttagtgaatggggataaattaaatgttattgcccaagatgcggctggaaataattctaaagttactgtggtaacaggaacaaaagatacaattgccccagatgttccacaagctcaattgagtgatgatggttccttattgacaggtaaagcagaagcaaatgcaaaaatcactgtttatgatgccactggcaaagtattaggaactgtttttgcgaataaagatggtatttattctttaaaacttactccaccattaaccagtgaagcgggcggtaaagtggttgccgaagatgctgcgggtaacaaatctgaggaagttaaaattattgcgggtaaagataccataccgccagcatctccttttgttgaagtaaataaagaagggtcggtaatacatggtaaaactgaagcaaatgcaaaagttcaaataaaagatgccgatggtaaagtgattggaagtgggaccgccgatgctcaaggtgaatttcaaattacactttcacctgctttaaaagaggcgcaaaagggcacagtggttgtggaagatgctgctggtaatgtatctaaaccagttgaaattacgccaggctttgactcgattgcaccagataaaccgactgttcaaattaatacagatggtacttctgtaaccggtacggctgaggcaaatgccaaaattgaaattaaagatacaacaggcaaggtaattggtagtggaacagcggatgcgaatggaaaatttacaatttctatttcaccagctttaacggataataaacatgcttcggtatcagctatagataatgctggaaataagtctgaagttgttgatattgtaggtacaaaagatacaacaccaccagcaaaacctatattaaatagcgtagatgatgatgtaggtgctgttaaaggagctataacggctggttctgaaactgacgatgctagaccaaaacttacaggctcaggtgaagcaaatgcaactcttactatttatgataatggtgttgcaattggagttgtgacggtaacaagtggtagatcttggtcatttacatttgataaagacttagctcttggtaagcatactattactttgactcaaactgatgcggcaggccttaccagtgaggcaagttctccatttaccttttatgtagttgctccaaaggctgcgagtctgtctgaaacttcagtagatattttaagtacagagggaccatctttggcagatagtgttggattgcatactttaaaggtagcgcaaaatacaacaactgagacgaataacccgcagaaatcggttcctttagatgatttattaaaaagttctacggctagtgaatcagacccaatcgcaaaacttctctcatcaacagcgttaaaaacgactcaggcatctgagccaatcgaagtaaatgcatcagttggtcagacaacatcaaatcctaatcatcctttacctgatacaacttcttcggttttacaaaaccttttagatcaaacttatccagttgtttag

SEQ ID NO: 53 - DNA, 2265 bp, Acinetobacter baumannii
  source 1..2265: mol_type = genomic DNA
atgtcgaagcgtattatccagtcagtgctttctgtttcagtactggcaagtatgatgtctatggcttttgctgcacaaaatgagcaagaacaagctgaacaaacattagaaaagcctgctgaacctgtgaaattggaaacaattttcgtaacagctgaagagcaagtgaagcaatcgctcggtgtatcggttattaccaaagaagatttagaaaaactaccagttcgtaatgatatttctgactatgtacgtcgtatgccaggtgtcaacctgacaggcaatagtgctactgggcagcgtggtaataatagacaaattgatattcgcggaatgggccctgaaaacacgcttattttagtggacggtaaaccaattaactctcgtaattcagtccgttatggctggaaaggagagcgtgatacacgaggcgactcaaactgggtaccagcagaagccatcgagtcgatcgaagttttacgcggaccagcagctgctcgatatggttctggtgcggcaggtggggtggttaacatcattactaaaaaagtgactaatgaaactcatggttcagtagagttttatacttcacagcctgaagactccaaagaaggttcatcaaatcgtgttggttttaacgtaagtgggccactaattaaggacgttttatcttatcgtttatatggtaattataataaaacagaagctgatgatgttgatattaataaatcaattggtagtaccgcagctggccgtgaaggtgttaaaaataaagatatttcaggccgtttagcttggcaagcaacagaccagcaaactgtcttgctcgatatttcttctagcaaacaaggtaatatttattctggtgactctcagttaaatgcaaatgctgaagcggatgcgatactttcgcagctcattggtaaagaaaccaataccatgtatcgtgatagctatgcattaacgcatgaaggtgattggtcttggggtaagagtaagttagttgctcaatatgataagacccataacaaacgtctacctgaaggcttggcgggaagtgtagaaggaaaaattaataatcttgatgataaagccacttcgcgtttagaaactcttcgctttaacggcgaggctaatattccttttgaatactatttaccccaagtattaactgtaggtaccgaatgggttgaagacagatttaaagataatgtctcgacaactcaaggtaaagacagcagtggttcaggttatggcgatcaattagcgaaaggtgatcgtagtaaaatggagtcacgtattgcttctgcatatattgaagataacctgaaagttacagacagcacagatgttgtattaggtttacgttttgatgaccatagtaaatctggttctaattggagtccaagcttaaatattactcaaaaactcaatgataatttcactttaaaaggtggggtagcaaaagcttataaagcaccaaatatgtatcaaaatgccgaagggtatttattaagtacaaatggcaatggctgtcctgctaatattgagtcgcgttgtttattacaaggtaatggtgatttaaaacctgaaacatcggtaaacaaagagctcggcattcagttccaaagagatatcgtgaatgcgagcttaacttggttccgtaatgattataaagataagattgttgcgggtactcatgttgtcggaacagttgatggctcaagtacaaatgcaaatacaggagctgtgaccaatacgaagtggaatattttgcgttgggaaaatacgcctaaagccttaattcaaggttttgaaggaagtttggggttagacttcggtgatatccgctggactaataactttacctacatgatggactcgaaagacaagcaaactgggaatccattatctttagttccaatctatacaattaactcaatttttgattatgacattactgatcaattggatgtaaattttgtatttactcaatatggtcgtcaaaaatcacgtcaatttgcagagaatagacttgaatccggtataggttcaggaggtgcgaattctgcgcttaagccaagtacggtaaaaagctatagtactgctggtattaatgttggttataagttttcagaccaaattagtacgcgtgttggtgtgagtaatctgtttgataaacaaattttaagagacagcaattctattagccaaacttataatgagccaggtcgagcttattatgcatctttaaaatattctttctaa

SEQ ID NO: 54 - DNA, 2721 bp, Acinetobacter baumannii
  source 1..2721: mol_type = genomic DNA
atgccttctaaaattaagtttaaacagtcaactctttctcactctatgcatttaatcttaaaaatgcagagtatacctaaacttatttgtagcagcttattattaagtttatgtgttactccttgttatgctcaaagttcggctgagaccgttataccagaagcaaaccagacggtaacagattcattggtacaacaaactaatacaaataacccaagtgatgttccaattaccgatgtcgctactcttgtaactcaagcacagcaacaacaagatagcttggctatattgcaacaacaagaacaatttccgaatcagattgaagaatttaagccaattacgcttgataatcttgaagacttacctgttatgcctgttgatcagaatatggcaaatgaaatttatcgggtagcagaagaggcaaaaaacgaggctcaaaacttccagaatggtacgcaaaaacaaccagaaatggtggtgagtgacgcatcacaagcagaattacatgaaattaatcaggcccctgtaaatattgaccagctcatgcatgagattcaatctgatagtaagattgtggttgaagccaatgaaacaggaaaaactttacctgagcttactgctgccgttgaagaaccacccgaggaaaaaggtttctttagacgtatattcaataaaatccgtccacctcgggtaattccaatggagcagataccccgtattactgctgaggttacgggtgcgccagatgatttagctaaaaatatcaaaggtaaattatctacatttacccaagaatcatttgaagattttaatgcagcgctaccgcaacttaggagcttaagtaatcaggctgctcaagctgtaggttattacaatgccgagtttcgttttgaaaagttaagtgccagtcgcgtacgtgttaatgtaacgccaaatgaaccagtacggattaatgaacaaaacattgaatttactggtgctggtgcaaaacagccacaatttcaggtcattcgtttagttcctgaccaagatgtaggtgatatttttaatcatggcctttacgaaaccacaaaaagccgaattgtcgatgctgcatcggataatggttattttgatgcttattggcgtttacatgacgtaaaagtgagccaacctgaaaataaagcggatattaacctcaagtatgagactggtgagcgttataagcttggtaaggttgagtttcgcatgagcgatccatcaaaaccattacctttaaatatgaatattcttgaaagcatggcaccgtggaaagagggtgatgactatgctttttggcgtgtaaatgttttagcaaataacctgactaactcacgctattttaactataccttggttgattcaattaaacccgacccaattgaaaaaccacttgagttaccacccgatttacaagcgttggtcgatcagcagaatgttgatattgacgaatcgaagctgcttcctttagagcaacaacaacttgccaaagcacgccagttggcttcctcaagtaaagaagtaacacaaaatgtggtagatgaaaaacaatttgccggaactgaaagtgtacaagccgcacctgcatctttaaaagctgcaactgtacaacatgaagaacaagagtctgaacaagaccgtttacaggctcaagctcgggaagaaaaacgtataccagtgattgtgacgttaaatgccgataaactaaatagtctggaaacaggtattggttatggtaccgacactggcgcccgtttacgtagccaatatagacgttcgattgtgaataaatacggtcattcatttgacgcaaacttggagctttcccaaattcgtcaatctatagatgggcgctatagtattccttataaacatccgttaaatgattactttaatattgtgggtggttatgagcgtgaaacgagggatgatattggtccggatgtaagtttactcacggaatcggcagttttagggggtgagcgagttattaaaaaaccactcggaaactggcaacatactattggggtacgttatcgtctcgaccgcctaactcaaaaggggaatgtggatatctctgagctaccagatgcatttaaaactgctgcatcagagcaagaagcattattatttagttatgagacctctaaaacttcaagtaatacacgcttaaacccgaccaaagcttttaaacaaacttataaattagaattaggtagcgaaagtttactttcagatgccaatatggcgattgccacagcgggttggagatttatttattctttaggtgaaaatgatgaccatcagtttgttgggcggtccgattttagttatatttttaccgatgagtttgataaagttccatacaatttaagattctttactggtggtgaccagacaattcgtggttttgattataaaagtctttcaccagaagataatggatataagattggtggacaggctctagcagtaggctctttagaatataactatcaattcaaagagggttggcgagcagctgttttttctgattttggtaatgcttacgataagagttttagtaatccgacggcctatagtgtgggtgttggtattcgttggaagtccccaattggaccaattcgtttagacgtggcttctggtatttctgatgataaccatccgattcgtttgcatttctttattggtccacaactttaa

SEQ ID NO: 55 - DNA, 2925 bp, Acinetobacter baumannii
  source 1..2925: mol_type = genomic DNA
atgtttattaaaagtattttatcttcgattactagtattatcccacttcctgaaaatagtaacacaagcagtaatttaggtaacggctctggtgacggcctacttaatggaatatcttccggaaatggtgaacacaactatggtattggcaatggtattgccgatgacgcaagcattaccgccccaattaccattcccctcaacctctctggtaactcaattactctcataggcaattcatcttcaagttcggtgaatagctctccaaccaccacttcaaataacgttaatgacaacgatgtaacaaataatggtaacggctctaccattggtagcggtacaggcaatggctctggtgacgggcttttaaatggcgccgcttctggtaatggtgaacacaactatggaatcggtaatggtattgccgatgacgcaagcattactgccccactttcaattccaattaaccttgcgggtaactctattaccctaattggtgactcatcttctagttcggtcaacaactctgcaaccaatacatcaaataccgtgaatgataacgacaccacctataacggcaatggctcaggtggtggtaatggttcgggcgatggcttgttaaatggaattggctctggcaatggcgagcaaaactacggtatcggaaatgggattgcggatgacgcaagtattacagccccaattacgcttcctattaacttgtctggtaactcaattactctaattggcaattcatctgcaagttctgttaattcctcgccaactacaacgtcaaatactgtgaatgacaacgacaccacctataacggtaatggtactggtgatagcggcgtgagcgctctgggcggttctggcaatggttcaggtgatggcgcaggtaatggtatcgcttctggcaatggtgaacataactatggtattggcaatggtaacggcgacgatgttgatattactgctccgattaccggcgttctaaacatttcagggaactcatttactctcattggtaattcttcatcaagttctgtcaataccgcaccaacaaccacatcaaatacagtgaacgacaatgacaccattgataatggtaatagcggcggcacaggtagtggttcaggcaatggctctggtgatggtttacttaatggcgctgcttctggcaatggcgagcataactacggcattggcaacggaaatggtgatgatgtagatattaccgcaccaattacaggtgtttttaatttctctggtaactctttctcaatcattggcaattcatcttctagttcaattaatactgctccaactacaacgaccaatacagttaatgacaatgacgtaactgataatggaaatgatggaggcggacttgttggtggaagttctggaaatggctctggtgacggactgttaaacggcgctgcttctggcaatggcgaacacaactacggtattggtaacggaaacggtgatgatgcagatttcaccttccctcttactggtgtacttaacttttccggcaactcgctttcaggctttggcagttcatctagtgactcggtaaatgtagcaccaaccacagcaaccaataccgtcaatgataatgacaccattgataatgccaatacaggcggccttggtgacggttcgggcaatggctctggcgatggtcttttaaatggtgcagcgtctggtaatggcgagcataactatggcattggtaacggaaacggtgacgatgcagactttacgcttccatttactggcggtttaaatattctgggcaatgctttatcaggtatcggcggttcttcgaccgactctattaatatttcaccaacaactacttcaaacacagtcaatgacaatgacactaccaacaacggcaacacttcaggtggtgtgattggttctggcgactcaggcaacggctctggtgatggcttattaaatggcatctcatcaggtaacggtgaacataattatggcattggcaacggtaatggcgatgatgttgacgttgttgcccctatcactacaccacttaatgtattaggcaactctttctcatttattggtggtgaaggtacaggcgatatcttaggtccgattactggcattattggtggtattggcggtgatggagatatcctaagtccaattactggcattatcggtggtattggtggtgatggagatatcctaagcccgattactggcattatcggtagcattggcggcatcgggggtgatctgggcgataacccacttacaggtattattcaaagcggtattgacgttctacaaaatttagaaagcctaaaaacaggtttgattaatacaggtattgatacgattgccggaacaattattggtgttttccctgatgcagagcaccctgtcggtgattttgcagatcttggaaaactacttttcgagacttcacgtgatagtgttaacggcacgcttgaagctatttctgaccttgcaggcgctgaccttgagggggcaagtggctcgattactggtgtaattgatacccttattaccaatggttcaacggcatctaccattattcagcatattgtaggtgatgacctagtcactgaaaacggtggcctcttgggttcaatcaccacgattattggtggtgttgacagcggcgacggtggtttactgggtggcctagatggcttaattagcattaactatggcgactcagacaatagtaattctatagatgtagaagatattttaggaaatatcctcggctcggttggttcaaatcaaggtattgctgttggtgaacctgatccaacgggcggtagtttgattcatacgatttcacttaacacagtaaatcagttaactgaccaacttttacatgctttaccgactgtctaa

SEQ ID NO: 56 - DNA, 3216 bp, Acinetobacter baumannii
  source 1..3216: mol_type = genomic DNA
atgtataaaccaaccacatttgtgtggcagccatctgcagcatcattattcaaaattacagtccttagctctgctttagctgcgttgggtattacaacaggctgttcttcgactccgcaatctgcaaaaacctcgaaaacaaaacaggtgagtggagcaggctatcttgatgcaagcagtctggactctttagaagacttactttcagcaacagatatgcgtgctgtagagggcgaccgtttacttattttaaaacacggtgatgtctggaagcgtatggctgtaggcttcaaaatggacctcaatcattgggatccacgtattgaagcgcagcgtagttggtttatctcacgtcagccctaccttgaccgcttaagtgctcgcgcaagtcgttatttatatcatactgttaaagaagcagaacgccgtggtcttccaaccgaacttgcgttattaccagtgattgaaagttcatatgacccagctgcaacaagtagtgcggcggcggcgggtttatggcaatttattcctagtactggtcgtatttatggtttacaacaaacgggtatgtacgacggacgccgtgatgttgtggaatctactcgtgctgcctatgagtttttaggaagcttatataaccagttcggttcatgggaattagctttggctgcttataacgctggtccgggtcgtattcaacaagcgattaaccgaaatcaggctgctggtttgccaacagactattggtcactaaaattaccgcaagaaaccatgaactatgttccgcgtttcttggctgtcgctcaaattattaaaaacccacgcgcttatggggtttcgttaccgccgattgcaaataggccccatttccgtgaagttaccttatctgcaccattatctttaaatgaaatcgcgtcggtgacgggtcttagccgtgctgagttatatgcattaaacccaggctatcgtggtgaaacagtagaccctgcaagtccaatgcgtattttgattccagctgatataagtccttcagtggataacaagttaaaaggaatgaaagcaggtggaagttcaggctggtgggcaagtgttacttcgccgtctaaaccgaccacaacaacttcaacctcagtcacagttagaactactccatcaactccagctcagccagtaagaccatcgacgcctgctaaaacaagtagcagctcggtaacggtaaaaactacaacacctcgtggttctgatgcgctagctgcttttgcagcgtcagctgatgtaccaagtgcaccacgtattcctgttgcggtcactccggctgcaaatatcaaacctgtgagaacggaaccgccaatttcagcaactgaacgcgagaaaattttagcggcagtgcgtgctgaaggggaaaaggaaacagttgatcaagctttagaacctcaagctactcaagctgaaaaagatcaagttgttgccgagttaaaagcacttgcacctcagggtacagaaattgtcgatccgtatgacggcaaaattaagttaaccgcaattcagaccagccaatctgttgctgagcagcaaggtaaagaagtaagtaaaggttttgcttatccaaaaactttagctgaagatgcaactcttgcaaactctgaagatgctcagcgcaataaagataagccttatattaaaactgatacagatgttgtggttgtacaacctaaaggtaagcgtagtacttatacagtacagcctggcgatactttagcagttattgccatgaagaatggtgtgaactggcgtgatgtagctaaatggaaccagattgaccctgaaaagactttatttgtaggaaccagtctatatctctatgatgctaagcctcaagaggcggaaactacggcaaaatcggcagctaaacctgatgtctatgttgttcaagcaaatgacagtttaacaggagtggcaaatcaatttaatttgtcagtgaaacagctagctgaatataacgatttgtctgtaacagatggcttgtttgtagggcaaaaattacagttaaaagaacctaaaggtaatcgtgctgctaaagtagagccaaaggcaattcaagcaagtacacgtcgtattgcaacaaagagttatacggttaaacgtggcgaatacttgaaattaattgcagaccgttatgcattatctaatcaagagttggcagacctaacaccagggttatcagctggtagtaatttaattgtaggccaaaaaattaatgtacctgcaaaagaaatcactgttgatgaagttgatgacagcaaggcttctggtaaatatgaaaagcttgcagcaggtccatcatataaaactgaaagttataaagtgcagcgtggtgatacattatcaagcattgcgaccaagtctaaaattagcctagctgagcttgctgaactgaacaatttaaaagcgaacagtcatgtccaactgggacaaactctaaaagttccggctggtgcatcagttcctgaccaatatgttgtgcagtcaggcgatagcttaaatgcaattgcagctaaatataacttacaaaccagttatttggctgatttgaatggtttgtcacgtactgcaggccttcgtgctggtcagcgtttaaaattaactggtgaagttgaaacaactagcaaagtttcggctaaaaataccaaagaagaaacgccagagacctacacagttaaatctggtgatagcttaggaaatatcgctaaccgctatcatttacagttagattacctcgctgcattgaacggtttgtctcgcaacagtaatgttcgtgttggtcaacgcttaaaattaacaggtgatttaccaacagtagaaacagctaaaacggatacggcgaaatcgtctccaaaagctgtagttgcaggaaaaaatactgaaaagtacacggttaaagctggtgagtcgttaaatgctattgcgagtcgtgcgggtatttcagttcgtgaacttgctgaaatgaatgcattaaaagcaaatgccaatttacagcgtggacaaaatattgtgattccaaaaactgtagtggaatacaaagtcaaacgtggtgataccttaattggtcttgcgagtaaatatggtttagaaaccactttattggcggaactcaacaacctaacaccgtcgactcaattgcgtattggcgatatcattaaagtgcctaatttatag

SEQ ID NO: 57 - DNA, 3333 bp, Acinetobacter baumannii
  source 1..3333: mol_type = genomic DNA
atgaaacgtatgttgattaatgcaactcatgccgaagaagttcgcgttgcacttatcactggtaatcgtctttacgattttgatttagagaatcgtacccgagaacagaaaaaatccaatatctataaaggccatgtcacccgcgtagaaccatctttagaagctgtttttgttgaatatggcgcaggtcgtcaaggcttcttgtctatgcgtgaaattgctaacagctattttcaggcagacccacgtcaaacttcaaacattcgtgaacttattactgaaggcacagagcttttagttcaggttgaaaaagaagaacgtggtaataaaggtgcagcactttctacgtttatttcccttgcaggccgttatttagttcttatgccaaataacccgaaaggtggtggtattagccgtcaaatttcaggttcagtacgtgaagaattaaaagaaattttagcttctttaaatgtacctcgtggtatgagcgttattgtacgtaccgctggtattggacgtacccaagaagaattacaactcgatttgcaacacttgcttgacctttgggcacaaatccaaggtacagccagctcaggcccttctccgatgcttgttcatcaagaagcgggtgtagtaacacgtgcaattcgtgattacttacgtgatgacgttgctgaaatcttaattgatagcgaacaagcttataacgaagcatataactttgttaaagcagtgatgccgcgtcaattagacaaattaaaaacttatactttaaacgagccattgtttgctcattttggtattgaaagccaaattcaaactgcttacgagcgtgaagtaaaacttccttctggcggttcaattgtgattgaccagactgaagctttagtttcaatcgatattaactctgcgaaatcgactcgtggacatgatgttgaagaaactgcactgaatactaaccttgaagcagcagaagaaattgctcgccaacttcgtttacgtgacattggcggtttagttgttatcgacttcatcgatatgactaaagaacgcaatcaacgtatggttgaagcaaaacttcgtgaagcaacacaaagcgatcgtgcacgtatccagttcggtcaattgtctcgttttggcttaatggaaatgagccgtcaacgcttacgcccatcgcttgaagaagcaacaggttatgtatgccctcgctgtcatggtacaggcatggttcgtgatttacgttcgctttctctttcaattatgcgtaaagttgaagagattgcacttcgtgaacgtcatggtgaagttcaagttgaagttccagttgaaattgcagcctttttgcttaatgaaaaacgccatagtctggtgtatttagagcaaacttctggtgtacgtgtgactgtattgccacacccgcacttagaaactccacattacgaaattgcatataacccagatggctttgctccatctagctatgaacgtacagaagcaacccgttctagcgagaaagagttaggttatgagtcttctgaatggcatttagaagaagcagatcatggccatgctcatgtaactgctacagcttcaactcatgctgcggctcagaaaaaagcaaatcatgcaactcaacctgtagcacaaccttctgctcaaaaagcagcaagcccatgtgcatggttagaaaacctgtttgttcaaaaacaagcgcaaacagttgatcaatctcgttcagcacaaaatgctgctgctgcgattgagcaaatggtgaatacaggcgcagtaagccgcggacagttcggtcaagtagccgtacctgctgttgcggaagttgcgccagttcaatcaaataacgcttatatttcacagtcacctgtgaaacaagacgttcgtgaacatgttgaaaaagatgataaatctcagcaacaacgtcagaacaacaaaaagcgtaaacataaagagcaacgtgaacaacatcaccaatcacatgaacagcaacatcaagttcatgaagaagtggttcaattgtcacgccaagaacaacgtgagttaaaacgtcagcaaaaacgtcaacagcagcaagatcagcaacatcaaaataatgatgtacaacacactgaaaatgctgtgccacgtcgtgaccgtaataatcaacaacgtccaaaccgtccaaatcgccaccgcgatccaagtgtattaaatgaaaatcaaaatacactggttgttgttgatgaaaaacaaattaaggttgatgtgattgatgcacctaagcatgatgtcatgaacactgctttaatcatcaatgttgatcaaggtcaaagtgagattgttgcacttacacctgagcgtcgtcacgttgagcgagttgaaacgacttctactgaagttgctcaagagccaactccagctcctgttgtagccgagaaagctgctgtagttgaaaccaaagaagaagctcagccaagccaagaagctgctcagccacaaatcaaacgtgctagcaatgaccctcgtatgcgtcgtcgtcagcaacgtgaggctaaacacgctaaggcagctacaccatctattgcgccatcgcaaattccaactttggcacaacacacaatcggtagtttaatccgtcatgtgtatggtgaagattgcaccgtattgattgaacaatttggtctagtcccaacgtttaatcgtgctttgcagaaatttgcagaacagtacgcaagtacgttagtagttgaagttactgctgaaacggaagagaaaaagccggtaactcgtgatgcagaacttccaagccataaaccagctgaagaagcagaacctgcaccagtacttccgcttactccgccgcaagcaccagctccacgtgttgcaaatgacccgcgtgagcgccgtcgtttagcaaaacttgctgcggaacaagcatttgagcaagtgaaacaacaacattctgctcaagaagaagttgctactcctgctcctgtagcagaagaaaccgttgctgctccaactgctgaaacacaagcaacagttgaaccagcacaacaaccgcttgaacttaatcagtcaactgaagtcgtacaacctgaagctgctcctgcagaggaaaaagctacagaagaaacagtagctgaagctcctgcggcaaaggaacctgcgccatcaaaagcagcaagcaaagcaaaagcagcggctgaagaaacagtagctccgactgaagcaacaactgatgccgaatcagaagacgttaaggcagataaggacaaaccgagtcgccctcgtcgccctcgtggccgtccgccaaaaaaagctaatcctgtagctgagtaa

SEQ ID NO: 58 - DNA, 3687 bp, Acinetobacter baumannii
  source 1..3687: mol_type = genomic DNA
atgtctacgcttgccaccctaaaagcgcttcttgctaaacgcattctgattattgatggtgcaatgggaaccatgatccagcgccataaattggaagaagctgactatcgtggtgagcgttttgctgattgggcacatgatttaaaaggtaacaatgaccttttggttctaacacagcctcaaatcattcaaggtattcatgaagcctacctcgatgctggtgcagatattattgaaaccaacagctttaatggcacacgtgtttcaatgtctgactaccacatggaagatcttgttccagagattaaccgtgaagcagcacgtttagccaaagcagcttgcgaaaaatattcgactccagacaaaccgcgttttgtggcaggtgtactggggccaacatctcgtacatgttcaatctctccagatgtgaacaaccctgcttttcgtaacattagctttgatgaactaaaagaaaattatattgaagcgactcatgcactaattgaaggtggtgcagacattatcctgattgaaaccgtatttgatactttaaattgtaaagcagcgatttttgcagtcaaagaagtattcaaacaaattggtcgcgaattaccaattatgatttcagggaccattaccgatgcatcaggccgtactttaacaggtcagacagcggaagctttctggaattcggttcgtcatggcgatttgctttcaatcggttttaactgtgcccttggtgcagatgccatgcgccctcacgtaaaaactatttccgatgtcgcagatacctttgtttcagcgcacccaaatgcaggcttaccaaacgcatttggtgaatatgacgaaactccagagcaaactgcagctttcttaaaagagtttgctgaaagcggtttgattaacattactggtggttgctgtggtacgacaccagaccatattcgagctattgccaatgcggtaaaagacattgcgcctcgccaagtgcctgaaaccgtacctgcttgccgcttaagtggtttagaaccatttaatatttatgatgattcattgtttgtaaacgttggtgaacgtactaacgttaccggttctaaaaaattcttacgtctcattcgtgaagagaactttgcagaagctttagaagttgcacagcaacaagtcgaagctggcgcacagattattgacatcaacatggatgaagggatgctcgactcgcaaaatgcgatggtgcattttttaaaccttgtagcatccgaacctgacatttcacgtgtaccgatcatgattgactcatcgaaatgggaaatcattgaagccggcttaaaatgcgtacaaggtaaaccggttgttaactcaatttccttaaaagaaggttatgacgagtttgttgaaaaggcccgcctctgccgtcaatatggtgctgcaatcattgtgatggcctttgacgaagtaggtcaggccgacactgctgaacgtaaacgtgaaatctgtaagcgctcttatgacattttagtaaacgaagtaggcttccctgctgaagatattatttttgacccgaacgtgtttgcagttgcgactggtattgaagaacacaacaactacgcagtcgattttattgaagcaacgggctggattaaacagaacttaccgcacgccatgatttctggtggtgtgtctaacgtttcgttctcattccgtggtaatgagccagttcgtgaagccattcactctgtattcttgtaccatgccatcaagcaaggcatgaccatgggtattgtgaacgcaggtcaaatggctatttatgatgatattcctaccgagctaaaagaagcggttgaagatgtcattttaaatcagaatcaaggtgagtctggtcaggctgcgactgaaaaattacttgaagttgcagaaaaataccgtggacaaggtggtgcaacaaaagaagccgaaaaccttgaatggcgtaatgagtcagttgaaaaacgtcttgaatatgccttggttaaaggtattacgacttatattgaccaagacaccgaagaagcccgcttaaaatcaaaacgtcctttagatgtaattgaagggccactgatggacggcatgaatgtggtcggtgacttgttcggttcaggcaaaatgttcttgccacaagttgtaaaatctgcccgagtcatgaaacaagcagtggcatggctcaacccgtacatcgaagctgaaaagacagaaggacagtctaaaggtaaagtcctaatggcaacggttaaaggtgacgtacacgatattggtaaaaatattgtaggcgtagtacttggctgtaatggctatgacattgttgaccttggcgtaatggtcccttgcgagaaaatcttgcaaactgcaattgatgaaaaatgtgacatcattgggttatctggtctgatcaccccatctttagatgaaatggtatttgttgctaaagaaatgcagcgtaaaggctttaacattcctttattgattggtggtgcaaccacttctaaagcccacacagcagtaaaaattgaccctcagtatcaaaacgatgcagtaatttatgttgctgatgcttctcgtgctgttggtgtagcgacaaccttgctttcgaaagaaatgcgtggtgcatttattgaagaacatcgtgctgaatatgccaaaattcgtgagcgtttagccaacaaacaaccaaaagcggccaaactgacttataaagagtcggttgaaaatggttttaaaattgatgaaagctatgtgccaccaaaaccaaatcttttgggaactcaagttttaaagaattatccgcttgcaacactcgtggattattttgactggacgccattctttatttcttggagtttaactggcaaattcccgaaaattttagaagatgaagtggtcggcgaagcagcaactgacttgtacaaccaagcacaagcaatgttgaaagatattatcgacaacaaccgttttgatgctcgtgctgtatttggtatgttccctgctcagcgtacagatgcagataccgtcagcgtatttgatgaagctggtcaaaatgttacgcatacttttgagcacttacgccagcaatctgacaaagtgacaggcaaaccaaacttgtctttagcagattacattcgtgctgaccgcgagcagcaagactacttgggcggattcactgtatcgatttttggtgcagaagaactggccaatgaatacaaagccaaaggtgatgactactctgcaattttagtacagtcattagctgaccgttttgctgaagcctttgcagaacatttacatgaacgtattcgtaaagagttctggggctataaagcggatgagcagctcagcaatgaagagctgattaaagagaaatatgtcggtattcgccctgcaccaggttatcctgcttgccctgagcactctgaaaaagcagtgttatttgactggttaggctctaccgacaaaattggtactaaactgactgagcactttgcaatgatgccgccatcttcggtaagcggtttctattattctcatcctcaaagtgaatactttaacgtgggtaaaatttctcaagaccaacttgaagattatgcaaaacgtaaaggttggacactggatgaagcgaagcgttggttagctccgaatttagatgactcgattgtttag

SEQ ID NO: 59 - DNA, 3855 bp, Acinetobacter baumannii
  source 1..3855: mol_type = genomic DNA
atgaaactaaaattgaagaattttaagccaaataatttatggtatgccgtctgttcaagctcaatgatattcacatggctgatgacaagctctgtggtgcaagcaagtgatttacaaatttatgcctcacctacagcaggtaaaaaaacaattgtgatgatgttggatacatcaggaagtatgaccaacaatagttatggtgaaaaccgcttagccatgcttaaaaatggtatgaatgcttttttggctagcaataaccctgtcttaaatgatacgcgagtaggtttaggaaatttctcggcaaatggtgacagccgaagtggacaaattttagtggctgctgctcctttaggtgatgcaagtactttaaatacggtaggctcacaacgttataaattaaaacaagctgttgctaatttaactgcgggaggctcaacaccttcagcccacgcttatgctgaagcagctgcttacttaatggggacaaccacatattcagagacgaattatgctattcgtaaagatagttatatcaaacgtgtaagaagatctgataatagaactgaatattcatattgtactaattaccgtgattcgcaaattgatacagccaacctatggcaaccttgtcgttcaaatagttattggagtagctggtcaacaaataatccaggtgtggataatgctactgcttatgatacttcatctgactggacttattactatacttattactacaccacttttaattatgctgtagcaaatgcagatagcggtattccaaaatctaaatcaaacgatacagctagcaatccgaatattgttgtagaccgaaatgctactaactccaatgcagtgtatcagtcacctttacctgcagtagcgaatcgccagagttgtgatggacaaggtatttacttcttatcggatggcgaaccgaacaatacaacaaatacacgttcggcaagtgtcatgtcgactgctttaggtagtacttttggagctgattttaattgttctggaggtttatctaacactacggcagactctggttgggcttgtatgggagagtttgcaaaaagattatttgataaaacaaagaaccctgccggagtttctattcaaacagcatttgtcggttttggtagtgacttttctagtttgaattcttctgatgtaaaaaatgcctgccgtttaagttcgagaacccagtctgaccgaaaaggagatgatgcgtgttcaccgaatcaatctacaaatgcggtagctgcaccaggttatggaaatggtggttttttccctactcagagttcacaaggcgtaacagacagcgttatcgcatttattaataatttagataaagttcctttagaacccttaaccacaggtgctatctcagttccatatgatgcattgaatcccaaaaatttacaggaatatggttatttacgagcttttgagccaaatccagctaacacgtacttaacatggcgtggaaatttaaagaaatatcatgttgttttatctggagcgaatgctggtgcttttgaggccaattctggcggattagtctacaatgcaagtggggcttttagaactggaacaaaggattattggaatagctctacttatactgacggaggcaaagtctttttaggtggttcatatgcgaatgtgcctttacccattgctggacaacctgaaacacgtgatgcagaagggaatatcacaaaatattattacgcagtacaaagtaaaatccgcaacttatttactgatgtttccgccgttgcagcagatggtagcttaacgaaaatttcaacttctggaactaatttgctaaaaattccagctgctccaccagaagaaactaatccttttgatacggtggctaatacagcaagttatgttttaggtaaatttgatccatcaactggacaaaatattttaaaagcttttcctataagcttgaaattaaaaatattaaattatttaggttattcaacagatattaatgcaacaactctgccttcatctttggttacatcgaatgaaccttatttatcgatggggggaagtattcactctttaccggttcaactaacctacaatggaaccctagatgataatgggaatttaacatctgcccgagaacaatccatcctctatggaactatggaaggcggattacatattgtggatgcttcatctggtattgagcaaatggtctttgttcctgcagatattttaaatgattcagttgcttccaaagctttagttgttggtcaaagtgatgcttcagctcccgctcatggtatggacggagcttgggtatctgatccagcctataatattactacagtgggaagtggtagctcggcagtatcaaaagtaactgcaaagcaaatgaatatttatggcggcatgcgtatgggaggaagtagctactacggactagatgtattaagccctacttcaccgaaactgctttttagaataggggcagaccagaatgactatagccgtatgggtcaaagctggtctaaacccgtactcgcgaacatccgttataacggttctattagacgcgtcctgattgttggcggtggttatgatcagtgttatgaaaaaccaaatattacgttgactgacgcttgctttaccaatggaaaagcaaaaggaaatgctgtctatattattgacgcaaaaactggtcagcgtttgtggtggacaagtgatacaggttctaatactgataacgccaatatgaagcatagtattgttagccgtattagtactttagaccgtgatgctgatggcttagtcgaccatctatattttggagatttaggcggacaaatttttcgcgtagaccttaataataatcagacaaaaaccaattcgacctatagcagttttggtgtcagagttgtgcgtttagcgaatttagcaacaaatgattcaacttatgatggcacaaatgattatacaggtgggaatgctcctcgtttttatgagcctccaacagtaacgatccacgattatggaattcacacttttattacagtaggaattgcatcaggagaccgtagtacacctttagatgtttacccactcacaggtcgtgaaggtatgacgcctgcaagtgcattaagtggacgtcctgtaaataatgtatatggaattattgatagagactttgttaaaaagaacttaatgtctttaactgataatcagcttgaaacaaaagatattacacggacaggcttaagaaaaaatccacaaattctaagaacaggtgaaacaagagtagctcaaattttcttcccaactacaggagtaggtaaaggtggttggtatcgctcgctttctagtacgagcgatggtacagaaaaagctaataatagttttcgtattaaaggaggactcaaagcttttgaggaaccaatggcaattacgggtaatttaattattctagtttatgaccctcaaggaacgggaattgttgcggcagatccttgcctacctcgtgttgtgggagaaacagaccgacaaacttattgtttaccatttggagcttgtcttaattctgatgggtcaatcgatcaaaataaagagaatcacagtgggtttgaaacacaaactggtactaattgcccagtaggggcttctgaatgtaataaaaacgttattggctctggtattcgtagcgtaacatttgtaccaacggaggataacccacctacgactaatagttgtgggaaattaaagctgtctggtaatgagcaagggactggacagtggcaatgtacgagtcatttagttcctacgcgttggtatgagcgttatcgttaa

SEQ ID NO: 60 - DNA, 9144 bp, Acinetobacter baumannii
  source 1..9144: mol_type = genomic DNA
atgacagacgctgctggcaatacttcggaacaggctgtacaaaaagtagttgtggatactaccgcgccgcaagcaggtgaactgactttatctgacttgagtgatacaggtatttcagcaacagatcagatcacgcaagataaaaacttcaatttaaagcttgaaggacaggaaagcggcagccgagtaacatatttagtttctaccgatgaaggaaaaacttggcaggaaaccacgatagcccaaaaagatttaactgatggtgtttaccaatataaagctgtagtgacagacgctgccggcaatacctcggaaacggctgtacaaaaagtggttgtggatactaccacaccacaagcaggtgaactgactctatctgacttgaatgatacaggcgtttcagtaacagatcagatcacgcaagataaaaacttcaatttaaagcttgaaggacaggaaactggtagccgagtaacatatttagtttccactgatgaaggaaaaacgtggcaggaaaccacgatagcccaaaaagatttggctgatggtgtttacaaatataaagctgtagtgacagacgctgccggcaatacctcggaaacggctgtacaaaaagtggttgtggatactaccgcaccgcaagcaggtaaactcactttatctgatttgaatgatacaggtgtttcagcaacagatcagattacgcaagataatagttttactttaaagctagctcaaccgattgtgattggggaacaagccgctttactagaccactatgaagtttcaaaagatgaaggaaaaacttggcaagagacaacagctgatcaaaaagatttagctgatggtatttaccaatataaagctatagtgacagaccttgcaggcaatatctcagaatctgctatacaaaaagttgttgtagataattccttaaatgttgaatcaaccacagtgattgtaaagccgattactgaagacaatacaataagtctagttgaaaaagatcaagttatttctataagacttgaaatagctaatttacccacagatttaaatagctcactgacatcagtaaatacgacattaggcaatgttacttataattttcattttgatgaagtcacgcaagaatgggttactgaaattccagcagagtttctttggtcagtagagccgcaaaccaatatatcaattgagattagtcttactgatcaagctggtaatacagccattatcaaacatacccaaaattataatgtggatcatactccgaattcaccaaccctagattcactgactttcaacaatatagatggagctatcatttcaggtagtgcatataaaggaagtaaggtcgatatctacaataaaaatggtgattggcttgcaagtacaataactaatgaagaaggtaaatttactttacaagatctttcaattaactcaaatcaagaagtttacgcagtcgctacttataatggttatagcagtgaaaattcatcaattgggttagttactgaagttccagctattagtattacacgaattagtccagaaggcgtgattagtggttatgcgactgaaggtagtcattttattgtaaaagatcagaatggaaatattttacaagaatttaattctaatgtatttgatagctctggtattacaccatttagtgtaatggcattaggcgaagtaagaccatttattttgtccctcgatcagcctttagaggaaggggctcaaattattatctctatagataaagataatatttcaggccatccacaatatattactgcagactatactccagcagtatttttagaaactccacaatttgatattagtggtgaaactttatcagtacatgttaatgaacctaacagctttattcgtgcattttctggagaaggcaatttaattgctacaggttttaccgatgagcaaggctttgcaagtttacaggtgttccaatttttaaaagaaggcgaaacggtttctgtacaagtagtagataaaaatcaaaatacgagtgaaaccctaatcgaggttccaaactttgcttatattccacatgttgaacgtattacacaagaaggcttaatttctggagttgctgaagataatagtacggtaattgtacgtgatgctgatggcaatgaattaggaaaggttacattaggtgatgataatagttggagtgactttagtcattttagcttgagtgtaaaccgccctttaattgatggagagaaaatttccgttcaaattattgataataaagggttaatgagtcctgagcaaaatatcatcgtagatctgactcctccacctgctccaacggagttaaattttaatgatgctggtgatttagtttatggtcatgcagaacctttctctgaaattttagttaaagatgggcaaggaaatattcttaataaatggttttggaataactggaccgatgagagtggaagtttctcaatagaactaggtacatttttaaccaatgctgagacggtttatgtcacggctaccgatgtaaatggaaacgtaagtttagcagctcagatacaagcacctaattatgcctttgctccatatgttgatagctttacttcggatggcgtgataagtgggcaagctgaaaataatagtactctcgttgttaaagacgccaaaggtgacgttgtagctgaaattaaagttggtgaagataacggttggaatggatcaagttattttaaactccagcttgatcgtcctcttgtcgatggtgagcagtttttcttatcaattaaggatgcacgtgggcaagtaagtgctgatactgtaattaccgctgatacggttgctcctacaccagccagcaatttagttttctcagaagatggttcatatcttacgggtgtagcagaactgaatactacgattcaggtttttgatcataatggtcagctagtgaatatatggaataacaccattaactctgacggtacatttactatttacttaggtagtaacaatttacatggagaagcattcacagtcactgttaaagaccaagccggaaatgtgagtgaggctatttcaataaacgcgccacttgacgatattgcaccgaatccaattaaaaatattttacttgatgcaaatggccaaaactttacagcacaagcagaagcaaatagtcagatcgaagtttttgattcattgggtaatcagacaggttggggttctacagatagtgcaggtaatgtttctggttctttcaatcaaacttatttacacggtgaggaactcacttttgttgttatagatcgagcaggtaatcgcagtatcgaatttaagcaaaatgctttaattgataccattgcaccaaatccgattgcaaatatcatcttcaacgaagatggccaaagttttacagctcaggcagaagccggaagttctattgatgtattagatcagactggaaataagattggttttggttacaccgatagctcaggtaatgtatctggttattttcaacaagtttacttacacggtgaggaacttacttttgtcgtgatcgatcgagcaggtaaccgcagtgcagaggtcaagcagagtgctttgaatgatgacgtcgtaccaaatccgattgaaaatattgtattagatctcaatggccagaactttacagctcaggcagaagcaaatagccaaattgaaatcaaaaataataacggtgacgtcgtaggttatggatcagcagatagtgcaggtaatgtttcaggatatttgtatcaagtgcatttacatggcgaagagctcacttttattgtagttgaccgagcgggtaaccgcagtacagaggttaagcagaatgccttgattgatgatattgcgccaaatccgattgaaaatattgtattagatatcaatgggcaaaattttacagcgcaagcagaagcaaatactcaaattgaagttaaaaatgctgttggtgagattgtaggtttaggttatgtggatggcgctggtaacgtgtcaggttatttatatcaagtctatttacatggagaagagctcacttttgttgtcgttgaccgagcgggtaaccgcagtacagaggttaagcagaatgccttgattgatgatattgcgccaaatccgattgaaaatattgtattagatatcaatgggcaaaattttacagcgcaagcagaagcaaatactcaaattgaagttaaaaatgctgttggtgagattgtaggtttaggttatgtggatggcgctggtaacgtgtcaggttatttatatcaagtctatttacatggagaagagctcacttttgttgtcgttgaccgagcgggtaaccgcagtacagaggtcaagcagaacgccttgattgatgatatcgcaccaaatccgattgagaatattttacttgatgcgaacggacagaactttacagctcaggcagaagcaaacactcaaattgaagtcaaaaatactgctggtgaagtcataggatctggttcaaccgatagtatgggtaatgtttctggttacttctatcaggtctatttacatggtgaagaactcacttttgttgtagttgatcgagctggtaaccgcagtacagaggtcaagcagaatgccttgattgatgacattgctccgaatgcgattgaaaacattatctttaatgaaaatggtcaaaactttacagcgcaagcagaagcaaatagcaaagttgaagttaaaaatgctgcgggtgaggttgtaggttctggttatgtggatagtgttggtaatgtgtcaggttacttgaatcaagtttatttaaaaggtgaggagctcacttttgttgtgattgatcaagctggtaatcgtagcattgaagtaaaacaaacagcctttcttgataatacagcaccggaaaatgcgactaatttagtatttagtgaagatggctcatatctaagtggtatggctgagccaaatgcgacgattcaaatatttgatcagtatggtcaattattaaatcagtggaataataatgttaattgggacggaacatttaacatctatttaaacagtaactacatgcatggagaagtatttaaagtagttgtagttgatcacgctggtaatttgagtggtgaggttactgtaaaagcaccgcttgatgatattgctcctgtagctgcaagtgatctggtctttaatgaagatggttcatccctttctggtgtagctgagccaaataccttcatccagatttttgatcaaaatggtcagcagatgaatacgtggagtcagagtgtaaatgctgatggtacatttactatttttttcggtacttacaatttacatggtgaagagtttacagtcattgttaaagaccttgctggaaatgtgagtgaagctgtttcagttaaggcgccgcttgatgatattgccccaaaaccgattaaaaatattgtatttgatgcaaatggccaaagctttacggcacaagcagaagcaaatagtcagattgaaatttttgactcatttggtagtcagataggttggggctctaccgatagcactggtagtgtgacgggttacttctatcaagtgtatttacatggagaagaattaacgttcgttgttatagaccgagtaggtaaccgtagtgatgaaatgaagttaaatgctttgatggataccattgcaccaaagccgattgaaaacatcatctttaatgaaaatgggcaaaattttacagcacaagcagaagccaatagttttattagtgtcaaaaatgctgcgggtgagtttgttggctatggttatgtcgatagtactggtaatgtgtccggtcacttcaatcaggtttacttaaaaggtgaggaactcacttttattgttatagataaagcaggtaatcaaagtattgaatataagcaaaatgctttaactgatgatattgcaccaaatccgattgaaaacatcgtcttaaataaaaatggacaaaactttacagcgcaagcggaagccgatagccaaatcgaagttaaaaatactgcgggtgaggtcgtaggttctggttatgtagatagtattggtaatgtgtcaggttcttttaatcaagtctatttacatggagaagagcttacttttgttgtagttgaccgagcaggtaaccgtagtacagaggtcaagcagaatgctttgattgatgatatcgcacctaatcaaattgagaatattgtttttgatgtaaatggtcagtactttacggggcatgctgaagcagatactcgaattgaagtactagatcaatttggcaatcgcgctggttggggatatgttgatagccaaggtaacgtgataggttatttcaatcaggtttatttacatggagaggaattaactttcatcgttgtagatatagctggtaaccgaagtgttgaagttaagcaaaatgctttaattgataatgtcgcaccgccagcagcagcaaatattacattaacctcagacggattgctttttggtgaggcggagccaaactcaactgttgaaattattgatcaatatggtgcagttatcacaacaacttatgtttggtacgatggcacatttaatcaatggattaatctgagtcaataccagacacaaaatttaagtattgttgtcaaagatcaagcaggtaaccgcagtgaagttgtacatgaattagtaccagtatttaccaactcaccaattgctgcaacagagctaaaactggatatagatggtcatattctgacaggtaaggcgacagtaggaatgtcggttgttgttacatctactgatggtcagaccattaacggagggtggaataatgctgtgaatgaagatggtagttttgctattcagttaaatgattactatctacagggacagacattacaagttcgagtttatgatcagaatacgaatcaatatagcctaatctctgaaattattgcgcctttagacaatattgctccagtaattaatgaagtggtaattaacaatgatggttatggtattactgggcaaactgattcaaaagcaatcattcaggtaatggatgcggatggagatttacgagcagaatttcaagcggatgagactggatattttaacgctagtatttatccacctatattacgtggagagcagttattcatcactgcgatagatttagccaaaaatataagtaagccatttaatattacttttaatgcagatactaatgcaccgccatcagcagagcatattgtggtatctgaaaatggtttttttattgagggaaccgctgtagcaattagtactgtgcatatttttgatgtgcatagtaaccatgttgccactaatgttgcagatgaagctggaaactttaatattcagttgtatccacctctagcgagtggacaaattttacgcattgttgtggaatacaatgggtatcaaagtgcttacaccgaaattacggcacctatagacactgtcgcaccaaatgcagcaactcagcttctcttagaagatggaaatgtactttcaggacaagcggaagcatattcaattgttaatatttttgatgctaataataatttggttggtcagactaatgttggaagtgatggtgctttcttaacacacctatggtatgagtattggcatggcgaaacattaacggtaaaagtagttgatgccaatcaaaatgtgagtgtggggacaacgattgttgctataaatgatacggtagttccagatgtagttacgcagcttgctatagatgaatggggttcgctcacagggagggtagaaagttatgctactgttgagcttacctatcattttactgaccaaccgctttctgtaacgagcactacagcattggcaaatggaatgttctttatctatctggatagaaatgcaacttcacttgaccttactgttattgatcgtgctggaaaccgtagtgaaaccattagccaaataattagtgatttaccgactgttattattgatcactttaaaggtgatgctacagataatacttataacattgatactatagatgattttgttcaagaatatattgttgagccgtatgcaatatataaagacgtctggattgataatagctatatgtattcagattgggtcattgaaggccattatgaacagatatggtttgttgatggatattatgaatcgcaatgggccacaagtggttattcaactgtacaaaatatttatcaaaatcaaaatggtataacttatattgataatggcacggcagatagcgattatagtcgatatgaacaacaatattatgatttcgtgaatggccaatggcaagaaggctatgaattaacttatatacgttcagaagaaggatgggttgatacaagtcattatgaagatgtttatattgatacaagtcattatgaagaggtttgggtagatactagccactatcaagatatttgggtagaaaatagttattgggagagtcaacttgttgaatctggccgtagagatgtggatttaggcgggcatgataaaattatcagttctgttaattatagtttggttggtttatatcaaacagtaaatgatccaacgactgtagattcgtttttggaaagtggtcgttatgtagaagacttggagctggttggttctgctcatctaaatgcaacaggcaatgctttagataatcttttaacaggtaactctggtaataacgttttgaatgggcgtgaaggtaacgatacctatatcactaatgaaggtactgataccattgtgttccaattgctaaatagccaagatgcaactggcgggaatggacatgacacggtgttagattttactttaggtgatataagaactaaccttcaagctgacaaaatcgatttaagtgaactattaattgactattctaaagatgtaagcgcattagctaaatttattactgtagagcaagatgctgggaacacgactattagtcttgaccgagatggcgaaggtacaatgtttaatagtgtgtcactgttaactctaaatcaagtaaatacaacattagacgaattactaaacaaccagcaaattattgtttaa

SEQ ID NO: 61 - RNA, 183 nt, Acinetobacter baumannii
  source 1..183: mol_type = mRNA
atgacacagctcattaataaaggtggctttcgtgaaagagccaatagaagccgaaaatatcaacaatctgaaaacaagcaagtagctctgccttcaaaaaagtatcaacctcaaactaaactccaagataatcaaagcgagatgattcaagcaaaagcaggtactgctgaaacttcagattaa

SEQ ID NO: 62 - RNA, 201 nt, Acinetobacter baumannii
  source 1..201: mol_type = mRNA
atgaaattagctaaaactttactcgctactaccctagctttaactgctgcttcaactttcgctgcttctaagcacgaccaagcacataacactgctggtgaagaaaaagttgttgtttcaactcaagagcaagcaaacactgcaaatgctgcttctgacgctgtaggttctgcttctgaagctgctcctgcaactcgttaa

SEQ ID NO: 63 - RNA, 201 nt, Acinetobacter baumannii
  source 1..201: mol_type = mRNA
atgatagatgaagaaaagcctttaaattttgaagatgatgacgagcctcttgattttgaagatgaagaattcatagacgataaaaaagaggacgaaatgtataactcgattactaaggatggctctagtgtcgatcctgctgatgatgggacacgacatattcgtcctgaagacggcgatccgattgaaattgatgagtaa

SEQ ID NO: 64 - RNA, 222 nt, Acinetobacter baumannii
  source 1..222: mol_type = mRNA
atgagcactactaataatcaagcaaatcaacgcaataatcagcagcaacaacaacagcaaaatgacaaccgtaatcaacagcaacacggtaaccaacagcaaaacgatcaacagcaacaaaataaccagcaacaacagcaaaatgataaccgcggtcaacaacaaggatctaatcaaaaagattctggccaacaaaactcaaataacaaccaacaacgttaa

SEQ ID NO: 65 - RNA, 327 nt, Acinetobacter baumannii
  source 1..327: mol_type = mRNA
atgtcagcaaaactagttgttactttgttagcaaccagtcttttaactgtaggctgtgttgcttatacagatgatccttattatcgcggtggttatggttaccatgatcatgacgacgatcgttatgaccgtaacgatggacgtcgatatagtgagtgggaacgcaaacgttgggaagagcgtaaaagattatatgaacaacaacgcaaagatattcgtgaacagcaaaaggatcgtcgtgaatgggaaaaacgacaccgtgaatgggagaagaaacgcttagaagaccgagatcatgatcatagagattatcgtcatgatgactaa

SEQ ID NO: 66 - RNA, 345 nt, Acinetobacter baumannii
  source 1..345: mol_type = mRNA
atgaataaattacttgttgctttaggtcttgctgcgactgttgcccttgttggttgtaataaagataaagctccagaaactggtgctactacaggtgaacacttagaaaatgctgctcaacaagcaacagcagatatcaaatctgctggtgatcaagctgcgagcgatattgcaactgcaacagacaacgcttcagctaaaattgatgctgctgctgaccatgctgctgatgcgactgctaaagctgcagctgaaacagaagctactgcacgtaaagcgactgctgatacagctcaagctgttgaaaatgcagctgctgatgtgaaaaaagatgctcaacactaa

SEQ ID NO: 67 - RNA, 381 nt, Acinetobacter baumannii
  source 1..381: mol_type = mRNA
atgaaaatgacggctaaaattgcattattcagtgcagcaattgtaactatgggtagtttggctgcttgtcaatcaacaactcaaccacctaagccagaacatggcatgatgcaagatggcccacgtgatggtcaccaccatcgtatgaaacaccgtgaatttacgcctgaacaaaaagcagcatgggaacaacaccgtgcagagcgtaaagctcgttttgagcaaattcaaaaagcatgtgaaggtaaagttgttggacaaactgtcaatgttcaagttggagataaaacacttgaaggtacatgtaacctccgctttgagccaaaacgtcctcaaccaccagtcaatgcccctgctccagtagcgactcaagcaaaataa

SEQ ID NO: 68 - RNA, 420 nt, Acinetobacter baumannii
  source 1..420: mol_type = mRNA
atgaaagccataaaaattttatgtattacaagctctatcttggtttcatcatctctctttgctgaaacacctcaacctcaacaagtgaatgaagcaacttctaagacaatgccttatggtgataatccaagcttaggccgcgtactgttatacaaaacaggaaaaggcattcaaaatttaggagattctattcaaggcgcttctgaaaaaacttcaaataaaattagtgaaaaatggaaagatacgaaagaatttaccgccgaaaaagcagaagttgttcaacaaaaagcggatacagccaaagtatttactgaacagaaaatagaacaagctaagcaaaatatcaccagcagtagaaacggtgaaaatattccaatcgaacaaggtgagctaagtaaatctagtacgactgccaattaa

SEQ ID NO: 69 - RNA, 480 nt, Acinetobacter baumannii
  source 1..480: mol_type = mRNA
atgaaaaaatcattattagcaattgcacttatgagtactcttcttgtcgcatgtaataaacatgaaaataaaacagaaacaacctctgatgcatctactcctgtacaaactgctcaatcaaataataatgaagcggtagatacagcacatacagccgagaattctttagattgggatggtaaatataaaggtacgcttccttgtgcagactgtgaaggcatcaaaactgaattagagttaaaagatgataaaacttatgagctaacagaaacctatcttggtaaaggtgatgcaaacccatttgaaacccatggtaagtttactttcgataaagacaatacttctgttattaccttagatgataaagctcaaaaccgtaaattctttattggtgaaaatacagcgacagctttagatatggaaggtaaaaaagttgaaggttctttagctgaacattatgttttgaaaaaagaagattaa

SEQ ID NO: 70 - RNA, 489 nt, Acinetobacter baumannii
  source 1..489: mol_type = mRNA
atggcaaataaaaaacttttaatctgtgctgcaattgcggctggactattgttaaccgcatgcgttaaaaaagagacaccgaaagaagaagagcaagacaaggttgaaactgctgtttcagaacctcaaccacagaagcctgcaaaatttgaatctttagagagtgtagatactcaagaagctcaagttcaagagcaaccacaagttgaagttcatcgtgaagaaactgctaacacgacaactgaaattcgccgtgaaactcgtcctgcgcgttctgatgaatcttcacagacacaagttgctgaacaacctaaatctgagacaccaaaagtagaacctaaaccagaaaaaaaacctgagcctaaagctgaacctaaaccggaaaaagctcagtcaaaaccagctgctaaggcaactgagcctgcgaacacagaagatgatgccgttgctgctgctattgctgctgcaacaccagcacttaagaactaa

SEQ ID NO: 71 - RNA, 501 nt, Acinetobacter baumannii
  source 1..501: mol_type = mRNA
atgactactgaaaataaattagatgaattaaaggcaaacgcggctgatgcaaaagtacaaggtgaaaaagctttggacgatttaaaagaaaatgtaaaagagaaacaaactgcgggtaaagaagcaattgcagataaagtagatgagctaaagaccaaagcggccgatgcaaaagtacaaggtgaaaaagccttggaagatttaaaggaaaatgtaaaagaaaaacaagctgctgcaaaagaggcagttgaagataaagctagtgatttgaaaggcaaacttgatgatgctcagcatagcctgcaagacaagtttgatcatttacgtactgaagcagcgcataaacttgatgatgctaaagccaaagctgcagaattaaaggaagaggcggctacaaaatttgatgagttaaaaactcaagcaactgcgaaatttgatgagttgaagaaaacagctaccgaaaaacttaataaattgaaaaatcatgattctgctgaataa

SEQ ID NO: 72 - RNA, 693 nt, Acinetobacter baumannii
  source 1..693: mol_type = mRNA
atgcacacgcgtcgtattttattagcgttttcacttgccgcttcggcagcatcggttgcttttgcagattatcaaaatattaaccaatctactgattcagatcgattggaacagttatcaaaaacactatctcaaggctcatatactcatcctgatgatttagatcttccggcgagtgcaaaagtttcagtgactttacgtgagaaaacagttgaactcaacaatgattcactggcaaagaaatacggcacaacgacagctaaaaactcatttaaaacctcttcttcaaatccttattcttggttagtttctcatcctcttcctgatacagtacgtgtttcttctaactttggtggtcgtaccatgggtggtcgtgcagagcatcatggtggtttagatatggctgcaccaagtggtacgccaatctatgccactggtccgggtattgtgactaaatcaggctggggcacaggttatggccaatatgttgaaattaaccatggtaatggttatttaacacgttatgcgcatgcttcacgcttaatggttcgggtaggtgatcaggtttctgcgggtgaccatattgctaatgttggttgtacaggccgctgtactggtccacatttacattacgaagtagttaaagatggtcaacgtaagaatccatcaacttacttagcaatgttgccttaa

SEQ ID NO: 73 - RNA, 717 nt, Acinetobacter baumannii
  source 1..717: mol_type = mRNA
atgggcatgacttttacagacatagaaaataaatctgccaaacgcctaattggtattgctgcagtcatttttctgcatcttcttgttgcctatattctgatgtcaggtttagcaaacaatattcaaaaaccagcagaaaaacctgtggaattacaaattattcaggatattaagccacctcctccaccaaagccggaagagccgaaaccaaaggaaaaaccacctgagccaccaaaaatggtagaaaaagttgccaaggttcctgagccaccaaaagaagtagagaaagtagcaacaccagtacaaaaaacgacgccagtagctcaaacgactaaagtcgctactccggctcctgctgcacctagtactccttctccgagccctgttgctgcaccagctccagtggcagctgctgcaccagcacttaaacccgctggtgtaactcgtggtgtttcagaaggctctgcgggctgcgaaaaaccagaatatccacgcgaagcactcatgaacgaagagcaaggtacggtgcgtatacgtgttttagttgatacttccggcaaagtcattgatgccaaagtaaaaaaatcgagtggtagcaaaaccttagataaagcagcaactaaagcctacagcttatgtacgttcaaaccagcaatgaaagatggcgtacctcagcaagactggtatgaaattgaatatccattcgtaattgaataa

SEQ ID NO: 74 - RNA, 762 nt, Acinetobacter baumannii
  source 1..762: mol_type = mRNA
atgaaaatgatgaaaacagctatcgttactgcaagtgtacttgcttctgcctctatttttgcacaaagtgcgggcgttaatgcaggtgcatctgctcaagtcaacgtacaaccaggtggtcttgttagtggcgtagccaatacagtcaaaaatactgcacatactgtagggaacacagcaaaacatgctggtcacgtagctgctgacacaaccgttaaagctacaaaaaaaacaacaggtaaagtaactgaactttcttctaaagctgctactggtactaaacatgtagcaagtgaagctgtaactggtactaaacattttgctaccgaagcagcaacaggtacaaaaaatttagcgactaaggctgcaactggcactaaaaacctagcagttgaagctaaagcagatacaaaagcacatcttgatgctgtaaaaactaaggtagcagaaaaacaagctgaccaaaaagaatttactgctgaaaaacaggccgatgctcaagcacgtgtagatgctgttaaagctcgtgtagcacaaaatcaagctgagcaaaaagaattcgttgctgacactaaagctgatgctcaagcaaaattaaatacagctcaaccagctcatggagttaacgctcaaacaggtgtaaacgttggtgttaatgttgctggtattaatgcaaatgcaaacgtaaatgcaggcgctcaagcgtctacacaaaaaggcgaaaagaaatcttttattaaaggtcttttcggtactaactaa

SEQ ID NO: 75 - RNA, 885 nt, Acinetobacter baumannii
  source 1..885: mol_type = mRNA
atgcaaatgaagaagcattctttattatttattgccttaatgagcaccacttctctatacgcaaatatccctatcgagtctcgtggtttaagtcaaaatgatggtagtgcatctaatacttcttcaagcaatatttcagttcctacaaatttaaactgggaactcatgcaaaaaaaccaacaattagaaaatgatattcgtacattaagaggtcagcttgaagaacaagctaatgatattgagcaattaaagaaagatctagcaaaccgatataccgacttagatcaacgtttagaattattacaccaaaaagttgaccctgacagtgctacacaagatgatagttcgaatgcaacgtccgataacactactcctgctagtgcaccagcccctcaaaccacggagagtaataaagtagctgccgtaccagccacacaaacttctgagcagcagcctagcgcaccaacaaccactactcaaccagcaccagctgctgcgcaaaaccagtcaaattccctcgaattagaaaaagctgcttatactgtagctttagatgcgtataaacaaggcggtgctaaaaaagcaattgctccaatgcaaaactttataaaaaaccatcctaatagtatctatacaggtaatgcttatttttggttagccgagtttcatttagcgacagatccagtaaattataatgaagcaaagaaaaactataatgtcgtagctaaccaatacccaaattcaagtaaggcacctcgagcattgtaccaactttatagtatcgctaaagatgttgataagaatactgtgtcagcaaatcaatacaagaacaagttacttagccaatatccaaaatctgaagaagcaaaattctttaacaaataa

SEQ ID NO: 76 - RNA, 1071 nt, Acinetobacter baumannii
  source 1..1071: mol_type = mRNA
atgtcaatgaataacaaacaacgctggatgggtggcgttgttttattaggtggtggtgttttattggcagcattacttctaaaaggaaatgaagaaataaaacaggtagatgttcaaccccaaacttcaacatcaccgaaattacaagctaagccaaagcaatcagcacaagaagggcagatggtacagctacagccacttgcagttgatgttgaaacagaaaaacgtcttcttgaagaacagcgccgttctcgtgaaaaagctgtagcagaacaagaggctcgtgctgccgaattcttggcaatgcaacaacaggcagaagcggatgcagctcgtaaagctgctgctgaatatgccgcgattaatgctcgccgagctgctgcacaagaaagttccgataatattcctcctgaagtcgcgggtagtgagaataaggcaaaaggacagcaaactgatacaaaaaagtcagtagatctggctaaagcggatgcagataaaaaagctgctgaagcgaaacgcttagctgaggcggataagaaagcggctgaggcaaaacgtcaggcagaagcagacaagaaagcagcagaggctaaacgccaagcagaagcagataagaaagcagctgaggcaaaacgtcaggcagaagcggataagaaagcagcagaggcgaagcgccaagcagaagcggacaaaaaggctgctgaagctaaacgtcaagcagaagcagataagaaagcagctgaagcgaaacgtaaggcagaggccgagaaaaaagctgaagcagagaaagcacgtgagttgcttgaaaatggtgataaaaaatggatggtacaggttgcattggctgctaaccaagcaaatgcagatgctgtagtctctaaattacgtgcaaaaggctataaggtcacgacgagtccaaccagtaaaggtatccgtattatggttggtcctgcaaaagatagagatacggcagatactacacgtaagaaaattacttctgacgccagtttaaatatgaagtcagcttgggtgattgactgggtgcctttagatcagcgtaagtcagattaa

SEQ ID NO: 77 - RNA, 1104 nt, Acinetobacter baumannii
  source 1..1104: mol_type = mRNA
atggctaatactagatatgaagatgataacaatagttcaggtacttcaaatcgtggatttgcaagtatggatcctgaaagagtcagagaaatagccagtaaaggtgggcgagctgcacatgccagtggcaatgcgcatgaatttacttcagaagaagctcgtgaagctggccgtgctgcacatgccagtggtaacgcacatgaatttacctcagaagaggctcgtgaagctggtgctttaagtcataaaaacgatgatcgtaatggtcgtggtcgcagccgttatgatgacgacgaagatgatgaccgcggccgttcaagtggtcgaggccgtggccgcagtcgttatgatgatgacgacgaagatgatgatcgcggtcgctcaggcggtcgtggccgtggtcgcggtcgtgatgatgacgacgaagatgatgatcgcggtcgctcaggtggccgaggccgtggtcgcagtcgtgatgatgacgatgaagatgatgatcgcggtcgttcaggtggccgaggtcgtggtcgcagccgccgtgatgatgacgatgaagatgatgatcgcggtcgttcaggcggtcgaggtcgtggccgcagccgtcgtgatgacgacgatgaagatgatgatcgcggtcgctcaggtggccgaggtcgtggtcgcagccgttatgatgacgacgatgaagatgatgaccgtggccgttcaggcggtcgaggtcgtggccgcagccgtcgtgacgatgatgacgaagatgatgatcgtggtcgttcaggtggccgaggccgtggccgcagtcgttatgatgacgacgatgaagatgatgatcgtggccgttcaggtggccgaggtcgtggccgcagccgtcgtgatgatgacgatgaagatgatgatcgcggtcgttcaggtggtcgaggccgtggccgcagtcgttatgatgatgacgatgaagatgatgatcgtggtcgttcaggtggccgaggccgtggtcgcagccgtcgtgatgacgatgacgacgatgatgaccgccgtggccgttcagacggtcgtggtcagaactctcgtaatcaaaaacgcgatgcttatggacgctttacgtcttaa

SEQ ID NO: 78 - RNA, 1263 nt, Acinetobacter baumannii
  source 1..1263: mol_type = mRNA
atgttatatgtgattccgtttatcatattactcgtggtcgccgttattttaaaaaaacgcgagaatagtcaaaaacaagaggccacttccccaaaaaatataaatagaaaatccggcaaaaaagcgagtgctaaatcaagcaaaagctcgcgtgaaaaaataaaagccaaggtcattgaggaaaatattcctgctatccctcaaagtaatcctgtaccggaagctttacgacacaatattcaacagctcattcaagaaaaacagttttcagcagctgaagctcaagtcaatcaggcgctaaaaaaagataatactcaacatgaactgtatttattacttcttgaaattcatattgctcagaaagatgaatttgcaattcaacagttgattagccatattcgtagcttgggtttaaatgaaatagcagctcaagcagaaactcgacaaaaagaatatgaatcatccagccaacctgatgctattgatttccctcaagctcaaacatatgaagaaccgaaaaacacagatacaacggctcagttcgatgaattaacaacaagttcttccgaggcttcttttgatgacctacagaaagactatacccctgtaaaacaagaacctgctattgaaattgaacctttagagtttaacttttcattcgaacagaattcggctacggaaaataccaaccaaccagcacaacaacctgagttatcatccactcaagaaacaaatgagctggccgatttagagttttcttttgacttggcccctctgcatgaaactgaggaaaaatctcaagcagtagaggtaaaagcagaccaagaaaatagcatcaatgcattagattttaactttgacttaaatccttcaagttcagagacgaaatctgttcaacaagctccctcattagatgaaattaagctcatagaacaagccccattagaagcgacttctatcgcacctcttgagttttcgttagatgagcctgccttagttccagcaccagagcttgaaactcaaaaccatatagatgtagtaaatgaagcagccactcaaacccaaatagaggatccgcttttagaagcttttccagaattaaaacaaataaatgaaaatgaactcgatttaaaattggctgaacaatacattaagtttggcgcaaaccaagcagcgcgtaatttactacagggtgatgagcaaaaattcaacacagaacaacaacaacatgcgaaaaacctacttaatcgcatagcttcttag

SEQ ID NO: 79 - RNA, 1338 nt, Acinetobacter baumannii
  source 1..1338: mol_type = mRNA
atgccaaaaataaagccaattaaactcgtaattattgtcgtctgtatcgccattattgctgtcttagcttggaaattcttaaagcctaaacaacagcaacctcaatatattacagcagaggtaactcgtggggatattgagaataatgtacttgcaaccggtacacttgatgcaaccaagctcattagtgtgggtgctcaggtatctggtcaggttaaaaagatgtatgtgcagcttggtgatcaagtaaaacaaggtcaacttattgcacaaattgactcgaccacccaagaaaacagtttaaaaacatctgatgctaatattaaaaatttagaggcacagcgtcttcagcaaatcgcttctttaaacgagaaacaactcgaatatcgtcgtcaacaacaaatgtatgctcaagatgcaacacctcgtgcagatttagagtcggctgaagctgcttataaaacagctcaggcacaagttaaagcattagatgcacaaattgagtctgcaaaaataacgcgttcaacagcacaaaccaatattggctatacccgtattgttgcgccaactgatggtacggttgttgcgattgtgactgaagaaggtcaaacggtaaacgcaaaccaaagtgctcctactatcgtcaaaattgcaaaacttcaaaatatgacgattaaagcacaggtgagtgaagccgatattatgaaagtggaaaaaggtcagcaggtctatttcacgaccttaggtgatgaaaccaagcgctatgcaaccttacgtcaaatcgaacctgctccagattcaatctctagtgaatcaaacagcaccacaagttcaacaacaagctcagctgtttactacaacgctttatttgatgttccaaatacggacggcaaattgcgtattgatatgactgcacaagtttatatcgtattaaattcagcaaaaaatgccttattggttccatcttctgcgttaagtagcaaacaattttctggccaaagaaaacaggggcaatcggcagataaagcaagttctactccaagtgcagaacgcaagcatcaaggtaacggcgtccgtttagaacgcttaaatttaactcctgaacaaaaacaacttattgaacaaggcaaagcaactctgagtgtagttcgcgttttacaagcagatggtacgactaaaccaacacaaattttggtaggtattaataaccgtgtaaatgcacaagtacttgccggattaaaacaaggtgaccaagttgtgattgctgatagttcagaaaactctgcagcttctgcaaacagtggtaataaccgccgccgtggcccaatgggaatgtaa

SEQ ID NO: 80 - RNA, 2136 nt, Acinetobacter baumannii
  source 1..2136: mol_type = mRNA
atgaatatcccacctcgaccatttaagttgtctgtaatcgcctgcgcgatttgttatgccaacctcacctatgcccaagatgcacaggtacaagctttgcaaacgattcaggtcaaagcatcaaatgcagagcagtcttctgaacagaccaaggcatataatgttaaaaactcaagtagtgcaaccaagcttaatattgaggctaaggaaacaccccaaaccattaatgtcgttactcgtcagcaaattgaagattttggccttaccagtactcgtgatgttttaagaaatacccctggtgtaacagtctctaatcaagaaactgagcgaaccacttatatggctcgtggttttgagatttcaaatatcttgacggatggcgtaggctttcctctttcaggttataactataataatacgaatcctgatacttacttttatgatcgtgtagaagtagttaaaggagctgactcgttaactaatgcctttggcgatccgagtgcaaccatcaataatattcgaaaacgtccaacccaagaatttcaagcaagcggtggtgtaagttacggttcatgggatacgcagcgctatgaagctgacgtatctggctcaattctaccaagtggaaaagtacgtggccgtattatgggctatgaacaaactggtgattcttacctagaccgatattctgcggaaaaaaacggctttgctggtattgttgaagccgatttaacagatagcactttacttaccgcaggttatagccaagaacaaaataaacccaatgcaaacaactggggtgcactacctttattagatgccaacggtaaacaaatttcctatgaccgctcatataatccaaaccctgattgggcacattgggataatgagacacaaaatgcttttgttgaattaaagcaaaaacttaatgaccaatggaatgctaaactgacttacaactatcttgatacgaagcataatagccgtcttctctattactatggttacccaaaagctgatgggtccggtgtttctctaacgccttggggtggacaagaacatcaagaaaaacatgctgtagattttaatctcgaagggacctataagctatttaaccgagaacatgaagcaactctaggctacagctatgtacgtaatcatcaacaagataaacaatctacaggaacgattaacgatagtaacgttataaagtcaactacgaccgattgggcaagttggacaccgcaatctataacttggtcagatttcacagaagcggctaactataaacaaaatattaactcaatttatgccgcgacacgtttacatcttaatgaagatttaaaacttttacttggtgcaaactatgttcaagctgagagtaaaggcgaaagttatagctcaccaatgtcatatagtgaaagtaaagtttctccatatgtcggattaacctataattttacacctgaatatacgggttacatgagttatacctctattttccgtccacaaactggtattgataaagataccaatcaagctttaaaacctattgagggtaaaagctatgaaatgggtgtaaaaagctcatggctagatgaccgtttaacaggcacactttcagtatttaaaactgaacaaaacaattaccctttacgtaactcggatggaaacccacttaaccgaaaagtaccaacgagtgatctagaatcgcaaggtgtagaagtcggtctatcaggtcaaattactgataacgtaaatctttctttcggctatgctcaatttagtattaaagacactaaaaatggtggcgaagcaagaacatacaatccaaaccagacacttaacttgctaactacctatactccgccagttttacctaagcttaaagttggtgcaggtttacagtggcaagatgggataaagttatatgactcaaatgtaaacggtacgatcaaacaagatgcatatgctttagtcaatttaatggcaagctatgaagtcaatgatcatattacgcttcaagcaaatggtaataatatttttgacaagaaatatttaaatagtttcccagatgggcaggctttttatggtgccccagccaactatacagttgctgtaaagtttaaatattaa

SEQ ID NO: 81 - RNA, 2184 nt, Acinetobacter baumannii
  source 1..2184: mol_type = mRNA
atgaaacttcaaactatagcttgtgcagtagcaatcgcgactggcggtttattcttctctcatacgatgaacgaagcaagagcagcaaccaatactgctgctgtttctcaatcgatccagccaacgcaagagcaagccttggtggctcgtcaactggcaactttagtagaccgccaacattatttaaatatgcgtctggatgcgaacacatctaaccgtattcttgatatgtacttagacagtcttgatccggaccactcattatttttagacgctgaagttcaaaactataaaaagctctatggttcaaattttggtgcttcattaaaagcggggaatttaactgggccatttgctattcaccagcaatatcgtgagcgcttaaagcagttttatgagttcatgcttgctgagttgaagaaaccacaaaacttaaaacagccaaatacttttattgaagtagatcgcgaaaaagcaccgtattttaaaacgtcagctgaacagcaaaatcactggcgtaaaatgctggtttctcagttaattaatttaacgattagccgtgaagaagagcaggcgaaacaaaaggcgttaaaagaaaacccttcacttgctgatggtcaagacttaacaggcccagaagatttaacgccagctcagactttgactaagcgttatacgcgtcagcttgaaagaattagtcgtgtgaaaagcgacgatgtgttggataaaacattaaatgcaatgttggcaacttatgatccgcacagtaactattatccgccaattgatgcgatagaactgaaccgccaaacaaccttacagcttgaaggtattggggtatcgattcgcccagaacgtggtaatgaagattacaccaagattgaaactattgtagaaggtggtcctgcaagtaagtctggtcaagtgaaatcaggagaccgaatcgttggtgtcgctcaagagggcggcaaaatgatcgatgtggtcggctggtcgagttcggaaattgttgggttgattcgtggtaagcgcggtacaaaagtaacgttgaagcttcttggtgctggtgcatcaatgagtcaagcacgcaatgtgactttggtacgtgatgttattcaagaagaagatgccggtgttcgttcacgtacagttgaagtgacacgtgatggtaaaaagcatctattaggtgtgattgaaattccatcgttctattttgactatcgttcacgtcgtgctggtcagcaatatcgctcagtttctgaagatactgcaaatgcgtttgaggcattaaaagccaagaaagttgaaggtattattattgacttgcgtaatgacccaggtggttcattagaagaagttgcacgtatgctcggacaagtgattaagtcaggtccagttgtgcaaattcgtgatggcaatggcaacgtaagtgtatttgaagacaacgatggtggtcagcaaatctatacaggtccactcgctgtattggtgaacttggcatcagcatctgcaagtgaaatttactctgctgcaattcaagattatgagcgtggcattatcattggtagtacaactacaggtaaaggtacagctcaggttcaactcgatactttggcatatggtcaagcaaccttaactcagcgtaaattctaccgtgtaacgggtggtagtacacaaaacaaaggtgtagtgccagatattaagcttgttgatatctataacgaagagtttggtgagcgtaaatcgaaaaatgcgttgaagtgggatacgattccgactgcaccatttaagcgtgaaggttcagtgcagccatatgtagcgaagctgtctcaactttcggaacaacgtgttgctgttgatccacagtttaagtatttaaataaacgtacggcaattgcgaaggttacgagtgaccagaaacaagttgtgcttgatattgataagcgccgtgcagagcttttgagtttagaaaagcaaactttagatgctgaaaatgaacgtcgtatagcaacaggtcaaaaacctttccctaactgggaaagctatcaggcttctctagatgctctagctgaatctcgtgccaaaatgaaagctaatcaacgtcctgcgttgccagaagaagaaacgttcgtaaatgaagctgcgaatgtattgatggattatgcgaaattgcagaaccgttaa

SEQ ID NO: 82 - RNA, 2187 nt, Acinetobacter baumannii
  source 1..2187: mol_type = mRNA
atgacaagaataattgtagcatccaaagagggtttggacgttctgcaagatggtcagctcaataaggtggttttaaaccaacctactattattcaaattggtgtaagtcaaaaagatatcgcatcgatggaaaagcagggtggaagtctagtcatccatttaaaaaatggagaaacaattgtcttagaaaatttctttaatgaagcaacgaatacaacagagcattcactcgtttttccaactgaacaaggaaaatttgttgaagcacaatttgatgctcaaggtaaggttatagattatagaggcttaaatcatgttacggatttggcctataccagtaccagcccttcagctgcaacaatggccgttgataatgatccaagcttttcgatgggtaatgtactaaaagcaggcttagcagttttagccgctgaaggtttatatctttgggcatttgataaagatgataaagatgattcacccagtactcccgatttaatagcacctgctgctcctacagctacgcttgctgatgatactgtgacagtgactggcaaaactgaagcaaatgcgaaaatctacattaaagatgcagcaggtaatacagtggcctcaggtgttgctgatgcgagcggaaattacacgattaaattagataagccgttagtgaatggggataaattaaatgttattgcccaagatgcggctggaaataattctaaagttactgtggtaacaggaacaaaagatacaattgccccagatgttccacaagctcaattgagtgatgatggttccttattgacaggtaaagcagaagcaaatgcaaaaatcactgtttatgatgccactggcaaagtattaggaactgtttttgcgaataaagatggtatttattctttaaaacttactccaccattaaccagtgaagcgggcggtaaagtggttgccgaagatgctgcgggtaacaaatctgaggaagttaaaattattgcgggtaaagataccataccgccagcatctccttttgttgaagtaaataaagaagggtcggtaatacatggtaaaactgaagcaaatgcaaaagttcaaataaaagatgccgatggtaaagtgattggaagtgggaccgccgatgctcaaggtgaatttcaaattacactttcacctgctttaaaagaggcgcaaaagggcacagtggttgtggaagatgctgctggtaatgtatctaaaccagttgaaattacgccaggctttgactcgattgcaccagataaaccgactgttcaaattaatacagatggtacttctgtaaccggtacggctgaggcaaatgccaaaattgaaattaaagatacaacaggcaaggtaattggtagtggaacagcggatgcgaatggaaaatttacaatttctatttcaccagctttaacggataataaacatgcttcggtatcagctatagataatgctggaaataagtctgaagttgttgatattgtaggtacaaaagatacaacaccaccagcaaaacctatattaaatagcgtagatgatgatgtaggtgctgttaaaggagctataacggctggttctgaaactgacgatgctagaccaaaacttacaggctcaggtgaagcaaatgcaactcttactatttatgataatggtgttgcaattggagttgtgacggtaacaagtggtagatcttggtcatttacatttgataaagacttagctcttggtaagcatactattactttgactcaaactgatgcggcaggccttaccagtgaggcaagttctccatttaccttttatgtagttgctccaaaggctgcgagtctgtctgaaacttcagtagatattttaagtacagagggaccatctttggcagatagtgttggattgcatactttaaaggtagcgcaaaatacaacaactgagacgaataacccgcagaaatcggttcctttagatgatttattaaaaagttctacggctagtgaatcagacccaatcgcaaaacttctctcatcaacagcgttaaaaacgactcaggcatctgagccaatcgaagtaaatgcatcagttggtcagacaacatcaaatcctaatcatcctttacctgatacaacttcttcggttttacaaaaccttttagatcaaacttatccagttgtttag

SEQ ID NO: 83 - RNA, 2265 nt, Acinetobacter baumannii
  source 1..2265: mol_type = mRNA
atgtcgaagcgtattatccagtcagtgctttctgtttcagtactggcaagtatgatgtctatggcttttgctgcacaaaatgagcaagaacaagctgaacaaacattagaaaagcctgctgaacctgtgaaattggaaacaattttcgtaacagctgaagagcaagtgaagcaatcgctcggtgtatcggttattaccaaagaagatttagaaaaactaccagttcgtaatgatatttctgactatgtacgtcgtatgccaggtgtcaacctgacaggcaatagtgctactgggcagcgtggtaataatagacaaattgatattcgcggaatgggccctgaaaacacgcttattttagtggacggtaaaccaattaactctcgtaattcagtccgttatggctggaaaggagagcgtgatacacgaggcgactcaaactgggtaccagcagaagccatcgagtcgatcgaagttttacgcggaccagcagctgctcgatatggttctggtgcggcaggtggggtggttaacatcattactaaaaaagtgactaatgaaactcatggttcagtagagttttatacttcacagcctgaagactccaaagaaggttcatcaaatcgtgttggttttaacgtaagtgggccactaattaaggacgttttatcttatcgtttatatggtaattataataaaacagaagctgatgatgttgatattaataaatcaattggtagtaccgcagctggccgtgaaggtgttaaaaataaagatatttcaggccgtttagcttggcaagcaacagaccagcaaactgtcttgctcgatatttcttctagcaaacaaggtaatatttattctggtgactctcagttaaatgcaaatgctgaagcggatgcgatactttcgcagctcattggtaaagaaaccaataccatgtatcgtgatagctatgcattaacgcatgaaggtgattggtcttggggtaagagtaagttagttgctcaatatgataagacccataacaaacgtctacctgaaggcttggcgggaagtgtagaaggaaaaattaataatcttgatgataaagccacttcgcgtttagaaactcttcgctttaacggcgaggctaatattccttttgaatactatttaccccaagtattaactgtaggtaccgaatgggttgaagacagatttaaagataatgtctcgacaactcaaggtaaagacagcagtggttcaggttatggcgatcaattagcgaaaggtgatcgtagtaaaatggagtcacgtattgcttctgcatatattgaagataacctgaaagttacagacagcacagatgttgtattaggtttacgttttgatgaccatagtaaatctggttctaattggagtccaagcttaaatattactcaaaaactcaatgataatttcactttaaaaggtggggtagcaaaagcttataaagcaccaaatatgtatcaaaatgccgaagggtatttattaagtacaaatggcaatggctgtcctgctaatattgagtcgcgttgtttattacaaggtaatggtgatttaaaacctgaaacatcggtaaacaaagagctcggcattcagttccaaagagatatcgtgaatgcgagcttaacttggttccgtaatgattataaagataagattgttgcgggtactcatgttgtcggaacagttgatggctcaagtacaaatgcaaatacaggagctgtgaccaatacgaagtggaatattttgcgttgggaaaatacgcctaaagccttaattcaaggttttgaaggaagtttggggttagacttcggtgatatccgctggactaataactttacctacatgatggactcgaaagacaagcaaactgggaatccattatctttagttccaatctatacaattaactcaatttttgattatgacattactgatcaattggatgtaaattttgtatttactcaatatggtcgtcaaaaatcacgtcaatttgcagagaatagacttgaatccggtataggttcaggaggtgcgaattctgcgcttaagccaagtacggtaaaaagctatagtactgctggtattaatgttggttataagttttcagaccaaattagtacgcgtgttggtgtgagtaatctgtttgataaacaaattttaagagacagcaattctattagccaaacttataatgagccaggtcgagcttattatgcatctttaaaatattctttctaa

SEQ ID NO: 84 - RNA, 2721 nt, Acinetobacter baumannii
  source 1..2721: mol_type = mRNA
atgccttctaaaattaagtttaaacagtcaactctttctcactctatgcatttaatcttaaaaatgcagagtatacctaaacttatttgtagcagcttattattaagtttatgtgttactccttgttatgctcaaagttcggctgagaccgttataccagaagcaaaccagacggtaacagattcattggtacaacaaactaatacaaataacccaagtgatgttccaattaccgatgtcgctactcttgtaactcaagcacagcaacaacaagatagcttggctatattgcaacaacaagaacaatttccgaatcagattgaagaatttaagccaattacgcttgataatcttgaagacttacctgttatgcctgttgatcagaatatggcaaatgaaatttatcgggtagcagaagaggcaaaaaacgaggctcaaaacttccagaatggtacgcaaaaacaaccagaaatggtggtgagtgacgcatcacaagcagaattacatgaaattaatcaggcccctgtaaatattgaccagctcatgcatgagattcaatctgatagtaagattgtggttgaagccaatgaaacaggaaaaactttacctgagcttactgctgccgttgaagaaccacccgaggaaaaaggtttctttagacgtatattcaataaaatccgtccacctcgggtaattccaatggagcagataccccgtattactgctgaggttacgggtgcgccagatgatttagctaaaaatatcaaaggtaaattatctacatttacccaagaatcatttgaagattttaatgcagcgctaccgcaacttaggagcttaagtaatcaggctgctcaagctgtaggttattacaatgccgagtttcgttttgaaaagttaagtgccagtcgcgtacgtgttaatgtaacgccaaatgaaccagtacggattaatgaacaaaacattgaatttactggtgctggtgcaaaacagccacaatttcaggtcattcgtttagttcctgaccaagatgtaggtgatatttttaatcatggcctttacgaaaccacaaaaagccgaattgtcgatgctgcatcggataatggttattttgatgcttattggcgtttacatgacgtaaaagtgagccaacctgaaaataaagcggatattaacctcaagtatgagactggtgagcgttataagcttggtaaggttgagtttcgcatgagcgatccatcaaaaccattacctttaaatatgaatattcttgaaagcatggcaccgtggaaagagggtgatgactatgctttttggcgtgtaaatgttttagcaaataacctgactaactcacgctattttaactataccttggttgattcaattaaacccgacccaattgaaaaaccacttgagttaccacccgatttacaagcgttggtcgatcagcagaatgttgatattgacgaatcgaagctgcttcctttagagcaacaacaacttgccaaagcacgccagttggcttcctcaagtaaagaagtaacacaaaatgtggtagatgaaaaacaatttgccggaactgaaagtgtacaagccgcacctgcatctttaaaagctgcaactgtacaacatgaagaacaagagtctgaacaagaccgtttacaggctcaagctcgggaagaaaaacgtataccagtgattgtgacgttaaatgccgataaactaaatagtctggaaacaggtattggttatggtaccgacactggcgcccgtttacgtagccaatatagacgttcgattgtgaataaatacggtcattcatttgacgcaaacttggagctttcccaaattcgtcaatctatagatgggcgctatagtattccttataaacatccgttaaatgattactttaatattgtgggtggttatgagcgtgaaacgagggatgatattggtccggatgtaagtttactcacggaatcggcagttttagggggtgagcgagttattaaaaaaccactcggaaactggcaacatactattggggtacgttatcgtctcgaccgcctaactcaaaaggggaatgtggatatctctgagctaccagatgcatttaaaactgctgcatcagagcaagaagcattattatttagttatgagacctctaaaacttcaagtaatacacgcttaaacccgaccaaagcttttaaacaaacttataaattagaattaggtagcgaaagtttactttcagatgccaatatggcgattgccacagcgggttggagatttatttattctttaggtgaaaatgatgaccatcagtttgttgggcggtccgattttagttatatttttaccgatgagtttgataaagttccatacaatttaagattctttactggtggtgaccagacaattcgtggttttgattataaaagtctttcaccagaagataatggatataagattggtggacaggctctagcagtaggctctttagaatataactatcaattcaaagagggttggcgagcagctgttttttctgattttggtaatgcttacgataagagttttagtaatccgacggcctatagtgtgggtgttggtattcgttggaagtccccaattggaccaattcgtttagacgtggcttctggtatttctgatgataaccatccgattcgtttgcatttctttattggtccacaactttaa

SEQ ID NO: 85 - RNA, 2925 nt, Acinetobacter baumannii
  source 1..2925: mol_type = mRNA
atgtttattaaaagtattttatcttcgattactagtattatcccacttcctgaaaatagtaacacaagcagtaatttaggtaacggctctggtgacggcctacttaatggaatatcttccggaaatggtgaacacaactatggtattggcaatggtattgccgatgacgcaagcattaccgccccaattaccattcccctcaacctctctggtaactcaattactctcataggcaattcatcttcaagttcggtgaatagctctccaaccaccacttcaaataacgttaatgacaacgatgtaacaaataatggtaacggctctaccattggtagcggtacaggcaatggctctggtgacgggcttttaaatggcgccgcttctggtaatggtgaacacaactatggaatcggtaatggtattgccgatgacgcaagcattactgccccactttcaattccaattaaccttgcgggtaactctattaccctaattggtgactcatcttctagttcggtcaacaactctgcaaccaatacatcaaataccgtgaatgataacgacaccacctataacggcaatggctcaggtggtggtaatggttcgggcgatggcttgttaaatggaattggctctggcaatggcgagcaaaactacggtatcggaaatgggattgcggatgacgcaagtattacagccccaattacgcttcctattaacttgtctggtaactcaattactctaattggcaattcatctgcaagttctgttaattcctcgccaactacaacgtcaaatactgtgaatgacaacgacaccacctataacggtaatggtactggtgatagcggcgtgagcgctctgggcggttctggcaatggttcaggtgatggcgcaggtaatggtatcgcttctggcaatggtgaacataactatggtattggcaatggtaacggcgacgatgttgatattactgctccgattaccggcgttctaaacatttcagggaactcatttactctcattggtaattcttcatcaagttctgtcaataccgcaccaacaaccacatcaaatacagtgaacgacaatgacaccattgataatggtaatagcggcggcacaggtagtggttcaggcaatggctctggtgatggtttacttaatggcgctgcttctggcaatggcgagcataactacggcattggcaacggaaatggtgatgatgtagatattaccgcaccaattacaggtgtttttaatttctctggtaactctttctcaatcattggcaattcatcttctagttcaattaatactgctccaactacaacgaccaatacagttaatgacaatgacgtaactgataatggaaatgatggaggcggacttgttggtggaagttctggaaatggctctggtgacggactgttaaacggcgctgcttctggcaatggcgaacacaactacggtattggtaacggaaacggtgatgatgcagatttcaccttccctcttactggtgtacttaacttttccggcaactcgctttcaggctttggcagttcatctagtgactcggtaaatgtagcaccaaccacagcaaccaataccgtcaatgataatgacaccattgataatgccaatacaggcggccttggtgacggttcgggcaatggctctggcgatggtcttttaaatggtgcagcgtctggtaatggcgagcataactatggcattggtaacggaaacggtgacgatgcagactttacgcttccatttactggcggtttaaatattctgggcaatgctttatcaggtatcggcggttcttcgaccgactctattaatatttcaccaacaactacttcaaacacagtcaatgacaatgacactaccaacaacggcaacacttcaggtggtgtgattggttctggcgactcaggcaacggctctggtgatggcttattaaatggcatctcatcaggtaacggtgaacataattatggcattggcaacggtaatggcgatgatgttgacgttgttgcccctatcactacaccacttaatgtattaggcaactctttctcatttattggtggtgaaggtacaggcgatatcttaggtccgattactggcattattggtggtattggcggtgatggagatatcctaagtccaattactggcattatcggtggtattggtggtgatggagatatcctaagcccgattactggcattatcggtagcattggcggcatcgggggtgatctgggcgataacccacttacaggtattattcaaagcggtattgacgttctacaaaatttagaaagcctaaaaacaggtttgattaatacaggtattgatacgattgccggaacaattattggtgttttccctgatgcagagcaccctgtcggtgattttgcagatcttggaaaactacttttcgagacttcacgtgatagtgttaacggcacgcttgaagctatttctgaccttgcaggcgctgaccttgagggggcaagtggctcgattactggtgtaattgatacccttattaccaatggttcaacggcatctaccattattcagcatattgtaggtgatgacctagtcactgaaaacggtggcctcttgggttcaatcaccacgattattggtggtgttgacagcggcgacggtggtttactgggtggcctagatggcttaattagcattaactatggcgactcagacaatagtaattctatagatgtagaagatattttaggaaatatcctcggctcggttggttcaaatcaaggtattgctgttggtgaacctgatccaacgggcggtagtttgattcatacgatttcacttaacacagtaaatcagttaactgaccaacttttacatgctttaccgactgtctaa

SEQ ID NO: 86 - RNA, 3216 nt, Acinetobacter baumannii
  source 1..3216: mol_type = mRNA
atgtataaaccaaccacatttgtgtggcagccatctgcagcatcattattcaaaattacagtccttagctctgctttagctgcgttgggtattacaacaggctgttcttcgactccgcaatctgcaaaaacctcgaaaacaaaacaggtgagtggagcaggctatcttgatgcaagcagtctggactctttagaagacttactttcagcaacagatatgcgtgctgtagagggcgaccgtttacttattttaaaacacggtgatgtctggaagcgtatggctgtaggcttcaaaatggacctcaatcattgggatccacgtattgaagcgcagcgtagttggtttatctcacgtcagccctaccttgaccgcttaagtgctcgcgcaagtcgttatttatatcatactgttaaagaagcagaacgccgtggtcttccaaccgaacttgcgttattaccagtgattgaaagttcatatgacccagctgcaacaagtagtgcggcggcggcgggtttatggcaatttattcctagtactggtcgtatttatggtttacaacaaacgggtatgtacgacggacgccgtgatgttgtggaatctactcgtgctgcctatgagtttttaggaagcttatataaccagttcggttcatgggaattagctttggctgcttataacgctggtccgggtcgtattcaacaagcgattaaccgaaatcaggctgctggtttgccaacagactattggtcactaaaattaccgcaagaaaccatgaactatgttccgcgtttcttggctgtcgctcaaattattaaaaacccacgcgcttatggggtttcgttaccgccgattgcaaataggccccatttccgtgaagttaccttatctgcaccattatctttaaatgaaatcgcgtcggtgacgggtcttagccgtgctgagttatatgcattaaacccaggctatcgtggtgaaacagtagaccctgcaagtccaatgcgtattttgattccagctgatataagtccttcagtggataacaagttaaaaggaatgaaagcaggtggaagttcaggctggtgggcaagtgttacttcgccgtctaaaccgaccacaacaacttcaacctcagtcacagttagaactactccatcaactccagctcagccagtaagaccatcgacgcctgctaaaacaagtagcagctcggtaacggtaaaaactacaacacctcgtggttctgatgcgctagctgcttttgcagcgtcagctgatgtaccaagtgcaccacgtattcctgttgcggtcactccggctgcaaatatcaaacctgtgagaacggaaccgccaatttcagcaactgaacgcgagaaaattttagcggcagtgcgtgctgaaggggaaaaggaaacagttgatcaagctttagaacctcaagctactcaagctgaaaaagatcaagttgttgccgagttaaaagcacttgcacctcagggtacagaaattgtcgatccgtatgacggcaaaattaagttaaccgcaattcagaccagccaatctgttgctgagcagcaaggtaaagaagtaagtaaaggttttgcttatccaaaaactttagctgaagatgcaactcttgcaaactctgaagatgctcagcgcaataaagataagccttatattaaaactgatacagatgttgtggttgtacaacctaaaggtaagcgtagtacttatacagtacagcctggcgatactttagcagttattgccatgaagaatggtgtgaactggcgtgatgtagctaaatggaaccagattgaccctgaaaagactttatttgtaggaaccagtctatatctctatgatgctaagcctcaagaggcggaaactacggcaaaatcggcagctaaacctgatgtctatgttgttcaagcaaatgacagtttaacaggagtggcaaatcaatttaatttgtcagtgaaacagctagctgaatataacgatttgtctgtaacagatggcttgtttgtagggcaaaaattacagttaaaagaacctaaaggtaatcgtgctgctaaagtagagccaaaggcaattcaagcaagtacacgtcgtattgcaacaaagagttatacggttaaacgtggcgaatacttgaaattaattgcagaccgttatgcattatctaatcaagagttggcagacctaacaccagggttatcagctggtagtaatttaattgtaggccaaaaaattaatgtacctgcaaaagaaatcactgttgatgaagttgatgacagcaaggcttctggtaaatatgaaaagcttgcagcaggtccatcatataaaactgaaagttataaagtgcagcgtggtgatacattatcaagcattgcgaccaagtctaaaattagcctagctgagcttgctgaactgaacaatttaaaagcgaacagtcatgtccaactgggacaaactctaaaagttccggctggtgcatcagttcctgaccaatatgttgtgcagtcaggcgatagcttaaatgcaattgcagctaaatataacttacaaaccagttatttggctgatttgaatggtttgtcacgtactgcaggccttcgtgctggtcagcgtttaaaattaactggtgaagttgaaacaactagcaaagtttcggctaaaaataccaaagaagaaacgccagagacctacacagttaaatctggtgatagcttaggaaatatcgctaaccgctatcatttacagttagattacctcgctgcattgaacggtttgtctcgcaacagtaatgttcgtgttggtcaacgcttaaaattaacaggtgatttaccaacagtagaaacagctaaaacggatacggcgaaatcgtctccaaaagctgtagttgcaggaaaaaatactgaaaagtacacggttaaagctggtgagtcgttaaatgctattgcgagtcgtgcgggtatttcagttcgtgaacttgctgaaatgaatgcattaaaagcaaatgccaatttacagcgtggacaaaatattgtgattccaaaaactgtagtggaatacaaagtcaaacgtggtgataccttaattggtcttgcgagtaaatatggtttagaaaccactttattggcggaactcaacaacctaacaccgtcgactcaattgcgtattggcgatatcattaaagtgcctaatttatag

SEQ ID NO: 87 - RNA, 3333 nt, Acinetobacter baumannii
  source 1..3333: mol_type = mRNA
atgaaacgtatgttgattaatgcaactcatgccgaagaagttcgcgttgcacttatcactggtaatcgtctttacgattttgatttagagaatcgtacccgagaacagaaaaaatccaatatctataaaggccatgtcacccgcgtagaaccatctttagaagctgtttttgttgaatatggcgcaggtcgtcaaggcttcttgtctatgcgtgaaattgctaacagctattttcaggcagacccacgtcaaacttcaaacattcgtgaacttattactgaaggcacagagcttttagttcaggttgaaaaagaagaacgtggtaataaaggtgcagcactttctacgtttatttcccttgcaggccgttatttagttcttatgccaaataacccgaaaggtggtggtattagccgtcaaatttcaggttcagtacgtgaagaattaaaagaaattttagcttctttaaatgtacctcgtggtatgagcgttattgtacgtaccgctggtattggacgtacccaagaagaattacaactcgatttgcaacacttgcttgacctttgggcacaaatccaaggtacagccagctcaggcccttctccgatgcttgttcatcaagaagcgggtgtagtaacacgtgcaattcgtgattacttacgtgatgacgttgctgaaatcttaattgatagcgaacaagcttataacgaagcatataactttgttaaagcagtgatgccgcgtcaattagacaaattaaaaacttatactttaaacgagccattgtttgctcattttggtattgaaagccaaattcaaactgcttacgagcgtgaagtaaaacttccttctggcggttcaattgtgattgaccagactgaagctttagtttcaatcgatattaactctgcgaaatcgactcgtggacatgatgttgaagaaactgcactgaatactaaccttgaagcagcagaagaaattgctcgccaacttcgtttacgtgacattggcggtttagttgttatcgacttcatcgatatgactaaagaacgcaatcaacgtatggttgaagcaaaacttcgtgaagcaacacaaagcgatcgtgcacgtatccagttcggtcaattgtctcgttttggcttaatggaaatgagccgtcaacgcttacgcccatcgcttgaagaagcaacaggttatgtatgccctcgctgtcatggtacaggcatggttcgtgatttacgttcgctttctctttcaattatgcgtaaagttgaagagattgcacttcgtgaacgtcatggtgaagttcaagttgaagttccagttgaaattgcagcctttttgcttaatgaaaaacgccatagtctggtgtatttagagcaaacttctggtgtacgtgtgactgtattgccacacccgcacttagaaactccacattacgaaattgcatataacccagatggctttgctccatctagctatgaacgtacagaagcaacccgttctagcgagaaagagttaggttatgagtcttctgaatggcatttagaagaagcagatcatggccatgctcatgtaactgctacagcttcaactcatgctgcggctcagaaaaaagcaaatcatgcaactcaacctgtagcacaaccttctgctcaaaaagcagcaagcccatgtgcatggttagaaaacctgtttgttcaaaaacaagcgcaaacagttgatcaatctcgttcagcacaaaatgctgctgctgcgattgagcaaatggtgaatacaggcgcagtaagccgcggacagttcggtcaagtagccgtacctgctgttgcggaagttgcgccagttcaatcaaataacgcttatatttcacagtcacctgtgaaacaagacgttcgtgaacatgttgaaaaagatgataaatctcagcaacaacgtcagaacaacaaaaagcgtaaacataaagagcaacgtgaacaacatcaccaatcacatgaacagcaacatcaagttcatgaagaagtggttcaattgtcacgccaagaacaacgtgagttaaaacgtcagcaaaaacgtcaacagcagcaagatcagcaacatcaaaataatgatgtacaacacactgaaaatgctgtgccacgtcgtgaccgtaataatcaacaacgtccaaaccgtccaaatcgccaccgcgatccaagtgtattaaatgaaaatcaaaatacactggttgttgttgatgaaaaacaaattaaggttgatgtgattgatgcacctaagcatgatgtcatgaacactgctttaatcatcaatgttgatcaaggtcaaagtgagattgttgcacttacacctgagcgtcgtcacgttgagcgagttgaaacgacttctactgaagttgctcaagagccaactccagctcctgttgtagccgagaaagctgctgtagttgaaaccaaagaagaagctcagccaagccaagaagctgctcagccacaaatcaaacgtgctagcaatgaccctcgtatgcgtcgtcgtcagcaacgtgaggctaaacacgctaaggcagctacaccatctattgcgccatcgcaaattccaactttggcacaacacacaatcggtagtttaatccgtcatgtgtatggtgaagattgcaccgtattgattgaacaatttggtctagtcccaacgtttaatcgtgctttgcagaaatttgcagaacagtacgcaagtacgttagtagttgaagttactgctgaaacggaagagaaaaagccggtaactcgtgatgcagaacttccaagccataaaccagctgaagaagcagaacctgcaccagtacttccgcttactccgccgcaagcaccagctccacgtgttgcaaatgacccgcgtgagcgccgtcgtttagcaaaacttgctgcggaacaagcatttgagcaagtgaaacaacaacattctgctcaagaagaagttgctactcctgctcctgtagcagaagaaaccgttgctgctccaactgctgaaacacaagcaacagttgaaccagcacaacaaccgcttgaacttaatcagtcaactgaagtcgtacaacctgaagctgctcctgcagaggaaaaagctacagaagaaacagtagctgaagctcctgcggcaaaggaacctgcgccatcaaaagcagcaagcaaagcaaaagcagcggctgaagaaacagtagctccgactgaagcaacaactgatgccgaatcagaagacgttaaggcagataaggacaaaccgagtcgccctcgtcgccctcgtggccgtccgccaaaaaaagctaatcctgtagctgagtaa

SEQ ID NO: 88 - RNA, 3687 nt, Acinetobacter baumannii
  source 1..3687: mol_type = mRNA
atgtctacgcttgccaccctaaaagcgcttcttgctaaacgcattctgattattgatggtgcaatgggaaccatgatccagcgccataaattggaagaagctgactatcgtggtgagcgttttgctgattgggcacatgatttaaaaggtaacaatgaccttttggttctaacacagcctcaaatcattcaaggtattcatgaagcctacctcgatgctggtgcagatattattgaaaccaacagctttaatggcacacgtgtttcaatgtctgactaccacatggaagatcttgttccagagattaaccgtgaagcagcacgtttagccaaagcagcttgcgaaaaatattcgactccagacaaaccgcgttttgtggcaggtgtactggggccaacatctcgtacatgttcaatctctccagatgtgaacaaccctgcttttcgtaacattagctttgatgaactaaaagaaaattatattgaagcgactcatgcactaattgaaggtggtgcagacattatcctgattgaaaccgtatttgatactttaaattgtaaagcagcgatttttgcagtcaaagaagtattcaaacaaattggtcgcgaattaccaattatgatttcagggaccattaccgatgcatcaggccgtactttaacaggtcagacagcggaagctttctggaattcggttcgtcatggcgatttgctttcaatcggttttaactgtgcccttggtgcagatgccatgcgccctcacgtaaaaactatttccgatgtcgcagatacctttgtttcagcgcacccaaatgcaggcttaccaaacgcatttggtgaatatgacgaaactccagagcaaactgcagctttcttaaaagagtttgctgaaagcggtttgattaacattactggtggttgctgtggtacgacaccagaccatattcgagctattgccaatgcggtaaaagacattgcgcctcgccaagtgcctgaaaccgtacctgcttgccgcttaagtggtttagaaccatttaatatttatgatgattcattgtttgtaaacgttggtgaacgtactaacgttaccggttctaaaaaattcttacgtctcattcgtgaagagaactttgcagaagctttagaagttgcacagcaacaagtcgaagctggcgcacagattattgacatcaacatggatgaagggatgctcgactcgcaaaatgcgatggtgcattttttaaaccttgtagcatccgaacctgacatttcacgtgtaccgatcatgattgactcatcgaaatgggaaatcattgaagccggcttaaaatgcgtacaaggtaaaccggttgttaactcaatttccttaaaagaaggttatgacgagtttgttgaaaaggcccgcctctgccgtcaatatggtgctgcaatcattgtgatggcctttgacgaagtaggtcaggccgacactgctgaacgtaaacgtgaaatctgtaagcgctcttatgacattttagtaaacgaagtaggcttccctgctgaagatattatttttgacccgaacgtgtttgcagttgcgactggtattgaagaacacaacaactacgcagtcgattttattgaagcaacgggctggattaaacagaacttaccgcacgccatgatttctggtggtgtgtctaacgtttcgttctcattccgtggtaatgagccagttcgtgaagccattcactctgtattcttgtaccatgccatcaagcaaggcatgaccatgggtattgtgaacgcaggtcaaatggctatttatgatgatattcctaccgagctaaaagaagcggttgaagatgtcattttaaatcagaatcaaggtgagtctggtcaggctgcgactgaaaaattacttgaagttgcagaaaaataccgtggacaaggtggtgcaacaaaagaagccgaaaaccttgaatggcgtaatgagtcagttgaaaaacgtcttgaatatgccttggttaaaggtattacgacttatattgaccaagacaccgaagaagcccgcttaaaatcaaaacgtcctttagatgtaattgaagggccactgatggacggcatgaatgtggtcggtgacttgttcggttcaggcaaaatgttcttgccacaagttgtaaaatctgcccgagtcatgaaacaagcagtggcatggctcaacccgtacatcgaagctgaaaagacagaaggacagtctaaaggtaaagtcctaatggcaacggttaaaggtgacgtacacgatattggtaaaaatattgtaggcgtagtacttggctgtaatggctatgacattgttgaccttggcgtaatggtcccttgcgagaaaatcttgcaaactgcaattgatgaaaaatgtgacatcattgggttatctggtctgatcaccccatctttagatgaaatggtatttgttgctaaagaaatgcagcgtaaaggctttaacattcctttattgattggtggtgcaaccacttctaaagcccacacagcagtaaaaattgaccctcagtatcaaaacgatgcagtaatttatgttgctgatgcttctcgtgctgttggtgtagcgacaaccttgctttcgaaagaaatgcgtggtgcatttattgaagaacatcgtgctgaatatgccaaaattcgtgagcgtttagccaacaaacaaccaaaagcggccaaactgacttataaagagtcggttgaaaatggttttaaaattgatgaaagctatgtgccaccaaaaccaaatcttttgggaactcaagttttaaagaattatccgcttgcaacactcgtggattattttgactggacgccattctttatttcttggagtttaactggcaaattcccgaaaattttagaagatgaagtggtcggcgaagcagcaactgacttgtacaaccaagcacaagcaatgttgaaagatattatcgacaacaaccgttttgatgctcgtgctgtatttggtatgttccctgctcagcgtacagatgcagataccgtcagcgtatttgatgaagctggtcaaaatgttacgcatacttttgagcacttacgccagcaatctgacaaagtgacaggcaaaccaaacttgtctttagcagattacattcgtgctgaccgcgagcagcaagactacttgggcggattcactgtatcgatttttggtgcagaagaactggccaatgaatacaaagccaaaggtgatgactactctgcaattttagtacagtcattagctgaccgttttgctgaagcctttgcagaacatttacatgaacgtattcgtaaagagttctggggctataaagcggatgagcagctcagcaatgaagagctgattaaagagaaatatgtcggtattcgccctgcaccaggttatcctgcttgccctgagcactctgaaaaagcagtgttatttgactggttaggctctaccgacaaaattggtactaaactgactgagcactttgcaatgatgccgccatcttcggtaagcggtttctattattctcatcctcaaagtgaatactttaacgtgggtaaaatttctcaagaccaacttgaagattatgcaaaacgtaaaggttggacactggatgaagcgaagcgttggttagctccgaatttagatgactcgattgtttag

SEQ ID NO: 89 - RNA, 3855 nt, Acinetobacter baumannii
  source 1..3855: mol_type = mRNA
atgaaactaaaattgaagaattttaagccaaataatttatggtatgccgtctgttcaagctcaatgatattcacatggctgatgacaagctctgtggtgcaagcaagtgatttacaaatttatgcctcacctacagcaggtaaaaaaacaattgtgatgatgttggatacatcaggaagtatgaccaacaatagttatggtgaaaaccgcttagccatgcttaaaaatggtatgaatgcttttttggctagcaataaccctgtcttaaatgatacgcgagtaggtttaggaaatttctcggcaaatggtgacagccgaagtggacaaattttagtggctgctgctcctttaggtgatgcaagtactttaaatacggtaggctcacaacgttataaattaaaacaagctgttgctaatttaactgcgggaggctcaacaccttcagcccacgcttatgctgaagcagctgcttacttaatggggacaaccacatattcagagacgaattatgctattcgtaaagatagttatatcaaacgtgtaagaagatctgataatagaactgaatattcatattgtactaattaccgtgattcgcaaattgatacagccaacctatggcaaccttgtcgttcaaatagttattggagtagctggtcaacaaataatccaggtgtggataatgctactgcttatgatacttcatctgactggacttattactatacttattactacaccacttttaattatgctgtagcaaatgcagatagcggtattccaaaatctaaatcaaacgatacagctagcaatccgaatattgttgtagaccgaaatgctactaactccaatgcagtgtatcagtcacctttacctgcagtagcgaatcgccagagttgtgatggacaaggtatttacttcttatcggatggcgaaccgaacaatacaacaaatacacgttcggcaagtgtcatgtcgactgctttaggtagtacttttggagctgattttaattgttctggaggtttatctaacactacggcagactctggttgggcttgtatgggagagtttgcaaaaagattatttgataaaacaaagaaccctgccggagtttctattcaaacagcatttgtcggttttggtagtgacttttctagtttgaattcttctgatgtaaaaaatgcctgccgtttaagttcgagaacccagtctgaccgaaaaggagatgatgcgtgttcaccgaatcaatctacaaatgcggtagctgcaccaggttatggaaatggtggttttttccctactcagagttcacaaggcgtaacagacagcgttatcgcatttattaataatttagataaagttcctttagaacccttaaccacaggtgctatctcagttccatatgatgcattgaatcccaaaaatttacaggaatatggttatttacgagcttttgagccaaatccagctaacacgtacttaacatggcgtggaaatttaaagaaatatcatgttgttttatctggagcgaatgctggtgcttttgaggccaattctggcggattagtctacaatgcaagtggggcttttagaactggaacaaaggattattggaatagctctacttatactgacggaggcaaagtctttttaggtggttcatatgcgaatgtgcctttacccattgctggacaacctgaaacacgtgatgcagaagggaatatcacaaaatattattacgcagtacaaagtaaaatccgcaacttatttactgatgtttccgccgttgcagcagatggtagcttaacgaaaatttcaacttctggaactaatttgctaaaaattccagctgctccaccagaagaaactaatccttttgatacggtggctaatacagcaagttatgttttaggtaaatttgatccatcaactggacaaaatattttaaaagcttttcctataagcttgaaattaaaaatattaaattatttaggttattcaacagatattaatgcaacaactctgccttcatctttggttacatcgaatgaaccttatttatcgatggggggaagtattcactctttaccggttcaactaacctacaatggaaccctagatgataatgggaatttaacatctgcccgagaacaatccatcctctatggaactatggaaggcggattacatattgtggatgcttcatctggtattgagcaaatggtctttgttcctgcagatattttaaatgattcagttgcttccaaagctttagttgttggtcaaagtgatgcttcagctcccgctcatggtatggacggagcttgggtatctgatccagcctataatattactacagtgggaagtggtagctcggcagtatcaaaagtaactgcaaagcaaatgaatatttatggcggcatgcgtatgggaggaagtagctactacggactagatgtattaagccctacttcaccgaaactgctttttagaataggggcagaccagaatgactatagccgtatgggtcaaagctggtctaaacccgtactcgcgaacatccgttataacggttctattagacgcgtcctgattgttggcggtggttatgatcagtgttatgaaaaaccaaatattacgttgactgacgcttgctttaccaatggaaaagcaaaaggaaatgctgtctatattattgacgcaaaaactggtcagcgtttgtggtggacaagtgatacaggttctaatactgataacgccaatatgaagcatagtattgttagccgtattagtactttagaccgtgatgctgatggcttagtcgaccatctatattttggagatttaggcggacaaatttttcgcgtagaccttaataataatcagacaaaaaccaattcgacctatagcagttttggtgtcagagttgtgcgtttagcgaatttagcaacaaatgattcaacttatgatggcacaaatgattatacaggtgggaatgctcctcgtttttatgagcctccaacagtaacgatccacgattatggaattcacacttttattacagtaggaattgcatcaggagaccgtagtacacctttagatgtttacccactcacaggtcgtgaaggtatgacgcctgcaagtgcattaagtggacgtcctgtaaataatgtatatggaattattgatagagactttgttaaaaagaacttaatgtctttaactgataatcagcttgaaacaaaagatattacacggacaggcttaagaaaaaatccacaaattctaagaacaggtgaaacaagagtagctcaaattttcttcccaactacaggagtaggtaaaggtggttggtatcgctcgctttctagtacgagcgatggtacagaaaaagctaataatagttttcgtattaaaggaggactcaaagcttttgaggaaccaatggcaattacgggtaatttaattattctagtttatgaccctcaaggaacgggaattgttgcggcagatccttgcctacctcgtgttgtgggagaaacagaccgacaaacttattgtttaccatttggagcttgtcttaattctgatgggtcaatcgatcaaaataaagagaatcacagtgggtttgaaacacaaactggtactaattgcccagtaggggcttctgaatgtaataaaaacgttattggctctggtattcgtagcgtaacatttgtaccaacggaggataacccacctacgactaatagttgtgggaaattaaagctgtctggtaatgagcaagggactggacagtggcaatgtacgagtcatttagttcctacgcgttggtatgagcgttatcgttaa

SEQ ID NO: 90 - RNA, 9144 nt, Acinetobacter baumannii
  source 1..9144: mol_type = mRNA
atgacagacgctgctggcaatacttcggaacaggctgtacaaaaagtagttgtggatactaccgcgccgcaagcaggtgaactgactttatctgacttgagtgatacaggtatttcagcaacagatcagatcacgcaagataaaaacttcaatttaaagcttgaaggacaggaaagcggcagccgagtaacatatttagtttctaccgatgaaggaaaaacttggcaggaaaccacgatagcccaaaaagatttaactgatggtgtttaccaatataaagctgtagtgacagacgctgccggcaatacctcggaaacggctgtacaaaaagtggttgtggatactaccacaccacaagcaggtgaactgactctatctgacttgaatgatacaggcgtttcagtaacagatcagatcacgcaagataaaaacttcaatttaaagcttgaaggacaggaaactggtagccgagtaacatatttagtttccactgatgaaggaaaaacgtggcaggaaaccacgatagcccaaaaagatttggctgatggtgtttacaaatataaagctgtagtgacagacgctgccggcaatacctcggaaacggctgtacaaaaagtggttgtggatactaccgcaccgcaagcaggtaaactcactttatctgatttgaatgatacaggtgtttcagcaacagatcagattacgcaagataatagttttactttaaagctagctcaaccgattgtgattggggaacaagccgctttactagaccactatgaagtttcaaaagatgaaggaaaaacttggcaagagacaacagctgatcaaaaagatttagctgatggtatttaccaatataaagctatagtgacagaccttgcaggcaatatctcagaatctgctatacaaaaagttgttgtagataattccttaaatgttgaatcaaccacagtgattgtaaagccgattactgaagacaatacaataagtctagttgaaaaagatcaagttatttctataagacttgaaatagctaatttacccacagatttaaatagctcactgacatcagtaaatacgacattaggcaatgttacttataattttcattttgatgaagtcacgcaagaatgggttactgaaattccagcagagtttctttggtcagtagagccgcaaaccaatatatcaattgagattagtcttactgatcaagctggtaatacagccattatcaaacatacccaaaattataatgtggatcatactccgaattcaccaaccctagattcactgactttcaacaatatagatggagctatcatttcaggtagtgcatataaaggaagtaaggtcgatatctacaataaaaatggtgattggcttgcaagtacaataactaatgaagaaggtaaatttactttacaagatctttcaattaactcaaatcaagaagtttacgcagtcgctacttataatggttatagcagtgaaaattcatcaattgggttagttactgaagttccagctattagtattacacgaattagtccagaaggcgtgattagtggttatgcgactgaaggtagtcattttattgtaaaagatcagaatggaaatattttacaagaatttaattctaatgtatttgatagctctggtattacaccatttagtgtaatggcattaggcgaagtaagaccatttattttgtccctcgatcagcctttagaggaaggggctcaaattattatctctatagataaagataatatttcaggccatccacaatatattactgcagactatactccagcagtatttttagaaactccacaatttgatattagtggtgaaactttatcagtacatgttaatgaacctaacagctttattcgtgcattttctggagaaggcaatttaattgctacaggttttaccgatgagcaaggctttgcaagtttacaggtgttccaatttttaaaagaaggcgaaacggtttctgtacaagtagtagataaaaatcaaaatacgagtgaaaccctaatcgaggttccaaactttgcttatattccacatgttgaacgtattacacaagaaggcttaatttctggagttgctgaagataatagtacggtaattgtacgtgatgctgatggcaatgaattaggaaaggttacattaggtgatgataatagttggagtgactttagtcattttagcttgagtgtaaaccgccctttaattgatggagagaaaatttccgttcaaattattgataataaagggttaatgagtcctgagcaaaatatcatcgtagatctgactcctccacctgctccaacggagttaaattttaatgatgctggtgatttagtttatggtcatgcagaacctttctctgaaattttagttaaagatgggcaaggaaatattcttaataaatggttttggaataactggaccgatgagagtggaagtttctcaatagaactaggtacatttttaaccaatgctgagacggtttatgtcacggctaccgatgtaaatggaaacgtaagtttagcagctcagatacaagcacctaattatgcctttgctccatatgttgatagctttacttcggatggcgtgataagtgggcaagctgaaaataatagtactctcgttgttaaagacgccaaaggtgacgttgtagctgaaattaaagttggtgaagataacggttggaatggatcaagttattttaaactccagcttgatcgtcctcttgtcgatggtgagcagtttttcttatcaattaaggatgcacgtgggcaagtaagtgctgatactgtaattaccgctgatacggttgctcctacaccagccagcaatttagttttctcagaagatggttcatatcttacgggtgtagcagaactgaatactacgattcaggtttttgatcataatggtcagctagtgaatatatggaataacaccattaactctgacggtacatttactatttacttaggtagtaacaatttacatggagaagcattcacagtcactgttaaagaccaagccggaaatgtgagtgaggctatttcaataaacgcgccacttgacgatattgcaccgaatccaattaaaaatattttacttgatgcaaatggccaaaactttacagcacaagcagaagcaaatagtcagatcgaagtttttgattcattgggtaatcagacaggttggggttctacagatagtgcaggtaatgtttctggttctttcaatcaaacttatttacacggtgaggaactcacttttgttgttatagatcgagcaggtaatcgcagtatcgaatttaagcaaaatgctttaattgataccattgcaccaaatccgattgcaaatatcatcttcaacgaagatggccaaagttttacagctcaggcagaagccggaagttctattgatgtattagatcagactggaaataagattggttttggttacaccgatagctcaggtaatgtatctggttattttcaacaagtttacttacacggtgaggaacttacttttgtcgtgatcgatcgagcaggtaaccgcagtgcagaggtcaagcagagtgctttgaatgatgacgtcgtaccaaatccgattgaaaatattgtattagatctcaatggccagaactttacagctcaggcagaagcaaatagccaaattgaaatcaaaaataataacggtgacgtcgtaggttatggatcagcagatagtgcaggtaatgtttcaggatatttgtatcaagtgcatttacatggcgaagagctcacttttattgtagttgaccgagcgggtaaccgcagtacagaggttaagcagaatgccttgattgatgatattgcgccaaatccgattgaaaatattgtattagatatcaatgggcaaaattttacagcgcaagcagaagcaaatactcaaattgaagttaaaaatgctgttggtgagattgtaggtttaggttatgtggatggcgctggtaacgtgtcaggttatttatatcaagtctatttacatggagaagagctcacttttgttgtcgttgaccgagcgggtaaccgcagtacagaggttaagcagaatgccttgattgatgatattgcgccaaatccgattgaaaatattgtattagatatcaatgggcaaaattttacagcgcaagcagaagcaaatactcaaattgaagttaaaaatgctgttggtgagattgtaggtttaggttatgtggatggcgctggtaacgtgtcaggttatttatatcaagtctatttacatggagaagagctcacttttgttgtcgttgaccgagcgggtaaccgcagtacagaggtcaagcagaacgccttgattgatgatatcgcaccaaatccgattgagaatattttacttgatgcgaacggacagaactttacagctcaggcagaagcaaacactcaaattgaagtcaaaaatactgctggtgaagtcataggatctggttcaaccgatagtatgggtaatgtttctggttacttctatcaggtctatttacatggtgaagaactcacttttgttgtagttgatcgagctggtaaccgcagtacagaggtcaagcagaatgccttgattgatgacattgctccgaatgcgattgaaaacattatctttaatgaaaatggtcaaaactttacagcgcaagcagaagcaaatagcaaagttgaagttaaaaatgctgcgggtgaggttgtaggttctggttatgtggatagtgttggtaatgtgtcaggttacttgaatcaagtttatttaaaaggtgaggagctcacttttgttgtgattgatcaagctggtaatcgtagcattgaagtaaaacaaacagcctttcttgataatacagcaccggaaaatgcgactaatttagtatttagtgaagatggctcatatctaagtggtatggctgagccaaatgcgacgattcaaatatttgatcagtatggtcaattattaaatcagtggaataataatgttaattgggacggaacatttaacatctatttaaacagtaactacatgcatggagaagtatttaaagtagttgtagttgatcacgctggtaatttgagtggtgaggttactgtaaaagcaccgcttgatgatattgctcctgtagctgcaagtgatctggtctttaatgaagatggttcatccctttctggtgtagctgagccaaataccttcatccagatttttgatcaaaatggtcagcagatgaatacgtggagtcagagtgtaaatgctgatggtacatttactatttttttcggtacttacaatttacatggtgaagagtttacagtcattgttaaagaccttgctggaaatgtgagtgaagctgtttcagttaaggcgccgcttgatgatattgccccaaaaccgattaaaaatattgtatttgatgcaaatggccaaagctttacggcacaagcagaagcaaatagtcagattgaaatttttgactcatttggtagtcagataggttggggctctaccgatagcactggtagtgtgacgggttacttctatcaagtgtatttacatggagaagaattaacgttcgttgttatagaccgagtaggtaaccgtagtgatgaaatgaagttaaatgctttgatggataccattgcaccaaagccgattgaaaacatcatctttaatgaaaatgggcaaaattttacagcacaagcagaagccaatagttttattagtgtcaaaaatgctgcgggtgagtttgttggctatggttatgtcgatagtactggtaatgtgtccggtcacttcaatcaggtttacttaaaaggtgaggaactcacttttattgttatagataaagcaggtaatcaaagtattgaatataagcaaaatgctttaactgatgatattgcaccaaatccgattgaaaacatcgtcttaaataaaaatggacaaaactttacagcgcaagcggaagccgatagccaaatcgaagttaaaaatactgcgggtgaggtcgtaggttctggttatgtagatagtattggtaatgtgtcaggttcttttaatcaagtctatttacatggagaagagcttacttttgttgtagttgaccgagcaggtaaccgtagtacagaggtcaagcagaatgctttgattgatgatatcgcacctaatcaaattgagaatattgtttttgatgtaaatggtcagtactttacggggcatgctgaagcagatactcgaattgaagtactagatcaatttggcaatcgcgctggttggggatatgttgatagccaaggtaacgtgataggttatttcaatcaggtttatttacatggagaggaattaactttcatcgttgtagatatagctggtaaccgaagtgttgaagttaagcaaaatgctttaattgataatgtcgcaccgccagcagcagcaaatattacattaacctcagacggattgctttttggtgaggcggagccaaactcaactgttgaaattattgatcaatatggtgcagttatcacaacaacttatgtttggtacgatggcacatttaatcaatggattaatctgagtcaataccagacacaaaatttaagtattgttgtcaaagatcaagcaggtaaccgcagtgaagttgtacatgaattagtaccagtatttaccaactcaccaattgctgcaacagagctaaaactggatatagatggtcatattctgacaggtaaggcgacagtaggaatgtcggttgttgttacatctactgatggtcagaccattaacggagggtggaataatgctgtgaatgaagatggtagttttgctattcagttaaatgattactatctacagggacagacattacaagttcgagtttatgatcagaatacgaatcaatatagcctaatctctgaaattattgcgcctttagacaatattgctccagtaattaatgaagtggtaattaacaatgatggttatggtattactgggcaaactgattcaaaagcaatcattcaggtaatggatgcggatggagatttacgagcagaatttcaagcggatgagactggatattttaacgctagtatttatccacctatattacgtggagagcagttattcatcactgcgatagatttagccaaaaatataagtaagccatttaatattacttttaatgcagatactaatgcaccgccatcagcagagcatattgtggtatctgaaaatggtttttttattgagggaaccgctgtagcaattagtactgtgcatatttttgatgtgcatagtaaccatgttgccactaatgttgcagatgaagctggaaactttaatattcagttgtatccacctctagcgagtggacaaattttacgcattgttgtggaatacaatgggtatcaaagtgcttacaccgaaattacggcacctatagacactgtcgcaccaaatgcagcaactcagcttctcttagaagatggaaatgtactttcaggacaagcggaagcatattcaattgttaatatttttgatgctaataataatttggttggtcagactaatgttggaagtgatggtgctttcttaacacacctatggtatgagtattggcatggcgaaacattaacggtaaaagtagttgatgccaatcaaaatgtgagtgtggggacaacgattgttgctataaatgatacggtagttccagatgtagttacgcagcttgctatagatgaatggggttcgctcacagggagggtagaaagttatgctactgttgagcttacctatcattttactgaccaaccgctttctgtaacgagcactacagcattggcaaatggaatgttctttatctatctggatagaaatgcaacttcacttgaccttactgttattgatcgtgctggaaaccgtagtgaaaccattagccaaataattagtgatttaccgactgttattattgatcactttaaaggtgatgctacagataatacttataacattgatactatagatgattttgttcaagaatatattgttgagccgtatgcaatatataaagacgtctggattgataatagctatatgtattcagattgggtcattgaaggccattatgaacagatatggtttgttgatggatattatgaatcgcaatgggccacaagtggttattcaactgtacaaaatatttatcaaaatcaaaatggtataacttatattgataatggcacggcagatagcgattatagtcgatatgaacaacaatattatgatttcgtgaatggccaatggcaagaaggctatgaattaacttatatacgttcagaagaaggatgggttgatacaagtcattatgaagatgtttatattgatacaagtcattatgaagaggtttgggtagatactagccactatcaagatatttgggtagaaaatagttattgggagagtcaacttgttgaatctggccgtagagatgtggatttaggcgggcatgataaaattatcagttctgttaattatagtttggttggtttatatcaaacagtaaatgatccaacgactgtagattcgtttttggaaagtggtcgttatgtagaagacttggagctggttggttctgctcatctaaatgcaacaggcaatgctttagataatcttttaacaggtaactctggtaataacgttttgaatgggcgtgaaggtaacgatacctatatcactaatgaaggtactgataccattgtgttccaattgctaaatagccaagatgcaactggcgggaatggacatgacacggtgttagattttactttaggtgatataagaactaaccttcaagctgacaaaatcgatttaagtgaactattaattgactattctaaagatgtaagcgcattagctaaatttattactgtagagcaagatgctgggaacacgactattagtcttgaccgagatggcgaaggtacaatgtttaatagtgtgtcactgttaactctaaatcaagtaaatacaacattagacgaattactaaacaaccagcaaattattgtttaa

SEQ ID NO: 91 - DNA, 10 bp, synthetic construct
  source 1..10: mol_type = other DNA
attcggaacc

SEQ ID NO: 92 - DNA, 10 bp, synthetic construct
  source 1..10: mol_type = other DNA
atacgggacc

(19) United States
(12) Patent Application Publication
(10) Pub. No.: US 2024/0226263 A1
(43) Pub. Date: Jul. 11, 2024

(54) PEPTIDES DERIVED FROM ACINETOBACTER BAUMANNII AND THEIR USE IN VACCINATION

(71) Applicant: Evaxion Biotech A/S, Hørsholm (DK)

(72) Inventors: Niels Iversen Møller, Hørsholm (DK); Andreas Holm Mattson, Hørsholm (DK)

(21) Appl. No.: 18/525,109

(22) Filed: Nov. 30, 2023

Related U.S. Application Data
(63) Continuation of application No. 16/809,011, filed on Mar. 4, 2020, now Pat. No. 11,857,615.
Application No. 16/809,011 is a continuation of application No. 15/526,219, filed on May 11, 2017, now abandoned.

(30) Foreign Application Priority Data
Nov. 13, 2014 (EP) 14193008.1

(51) Int. Cl. A61K 39/104; A61K 39/00; C07K 14/21

(52) U.S. Cl. CPC A61K 39/104; C07K 14/212; A61K 2039/543; A61K 2039/55505; A61K 2039/55566; G01N 2333/22; G01N 2469/20

(57) ABSTRACT

The present invention relates to proteins derived from Acinetobacter baumanii, nucleic acids encoding the proteins, antibodies specific for the proteins as well as methods of therapy, prophylaxis, and diagnosis that utilise the proteins, nucleic acids, and antibodies.

### CROSS REFERENCE TO RELATED APPLICATIONS

In accordance with 37 C.F.R. 1.76, a claim of priority is included in an Application Data Sheet filed concurrently herewith. Accordingly, the present invention claims priority as a continuation of U.S. patent application Ser. No. 16/809,011, filed Mar. 4, 2020, which is a continuation of U.S. patent application Ser. No. 15/526,219, filed May 11, 2017, which is a § 371 national stage entry of International Application No. PCT/EP2015/076598, filed Nov. 13, 2015, which claims the benefit of the priority of European Patent Application No. 14193008.1, filed Nov. 13, 2014, the entire contents of each are incorporated herein by reference,

### REFERENCE TO AN ELECTRONIC SEQUENCE LISTING

The contents of the electronic sequence listing (4564000168Sequencelisting.xml; Size: 191 KB and Date of Creation: Nov. 27, 2023) is herein incorporated by reference in its entirety.

### FIELD OF THE INVENTION

The present invention relates to the field of antimicrobial prophylaxis and therapy. In particular the present invention relates to novel proteins and polynucleotides derived from Acinetobacter baumannii. The invention further relates to vectors comprising the polynucleotides, transformed host organisms expressing the polynucleotides, antibodies (mono- or polyclonal) specific for the polypeptides as well as diagnostic, prophylactic and therapeutic uses and methods. Finally, also methods of preparation are part of the invention.

### BACKGROUND OF THE INVENTION

Antibiotic resistance is recognized as one of the greatest threats to human health on the planet (2009; Choffnes et al., Antibiotic Resistance: Implications for Global Health and Novel Intervention Strategies, The National Academic Press, Washington, D.C., (2010); Smolinski et al., Microbial Threats to Health: Emergence, Detection, and Response, The institute of Medicine, Washington D.C., (2003); Spellberg et al., Clin Infect Dis 52(55):397-428 (2011); Spellberg et al., Clin Infect Dis 46:155-164 (2008); Walker et at., Science 325-1345-1346 (2009). In the last decade, Acinetobacter baumannii has emerged as one of the most common and highly antibiotic-resistant pathogens in the United States (US) and throughout the world (Doi et al., Emerg Infect Dis 15:980-982 (2009); Higgins et al., J Antimicrob Chemother 65-233-238 (2010); Perez et al., Antimicrob Agents Chemother 51:3471-3484 (2007). Indeed, 50-70% of A. baumannii clinical isolates are now extensively drug resistant (XDR; i.e. resistant to carbapenems and all other antibiotics except colistin or tigecycline), reflecting a >15-fold increase in just the past 10 years (Dizbay et al., Scand J Infect Dis (2010); Hidron et al., Infect Control Hosp Epidemiol 29:996-1011 (2008); Hoffmann et al., Infect Control Hosp Epidemiol 31:196-197 (2010); Kallen et at., Infect Control Hosp Epidemiol 31:528-531 (2010); Lautenbach et al., Infect Control Hosp Epidemolo 30:1186-1192 (2009); Mera et al., Drug Resist 16:209-215 (2010); Perez et al., Am J Infect Control 38:63-65 (2010); Rosenthal et al., Am J Infect Control 38:95-104 e102 (2010). Infections caused by carbapenem-resistant, XDR A. baumannii are associated with prolonged hospitalization, tremendous health care costs, and high rates of death despite treatment (Doi et al., Emerr Infect Ds 15:980-982 (2009); Falagas et al., Int J Antimicrob Agents 32:450-454 (2008); Gordon and Wareham, J Antimicrob Chemother 63:775-780 (2009); Lautenbach et at., Infect Control Hosp Epidemiol 30:1186-1192 (2009); Metan et al., Eur J Intern Med 20:540-544 (2009); Park et al., Diagn Microbiol Infect Dis 64:43-S1 (2009); Perez at al., Am J Infect Control 38:63-65 (2007); Sunenshine et al., Emerg Infect Dis 13:97-103 (2007). Indeed, bloodstream infections caused by XDR A. baumannii cause >50-60% mortality rates despite antibiotic therapy (Gordon and Wareham, J Antimicrob Chemother 63:775-780 (2009); Metan et al., Eur J Intern Med 20:540-544 (2009); Munoz-Price et at., Infect Control Hosp Epidemiol 1(10):1057-62 (2010); Park et al., Diagn Microbiol Infect Dis 64:43-51 (2009); Tseng et al., Diagn Microbiol Infect Dis 59:181-190 (2007). A major reason for these high mortality rates is that XDR A. baumannii infections are treatable only with suboptimal second-line antibacterial agents, such as tigecycline and colistin. Even more concerning is the increasing resistance of A. baumannii to both colistin and tigecycline (Adams et al., Antimicrob Agents Chemother 53:3628-3634 (2009); Doi et al., Emerg Infect Dis 15:980-982 (2009); Falagas et al., Int J Antimicrob Agents 32:450-454 (2008); Hernan et al., Diagn Microbiol infect Dis 65:188-191 (2009); Livermore et al., Int J Antimicrob Agents 35:19-24 (2010); Park et al., Diagn Microbiol Infect Dis 64:43-51 (2009); Valencia et at., Infect Control Hasp Epidemiol 30:257-263 (2009); Wang and Dowzicky, Diagn Microbiol Infect Dis 68:73-79 (2010). Such pan-drug resistant (PDR) A. baumannii Infections are resistant to every FDA approved antibiotic, and are hence untreatable.

New methods to prevent and treat A. baumannii Infections are critically needed, especially since no new drugs to treat these Infections are in the antibacterial pipeline for the coming decade (Boucher et al., Clin Infect Dis 48:1-12 (2009); Spellberg et al., Clin Infect Dis 46:155-164 (2008). Since risk factors for A. baumannii infections are understood (Beavers et al., 2009; Caricato et al, Intensive Care Med 35:1964-1969 (2009); D'Agata et al., Infect Control Hosp Epidemiol 21:588-591 (2000); Furniss et al., J Burn Care Rehabil 26:405-408 (2005); Metan et at., Eur J Intern Med 20:540-544 (2009); Zakuan et al., Trop Biomed 26:123-129 (2009), vaccination of acutely at-risk patients is a promising method to prevent such infections, and antibody-based immunotherapy has promise to improve outcomes from infection.

The first full-genome sequence of A. baumannii was performed in 2007 (strain ATCC 17978) with a chromosome of 3976747 base pairs

Vaccination is considered to be a very effective method of preventing infectious diseases in human and veterinary health care. Vaccination is the administration of Immunogenically effective amounts of antigenic material (the vaccine) to produce immunity to a disease/disease-causing pathogenic agent. Vaccines have contributed to the eradication of smallpox, the near eradication of polio, and the control of a variety of diseases, including rubella, measles, mumps, chickenpox, typhoid fever.

Before “the genomic era”, vaccines were based on killed or live attenuated, microorganisms, or parts purified from them. Subunit vaccines are considered as a modern upgrade of these types of vaccine, as the subunit vaccines contain one or more protective antigens, which are more or less the weak spot of the pathogen. Hence, in order to develop subunit vaccines, it is critical to identify the proteins, which are important for inducing protection and to eliminate others.

An antigen is said to be protective if it is able to induce protection from subsequent challenge by a disease-causing infectious agent in an appropriate animal model following immunization.

The empirical approach to subunit vaccine development, which includes several steps, begins with pathogen cultivation, followed by purification into components, and then testing of antigens for protection. Apart from being time and labour consuming, this approach has several limitations that can lead to failure. It is not possible to develop vaccines using this approach for microorganisms, which cannot easily be cultured and only allows for the identification of the antigens, which can be obtained in sufficient quantities. The empirical approach has a tendency to focus on the most abundant proteins, which in some cases are not immuno-protective. In other cases, the antigen expressed during in vivo infection is not expressed during In vitro cultivation. Furthermore, antigen discovery by use of the empirical approach demands an extreme amount of proteins in order to discover the protective antigens, which are like finding needles in the haystack. This renders It a very expensive approach, and it limits the vaccine development around diseases, which is caused by pathogens with a large genome or disease areas, which perform badly in a cost-effective perspective.

### Object of the Invention

It is an object of embodiments of the invention to provide A. baumannii derived antigenic polypeptides that may serve as constituents in vaccines against A. baumannii infections and in diagnosis of A. baumannii infections. It is also an object to provide nucleic adds, vectors, transformed cells, vaccine compositions, and other useful means for molecular cloning as well as for therapy and diagnosis with relevance for A. baumannii.

### SUMMARY OF THE INVENTION

It has been found by the present inventor(s) that A. baumannii, expresses a number of hitherto unknown putatively surface exposed proteins which are candidates as vaccine targets as well as candidates as immunizing agents for preparation of antibodies that target A. baumannii.

So, in a first aspect the present invention relates to a polypeptide comprising
a) an amino add sequence selected from the group consisting of any one of SEQ ID NOs: 1-30, or
b) an amino add sequence consisting of at least 5 contiguous amino acid residues from any one of SEQ ID NOs: 1-30, or
c) an amino acid sequence having a sequence identity of at least 60% with the amino acid sequence of a),
d) an amino acid sequence having a sequence identity of at least 60% with the amino add sequence of b), or
e) an assembly of amino acids derived from any one of SEQ 10 NOs: 1-30 which has essentially the same 3D conformation as in the protein from which said assembly is derived so as to constitute a B-cell epitope, said polypeptide being antigenic in a mammal.

In another aspect, the invention relates to an isolated nucleic acid fragment, which comprises
i) a nucleotide sequence encoding a polypeptide of the invention, or
ii) a nucleotide sequence consisting of any one of SEQ ID NOs: 31-60.
iii) a nucleotide sequence consisting of at least 10 consecutive nucleotides in any one of SEQ ID NOs: 31-60,
iv) a nucleotide sequence having a sequence identity of at least 60% with the nucleotide sequence in i) or ii),
v) a nucleotide sequence having a sequence identity of at least 60% with the nucleotide sequence in iii),
vi) a nucleotide sequence complementary to the nucleotide sequence in i)-v), or
vii) a nucleotide sequence which hybridizes under stringent conditions with the nucleotide sequence in i)-vi).

In a third aspect, the invention relates to a vector comprising the nucleic acid of the invention, such as a cloning vector or an expression vector.

In fourth aspect, the invention relates to a call which is transformed so as to carry the vector of the invention.

In a fifth aspect, the invention relates to a pharmaceutical composition comprising a polypeptide of the invention, a nucleic acid fragment of the invention, a vector of the invention, or a transformed cell of the invention, and a pharmaceutically acceptable carrier, vehicle or diluent.

In a sixth aspect, the invention relates to a method for inducing immunity in an animal by administering at least once an immunogenically effective amount of a polypeptide of the invention, a nucleic acid fragment of the invention, a vector of the invention, a transformed cell of the invention, or a pharmaceutical composition of the fifth aspect of the invention so as to induce adaptive immunity against A. baumannii in humans or animals.

In a seventh and eighth aspect, the invention relates to 1) a polyclonal antibody In which the antibodies specifically bind to at least one polypeptide of the invention, and which is essentially free from antibodies binding specifically to other A. baumannii polypeptides, and to 2) an isolated monoclonal antibody or antibody analogue which binds specifically to a polypeptide of the invention. In a related ninth aspect, the invention relates to a pharmaceutical composition comprising such a polyclonal or monoclona antibody and a pharmaceutically acceptable carrier, vehicle or diluent.

In a 10^{th} aspect, the invention relates to a method for prophylaxis, treatment or amelioration of infection with A. baumannii, comprising administering a therapeutically effective amount of an antibody of the 7^{th} or 8^{th} aspect of the invention or a pharmaceutical composition of the eighth aspect to an individual in need thereof.

In an 11^{th} aspect, the invention relates to a method for determining, quantitatively or qualitatively, the presence of A. baumannii, in particular the presence of multi-resistant A. baumannii, in a sample, the method comprising contacting the sample with an antibody of aspects 8 or 9 of the invention and detecting the presence of antibody bound to material in the sample.

In an 12^{th} aspect of the invention is provided a method for determining, quantitatively or qualitatively, the presence of antibodies specific for A. baumannii, in particular the presence of antibodies specific for multi-resistant A. baumannii, in a sample, the method comprising contacting the sample with a polypeptide of the invention and detecting the presence of antibody that specifically bind said polypeptide.

In a 13^{th} aspect, the invention relates to a method for determining, quantitatively or qualitatively, the presence of a nucleic acid characteristic of A. baumannii, in particular the presence of a nucleic acid characteristic of multi-resistant A. baumannii, in a sample, the method comprising contacting the sample with a nucleic acid fragment of the invention and detecting the presence of nucleic acid in the sample that hybridizes to said nucleic acid fragment.

In a 14^{th} aspect, the invention relates to a method for the preparation of the polypeptide of the invention, comprising
culturing a transformed cell of the present invention, which is capable of expressing the nucleic acid of the invention, under conditions that facilitate that the transformed cell expresses the nucleic acid fragment of the invention, which encodes a polypeptide of the invention, and subsequently recovering said polypeptide, or
preparing said polypeptide by means of solid or liquid phase peptide synthesis.

In a 15^{th} aspect, the invention relates to a method for determining whether a substance, such as an antibody, is potentially useful for treating infection with A. baumannii, the method comprising contacting the polypeptide of the invention with the substance and subsequently establishing whether the substance has at least one of the following characteristics:
1) the ability to bind specifically to said polypeptide,
2) the ability to compeed with said polypeptide for specific binding to a ligand/receptor, and
3) the ability to specifically inactivate said polypeptide.

Finally, in a 16^{th} aspect, the invention relates to a method for determining whether a substance, such as a nucleic acid, is potentially useful for treating infection with A. baumannii, the method comprising contacting the substance with the nucleic acid fragment of claim of the invention and subsequently establishing whether the substance has either the ability to
1) bind specifically to the nucleic acid fragment, or
2) bind specifically to a nucleic acid that hybridizes specifically with the nucleic acid fragment.

### DETAILED DISCLOSURE OF THE INVENTION

### Definitions

The term “polypeptide” is in the present context intended to mean both short peptides of from 2 to 10 amino acid residues, oligopeptides of from 11 to 100 amino acid residues, and polypeptides of more than 100 amino acid residues. Further-more, the term is also intended to include proteins, i.e. functional biomolecules comprising at least one polypeptide; when comprising at least two polypeptides, these may form complexes, be covalently linked, or may be non-covalently linked. The polypeptide (s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring amino acid sequence or nucleic acid sequence, respectively

The term “amino acid sequence” s the order in which amino acid residues, connected by peptide bonds, lie in the chain in peptides and proteins.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Sequence identity” is in the context of the present invention determined by comparing 2 optimally aligned sequences of equal length (e.g. DNA, RNA or amino acid) according to the following formula: (N_{ref}−N_{dif})·100/N_{ref}, wherein N_{ref} is the number of residues in one of the 2 sequences and N_{dif} is the number of residues which are non-identical in the two sequences when they are aligned over their entire lengths and in the same direction. So, two sequences 5′-ATTCGGAACC-3′ (SEQ ID No. 91) and 5′-ATACGGGACC-3′ (SEQ ID NO. 92) will provide the sequence identity 80% (N_{ref}=10 and N_{dif}=2).

An “assembly of amino acids” means two or more amino acids bound together by physical or chemical means.

The “3D conformation” is the 3 dimensional structure of a biomolecule such as a protein. In monomeric polypeptides/proteins, the 3D conformation is also termed “the tertiary structure” and denotes the relative locations in 3 dimensional space of the amino acid residues forming the polypeptide.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immunogenic by 5-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class 1 molecule) In an antigen-presenting cell and which stimulates T-helper lymphocytes in an animal species as a consequence of T-cell receptor recognition of the complex between the peptide and the MHC Class II molecule prese

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing Immunity—an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate,

An “adaptive immune response” is an immune response in response to confrontation with an antigen or immunogen, where the immune response is specific for antigene determinants of the antigen/immunogen—examples of adaptive immune responses are induction of antigen specific antibody production or antigen specific induction/activation of T helper lymphocytes or cytotoxic lymphocytes,

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

Hybridization under “stringent conditions” is herein defined as hybridization performed under conditions by which a probe will hybridize to its target sequence, to a detectably greater degree than to other sequences. Stringent conditions are target-sequence-dependent and will differ depending on the structure of the polynucleotide. By controlling the stringency of the hybridization and/or washing conditions, target sequences can be identified which are 100% complementary to a probe (homologous probing). Alternatively, stringency conditions can be adjusted to allow some mismatching in sequences so that lower degrees of similarity are detected (heterologous probing). Specificity is typically the function of post-hybridization washes, the critical factors being the ionic strength and temperature of the final wash solution. Generally, stringent wash temperature conditions are selected to be about 5° C. to about 2° C. lower than the melting point (Tm) for the specific sequence at a defined ionic strength and pH. The melting point, or denaturation, of DNA occurs over a narrow temperature range and represents the disruption of the double helix into its complementary single strands. The process is described by the temperature of the midpoint of transition, Tm, which is also called the melting temperature. Formulas are available in the art for the determination of melting temperatures.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention substantially all will mount an immune response against the immunogen of the present invention.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

“Specific binding” denotes binding between two substances which goes beyond binding of either substance to randomly chosen substances and also goes beyond simple association between substances that tend to aggregate because they share the same overall hydrophobicity or hydrophilicity. As such, specific binding usually involves a combination of electrostatic and other interactions between two conformationally complementary areas on the two substances, meaning that the substances can “recognize” each other in a complex mixture.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage—both these infectious agents are capable of introducing a heterologous nucleic acid sequence

The term “expression vector” refers to a vector containing a nucleic acid sequence coding for at least part of a gene product capable of being transcribed. In some cases, when the transcription product is an mRNA molecule, this is In turn translated into a protein, polypeptide, or peptide.

### SPECIFIC EMBODIMENTS OF THE INVENTION

### The Polypeptides of the Invention

In some embodiments the at least 5 contiguous amino acids referred to in option b) in the definition of the first aspect of the invention may preferably constitute at least or exactly or at most 6, such as at least or exactly or at most 7, at least or exactly or at most 8, at least or exactly or at most 9, at least or exactly or at most 10, at least or exactly or at most 11, at least or exactly or at most 12, at least or exactly or at most 13, at least or exactly or at most 14, at least or exactly or at most 15, at least or exactly or at most 16, at least or exactly or at most 17, at least or exactly or at most 18, at least or exactly or at most 19, at least or exactly or at most 20, at least or exactly or at most 21, at least or exactly or at most 22, at least or exactly or at most 23, at least or exactly or at most 24, at least or exactly or at most 25, at least or exactly or at most 26, at least or exactly or at most 27 at least or exactly or at most 28, at least or exactly or at most 29, at least or exactly or at most 30, at least or exactly or at most 31, at least or exactly or at most 32, at least or exactly or at most 33, at least or exactly or at most 34, at least or exactly or at most 35 and at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55 and at least or exactly or at most 56, at least or exactly or at most 57, at least or exactly or at most 58, at least or exactly or at most 59, or at least or exactly or at most 60 contiguous amino add residues.

The number may, where applicable, be higher, Another way to phrase this is that for each of SEQ ID NOs: 1-30, the number of the contiguous amino add residues is at least N−n, where N is the length of the sequence ID in question and n is any integer between 6 and N−1; that is, the at least 5 contiguous amino acids can be at least any number between 5 and the length of the reference sequence minus one, in increments of one. Consequently:

Insofar as embodiment b relates to SEQ ID NO: 2-30, the at least 5 contiguous amino acids referred to in option b) in the definition of the first aspect of the invention will preferably constitute at least or exactly or at most 61, at least or exactly or at most 62, at least or exactly or at most 63, at least or exactly or at most 64, at least or exactly or at most 65, at least or exactly or at most 66 contiguous amino acid residues.

Insofar as embodiment b relates to SEQ ID NO: 4-30, the at least 5 contiguous amino acids referred to in option b) in the definition of the first aspect of the invention will preferably constitute at least or exactly or at most 67, at least or exactly or at most 68, at least or exactly or at most 69, at least or exactly or at most 70, at least or exactly or at most 71, at least or exactly or at most 72, at least or exactly or at most 73 contiguous amino acid residues.

Insofar as embodiment b relates to SEQ MD NO: 5-30, the at least 5 contiguous amino acids referred to in option b) in the definition of the first aspect of the invention will preferably constitute at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94, at least or exactly or at most 95, at least or exactly or at most 96, at least or exactly or at most 97, at least or exactly or at most 98, at least or exactly or at most 99, at least or exactly or at most 100, at least or exactly or at most 101, at least or exactly or at most 102, at least or exactly or at most 103, at least or exactly or at most 104, at least or exactly or at most 105, at least or exactly or at most 106, at least or exactly or at most 107, at least or exactly or at most 108 contiguous amino acid residues.

Insofar as embodiment b relates to SEQ ID NO: 6-30, the at least 5 contiguous amino adds referred to in option b) in the definition of the first aspect of the invention will preferably constitute at least or exactly or at most 109, at least or exactly or at most 110, at least or exactly or at most 111, at least or exactly or at most 112, at least or exactly or at most 113, at least or exactly or at most 114 contiguous amino acid residues.

Insofar as embodiment b relates to SEQ ID NO: 7-30, the at least 5 contiguous amino acids referred to in option b) in the definition of the first aspect of the invention will preferably constitute at least or exactly or at most 115, at least or exactly or at most 116, at least or exactly or at most 117, at least or exactly or at most 118, at least or exactly or at most 119, at least or exactly or at most 120, at least or exactly or at most 121, at least or exactly or at most 122, at least or exactly or at most 123, at least or exactly or at most 124, at least or exactly or at most 125, at least or exactly or at most 126 contiguous amino acid residues.

Insofar as embodiment b relates to SEQ ID NO: 8-30, the at least 5 contiguous amino acids referred to in option b) in the definition of the first aspect of the invention will preferably constitute at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, at least or exactly or at most 139 contiguous amino acid residues.

Insofar as embodiment b relates to SEQ ID NO: 9-30, the at least 5 contiguous amino acids referred to In option b) in the definition of the first aspect of the invention will preferably constitute at least or exactly or at most 140, at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, at least or exactly or at most 150, at least or exactly or at most 151, at least or exactly or at most 152, at least or exactly or at most 153, at least or exactly or at most 154, at least or exactly or at most 155, at least or exactly or at most 156, at least or exactly or at most 157, at least or exactly or at most 158, at least or exactly or at most 159 contiguous amino acid residues.

Insofar as embodiment b relates to SEQ ID NO: 10-30, the at least 5 contiguous amino acids referred to in option b) in the definition of the first aspect of the invention will preferably constitute at least or exactly or at most 160, at least or exactly or at most 161, at least or exactly or at most 162 contiguous amino acid residues.

Insofar as embodiment b relates to SEQ ID NO: 11-30, the at least 5 contiguous amino acids referred to in option b) In the definition of the first aspect of the invention will preferably constitute at least or exactly or at most 163, at least or exactly or at most 164, at least or exactly or at most 165, at least or exactly or at most 166 contiguous amino acid residues.

Insofar as embodiment b relates to SEQ ID NO: 12-30, the at least 5 contiguous amino acids referred to In option b) In the definition of the first aspect of the invention will preferably constitute at least or exactly or at most 167, at least or exactly or at most 168, at least or exactly or at most 169, at least or exactly or at most 170, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182, at least or exactly or at most 183, at least or exactly or at most 184, at least or exactly or at most 185, at least or exactly or at most 186, at least or exactly or at most 187, at least or exactly or at most 188, at least or exactly or at most 189, at least or exactly or at most 190, at least or exactly or at most 191, at least or exactly or at most 192, at least or exactly or at most 193, at least or exactly or at most 194, at least or exactly or at most 195, at least or exactly or at most 196, at least or exactly or at most 197, at least or exactly or at most 198, at least or exactly or at most 199, at least or exactly or at most 200, at least or exactly or at most 201, at least or exactly or at most 202, at least or exactly or at most 203, at least or exactly or at most 204, at least or exactly or at most 205, at least or exactly or at most 206, at least or exactly or at most 207, at least or exactly or at most 208, at least or exactly or at most 209, at least or exactly or at most 210, at least or exactly or at most 211, at least or exactly or at most 212, at least or exactly or at most 213, at least or exactly or at most 214, at least or exactly or at most 215, at least or exactly or at most 216, at least or exactly or at most 217, at least or exactly or at most 218, at least or exactly or at most 219, at least or exactly or at most 220, at least or exactly or at most 221, at least or exactly or at most 222, at least or exactly or at most 223, at least or exactly or at most 224, at least or exactly or at most 225, at least or exactly or at most 226, at least or exactly or at most 227, at least or exactly or at most 228, at least or exactly or at most 229, at least or exactly or at most 230 contiguous amino acid residues.

Insofar as embodiment b relates to SEQ ID NO: 13-30, the at least 5 Contiguous amino acids referred to in option b) in the definition of the first aspect of the invention will preferably constitute at least or exactly or at most 231, at least or exactly or at most 232, at least or exactly or at most 233, at least or exactly or at most 234, at least or exactly or at most 235, at least or exactly or at most 236, at least or exactly or at most 237, at least or exactly or at most 238 contiguous amino acid residues.

Insofar as embodiment b relates to SEQ ID NO: 14-30, the at least 5 contiguous amino acids referred to in option b) in the definition of the first aspect of the invention will preferably constitute at least or exactly or at most 239, at least or exactly or at most 240, at least or exactly or at most 241, at least or exactly or at most 242, at least or exactly or at most 243, at least or exactly or at most 244, at least or exactly or at most 245, at least or exactly or at most 246, at least or exactly or at most 247, at least or exactly or at most 248, at least or exactly or at most 249, at least or exactly or at most 250, at least or exactly or at most 251, at least or exactly or at most 252, at least or exactly or at most 253 contiguous amino acid residues.

Insofar as embodiment b relates to SEQ ID NO: 15-30, the at least 5 contiguous amino acids referred to in option b) in the definition of the first aspect of the invention will preferably constitute at least or exactly or at most 254, at least or exactly or at most 255, at least or exactly or at most 256, at least or exactly or at most 257, at least or exactly or at most 258, at least or exactly or at most 259, at least or exactly or at most 260, at least or exactly or at most 261, at least or exactly or at most 262, at least or exactly or at most 263, at least or exactly or at most 264, at least or exactly or at most 265, at least or exactly or at most 266, at least or exactly or at most 267, at least or exactly or at most 268, at least or exactly or at most 269, at least or exactly or at most 270, at least or exactly or at most 271, at least or exactly or at most 272, at least or exactly or at most 273, at least or exactly or at most 274, at least or exactly or at most 275, at least or exactly or at most 276, at least or exactly or at most 277, at least or exactly or at most 278, at least or exactly or at most 279, at least or exactly or at most 280, at least or exactly or at most 281, at least or exactly or at most 282, at least or exactly or at most 283, at least or exactly or at most 284, at least or exactly or at most 285, at least or exactly or at most 286, at least or exactly or at most 287, at least or exactly or at most 288, at least or exactly or at most 289, at least or exactly or at most 290, at least or exactly or at most 291, at least or exactly or at most 292, at least or exactly or at most 293, at least or exactly or at most 294 contiguous amino acid residues.

Insofar as embodiment b relates to SEQ 10 NO: 16-30, the at least 5 contiguous amino acids referred to in option b) in the definition of the first aspect of the invention will preferably constitute at least or exactly or at most 295, at least or exactly or at most 296, at least or exactly or at most 297, at least or exactly or at most 298, at least or exactly or at most 299, at least or exactly or at most 300, at least or exactly or at most 301, at least or exactly or at most 302, at least or exactly or at most 303, at least or exactly or at most 304, at least or exactly or at most 305, at least or exactly or at most 306, at least or exactly or at most 307, at least or exactly or at most 308, at least or exactly or at most 309, at least or exactly or at most 310, at least or exactly or at most 311, at least or exactly or at most 312, at least or exactly or at most 313, at least or exactly or at most 314, at least or exactly or at most 315, at least or exactly or at most 316, at least or exactly or at most 317, at least or exactly or at most 318, at least or exactly or at most 319, at least or exactly or at most 320, at least or exactly or at most 321, at least or exactly or at most 322, at least or exactly or at most 323, at least or exactly or at most 324, at least or exactly or at most 325, at least or exactly or at most 326, at least or exactly or at most 327, at least or exactly or at most 328, at least or exactly or at most 329, at least or exactly or at most 330, at least or exactly or at most 331, at least or exactly or at most 332, at least or exactly or at most 333, at least or exactly or at most 334, at least or exactly or at most 335, at least or exactly or at most 336, at least or exactly or at most 337, at least or exactly or at most 338, at least or exactly or at most 339, at least or exactly or at most 340, at least or exactly or at most 341, at least or exactly or at most 342, at least or exactly or at most 343, at least or exactly or at most 344, at least or exactly or at most 345, at least or exactly or at most 346, at least or exactly or at most 347, at least or exactly or at most 348, at least or exactly or at most 349, at least or exactly or at most 350, at least or exactly or at most 351, at least or exactly or at most 352, at least or exactly or at most 353, at least or exactly or at most 354, at least or exactly or at most 355, at least or exactly or at most 356 contiguous amino acid residues.

Insofar as embodiment b relates to SEQ ID NO: 17-30, the at least 5 contiguous amino acids referred to In option b) In the definition of the first aspect of the invention will preferably constitute at least or exactly or at most 357, at least or exactly or at most 358, at least or exactly or at most 359, at least or exactly or at most 360, at least or exactly or at most 361, at least or exactly or at most 362, at least or exactly or at most 363, at least or exactly or at most 364, at least or exactly or at most 365, at least or exactly or at most 366, at least or exactly or at most 367 contiguous amino acid residues.

Insofar as embodiment b relates to SEQ ID NO: 18-30, the at least 5 contiguous amino acids referred to In option b) in the definition of the first aspect of the invention will preferably constitute at least or exactly or at most 368, at least or exactly or at most 369, at least or exactly or at most 370, at least or exactly or at most 371, at least or exactly or at most 372, at least or exactly or at most 373, at least or exactly or at most 374, at least or exactly or at most 375, at least or exactly or at most 376, at least or exactly or at most 377, at least or exactly or at most 378, at least or exactly or at most 379, at least or exactly or at most 380, at least or exactly or at most 381, at least or exactly or at most 382, at least or exactly or at most 383, at least or exactly or at most 384, at least or exactly or at most 385, at least or exactly or at most 386, at least or exactly or at most 387, at least or exactly or at most 388, at least or exactly or at most 389, at least or exactly or at most 390, at least or exactly or at most 391, at least or exactly or at most 392, at least or exactly or at most 393, at least or exactly or at most 394, at least or exactly or at most 395, at least or exactly or at most 396, at least or exactly or at most 397, at least or exactly or at most 398, at least or exactly or at most 399, at least or exactly or at most 400, at least or exactly or at most 401, at least or exactly or at most 402, at least or exactly or at most 403, at least or exactly or at most 404, at least or exactly or at most 405, at least or exactly or at most 406, at least or exactly or at most 407, at least or exactly or at most 408, at least or exactly or at most 409, at least or exactly or at most 410, at least or exactly or at most 411, at least or exactly or at most 412, at least or exactly or at most 413, at least or exactly or at most 414, at least or exactly or at most 415, at least or exactly or at most 416, at least or exactly or at most 417, at least or exactly or at most 418, at least or exactly or at most 419, at least or exactly or at most 420 contiguous amino acid residues.

Insofar as embodiment b relates to SEQ ID NO: 19-30, the at least 5 contiguous amino acids referred to in option b) in the definition of the first aspect of the invention will preferably constitute at least or exactly or at most 421, at least or exactly or at most 422, at least or exactly or at most 423, at least or exactly or at most 424, at least or exactly or at most 425, at least or exactly or at most 426, at least or exactly or at most 427, at least or exactly or at most 428, at least or exactly or at most 429, at least or exactly or at most 430, at least or exactly or at most 431, at least or exactly or at most 432, at least or exactly or at most 433, at least or exactly or at most 434, at least or exactly or at most 435, at least or exactly or at most 436, at least or exactly or at most 437, at least or exactly or at most 438, at least or exactly or at most 439, at least or exactly or at most 440, at least or exactly or at most 441, at least or exactly or at most 442, at least or exactly or at most 443, at least or exactly or at most 444, at least or exactly or at most 445 contiguous amino acid residues.

Insofar as embodiment b relates to SEQ ID NO: 20-30, the at least 5 contiguous amino acids referred to In option b) in the definition of the first aspect of the invention will preferably constitute at least or exactly or at most 446, at least or exactly or at most 447, at least or exactly or at most 448, at least or exactly or at most 449, at least or exactly or at most 450, at least or exactly or at most 451, at least or exactly or at most 452, at least or exactly or at most 453, at least or exactly or at most 454, at least or exactly or at most 455, at least or exactly or at most 456, at least or exactly or at most 457, at least or exactly or at most 458, at least or exactly or at most 459, at least or exactly or at most 460, at least or exactly or at most 461, at least or exactly or at most 462, at least or exactly or at most 463, at least or exactly or at most 464, at least or exactly or at most 465, at least or exactly or at most 466, at least or exactly or at most 467, at least or exactly or at most 468, at least or exactly or at most 469, at least or exactly or at most 470, at least or exactly or at most 471, at least or exactly or at most 472, at least or exactly or at most 473, at least or exactly or at most 474, at least or exactly or at most 475, at least or exactly or at most 476, at least or exactly or at most 477, at least or exactly or at most 478, at least or exactly or at most 479, at least or exactly or at most 480, at least or exactly or at most 481, at least or exactly or at most 482, at least or exactly or at most 483, at least or exactly or at most 484, at least or exactly or at most 485, at least or exactly or at most 486, at least or exactly or at most 487, at least or exactly or at most 488, at least or exactly or at most 489, at least or exactly or at most 490, at least or exactly or at most 491, at least or exactly or at most 492, at least or exactly or at most 493, at least or exactly or at most 494, at least or exactly or at most 495, at least or exactly or at most 496, at least or exactly or at most 497, at least or exactly or at most 498, at least or exactly or at most 499, at least or exactly or at most 500, at least or exactly or at most 501, at least or exactly or at most 502, at least or exactly or at most 503, at least or exactly or at most 504, at least or exactly or at most 505, at least or exactly or at most 506, at least or exactly or at most 507, at least or exactly or at most 508, at least or exactly or at most 509, at least or exactly or at most 510, at least or exactly or at most 511, at least or exactly or at most 512, at least or exactly or at most 513, at least or exactly or at most 514, at least or exactly or at most 515, at least or exactly or at most 516, at least or exactly or at most 517, at least or exactly or at most 518, at least or exactly or at most 519, at least or exactly or at most 520, at least or exactly or at most 521, at least or exactly or at most 522, at least or exactly or at most 523, at least or exactly or at most 524, at least or exactly or at most 525, at least or exactly or at most 526, at least or exactly or at most 527, at least or exactly or at most 528, at least or exactly or at most 529, at least or exactly or at most 530, at least or exactly or at most 531, at least or exactly or at most 532, at least or exactly or at most 533, at least or exactly or at most 534, at least or exactly or at most 535, at least or exactly or at most 536, at least or exactly or at most 537, at least or exactly or at most 538, at least or exactly or at most 539, at least or exactly or at most 540, at least or exactly or at most 541, at least or exactly or at most 542, at least or exactly or at most 543, at least or exactly or at most 544, at least or exactly or at most 545, at least or exactly or at most 546, at least or exactly or at most 547, at least or exactly or at most 548, at least or exactly or at most 549, at least or exactly or at most 550, at least or exactly or at most 551, at least or exactly or at most 552, at least or exactly or at most 553, at least or exactly or at most 554, at least or exactly or at most 555, at least or exactly or at most 556, at least or exactly or at most 557, at least or exactly or at most 558, at least or exactly or at most $59, at least or exactly or at most 560, at least or exactly or at most 561, at least or exactly or at most 562, at least or exactly or at most 563, at least or exactly or at most 564, at least or exactly or at most 565, at least or exactly or at most 566, at least or exactly or at most 567, at least or exactly or at most 568, at least or exactly or at most 569, at least or exactly or at most 570, at least or exactly or at most 571, at least or exactly or at most 572, at least or exactly or at most 573, at least or exactly or at most 574, at least or exactly or at most 575, at least or exactly or at most 576, at least or exactly or at most 577, at least or exactly or at most 578, at least or exactly or at most 579, at least or exactly or at most 580, at least or exactly or at most 581, at least or exactly or at most 582, at least or exactly or at most 583, at least or exactly or at most 584, at least or exactly or at most 585, at least or exactly or at most 586, at least or exactly or at most 587, at least or exactly or at most 588, at least or exactly or at most 589, at least or exactly or at most 590, at least or exactly or at most 591, at least or exactly or at most 592, at least or exactly or at most 593, at least or exactly or at most 594, at least or exactly or at most 595, at least or exactly or at most 596, at least or exactly or at most 597, at least or exactly or at most 598, at least or exactly or at most 599, at least or exactly or at most 600, at least or exactly or at most 601, at least or exactly or at most 602, at least or exactly or at most 603, at least or exactly or at most 604, at least or exactly or at most 605, at least or exactly or at most 606, at least or exactly or at most 607, at least or exactly or at most 608, at least or exactly or at most 609, at least or exactly or at most 610, at least or exactly or at most 611, at least or exactly or at most 612, at least or exactly or at most 613, at least or exactly or at most 614, at least or exactly or at most 615, at least or exactly or at most 616, at least or exactly or at most 617, at least or exactly or at most 618, at least or exactly or at most 619, at least or exactly or at most 620, at least or exactly or at most 621, at least or exactly or at most 622, at least or exactly or at most 623, at least or exactly or at most 624, at least or exactly or at most 625, at least or exactly or at most 626, at least or exactly or at most 627, at least or exactly or at most 628, at least or exactly or at most 629, at least or exactly or at most 630, at least or exactly or at most 631, at least or exactly or at most 632, at least or exactly or at most 633, at least or exactly or at most 634, at least or exactly or at most 635, at least or exactly or at most 636, at least or exactly or at most 637, at least or exactly or at most 638, at least or exactly or at most 639, at least or exactly or at most 640, at least or exactly or at most 641, at least or exactly or at most 642, at least or exactly or at most 643, at least or exactly or at most 644, at least or exactly or at most 645, at least or exactly or at most 646, at least or exactly or at most 647, at least or exactly or at most 648, at least or exactly or at most 649, at least or exactly or at most 650, at least or exactly or at most 651, at least or exactly or at most 652, at least or exactly or at most 653, at least or exactly or at most 654, at least or exactly or at most 655, at least or exactly or at most 656, at least or exactly or at most 657, at least or exactly or at most 658, at least or exactly or at most 659, at least or exactly or at most 660, at least or exactly or at most 661, at least or exactly or at most 662, at least or exactly or at most 663, at least or exactly or at most 664, at least or exactly or at most 665, at least or exactly or at most 666, at least or exactly or at most 667, at least or exactly or at most 668, at least or exactly or at most 669, at least or exactly or at most 670, at least or exactly or at most 671, at least or exactly or at most 672, at least or exactly or at most 673, at least or exactly or at most 674, at least or exactly or at most 675, at least or exactly or at most 676, at least or exactly or at most 677, at least or exactly or at most 678, at least or exactly or at most 679, at least or exactly or at most 680, at least or exactly or at most 681, at least or exactly or at most 682, at least or exactly or at most 683, at least or exactly or at most 684, at least or exactly or at most 685, at least or exactly or at most 686, at least or exactly or at most 687, at least or exactly or at most 688, at least or exactly or at most 689, at least or exactly or at most 690, at least or exactly or at most 691, at least or exactly or at most 692, at least or exactly or at most 693, at least or exactly or at most 694, at least or exactly or at most 695, at least or exactly or at most 696, at least or exactly or at most 697, at least or exactly or at most 698, at least or exactly or at most 699, at least or exactly or at most 700, at least or exactly or at most 701, at least or exactly or at most 702, at least or exactly or at most 703, at least or exactly or at most 704, at least or exactly or at most 705, at least or exactly or at most 706, at least or exactly or at most 707, at least or exactly or at most 708, at least or exactly or at most 709, at least or exactly or at most 710, at least or exactly or at most 711 contiguous amino acid residues.

Insofar as embodiment b relates to SEQ ID NO: 21-30, the at least 5 contiguous amino acids referred to in option b) in the definition of the first aspect of the invention will preferably constitute at least or exactly or at most 712, at least or exactly or at most 713, at least or exactly or at most 714, at least or exactly or at most 715, at least or exactly or at most 716, at least or exactly or at most 717, at least or exactly or at most 718, at least or exactly or at most 719, at least or exactly or at most 720, at least or exactly or at most 721, at least or exactly or at most 722, at least or exactly or at most 723, at least or exactly or at most 724, at least or exactly or at most 725, at least or exactly or at most 726, at least or exactly or at most 727 contiguous amino acid residues.

Insofar as embodiment b relates to SEQ ID NO: 22-30, the at least 5 contiguous amino acids referred to in option b) in the definition of the first aspect of the invention will preferably constitute at least or exactly or at most 728 contiguous amino acid residues.

Insofar as embodiment b relates to SEQ ID NO: 23-30, the at least 5 contiguous amino acids referred to in option b) in the definition of the first aspect of the invention will preferably constitute at least or exactly or at most 729, at least or exactly or at most 730, at least or exactly or at most 731, at least or exactly or at most 732, at least or exactly or at most 733, at least or exactly or at most 734, at least or exactly or at most 735, at least or exactly or at most 736, at least or exactly or at most 737, at least or exactly or at most 738, at least or exactly or at most 739, at least or exactly or at most 740, at least or exactly or at most 741, at least or exactly or at most 742, at least or exactly or at most 743, at least or exactly or at most 744, at least or exactly or at most 745, at least or exactly or at most 746, at least or exactly or at most 747, at least or exactly or at most 748, at least or exactly or at most 749, at least or exactly or at most 750, at least or exactly or at most 751, at least or exactly or at most 752, at least or exactly or at most 753, at least or exactly or at most 754 contiguous amino acid residues.

Insofar as embodiment b relates to SEQ ID NO: 24-30, the at least 5 contiguous amino acids referred to in option b) in the definition of the first aspect of the invention will preferably constitute at least or exactly or at most 755, at least or exactly or at most 756, at least or exactly or at most 757, at least or exactly or at most 758, at least or exactly or at most 759, at least or exactly or at most 760, at least or exactly or at most 761, at least or exactly or at most 762, at least or exactly or at most 763, at least or exactly or at most 764, at least or exactly or at most 765, at least or exactly or at most 766, at least or exactly or at most 767, at least or exactly or at most 768, at least or exactly or at most 769, at least or exactly or at most 770, at least or exactly or at most 771, at least or exactly or at most 772, at least or exactly or at most 773, at least or exactly or at most 774, at least or exactly or at most 775, at least or exactly or at most 776, at least or exactly or at most 777, at least or exactly or at most 778, at least or exactly or at most 779, at least or exactly or at most 780, at least or exactly or at most 781, at least or exactly or at most 782, at least or exactly or at most 783, at least or exactly or at most 784, at least or exactly or at most 785, at least or exactly or at most 786, at least or exactly or at most 787, at least or exactly or at most 788, at least or exactly or at most 789, at least or exactly or at most 790, at least or exactly or at most 791, at least or exactly or at most 792, at least or exactly or at most 793, at least or exactly or at most 794, at least or exactly or at most 795, at least or exactly or at most 796, at least or exactly or at most 797, at least or exactly or at most 798, at least or exactly or at most 799, at least or exactly or at most 800, at least or exactly or at most 801, at least or exactly or at most 802, at least or exactly or at most 803, at least or exactly or at most 804, at least or exactly or at most 805, at least or exactly or at most 806, at least or exactly or at most 807, at least or exactly or at most 808, at least or exactly or at most 809, at least or exactly or at most 810, at least or exactly or at most 811, at least or exactly or at most 812, at least or exactly or at most 813, at least or exactly or at most 814, at least or exactly or at most 815, at least or exactly or at most 816, at least or exactly or at most 817, at least or exactly or at most 818, at least or exactly or at most 819, at least or exactly or at most 820, at least or exactly or at most 821, at least or exactly or at most 822, at least or exactly or at most 823, at least or exactly or at most 824, at least or exactly or at most 825, at least or exactly or at most 826, at least or exactly or at most 827, at least or exactly or at most 828, at least or exactly or at most 829, at least or exactly or at most 830, at least or exactly or at most 831, at least or exactly or at most 832, at least or exactly or at most 833, at least or exactly or at most 834, at least or exactly or at most 835, at least or exactly or at most 836, at least or exactly or at most 837, at least or exactly or at most 838, at least or exactly or at most 839, at least or exactly or at most 840, at least or exactly or at most 841, at least or exactly or at most 842, at least or exactly or at most 843, at least or exactly or at most 844, at least or exactly or at most 845, at least or exactly or at most 846, at least or exactly or at most 847, at least or exactly or at most 848, at least or exactly or at most 849, at least or exactly or at most 850, at least or exactly or at most 851, at least or exactly or at most 852, at least or exactly or at most 853, at least or exactly or at most 854, at least or exactly or at most 855, at least or exactly or at most 856, at least or exactly or at most 857, at least or exactly or at most 858, at least or exactly or at most 859, at least or exactly or at most 860, at least or exactly or at most 861, at least or exactly or at most 862, at least or exactly or at most 863, at least or exactly or at most 864, at least or exactly or at most 865, at least or exactly or at most 866, at least or exactly or at most 867, at least or exactly or at most 868, at least or exactly or at most 869, at least or exactly or at most 870, at least or exactly or at most 871, at least or exactly or at most 872, at least or exactly or at most 873, at least or exactly or at most 874, at least or exactly or at most 875, at least or exactly or at most 876, at least or exactly or at most 877, at least or exactly or at most 878, at least or exactly or at most 879, at least or exactly or at most 880, at least or exactly or at most 881, at least or exactly or at most 882, at least or exactly or at most 883, at least or exactly or at most 884, at least or exactly or at most 885, at least or exactly or at most 886, at least or exactly or at most 887, at least or exactly or at most 888, at least or exactly or at most 889, at least or exactly or at most 890, at least or exactly or at most 891, at least or exactly or at most 892, at least or exactly or at most 893, at least or exactly or at most 894, at least or exactly or at most 895, at least or exactly or at most 896, at least or exactly or at most 897, at least or exactly or at most 898, at least or exactly or at most 899, at least or exactly or at most 900, at least or exactly or at most 901, at least or exactly or at most 902, at least or exactly or at most 903, at least or exactly or at most 904, at least or exactly or at most 905, at least or exactly or at most 906 contiguous amino acid residues.

Insofar as embodiment b relates to SEQ ID NO: 25-30, the at least 5 contiguous amino acids referred to in option b) in the definition of the first aspect of the invention will preferably constitute at least or exactly or at most 907, at least or exactly or at most 908, at least or exactly or at most 909, at least or exactly or at most 910, at least or exactly or at most 911, at least or exactly or at most 912, at least or exactly or at most 913, at least or exactly or at most 914, at least or exactly or at most 915, at least or exactly or at most 916, at least or exactly or at most 917, at least or exactly or at most 918, at least or exactly or at most 919, at least or exactly or at most 920, at least or exactly or at most 921, at least or exactly or at most 922, at least or exactly or at most 923, at least or exactly or at most 924, at least or exactly or at most 925, at least or exactly or at most 926, at least or exactly or at most 927, at least or exactly or at most 928, at least or exactly or at most 929, at least or exactly or at most 930, at least or exactly or at most 931, at least or exactly or at most 932, at least or exactly or at most 933, at least or exactly or at most 934, at least or exactly or at most 935, at least or exactly or at most 936, at least or exactly or at most 937, at least or exactly or at most 938, at least or exactly or at most 939, at least or exactly or at most 940, at least or exactly or at most 941, at least or exactly or at most 942, at least or exactly or at most 943, at least or exactly or at most 944, at least or exactly or at most 945, at least or exactly or at most 946, at least or exactly or at most 947, at least or exactly or at most 948, at least or exactly or at most 949, at least or exactly or at most 950, at least or exactly or at most 951, at least or exactly or at most 952, at least or exactly or at most 953, at least or exactly or at most 954, at least or exactly or at most 955, at least or exactly or at most 956, at least or exactly or at most 957, at least or exactly or at most 958, at least or exactly or at most 959, at least or exactly or at most 960, at least or exactly or at most 961, at least or exactly or at most 962, at least or exactly or at most 963, at least or exactly or at most 964, at least or exactly or at most 965, at least or exactly or at most 966, at least or exactly or at most 967, at least or exactly or at most 968, at least, or exactly or at most 969, at least or exactly or at most 970, at least or exactly or at most 971, at least or exactly or at most 972, at least or exactly or at most 973, at least or exactly or at most 974 contiguous amino acid residues.

Insofar as embodiment b relates to SEQ ID NO. 26-30, the at least 5 contiguous amino acids referred to in option b) in the definition of the first aspect of the invention will preferably constitute at least or exactly or at most 975, at least or exactly or at most 976, at least or exactly or at most 977, at least or exactly or at most 978, at least or exactly or at most 979, at least or exactly or at most 980, at least or exactly or at most 981, at least or exactly or at most 982, at least or exactly or at most 983, at least or exactly or at most 984, at least or exactly or at most 985, at least or exactly or at most 986, at least or exactly or at most 987, at least or exactly or at most 988, at least or exactly or at most 989, at least or exactly or at most 990, at least or exactly or at most 991, at least or exactly or at most 992, at least or exactly or at most 993, at least or exactly or at most 994, at least or exactly or at most 995, at least or exactly or at most 996, at least or exactly or at most 997, at least or exactly or at most 998, at least or exactly or at most 999, at least or exactly or at most 1000, at least or exactly or at most 1001, at least or exactly or at most 1002, at least or exactly or at most 1003, at least or exactly or at most 1004, at least or exactly or at most 1005, at least or exactly or at most 1006, at least or exactly or at most 1007, at least or exactly or at most 1008, at least or exactly or at most 1009, at least or exactly or at most 1010, at least or exactly or at most 1011, at least or exactly or at most 1012, at least or exactly or at most 1013, at least or exactly or at most 1014, at least or exactly or at most 1015, at least or exactly or at most 1016, at least or exactly or at most 1017, at least or exactly or at most 1018, at least or exactly or at most 1019, at least or exactly or at most 1020, at least or exactly or at most 1021, at least or exactly or at most 1022, at least or exactly or at most 1023, at least or exactly or at most 1024, at least or exactly or at most 1025, at least or exactly or at most 1026, at least or exactly or at most 1027, at least or exactly or at most 1028, at least or exactly or at most 1029, at least or exactly or at most 1030, at least or exactly or at most 1031, at least or exactly or at most 1032, at least or exactly or at most 1033, at least or exactly or at most 1034, at least or exactly or at most 1035, at least or exactly or at most 1036, at least or exactly or at most 1037, at least or exactly or at most 1038, at least or exactly or at most 1039, at least or exactly or at most 1040, at least or exactly or at most 1041, at least or exactly or at most 1042, at least or exactly or at most 1043, at least or exactly or at most 1044, at least or exactly or at most 1045, at least or exactly or at most 1046, at least or exactly or at most 1047, at least or exactly or at most 1048, at least or exactly or at most 1049, at least or exactly or at most 1050, at least or exactly or at most 1051, at least or exactly or at most 1052, at least or exactly or at most 1053, at least or exactly or at most 1054, at least or exactly or at most 1055, at least or exactly or at most 1056, at least or exactly or at most 1057, at least or exactly or at most 1058, at least or exactly or at most 1059, at least or exactly or at most 1060, at least or exactly or at most 1061, at least or exactly or at most 1062, at least or exactly or at most 1063, at least or exactly or at most 1064, at least or exactly or at most 1065, at least or exactly or at most 1066, at least or exactly or at most 1067, at least or exactly or at most 1068, at least or exactly or at most 1069, at least or exactly or at most 1070, at least or exactly or at most 1071 contiguous amino acid residues.

Insofar as embodiment b relates to SEQ ID NO: 27-30, the at least 5 contiguous amino acids referred to in option b) n the definition of the first aspect of the invention will preferably constitute at least or exactly or at most 1072, at least or exactly or at most 1073, at least or exactly or at most 1074, at least or exactly or at most 1075, at least or exactly or at most 1076, at least or exactly or at most 1077, at least or exactly or at most 1078, at least or exactly or at most 1079, at least or exactly or at most 1080, at least or exactly or at most 1081, at least or exactly or at most 1082, at least or exactly or at most 1083, at least or exactly or at most 1084, at least or exactly or at most 1085, at least or exactly or at most 1086, at least or exactly or at most 1087, at least or exactly or at most 1088, at least or exactly or at most 1089, at least or exactly or at most 1090, at least or exactly or at most 1091, at least or exactly or at most 1092, at least or exactly or at most 1093, at least or exactly or at most 1094, at least or exactly or at most 1095, at least or exactly or at most 1096, at least or exactly or at most 1097, at least or exactly or at most 1098, at least or exactly or at most 1099, at least or exactly or at most 1100, at least or exactly or at most 1101, at least or exactly or at most 1102, at least or exactly or at most 1103, at least or exactly or at most 1104, at least or exactly or at most 1105, at least or exactly or at most 1106, at least or exactly or at most 1107, at least or exactly or at most 1108, at least or exactly or at most 1109, at least or exactly or at most 1110 contiguous amino acid residues.

Insofar as embodiment b relates to SEQ 10 NO: 28-30, the at least 5 contiguous amino acids referred to in option b) in the definition of the first aspect of the invention will preferably constitute at least or exactly or at most 1111, at least or exactly or at most 1112, at least or exactly or at most 1113, at least or exactly or at most 1114, at least or exactly or at most 1115, at least or exactly or at most 1116, at least or exactly or at most 1117, at least or exactly or at most 1118, at least or exactly or at most 1119, at least or exactly or at most 1120, at least or exactly or at most 1121, at least or exactly or at most 0.1122, at least or exactly or at most 1123, at least or exactly or at most 1124, at least or exactly or at most 1125, at least or exactly or at most 1126, at least or exactly or at most 1127, at least or exactly or at most 1128, at least or exactly or at most 1129, at least or exactly or at most 1130, at least or exactly or at most 1131, at least or exactly or at most 1132, at least or exactly or at most 1133, at least or exactly or at most 1134, at least or exactly or at most 1135, at least or exactly or at most 1136, at least or exactly or at most 1137, at least or exactly or at most 1138, at least or exactly or at most 1139, at least or exactly or at most 1140, at least or exactly or at most 1141, at least or exactly or at most 1142, at least or exactly or at most 1143, at least or exactly or at most 1144, at least or exactly or at most 1145, at least or exactly or at most 1146, at least or exactly or at most 1147, at least or exactly or at most 1148, at least or exactly or at most 1149, at least or exactly or at most 1150, at least or exactly or at most 1151, at least or exactly or at most 1152, at least or exactly or at most 1153, at least or exactly or at most 1154, at least or exactly or at most 1155, at least or exactly or at most 1156, at least or exactly or at most 1157, at least or exactly or at most 1158, at least or exactly or at most 1159, at least or exactly or at most 1160, at least or exactly or at most 1161, at least or exactly or at most 1162, at least or exactly or at most 1163, at least or exactly or at most 1164, at least or exactly or at most 1165, at least or exactly or at most 1166, at least or exactly or at most 1167, at least or exactly or at most 1168, at least or exactly or at most 1169, at least or exactly or at most 1170, at least or exactly or at most 1171, at least or exactly or at most 1172, at least or exactly or at most 1173, at least or exactly or at most 1174, at least or exactly or at most 1175, at least or exactly or at most 1176, at least or exactly or at most 1177, at least or exactly or at most 1178, at least or exactly or at most 1179, at least or exactly or at most 1180, at least or exactly or at most 1181, at least or exactly or at most 1182, at least or exactly or at most 1183, at least or exactly or at most 1184, at least or exactly or at most 1185, at least or exactly or at most 1186, at least or exactly or at most 1187, at least or exactly or at most 1188, at least or exactly or at most 1189, at least or exactly or at most 1190, at least or exactly or at most 1191, at least or exactly or at most 1192, at least or exactly or at most 1193, at least or exactly or at most 1194, at least or exactly or at most 1195, at least or exactly or at most 1196, at least or exactly or at most 1197, at least or exactly or at most 1198, at least or exactly or at most 1199, at least or exactly or at most 1200, at least or exactly or at most 1201, at least or exactly or at most 1202, at least or exactly or at most 1203, at least or exactly or at most 1204, at least or exactly or at most 1205, at least or exactly or at most 1206, at least or exactly or at most 1207, at least or exactly or at most 1208, at least or exactly or at most 1209, at least or exactly or at most 1210, at least or exactly or at most 1211, at least or exactly or at most 1212, at least or exactly or at most 1213, at least or exactly or at most 1214, at least or exactly or at most 1215, at least or exactly or at most 1216, at least or exactly or at most 1217, at least or exactly or at most 1218, at least or exactly or at most 1219, at least or exactly or at most 1220, at least or exactly or at most 1221, at least or exactly or at most 1222, at least or exactly or at most 1223, at least or exactly or at most 1224, at least or exactly or at most 1225, at least or exactly or at most 1226, at least or exactly or at most 1227, at least or exactly or at most 1228 contiguous amino acid residues.

Insofar as embodiment b relates to SEQ ID NO: 29-30, the at least 5 contiguous amino acids referred to in option b) in the definition of the first aspect of the invention will preferably constitute at least or exactly or at most 1229, at least or exactly or at most 1230, at least or exactly or at most 1231, at least or exactly or at most 1232, at least or exactly or at most 1233, at least or exactly or at most 1234, at least or exactly or at most 1235, at least or exactly or at most 1236, at least or exactly or at most 1237, at least or exactly or at most 1238, at least or exactly or at most 1239, at least or exactly or at most 1240, at least or exactly or at most 1241, at least or exactly or at most 1242, at least or exactly or at most 1243, at least or exactly or at most 1244, at least or exactly or at most 1245, at least or exactly or at most 1246, at least or exactly or at most 1247, at least or exactly or at most 1248, at least or exactly or at most 1249, at least or exactly or at most 1250, at least or exactly or at most 1251, at least or, exactly or at most 1252, at least or exactly or at most 1253, at least or exactly or at most 1254, at least or exactly or at most 1255, at least or exactly or at most 1256, at least or exactly or at most 1257, at least or exactly or at most 1258, at least or exactly or at most 1259, at least or exactly or at most 1260, at least or exactly or at most 1261, at least or exactly or at most 1262, at least or exactly or at most 1263, at least or exactly or at most 1264, at least or exactly or at most 1265, at least or exactly or at most 1266, at least or exactly or at most 1267, at least or exactly or at most 1268, at least or exactly or at most 1269, at least or exactly or at most 1270, at least or exactly or at most 1271, at least or exactly or at most 1272, at least or exactly or at most 1273, at least or exactly or at most 1274, at least or exactly or at most 1275, at least or exactly or at most 1276, at least or exactly or at most 1277, at least or exactly or at most 1278, at least or exactly or at most 1279, at least or exactly or at most 1280, at least or exactly or at most 1281, at least or exactly or at most 1282, at least or exactly or at most 1283, at least or exactly or at most 1284 contiguous amino acid residues.

Insofar as embodiment b relates to SEQ 10 NO: 30, the at least 5 contiguous amino acids referred to in option b) in the definition of the first aspect of the invention will preferably constitute at least or exactly or at most 1285, at least or exactly or at most 1286, at least or exactly or at most 1287, at least or exactly or at most 1288, at least or exactly or at most 1289, at least or exactly or at most 1290, at least or exactly or at most 1291, at least or exactly or at most 1292, at least or exactly or at most 1293, at least or exactly or at most 1294, at least or exactly or at most 1295, at least or exactly or at most 1296, at least or exactly or at most 1297, at least or exactly or at most 1298, at least or exactly or at most 1299, at least or exactly or at most 1300, at least or exactly or at most 1301, at least or exactly or at most 1302, at least or exactly or at most 1303, at least or exactly or at most 1304, at least or exactly or at most 1305, at least or exactly or at most 1306, at least or exactly or at most 1307, at least or exactly or at most 1308, at least or exactly or at most 1309, at least or exactly or at most 1310, at least or exactly or at most 1311, at least or exactly or at most 1312, at least or exactly or at most 1313, at least or exactly or at most 1314, at least or exactly or at most 1315, at least or exactly or at most 1316, at least or exactly or at most 1317, at least or exactly or at most 1318, at least or exactly or at most 1319, at least or exactly or at most 1320, at least or exactly or at most 1321, at least or exactly or at most 1322, at least or exactly or at most 1323, at least or exactly or at most 1324, at least or exactly or at most 1325, at least or exactly or at most 1326, at least or exactly or at most 1327, at least or exactly or at most 1328, at least or exactly or at most 1329, at least or exactly or at most 1330, at least or exactly or at most 1331, at least or exactly or at most 1332, at least or exactly or at most 1333, at least or exactly or at most 1334, at least or exactly or at most 1335, at least or exactly or at most 1336, at least or exactly or at most 1337, at least or exactly or at most 1338, at least or exactly or at most 1339, at least or exactly or at most 1340, at least or exactly or at most 1341, at least or exactly or at most 1342, at least or exactly or at most 1343, at least or exactly or at most 1344, at least or exactly or at most 1345, at least or exactly or at most 1346, at least or exactly or at most 1347, at least or exactly or at most 1348, at least or exactly or at most 1349, at least or exactly or at most 1350, at least or exactly or at most 1351, at least or exactly or at most 1352, at least or exactly or at most 1353, at least or exactly or at most 1354, at least or exactly or at most 1355, at least or exactly or at most 1356, at least or exactly or at most 1357, at least or exactly or at most 1358, at least or exactly or at most 1359, at least or exactly or at most 1360, at least or exactly or at most 1361, at least or exactly or at most 1362, at least or exactly or at most 1363, at least or exactly or at most 1364, at least or exactly or at most 1365, at least or exactly or at most 1366, at least or exactly or at most 1367, at least or exactly or at most 1368, at least or exactly or at most 1369, at least or exactly or at most 1370, at least or exactly or at most 1371, at least or exactly or at most 1372, at least or exactly or at most 1373, at least or exactly or at most 1374, at least or exactly or at most 1375, at least or exactly or at most 1376, at least or exactly or at most 1377, at least or exactly or at most 1378, at least or exactly or at most 1379, at least or exactly or at most 1380, at least or exactly or at most 1381, at least or exactly or at most 1382, at least or exactly or at most 1383, at least or exactly or at most 1384, at least or exactly or at most 1385, at least or exactly or at most 1386, at least or exactly or at most 1387, at least or exactly or at most 1388, at least or exactly or at most 1389, at least or exactly or at most 1390, at least or exactly or at most 1391, at least or exactly or at most 1392, at least or exactly or at most 1393, at least or exactly or at most 1394, at least or exactly or at most 1395, at least or exactly or at most 1396, at least or exactly or at most 1397, at least or exactly or at most 1398, at least or exactly or at most 1399, at least or exactly or at most 1400, at least or exactly or at most 1401, at least or exactly or at most 1402, at least or exactly or at most 1403, at least or exactly or at most 1404, at least or exactly or at most 1405, at least or exactly or at most 1406, at least or exactly or at most 1407, at least or exactly or at most 1408, at least or exactly or at most 1409, at least or exactly or at most 1410, at least or exactly or at most 1411, at least or exactly or at most 1412, at least or exactly or at most 1413, at least or exactly or at most 1414, at least or exactly or at most 1415, at least or exactly or at most 1416, at least or exactly or at most 1417, at least or exactly or at most 1418, at least or exactly or at most 1419, at least or exactly or at most 1420, at least or exactly or at most 1421, at least or exactly or at most 1422, at least or exactly or at most 1423, at least or exactly or at most 1424, at least or exactly or at most 1425, at least or exactly or at most 1426, at least or exactly or at most 1427, at least or exactly or at most 1428, at least or exactly or at most 1429, at least or exactly or at most 1430, at least or exactly or at most 1431, at least or exactly or at most 1432, at least or exactly or at most 1433, at least or exactly or at most 1434, at least or exactly or at most 1435, at least or exactly or at most 1436, at least or exactly or at most 1437, at least or exactly or at most 1438, at least or exactly or at most 1439, at least or exactly or at most 1440, at least or exactly or at most 1441, at least or exactly or at most 1442, at least or exactly or at most 1443, at least or exactly or at most 1444, at least or exactly or at most 1445, at least or exactly or at most 1446, at least or exactly or at most 1447, at least or exactly or at most 1448, at least or exactly or at most 1449, at least or exactly or at most 1450, at least or exactly or at most 0.1451, at least or exactly or at most 1452, at least or exactly or at most 1453, at least or exactly or at most 1454, at least or exactly or at most 1455, at least or exactly or at most 1456, at least or exactly or at most 1457, at least or exactly or at most 1458, at least or exactly or at most 1459, at least or exactly or at most 1460, at least or exactly or at most 1461, at least or exactly or at most 1462, at least or exactly or at most 1463, at least or exactly or at most 1464, at least or exactly or at most 1465, at least or exactly or at most 1466, at least or exactly or at most 1467, at least or exactly or at most 1468, at least or exactly or at most 1469, at least or exactly or at most 1470, at least or exactly or at most 1471, at least or exactly or at most 1472, at least, or exactly or at most 1473, at least or exactly or at most 1474, at least or exactly or at most 1475, at least or exactly or at most 1476, at least or exactly or at most 1477, at least or exactly or at most 1478, at least or exactly or at most 1479, at least or exactly or at most 1480, at least or exactly or at most 1481, at least or exactly or at most 1482, at least or exactly or at most 1483, at least or exactly or at most 1484, at least or exactly or at most 1485, at least or exactly or at most 1486, at least or exactly or at most 1487, at least or exactly or at most 1488, at least or exactly or at most 1489, at least or exactly or at most 1490, at least or exactly or at most 1491, at least or exactly or at most 1492, at least or exactly or at most 1493, at least or exactly or at most 1494, at least or exactly or at most 1495, at least or exactly or at most 1496, at least or exactly or at most 1497, at least or exactly or at most 1498, at least or exactly or at most 1499, at least or exactly or at most 1500, at least or exactly or at most 1501, at least or exactly or at most 1502, at least or exactly or at most 1503, at least or exactly or at most 1504, at least or exactly or at most 1505, at least or exactly or at most 1506, at least or exactly or at most 1507, at least or exactly or at most 1508, at least or exactly or at most 1509, at least or exactly or at most 1510, at least or exactly or at most 1511, at least or exactly or at most 1512, at least or exactly or at most 1513, at least or exactly or at most 1514, at least or exactly or at most 1515, at least or exactly or at most 1516, at least or exactly or at most 1517, at least or exactly or at most 1518, at least or exactly or at most 1519, at least or exactly or at most 1520, at least or exactly or at most 1521, at least or exactly or at most 1522, at least or exactly or at most 1523, at least or exactly or at most 1524, at least or exactly or at most 1525, at least or exactly or at most 1526, at least or exactly or at most 1527, at least or exactly or at most 1520, at least or exactly or at most 1529, at least or exactly or at most 1530, at least or exactly or at most 1531, at least or exactly or at most 1532, at least or exactly or at most 1533, at least or exactly or at most 1534, at least or exactly or at most 1535, at least or exactly or at most 1536, at least or exactly or at most 1537, at least or exactly or at most 1538, at least or exactly or at most 1539, at least or exactly or at most 1540, at least or exactly or at most 1541, at least or exactly or at most 1542, at least or exactly or at most 1543, at least or exactly or at most 1544, at least or exactly or at most 1545, at least or exactly or at most 1546, at least or exactly or at most 1547, at least or exactly or at most 1548, at least or exactly or at most 1549, at least or exactly or at most 1550, at least or exactly or at most 1551, at least or exactly or at most 1552, at least or exactly or at most 1553, at least or exactly or at most 1554, at feast or exactly or at most 1555, at least or exactly or at most 1556, at least or exactly or at most 1557, at least or exactly or at most 1558, at least or exactly or at most 1559, at least or exactly or at most 1560, at least or exactly or at most 1561, at least or exactly or at most 1562, at least or exactly or at most 1563, at least or exactly or at most 1564, at least or exactly or at most 1565, at least or exactly or at most 1566, at least or exactly or at most 1567, at least or exactly or at most 1568, at least or exactly or at most 1569, at least or exactly or at most 1570, at least or exactly or at most 1571, at least or exactly or at most 1572, at least or exactly or at most 1573, at least or exactly or at most 1574, at least or exactly or at most 1575, at least or exactly or at most 1576, at least or exactly or at most 1577, at least or exactly or at most 1578, at least or exactly or at most 1579, at least or exactly or at most 1580, at least or exactly or at most 1581, at least or exactly or at most 1582, at least or exactly or at most 1583, at least or exactly or at most 1584, at least or exactly or at most 1585, at least or exactly or at most 1586, at least or exactly or at most 1587, at least or exactly or at most 1588, at least or exactly or at most 1589, at least or exactly or at most 1590, at least or exactly or at most 1591, at least or exactly or at most 1592, at least or exactly or at most 1593, at least or exactly or at most 1594, at least or exactly or at most 1595, at least or exactly or at most 1596, at least or exactly or at most 1597, at least or exactly or at most 1598, at least or exactly or at most 1599, at least or exactly or at most 1600, at least or exactly or at most 1601, at least or exactly or at most 1602, at least or exactly or at most 1603, at least or exactly or at most 1604, at least or exactly or at most 1605, at least or exactly or at most 1606, at least or exactly or at most 1607, at least or exactly or at most 1608, at least or exactly or at most 1609, at least or exactly or at most 1610, at least or exactly or at most 1611, at least or exactly or at most 1612, at least or exactly or at most 1613, at least or exactly or at most 1614, at least or exactly or at most 1615, at least or exactly or at most 1616, at least or exactly or at most 1617, at least or exactly or at most 1618, at least or exactly or at most 1619, at least or exactly or at most 1620, at least or exactly or at most 1621, at least or exactly or at most 1622, at least or exactly or at most 1623, at least or exactly or at most 1624, at least or exactly or at most 1625, at least or exactly or at most 1626, at least or exactly or at most 1627, at least or exactly or at most 1628, at least or exactly or at most 1629, at least or exactly or at most 1630, at least or exactly or at most 1631, at least or exactly or at most 1632, at least or exactly or at most 1633, at least or exactly or at most 1634, at least or exactly or at most 1635, at least or exactly or at most 1636, at least or exactly or at most 1637, at least or exactly or at most 1638, at least or exactly or at most 1639, at least or exactly or at most 1640, at least or exactly or at most 1641, at least or exactly or at most 1642, at least or exactly or at most 1643, at least or exactly or at most 1644, at least or exactly or at most 1645, at least or exactly or at most 1646, at least or exactly or at most 1647, at least or exactly or at most 1648, at least or exactly or at most 1649, at least or exactly or at most 1650, at least or exactly or at most 1651, at least or exactly or at most 1652, at least or exactly or at most 1653, at least or exactly or at most 1654, at least or exactly or at most 1655, at least or exactly or at most 1656, at least or exactly or at most 1657, at least or exactly or at most 1658, at least or exactly or at most 1659, at least or exactly or at most 1660, at least or exactly or at most 1661, at least or exactly or at most 1662, at least or exactly or at most 1663, at least or exactly or at most 1664, at least or exactly or at most 1665, at least or exactly or at most 1666, at least or exactly or at most 1667, at least or exactly or at most 1668, at least or exactly or at most 1669, at least or exactly or at most 1670, at least or exactly or at most 1671, at least or exactly or at most 1672, at least or exactly or at most 1673, at least or exactly or at most 1674, at least or exactly or at most 1675, at least or exactly or at most 1676, at least or exactly or at most 1677, at least or exactly or at most 1678, at least or exactly or at most 1679, at least or exactly or at most 1680, at least or exactly or at most 1681, at least or exactly or at most 1682, at least or exactly or at most 1683, at least or exactly or at most 1684, at least or exactly or at most 1685, at least or exactly or at most 1686, at least or exactly or at most 1687, at least or exactly or at most 1688, at least or exactly or at most 1689, at least or exactly or at most 1690, at least or exactly or at most 1691, at least or exactly or at most 1692, at least or exactly or at most 1693, at least or exactly or at most 1694, at least or exactly or at most 1695, at least or exactly or at most 1696, at least or exactly or at most 1697, at least or exactly or at most 1698, at least or exactly or at most 1699, at least or exactly or at most 1700, at least or exactly or at most 1701, at least or exactly or at most 1702, at least or exactly or at most 1703, at least or exactly or at most 1704, at least or exactly or at most 1705, at least or exactly or at most 1706, at least or exactly or at most 1707, at least or exactly or at most 1708, at least or exactly or at most 1709, at least or exactly or at most 1710, at least or exactly or at most 1711, at least or exactly or at most 1712, at least or exactly or at most 1713, at least or exactly or at most 1714, at least or exactly or at most 1715, at least or exactly or at most 1716, at least or exactly or at most 1717, at least or exactly or at most 1718, at least or exactly or at most 1719, at least or exactly or at most 1720, at least or exactly or at most 1721, at least or exactly or at most 1722, at least or exactly or at most 1723, at least or exactly or at most 1724, at least or exactly or at most 1725, at least or exactly or at most 1726, at least or exactly or at most 1727, at least or exactly or at most 1728, at least or exactly or at most 1729, at least or exactly or at most 1730, at least or exactly or at most 1731, at least or exactly or at most 1732, at least or exactly or at most 1733, at least or exactly or at most 1734, at least or exactly or at most 1735, at least or exactly or at most 1736, at least or exactly or at most 1737, at least or exactly or at most 1738, at least or exactly or at most 1739, at least or exactly or at most 1740, at least or exactly or at most 1741, at least or exactly or at most 1742, at least or exactly or at most 1743, at least or exactly or at most 1744, at least or exactly or at most 1745, at least or exactly or at most 1746, at least or exactly or at most 1747, at least or exactly or at most 1748, at least or exactly or at most 1749, at least or exactly or at most 1750, at least or exactly or at most 1751, at least or exactly or at most 1752, at least or exactly or at most 1753, at least or exactly or at most 1754, at least or exactly or at most 1755, at least or exactly or at most 1756, at least or exactly or at most 1757, at least or exactly or at most 1758, at least or exactly or at most 1759, at least or exactly or at most 1760, at least or exactly or at most 1761, at least or exactly or at most 1762, at least or exactly or at most 1763, at least or exactly or at most 1764, at least or exactly or at most 1765, at least or exactly or at most 1766, at least or exactly or at most 1767, at least or exactly or at most 1768, at least or exactly or at most 1769, at least or exactly or at most 1770, at least or exactly or at most 1771, at least or exactly or at most 1772, at least or exactly or at most 1773, at least or exactly or at most 1774, at least or exactly or at most 1775, at least or exactly or at most 1776, at least or exactly or at most 1777, at least or exactly or at most 1778, at least or exactly or at most 1779, at least or exactly or at most 1780, at least or exactly or at most 1781, at least or exactly or at most 1782, at least or exactly or at most 1783, at least or exactly or at most 1784, at least or exactly or at most 1785, at least or exactly or at most 1786, at least or exactly or at most 1787, at least or exactly or at most 1788, at least or exactly or at most 1789, at least or exactly or at most 1790, at least or exactly or at most 1791, at least or exactly or at most 1792, at least or exactly or at most 1793, at least or exactly or at most 1794, at least or exactly or at most 1795, at least or exactly or at most 1796, at least or exactly or at most 1797, at least or exactly or at most 1798, at least or exactly or at most 1799, at least or exactly or at most 1800, at least or exactly or at most 1801, at least or exactly or at most 1802, at least or exactly or at most 1803, at least or exactly or at most 1804, at least or exactly or at most 1805, at least or exactly or at most 1806, at least or exactly or at most 1807, at least or exactly or at most 1808, at least or exactly or at most 1809, at least or exactly or at most 1810, at least or exactly or at most 1811, at least or exactly or at most 1812, at least or exactly or at most 1813, at least or exactly or at most 1814, at least or exactly or at most 1815, at least or exactly or at most 1816, at least or exactly or at most 1817, at least or exactly or at most 1818, at least or exactly or at most 1819, at least or exactly or at most 1820, at least or exactly or at most 1821, at least or exactly or at most 1822, at least or exactly or at most 1823, at least or exactly or at most 1824, at least or exactly or at most 1825, at least or exactly or at most 1826, at least or exactly or at most 1827, at least or exactly or at most 1828, at least or exactly or at most 1829, at least or exactly or at most 1830, at least or exactly or at most 1831, at least or exactly or at most 1832, at least or exactly or at most 1833, at least or exactly or at most 1834, at least or exactly or at most 1835, at least or exactly or at most 1836, at least or exactly or at most 1837, at least or exactly or at most 1838, at least or exactly or at most 1839, at least or exactly or at most 1840, at least or exactly or at most 1841, at least or exactly or at most 1842, at least or exactly or at most 1843, at least or exactly or at most 1844, at least or exactly or at most 1845, at least or exactly or at most 1846, at least or exactly or at most 1847, at least or exactly or at most 1848, at least or exactly or at most 1849, at least or exactly or at most 1850, at least or exactly or at most 1851, at least or exactly or at most 1852, at least or exactly or at most 1853, at least or exactly or at most 1854, at least or exactly or at most 1855, at least or exactly or at most 1856, at least or exactly or at most 1857, at least or exactly or at most 1858, at least or exactly or at most 1859, at least or exactly or at most 1860, at least or exactly or at most 1861, at least or exactly or at most 1862, at least or exactly or at most 1863, at least or exactly or at most 1864, at least or exactly or at most 1865, at least or exactly or at most 1866, at least or exactly or at most 1867, at least or exactly or at most 1868, at least or exactly or at most 1869, at least or exactly or at most 1870, at least or exactly or at most 1871, at least or exactly or at most 1872, at least or exactly or at most 1873, at least or exactly or at most 1874, at least or exactly or at most 1875, at least or exactly or at most 1876, at least or exactly or at most 1877, at least or exactly or at most 1878, at least or exactly or at most 1879, at least or exactly or at most 1880, at least or exactly or at most 1881, at least or exactly or at most 1882, at least or exactly or at most 1883, at least or exactly or at most 1884, at least or exactly or at most 1885, at least or exactly or at most 1886, at least or exactly or at most 1887, at least or exactly or at most 1888, at least or exactly or at most 1889, at least or exactly or at most 1890, at least or exactly or at most 1891, at least or exactly or at most 1892, at least or exactly or at most 1893, at least or exactly or at most 1894, at least or exactly or at most 1895, at least or exactly or at most 1896, at least or exactly or at most 1897, at least or exactly or at most 1898, at least or exactly or at most 1899, at least or exactly or at most 1900, at least or exactly or at most 1901, at least or exactly or at most 1902, at least or exactly or at most 1903, at least or exactly or at most 1904, at least or exactly or at most 1905, at least or exactly or at most 1906, at least or exactly or at most 1907, at least or exactly or at most 1908, at least or exactly or at most 1909, at least or exactly or at most 1910, at least or exactly or at most 1911, at least or exactly or at most 1912, at least or exactly or at most 1913, at least or exactly or at most 1914, at least or exactly or at most 1915, at least or exactly or at most 1916, at least or exactly or at most 1917, at least or exactly or at most 1918, at least or exactly or at most 1919, at least or exactly or at most 1920, at least or exactly or at most 1921, at least or exactly or at most 1922, at least or exactly or at most 1923, at least or exactly or at most 1924, at least or exactly or at most 1925, at least or exactly or at most 1926, at least or exactly or at most 1927, at least or exactly or at most 1928, at least or exactly or at most 1929, at least or exactly or at most 1930, at least or exactly or at most 1931, at least or exactly or at most 1932, at least or exactly or at most 1933, at least or exactly or at most 1934, at least or exactly or at most 1935, at least or exactly or at most 1936, at least or exactly or at most 1937, at least or exactly or at most 1938, at least or exactly or at most 1939, at least or exactly or at most 1940, at least or exactly or at most 1941, at least or exactly or at most 1942, at least or exactly or at most 1943, at least or exactly or at most 1944, at least or exactly or at most 1945, at least or exactly or at most 1946, at least or exactly or at most 1947, at least or exactly or at most 1948, at least or exactly or at most 1949, at least or exactly or at most 1950, at least or exactly or at most 1951, at least or exactly or at most 1952, at least or exactly or at most 1953, at least or exactly or at most 1954, at least or exactly or at most 1955, at least or exactly or at most 1956, at least or exactly or at most 1957, at least or exactly or at most 1958, at least or exactly or at most 1959, at least or exactly or at most 1960, at least or exactly or at most 1961, at least or exactly or at most 1962, at least or exactly or at most 1963, at least or exactly or at most 1964, at least or exactly or at most 1965, at least or exactly or at most 1966, at least or exactly or at most 1967, at least or exactly or at most 1968, at least or exactly or at most 1969, at least or exactly or at most 1970, at least or exactly or at most 1971, at least or exactly or at most 1972, at least or exactly or at most 1973, at least or exactly or at most 1974, at least or exactly or at most 1975, at least or exactly or at most 1976, at least or exactly or at most 1977, at least or exactly or at most 1978, at least or exactly or at most 1979, at least or exactly or at most 1980, at least or exactly or at most 1981, at least or exactly or at most 1982, at least or exactly or at most 1983, at least or exactly or at most 1984, at least or exactly or at most 1985, at least or exactly or at most 1986, at least or exactly or at most 1987, at least or exactly or at most 1988, at least or exactly or at most 1989, at least or exactly or at most 1990, at least or exactly or at most 1991, at least or exactly or at most 1992, at least or exactly or at most 1993, at least or exactly or at most 1994, at least or exactly or at most 1995, at least or exactly or at most 1996, at least or exactly or at most 1997, at least or exactly or at most 1998, at least or exactly or at most 1999, at least or exactly or at most 2000, at least or exactly or at most 2001, at least or exactly or at most 2002, at least or exactly or at most 2003, at least or exactly or at most 2004, at least or exactly or at most 2005, at least or exactly or at most 2006, at least or exactly or at most 2007, at least or exactly or at most 2008, at least or exactly or at most 2009, at least or exactly or at most 2010, at least or exactly or at most 2011, at least or exactly or at most 2012, at least or exactly or at most 2013, at least or exactly or at most 2014, at least or exactly or at most 2015, at least or exactly or at most 2016, at least or exactly or at most 2017, at least or exactly or at most 2018, at least or exactly or at most 2019, at least or exactly or at most 2020, at least or exactly or at most 2021, at least or exactly or at most 2022, at least or exactly or at most 2023, at least or exactly or at most 2024, at least or exactly or at most 2025, at least or exactly or at most 2026, at least or exactly or at most 2027, at least or exactly or at most 2028, at least or exactly or at most 2029, at least or exactly or at most 2030, at least or exactly or at most 2031, at least or exactly or at most 2032, at least or exactly or at most 2033, at least or exactly or at most 2034, at least or exactly or at most 2035, at least or exactly or at most 2036, at least or exactly or at most 2037, at least or exactly or at most 2038, at least or exactly or at most 2039, at least or exactly or at most 2040, at least or exactly or at most 2041, at least or exactly or at most 2042, at least or exactly or at most 2043, at least or exactly or at most 2044, at least or exactly or at most 2045, at least or exactly or at most 2046, at least or exactly or at most 2047, at least or exactly or at most 2048, at least or exactly or at most 2049, at least or exactly or at most 2050, at least or exactly or at most 2051, at least or exactly or at most 2052, at least or exactly or at most 2053, at least or exactly or at most 2054, at least or exactly or at most 2055, at least or exactly or at most 2056, at least or exactly or at most 2057, at least or exactly or at most 2058, at least or exactly or at most 2059, at least or exactly or at most 2060, at least or exactly or at most 2061, at least or exactly or at most 2062, at least or exactly or at most 2063, at least or exactly or at most 2064, at least or exactly or at most 2065, at least or exactly or at most 2066, at least or exactly or at most 2067, at least or exactly or at most 2068, at least or exactly or at most 2069, at least or exactly or at most 2070, at least or exactly or at most 2071, at least or exactly or at most 2072, at least or exactly or at most 2073, at least or exactly or at most 2074, at least or exactly or at most 2075, at least or exactly or at most 2076, at least or exactly or at most 2077, at least or exactly or at most 2078, at least or exactly or at most 2079, at least or exactly or at most 2080, at least or exactly or at most 2081, at least or exactly or at most 2082, at least or exactly or at most 2083, at least or exactly or at most 2084, at least or exactly or at most 2085, at least or exactly or at most 2086, at least or exactly or at most 2087, at least or exactly or at most 2088, at least or exactly or at most 2089, at least or exactly or at most 2090, at least or exactly or at most 2091, at least or exactly or at most 2092, at least or exactly or at most 2093, at least or exactly or at most 2094, at least or exactly or at most 2095, at least or exactly or at most 2096, at least or exactly or at most 2097, at least or exactly or at most 2098, at least or exactly or at most 2099, at least or exactly or at most 2100, at least or exactly or at most 2101, at least or exactly or at most 2102, at least or exactly or at most 2103, at least or exactly or at most 2104, at least or exactly or at most 2105, at least or exactly or at most 2106, at least or exactly or at most 2107, at least or exactly or at most 2108, at least or exactly or at most 2109, at least or exactly or at most 2110, at least or exactly or at most 2111, at least or exactly or at most 2112, at least or exactly or at most 2113, at least or exactly or at most 2114, at least or exactly or at most 2115, at least or exactly or at most 2116, at least or exactly or at most 2117, at least or exactly or at most 2118, at least or exactly or at most 2119, at least or exactly or at most 2120, at least or exactly or at most 2121, at least or exactly or at most 2122, at least or exactly or at most 2123, at least or exactly or at most 2124, at least or exactly or at most 2125, at least or exactly or at most 2126, at least or exactly or at most 2127, at least or exactly or at most 2128, at least or exactly or at most 2129, at least or exactly or at most 2130, at least or exactly or at most 2131, at least or exactly or at most 2132, at least or exactly or at most 2133, at least or exactly or at most 2134, at least or exactly or at most 2135, at least or exactly or at most 2136, at least or exactly or at most 2137, at least or exactly or at most 2138, at least or exactly or at most 2139, at least or exactly or at most 2140, at least or exactly or at most 2141, at least or exactly or at most 2142, at least or exactly or at most 2143, at least or exactly or at most 2144, at least or exactly or at most 2145, at least or exactly or at most 2146, at least or exactly or at most 2147, at least or exactly or at most 2148, at least or exactly or at most 2149, at least or exactly or at most 2150, at least or exactly or at most 2151, at least or exactly or at most 2152, at least or exactly or at most 2153, at least or exactly or at most 2154, at least or exactly or at most 2155, at least or exactly or at most 2156, at least or exactly or at most 2157, at least or exactly or at most 2158, at least or exactly or at most 2159, at least or exactly or at most 2160, at least or exactly or at most 2161, at least or exactly or at most 2162, at least or exactly or at most 2163, at least or exactly or at most 2164, at least or exactly or at most 2165, at least or exactly or at most 2166, at least or exactly or at most 2167, at least or exactly or at most 2168, at least or exactly or at most 2169, at least or exactly or at most 2170, at least or exactly or at most 2171, at least or exactly or at most 2172, at least or exactly or at most 2173, at least or exactly or at most 2174, at least or exactly or at most 2175, at least or exactly or at most 2176, at least or exactly or at most 2177, at least or exactly or at most 2178, at least or exactly or at most 2179, at least or exactly or at most 2180, at least or exactly or at most 2181, at least or exactly or at most 2182, at least or exactly or at most 2183, at least or exactly or at most 2184, at least or exactly or at most 2185, at least or exactly or at most 2186, at least or exactly or at most 2187, at least or exactly or at most 2188, at least or exactly or at most 2189, at least or exactly or at most 2190, at least or exactly or at most 2191, at least or exactly or at most 2192, at least or exactly or at most 2193, at least or exactly or at most 2194, at least or exactly or at most 2195, at least or exactly or at most 2196, at least or exactly or at most 2197, at least or exactly or at most 2198, at least or exactly or at most 2199, at least or exactly or at most 2200, at least or exactly or at most 2201, at least or exactly or at most 2202, at least or exactly or at most 2203, at least or exactly or at most 2204, at least or exactly or at most 2205, at least or exactly or at most 2206, at least or exactly or at most 2207, at least or exactly or at most 2208, at least or exactly or at most 2209, at least or exactly or at most 2210, at least or exactly or at most 2211, at least or exactly or at most 2212, at least or exactly or at most 2213, at least or exactly or at most 2214, at least or exactly or at most 2215, at least or exactly or at most 2216, at least or exactly or at most 2217, at least or exactly or at most 2218, at least or exactly or at most 2219, at least or exactly or at most 2220, at least or exactly or at most 2221, at least or exactly or at most 2222, at least or exactly or at most 2223, at least or exactly or at most 2224, at least or exactly or at most 2225, at least or exactly or at most 2226, at least or exactly or at most 2227, at least or exactly or at most 2228, at least or exactly or at most 2229, at least or exactly or at most 2230, at least or exactly or at most 2231, at least or exactly or at most 2232, at least or exactly or at most 2233, at least or exactly or at most 2234, at least or exactly or at most 2235, at least or exactly or at most 2236, at least or exactly or at most 2237, at least or exactly or at most 2238, at least or exactly or at most 2239, at least or exactly or at most 2240, at least or exactly or at most 2241, at least or exactly or at most 2242, at least or exactly or at most 2243, at least or exactly or at most 2244, at least or exactly or at most 2245, at least or exactly or at most 2246, at least or exactly or at most 2247, at least or exactly or at most 2248, at least or exactly or at most 2249, at least or exactly or at most 2250, at least or exactly or at most 2251, at least or exactly or at most 2252, at least or exactly or at most 2253, at least or exactly or at most 2254, at least or exactly or at most 2255, at least or exactly or at most 2256, at least or exactly or at most 2257, at least or exactly or at most 2258, at least or exactly or at most 2259, at least or exactly or at most 2260, at least or exactly or at most 2261, at least or exactly or at most 2262, at least or exactly or at most 2263, at least or exactly or at most 2264, at least or exactly or at most 2265, at least or exactly or at most 2266, at least or exactly or at most 2267, at least or exactly or at most 2268, at least or exactly or at most 2269, at least or exactly or at most 2270, at least or exactly or at most 2271, at least or exactly or at most 2272, at least or exactly or at most 2273, at least or exactly or at most 2274, at least or exactly or at most 2275, at least or exactly or at most 2276, at least or exactly or at most 2277, at least or exactly or at most 2278, at least or exactly or at most 2279, at least or exactly or at most 2280, at least or exactly or at most 2281, at least or exactly or at most 2282, at least or exactly or at most 2283, at least or exactly or at most 2284, at least or exactly or at most 2285, at least or exactly or at most 2286, at least or exactly or at most 2287, at least or exactly or at most 2288, at least or exactly or at most 2289, at least or exactly or at most 2290, at least or exactly or at most 2291, at least or exactly or at most 2292, at least or exactly or at most 2293, at least or exactly or at most 2294, at least or exactly or at most 2295, at least or exactly or at most 2296, at least or exactly or at most 2297, at least or exactly or at most 2298, at least or exactly or at most 2299, at least or exactly or at most 2300, at least or exactly or at most 2301, at least or exactly or at most 2302, at least or exactly or at most 2303, at least or exactly or at most 2304, at least or exactly or at most 2305, at least or exactly or at most 2306, at least or exactly or at most 2307, at least or exactly or at most 2308, at least or exactly or at most 2309, at least or exactly or at most 2310, at least or exactly or at most 2311, at least or exactly or at most 2312, at least or exactly or at most 2313, at least or exactly or at most 2314, at least or exactly or at most 2315, at least or exactly or at most 2316, at least or exactly or at most 2317, at least or exactly or at most 2318, at least or exactly or at most 2319, at least or exactly or at most 2320, at least or exactly or at most 2321, at least or exactly or at most 2322, at least or exactly or at most 2323, at least or exactly or at most 2324, at least or exactly or at most 2325, at least or exactly or at most 2326, at least or exactly or at most 2327, at least or exactly or at most 2328, at least or exactly or at most 2329, at least or exactly or at most 2330, at least or exactly or at most 2331, at least or exactly or at most 2332, at least or exactly or at most 2333, at least or exactly or at most 2334, at least or exactly or at most 2335, at least or exactly or at most 2336, at least or exactly or at most 2337, at least or exactly or at most 2338, at least or exactly or at most 2339, at least or exactly or at most 2340, at least or exactly or at most 2341, at least or exactly or at most 2342, at least or exactly or at most 2343, at least or exactly or at most 2344, at least or exactly or at most 2345, at least or exactly or at most 2346, at least or exactly or at most 2347, at least or exactly or at most 2348, at least or exactly or at most 2349, at least or exactly or at most 2350, at least or exactly or at most 2351, at least or exactly or at most 2352, at least or exactly or at most 2353, at least or exactly or at most 2354, at least or exactly or at most 2355, at least or exactly or at most 2356, at least or exactly or at most 2357, at least or exactly or at most 2358, at least or exactly or at most 2359, at least or exactly or at most 2360, at least or exactly or at most 2361, at least or exactly or at most 2362, at least or exactly or at most 2363, at least or exactly or at most 2364, at least or exactly or at most 2365, at least or exactly or at most 2366, at least or exactly or at most 2367, at least or exactly or at most 2368, at least or exactly or at most 2369, at least or exactly or at most 2370, at least or exactly or at most 2371, at least or exactly or at most 2372, at least or exactly or at most 2373, at least or exactly or at most 2374, at least or exactly or at most 2375, at least or exactly or at most 2376, at least or exactly or at most 2377, at least or exactly or at most 2378, at least or exactly or at most 0.2379, at least or exactly or at most 2380, at least or exactly or at most 2381, at least or exactly or at most 2382, at least or exactly or at most 2383, at least or exactly or at most 2384, at least or exactly or at most 2385, at least or exactly or at most 2386, at least or exactly or at most 2387, at least or exactly or at most 2388, at least or exactly or at most 2389, at least or exactly or at most 2390, at least or exactly or at most 2391, at least or exactly or at most 2392, at least or exactly or at most 2393, at least or exactly or at most 2394, at least or exactly or at most 2395, at least or exactly or at most 2396, at least or exactly or at most 2397, at least or exactly or at most 2398, at least or exactly or at most 2399, at least or exactly or at most 2400, at least or exactly or at most 2401, at least or exactly or at most 2402, at least or exactly or at most 2403, at least or exactly or at most 2404, at least or exactly or at most 2405, at least or exactly or at most 2406, at least or exactly or at most 2407, at least or exactly or at most 2408, at least or exactly or at most 2409, at least or exactly or at most 2410, at least or exactly or at most 2411, at least or exactly or at most 2412, at least or exactly or at most 2413, at least or exactly or at most 2414, at least or exactly or at most 2415, at least or exactly or at most 2416, at least or exactly or at most 2417, at least or exactly or at most 2418, at least or exactly or at most 2419, at least or exactly or at most 2420, at least or exactly or at most 2421, at least or exactly or at most 2422, at least or exactly or at most 2423, at least or exactly or at most 2424, at least or exactly or at most 2425, at least or exactly or at most 2426, at least or exactly or at most 2427, at least or exactly or at most 2428, at least or exactly or at most 2429, at least or exactly or at most 2430, at least or exactly or at most 2431, at least or exactly or at most 2432, at least or exactly or at most 2433, at least or exactly or at most 2434, at least or exactly or at most 2435, at least or exactly or at most 2436, at least or exactly or at most 2437, at least or exactly or at most 2438, at least or exactly or at most 2439, at least or exactly or at most 2440, at least or exactly or at most 2441, at least or exactly or at most 2442, at least or exactly or at most 2443, at least or exactly or at most 2444, at least or exactly or at most 2445, at least or exactly or at most 2446, at least or exactly or at most 2447, at least or exactly or at most 2448, at least or exactly or at most 2449, at least or exactly or at most 2450, at least or exactly or at most 2451, at least or exactly or at most 2452, at least or exactly or at most 2453, at least or exactly or at most 2454, at least or exactly or at most 2455, at least or exactly or at most 2456, at least or exactly or at most 2457, at least or exactly or at most 2458, at least or exactly or at most 2459, at least or exactly or at most 2460, at least or exactly or at most 2461, at least or exactly or at most 2462, at least or exactly or at most 2463, at least or exactly or at most 2464, at least or exactly or at most 2465, at least or exactly or at most 2466, at least or exactly or at most 2467, at least or exactly or at most 2468, at least or exactly or at most 2469, at least or exactly or at most 2470, at least or exactly or at most 2471, at least or exactly or at most 2472, at least or exactly or at most 2473, at least or exactly or at most 2474, at least or exactly or at most 2475, at least or exactly or at most 2476, at least or exactly or at most 2477, at least or exactly or at most 2478, at least or exactly or at most 2479, at least or exactly or at most 2480, at least or exactly or at most 2481, at least or exactly or at most 2482, at least or exactly or at most 2483, at least or exactly or at most 2484, at least or exactly or at most 2485, at least or exactly or at most 2486, at least or exactly or at most 2487, at least or exactly or at most 2488, at least or exactly or at most 2489, at least or exactly or at most 2490, at least or exactly or at most 2491, at least or exactly or at most 2492, at least or exactly or at most 2493, at least or exactly or at most 2494, at least or exactly or at most 2495, at least or exactly or at most 2496, at least or exactly or at most 2497, at least or exactly or at most 2498, at least or exactly or at most 2499, at least or exactly or at most 2500, at least or exactly or at most 2501, at least or exactly or at most 2502, at least or exactly or at most 2503, at least or exactly or at most 2504, at least or exactly or at most 2505, at least or exactly or at most 2506, at least or exactly or at most 2507, at least or exactly or at most 2508, at least or exactly or at most 2509, at least or exactly or at most 2510, at least or exactly or at most 2511, at least or exactly or at most 2512, at least or exactly or at most 2513, at least or exactly or at most 2514, at least or exactly or at most 2515, at least or exactly or at most 2516, at least or exactly or at most 2517, at least or exactly or at most 2518, at least or exactly or at most 2519, at least or exactly or at most 2520, at least or exactly or at most 2521, at least or exactly or at most 2522, at least or exactly or at most 2523, at least or exactly or at most 2524, at least or exactly or at most 2525, at least or exactly or at most 2526, at least or exactly or at most 2527, at least or exactly or at most 2528, at least or exactly or at most 2529, at least or exactly or at most 2530, at least or exactly or at most 2531, at least or exactly or at most 2532, at least or exactly or at most 2533, at least or exactly or at most 2534, at least or exactly or at most 2535, at least or exactly or at most 2536, at least or exactly or at most 2537, at least or exactly or at most 2538, at least or exactly or at most 2539, at least or exactly or at most 2540, at least or exactly or at most 2541, at least or exactly or at most 2542, at least or exactly or at most 2543, at least or exactly or at most 2544, at least or exactly or at most 2545, at least or exactly or at most 2546, at least or exactly or at most 2547, at least or exactly or at most 2548, at least or exactly or at most 2549, at least or exactly or at most 2550, at least or exactly or at most 2551, at least or exactly or at most 2552, at least or exactly or at most 2553, at least or exactly or at most 2554, at least or exactly or at most 2555, at least or exactly or at most 2556, at least or exactly or at most 2557, at least or exactly or at most 2558, at least or exactly or at most 2559, at least or exactly or at most 2560, at least or exactly or at most 2561, at least or exactly or at most 2562., at least or exactly or at most 2563, at least or exactly or at most 2564, at least or exactly or at most 2565, at least or exactly or at most 2566, at least or exactly or at most 2567, at least or exactly or at most 2568, at least or exactly or at most 2569, at least or exactly or at most 2570, at least or exactly or at most 2571, at least or exactly or at most 2572, at least or exactly or at most 2573, at least or exactly or at most 2574, at least or exactly or at most 2575, at least or exactly or at most 2576, at least or exactly or at most 2577, at least or exactly or at most 2578, at least or exactly or at most 2579, at least or exactly or at most 2580, at least or exactly or at most 2581, at least or exactly or at most 2582, at least or exactly or at most 2583, at least or exactly or at most 2584, at least or exactly or at most 2585, at least or exactly or at most 2586, at least or exactly or at most 2587, at least or exactly or at most 2588, at least or exactly or at most 2589, at least or exactly or at most 2590, at least or exactly or at most 2591, at least or exactly or at most 2592, at least or exactly or at most 2593, at least or exactly or at most 2594, at least or exactly or at most 2595, at least or exactly or at most 2596, at least or exactly or at most 2597, at least or exactly or at most 2598, at least or exactly or at most 2599, at least or exactly or at most 2600, at least or exactly or at most 2601, at least or exactly or at most 2602, at least or exactly or at most 2603, at least or exactly or at most 2604, at least or exactly or at most 2605, at least or exactly or at most 2606, at least or exactly or at most 2607, at least or exactly or at most 2608, at least or exactly or at most 2609, at least or exactly or at most 2610, at least or exactly or at most 2611, at least or exactly or at most 2612, at least or exactly or at most 2613, at least or exactly or at most 2614, at least or exactly or at most 2615, at least or exactly or at most 2616, at least or exactly or at most 2617, at least or exactly or at most 2618, at least or exactly or at most 2619, at least or exactly or at most 2620, at least or exactly or at most 2621, at least or exactly or at most 2622, at least or exactly or at most 2623, at least or exactly or at most 2624, at least or exactly or at most 2625, at least or exactly or at most 2626, at least or exactly or at most 2627, at least or exactly or at most 2628, at least or exactly or at most 2629, at least or exactly or at most 2630, at least or exactly or at most 2631, at least or exactly or at most 2632, at least or exactly or at most 2633, at least or exactly or at most 2634, at least or exactly or at most 2635, at least or exactly or at most 2636, at least or exactly or at most 2637, at least or exactly or at most 2638, at least or exactly or at most 2639, at least or exactly or at most 2640, at least or exactly or at most 2641, at least or exactly or at most 2642, at least or exactly or at most 2643, at least or exactly or at most 2644, at least or exactly or at most 2645, at least or exactly or at most 2646, at least or exactly or at most 2647, at least or exactly or at most 2648, at least or exactly or at most 2649, at least or exactly or at most 2650, at least or exactly or at most 2651, at least or exactly or at most 2652, at least or exactly or at most 2653, at least or exactly or at most 2654, at least or exactly or at most 2655, at least or exactly or at most 2656, at least or exactly or at most 2657, at least or exactly or at most 2658, at least or exactly or at most 2659, at least or exactly or at most 2660, at least or exactly or at most 2661, at least or exactly or at most 2662, at least or exactly or at most 2663, at least or exactly or at most 2664, at least or exactly or at most 2665, at least or exactly or at most 2666, at least or exactly or at most 2667, at least or exactly or at most 2668, at least or exactly or at most 2669, at least or exactly or at most 2670, at least or exactly or at most 2671, at least or exactly or at most 2672, at least or exactly or at most 2673, at least or exactly or at most 2674, at least or exactly or at most 2675, at least or exactly or at most 2676, at least or exactly or at most 2677, at least or exactly or at most 2678, at least or exactly or at most 2679, at least or exactly or at most 2680, at least or exactly or at most 2681, at least or exactly or at most 2682, at least or exactly or at most 2683, at least or exactly or at most 2684, at least or exactly or at most 2685, at least or exactly or at most 2686, at least or exactly or at most 2687, at least or exactly or at most 2688, at least or exactly or at most 2689, at least or exactly or at most 2690, at least or exactly or at most 2691, at least or exactly or at most 2692, at least or exactly or at most 2693, at least or exactly or at most 2694, at least or exactly or at most 2695, at least or exactly or at most 2696, at least or exactly or at most 2697, at least or exactly or at most 2698, at least or exactly or at most 2699, at least or exactly or at most 2700, at least or exactly or at most 2701, at least or exactly or at most 2702, at least or exactly or at most 2703, at least or exactly or at most 2704, at least or exactly or at most 2705, at least or exactly or at most 2706, at least or exactly or at most 2707, at least or exactly or at most 2708, at least or exactly or at most 2709, at least or exactly or at most 2710, at least or exactly or at most 2711, at least or exactly or at most 2712, at least or exactly or at most 2713, at least or exactly or at most 2714, at least or exactly or at most 2715, at least or exactly or at most 2716, at least or exactly or at most 2717, at least or exactly or at most 2718, at least or exactly or at most 2719, at least or exactly or at most 2720, at least or exactly or at most 2721, at least or exactly or at most 2722, at least or exactly or at most 2723, at least or exactly or at most 2724, at least or exactly or at most 2725, at least or exactly or at most 2726, at least or exactly or at most 2727, at least or exactly or at most 2728, at least or exactly or at most 2729, at least or exactly or at most 2730, at least or exactly or at most 2731, at least or exactly or at most 2732, at least or exactly or at most 2733, at least or exactly or at most 2734, at least or exactly or at most 2735, at least or exactly or at most 2736, at least or exactly or at most 2737, at least or exactly or at most 2738, at least or exactly or at most 2739, at least or exactly or at most 2740, at least or exactly or at most 2741, at least or exactly or at most 2742, at least or exactly or at most 2743, at least or exactly or at most 2744, at least or exactly or at most 2745, at least or exactly or at most 2746, at least or exactly or at most 2747, at least or exactly or at most 2748, at least or exactly or at most 2749, at least or exactly or at most 2750, at least or exactly or at most 2751, at least or exactly or at most 2752, at least or exactly or at most 2753, at least or exactly or at most 2754, at least or exactly or at most 2755, at least or exactly or at most 2756, at least or exactly or at most 2757, at least or exactly or at most 2758, at least or exactly or at most 2759, at least or exactly or at most 2760, at least or exactly or at most 2761, at least or exactly or at most 2762, at least or exactly or at most 2763, at least or exactly or at most 2764, at least or exactly or at most 2765, at least or exactly or at most 2766, at least or exactly or at most 2767, at least or exactly or at most 2768, at least or exactly or at most 2769, at least or exactly or at most 2770, at least or exactly or at most 2771, at least or exactly or at most 2772, at least or exactly or at most 2773, at least or exactly or at most 2774, at least or exactly or at most 2775, at least or exactly or at most 2776, at least or exactly or at most 2777, at least or exactly or at most 2778, at least or exactly or at most 2779, at least or exactly or at most 2780, at least or exactly or at most 2781, at least or exactly or at most 2782, at least or exactly or at most 2783, at least or exactly or at most 2784, at least or exactly or at most 2785, at least or exactly or at most 2786, at least or exactly or at most 2787, at least or exactly or at most 2788, at least or exactly or at most 2789, at least or exactly or at most 2790, at least or exactly or at most 2791, at least or exactly or at most 2792, at least or exactly or at most 2793, at least or exactly or at most 2794, at least or exactly or at most 2795, at least or exactly or at most 2796, at least or exactly or at most 2797, at least or exactly or at most 2798, at least or exactly or at most 2799, at least or exactly or at most 2800, at least or exactly or at most 2801, at least or exactly or at most 2802, at least or exactly or at most 2803, at least or exactly or at most 2804, at least or exactly or at most 2805, at least or exactly or at most 2806, at least or exactly or at most 2807, at least or exactly or at most 2808, at least or exactly or at most 2809, at least or exactly or at most 2810, at least or exactly or at most 2811, at least or exactly or at most 2812, at least or exactly or at most 2813, at least or exactly or at most 2814, at least or exactly or at most 2815, at least or exactly or at most 2816, at least or exactly or at most 2817, at least or exactly or at most 2818, at least or exactly or at most 2819, at least or exactly or at most 2820, at least or exactly or at most 2821, at least or exactly or at most 2822, at least or exactly or at most 2823, at least or exactly or at most 2824, at least or exactly or at most 2825, at least or exactly or at most 2826, at least or exactly or at most 2827, at least or exactly or at most 2828, at least or exactly or at most 2829, at least or exactly or at most 2830, at least or exactly or at most 2831, at least or exactly or at most 2832, at least or exactly or at most 2833, at least or exactly or at most 2834, at least or exactly or at most 2835, at least or exactly or at most 2836, at least or exactly or at most 2837, at least or exactly or at most 2838, at least or exactly or at most 2839, at least or exactly or at most 2840, at least or exactly or at most 2841, at least or exactly or at most 2842, at least or exactly or at most 2843, at least or exactly or at most 2844, at least or exactly or at most 2845, at least or exactly or at most 2846, at least or exactly or at most 2847, at least or exactly or at most 2848, at least or exactly or at most 2849, at least or exactly or at most 2850, at least or exactly or at most 2851, at least or exactly or at most 2852, at least or exactly or at most 2853, at least or exactly or at most 2854, at least or exactly or at most 2855, at least or exactly or at most 2856, at least or exactly or at most 2857, at least or exactly or at most 2858, at least or exactly or at most 2859, at least or exactly or at most 2860, at least or exactly or at most 2861, at least or exactly or at most 2862, at least or exactly or at most 2863, at least or exactly or at most 2864, at least or exactly or at most 2865, at least or exactly or at most 2866, at least or exactly or at most 2867, at least or exactly or at most 2868, at least or exactly or at most 2869, at least or exactly or at most 2870, at least or exactly or at most 2871, at least or exactly or at most 2872, at least or exactly or at most 2873, at least or exactly or at most 2874, at least or exactly or at most 2875, at least or exactly or at most 2876, at least or exactly or at most 2877, at least or exactly or at most 2878, at least or exactly or at most 2879, at least or exactly or at most 2880, at least or exactly or at most 2881, at least or exactly or at most 2882, at least or exactly or at most 2883, at least or exactly or at most 2884, at least or exactly or at most 2885, at least or exactly or at most 2886, at least or exactly or at most 2887, at least or exactly or at most 2888, at least or exactly or at most 2889, at least or exactly or at most 2890, at least or exactly or at most 2891, at least or exactly or at most 2892, at least or exactly or at most 2893, at least or exactly or at most 2894, at least or exactly or at most 2895, at least or exactly or at most 2896, at least or exactly or at most 2897, at least or exactly or at most 2898, at least or exactly or at most 2899, at least or exactly or at most 2900, at least or exactly or at most 2901, at least or exactly or at most 2902, at least or exactly or at most 2903, at least or exactly or at most 2904, at least or exactly or at most 2905, at least or exactly or at most 2906, at least or exactly or at most 2907, at least or exactly or at most 2908, at least or exactly or at most 2909, at least or exactly or at most 2910, at least or exactly or at most 2911, at least or exactly or at most 2912, at least or exactly or at most 2913, at least or exactly or at most 2914, at least or exactly or at most 2915, at least or exactly or at most 2916, at least or exactly or at most 2917, at least or exactly or at most 2918, at least or exactly or at most 2919, at least or exactly or at most 2920, at least or exactly or at most 2921, at least or exactly or at most 2922, at least or exactly or at most 2923, at least or exactly or at most 2924, at least or exactly or at most 2925, at least or exactly or at most 2926, at least or exactly or at most 2927, at least or exactly or at most 2928, at least or exactly or at most 2929, at least or exactly or at most 2930, at least or exactly or at most 2931, at least or exactly or at most 2932, at least or exactly or at most 2933, at least or exactly or at most 2934, at least or exactly or at most 2935, at least or exactly or at most 2936, at least or exactly or at most 2937, at least or exactly or at most 2938, at least or exactly or at most 2939, at least or exactly or at most 2940, at least or exactly or at most 2941, at least or exactly or at most 2942, at least or exactly or at most 2943, at least or exactly or at most 2944, at least or exactly or at most 2945, at least or exactly or at most 2946, at least or exactly or at most 2947, at least or exactly or at most 2948, at least or exactly or at most 2949, at least or exactly or at most 2950, at least or exactly or at most 2951, at least or exactly or at most 2952, at least or exactly or at most 2953, at least or exactly or at most 2954, at least or exactly or at most 2955, at least or exactly or at most 2956, at least or exactly or at most 2957, at least or exactly or at most 2958, at least or exactly or at most 2959, at least or exactly or at most 2960, at least or exactly or at most 2961, at least or exactly or at most 2962, at least or exactly or at most 2963, at least or exactly or at most 2964, at least or exactly or at most 2965, at least or exactly or at most 2966, at least or exactly or at most 2967, at least or exactly or at most 2968, at least or exactly or at most 2969, at least or exactly or at most 2970, at least or exactly or at most 2971, at least or exactly or at most 2972, at least or exactly or at most 2973, at least or exactly or at most 2974, at least or exactly or at most 2975, at least or exactly or at most 2976, at least or exactly or at most 2977, at least or exactly or at most 2978, at least or exactly or at most 2979, at least or exactly or at most 2980, at least or exactly or at most 2981, at least or exactly or at most 2982, at least or exactly or at most 2983, at least or exactly or at most 2984, at least or exactly or at most 2985, at least or exactly or at most 2986, at least or exactly or at most 2987, at least or exactly or at most 2988, at least or exactly or at most 2989, at least or exactly or at most 2990, at least or exactly or at most 2991, at least or exactly or at most 2992, at least or exactly or at most 2993, at least or exactly or at most 2994, at least or exactly or at most 2995, at least or exactly or at most 2996, at least or exactly or at most 2997, at least or exactly or at most 2998, at least or exactly or at most 2999, at least or exactly or at most 3000, at least or exactly or at most 3001, at least or exactly or at most 3002, at least or exactly or at most 3003, at least or exactly or at most 3004, at least or exactly or at most 3005, at least or exactly or at most 3006, at least or exactly or at most 3007, at least or exactly or at most 3008, at least or exactly or at most 3009, at least or exactly or at most 3010, at least or exactly or at most 3011, at least or exactly or at most 3012, at least or exactly or at most 3013, at least or exactly or at most 3014, at least or exactly or at most 3015, at least or exactly or at most 3016, at least or exactly or at most 3017, at least or exactly or at most 3018, at least or exactly or at most 3019, at least or exactly or at most 3020, at least or exactly or at most 3021, at least or exactly or at most 3022, at least or exactly or at most 3023, at least or exactly or at most 3024, at least or exactly or at most 3025, at least or exactly or at most 3026, at least or exactly or at most 3027, at least or exactly or at most 3028, at least or exactly or at most 3029, at least or exactly or at most 3030, at least or exactly or at most 3031, at least or exactly or at most 3032, at least or exactly or at most 3033, at least or exactly or at most 3034, at least or exactly or at most 3035, at least or exactly or at most 3036, at least or exactly or at most 3037, at least or exactly or at most 3038, at least or exactly or at most 3039, at least or exactly or at most 3040, at least or exactly or at most 3041, at least or exactly or at most 3042, at least or exactly or at most 3043, at least or exactly or at most 3044, at least or exactly or at most 3045, at least or exactly or at most 3046, at least or exactly or at most 3047 contiguous amino acid residues.

In some embodiments, the polypeptide of the invention also has a sequence identity with the amino acid sequence of a) defined above of at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%. Similarly, the polypeptide of the invention in some embodiments also has a sequence identity with the amino add sequence of b) defined above of at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, and at least 99%.

In the embodiments defined by option b) above, the polypeptide of the invention is also one that has at least 5 contiguous amino acid residues defined for option b) above and also has its N-terminal amino acid residue corresponding to any one of amino acid residues 0.1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 38, 39, 40, 42, 43, 44, 45, 46, 47, 48, 49, 50, 51, 52, 53, 54, 55, and 56 in any one of SEQ 3D NOs: 1-30, if the length of the at least 5 amino acid residues so permit—if the length of the at least 5 amino adds are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 57, 58, 59, 60, 61 and 62 in any on of SEQ ID NOs: 2-30, If the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 63, 64, 65, 66, 67, 68, and 69 in any one of SEQ ID NOs: 4-30, If the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, and 104 in any one of SEQ ID NOs: 5-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 105, 106, 107, 108, 109, 110, and 110 in any one of SEQ ID NOs: 6-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 111, 112, 113, 114, 115, 116, 117, 118, 119, 120, 121, and 122 in any one of SEQ ID NOs: 7-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 123, 124, 125, 126, 127, 128, 129, 130, 131, 132, 133, 134, and 135 in any one of SEQ ID NOs: 8-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 136, 137, 138, 139, 140, 141, 142, 143, 144, 145, 146, 147, 148, 149, 150, 151, 152, 153, 154, and 155 in any one of SEQ ID NOs: 9-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 156, 157, and 158 in any one of SEQ ID NOs: 10-30, If the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 159, 160, 161, and 162 in any one of SEQ ID NOs: 11-30, if the length of the at least 5 amino acid residues so permit—if the length of the at least 5 amino adds are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino add residues 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, and 226 in any one of SEQ ID NOs: 12-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino add residue corresponding to any one of amino add residues 227, 228, 229, 230, 231, 232, 233, and 234 in any one of SEQ ID NOS: 13-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 235, 236, 237, 238, 239, 240, 241, 242, 243, 244, 245, 246, 247, 248, and 249 in SEQ ID NOs: 14-30, if the length of the at least 5 amino acid residues so permit—If the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino add residues 250, 251, 252, 253, 254, 255, 256, 257, 258, 259, 260, 271, 272, 273, 274, 275, 276, 277, 278, 279, 280, 281, 282, 283, 284, 285, 286, 287, 288, 289, and 290 In any one of SEQ ID NOs: 15-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, and 352 in any one of SEQ ID NOs: 16-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino add residues In the embodiments defined by option b) above, the polypeptide of the invention is also one that has at least 5 contiguous amino acid residues defined for option b) above and also has its N-terminal amino acid residue corresponding to any one of amino acid residues 353, 354, 355, 356, 357, 358, 359, 360, 361, 362, and 363 in any one of SEQ ID NOs: 17-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino add residues In the embodiments defined by option b) above, the polypeptide of the invention is also one that has at least 5 contiguous amino acid residues defined for option b) above and also has its N-terminal amino acid residue corresponding to any one of amino acid residues 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, and 416 in any one of SEQ ID NOs: 18-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention is also one that has at least 5 contiguous amino acid residues defined for option b) above and also has its N-terminal amino acid residue corresponding to any one of amino acid residues 417, 418, 419, 420, 421, 422, 423, 424, 425, 426, 427, 428, 429, 430, 431, 432, 433, 434, 435, 436, 437, 438, 439, 440, and 441 in any one of SEQ ID NOs: 19-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention Is also one that has at least 5 contiguous amino acid residues defined for option b) above and also has its N-terminal amino acid residue corresponding to any one of amino acid residues 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, and 707 in any one of SEQ ID NOs: 20-30, if the length of the at least 5 amino acid residues so permit—if the length of the at least amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention is also one that has at least 5 contiguous amino acid residues defined for option b) above and also has its N-terminal amino acid residue corresponding to any one of amino acid residues 708, 709, 710, 711, 712, 713, 714, 715, 716, 717, 718, 719, 720, 721, 722 and 723 in any one of SEQ ID NOs: 21-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention Is also one that has at least 5 contiguous amino acid residues defined for option b) above and also has its N-terminal amino acid residue corresponding to amino acid residue 724 in any one of SEQ ID NOs: 22.30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention is also one that has at least 5 contiguous amino add residues defined for option b) above and also has its N-terminal amino acid residue corresponding to any one of amino acid residues 725, 726, 727, 728, 729, 730, 731, 732, 733, 734, 735, 736, 737, 738, 739, 740, 741, 742, 743, 744, 745, 746, 747, 748, 749, and 750 in any one of SEQ ID NOs: 23-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention Is also one that has at least 5 contiguous amino acid residues defined for option b) above and also has its N-terminal amino acid residue corresponding to any one of amino acid residues 751, 752, 753, 754, 755, 756, 757, 758, 759, 760, 761, 762, 763, 764, 765, 766, 767, 768, 769, 770, 77.1, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, and 902 in any one of SEQ ID NOs: 24-30, if the length of the at least 5 amino acid residues so permit—if the length of the at least 5 amino acids are higher than 5, the N-terminal first residue will not be higher numbered than N−L+1, where N is the number of amino add residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention is also one that has at least 5 contiguous amino acid residues defined for option b) above and also has its N-terminal amino acid residue corresponding to any one of amino acid residues 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, and 970 in any one of SEQ ID NOs: 25-30, if the length of the at least 5 amino acid residues so permit—if the length of the at least amino acids are higher than 5, the N-terminal first residue will not be higher numbered than N−L+1, where N is the number of amino add residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention is also one that has at least 5 contiguous amino add residues defined for option b) above and also has its N-terminal amino acid residue corresponding to any one of amino acid residues 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, and 1067 in any one of SEQ ID NOs: 26-30, if the length of the at least 5 amino add residues so permit—If the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention is also one that has at least 5 contiguous amino acid residues defined for option b) above and also has its N-terminal amino acid residue corresponding to any one of amino acid residues 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, and 1106 in any one of SEQ ID NOs: 27-30, if the length of the at least 5 amino acid residues so permit—if the length of the at least amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention is also one that has at least 5 contiguous amino acid residues defined for option b) above and also has its N-terminal amino acid residue corresponding to any one of amino acid residues 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, and 1224 in any one of SEQ ID NOs: 28-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues In the embodiments defined by option b) above, the polypeptide of the invention is also one that has at least 5 contiguous amino acid residues defined for option b) above and also has its N-terminal amino acid residue corresponding to any one of amino acid residues 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, and 1280 in any one of SEQ ID NOs: 29-30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least 5 contiguous amino acid residues defined for option b) above and also has its N-terminal amino acid residue corresponding to any one of amino acid residues 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2030, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3042, and 3043 in any one of SEQ ID NOs: 30, if the length of the at least 5 amino acid residues so permit—if the length of the at least 5 amino acids are higher than 5, the N-terminal first residue will not be higher numbered than N−L+1, where N is the number of amino acid residues of the reference sequence and L is the number of amino acids defined for option b.

The polypeptide of the invention is in certain embodiments also fused or conjugated to an immunogenic carrier molecule; or, phrased otherwise, the polypeptide of the invention also includes such an immunogenic carrier molecule in addition to the material derived from SEQ ID NOs. 1-30. The immunogenic carrier molecule is a typically polypeptide that induces T-helper lymphocyte responses in a majority of humans, such as Immunogenic carrier proteins selected from the group consisting of keyhole limpet hemocyanino or a fragment thereof, tetanus toxoid or a fragment thereof, diphtheria toxoid or a fragment thereof. Other suitable carrier molecules are discussed infra. One further fusion partner, which is preferably incorporated is a “His tag”, i.e. a stretch of amino adds, which is rich or only consists of histidinyl residues so as to facilitate protein purification.

In preferred embodiments, the polypeptide of the invention detailed above is capable of inducing an adaptive immune response against the polypeptide in a mammal, in particular in a human being. Preferably, the adaptive immune response is a protective adaptive immune response against infection with A baumanii. The polypeptide may in these cases induce a humeral and/or a cellular immune response.

Epitopes SEQ 10 NOs: 1-30 include antigenic determinants (epitopes) that are as such recognized by antibodies and/or when bound to MHC molecules by T-cell receptors. For the purposes of the present invention, B-cell epitopes (i.e. antibody binding epitopes) are of particular relevance.

It is relatively uncomplicated to identify linear B-cell epitopes—one very simple approach entails that antibodies raised agains A. baumannii or A baumannii derived proteins disclosed herein are tested for binding to overlapping oligomeric peptides derived from any one of SEQ ID NO: 1-30. Thereby, the regions of the A. baumannii polypeptide which are responsible for or contribute to binding to the antibodies can be identified.

Alternatively, or additionally, one can produce mutated versions of the polypeptides of the invention, e.g. version where each single non-alanine residue in SEQ ID NOs.: 1-30 are point mutated to alanine—this method also assists In identifying complex assembled B-cell epitopes; this is the case when binding of the same antibody is modified by exchanging amino acids in different areas of the full-length polypeptide.

Also, in silico methods for B-cell epitope prediction can be employed: useful state-of-the-art systems for β-turn prediction is provided In Petersen B et al. (November 2010), Plos One 5(11): e15079; prediction of linear B-cell epitopes, cf: Larsen 3 E P et al. (April 2006), Immunome Research, 2:2; prediction of solvent exposed amino adds: Petersen B et al (July 2009), BMC Structural Biology, 9:51,

### The Nucleic Acid Fragments of the Invention

The nucleic add fragment of the invention referred to above is preferably is a DNA fragment (such as SEQ ID NOs: 31-60) or an RNA fragment (such as SEQ ID NOs 61-90).

The nucleic acid fragment of the invention typically consists of at least or exactly or at most 11, such as at least or exactly or at most 12, at least or exactly or at most 13, at least or exactly or at most 14, at least or exactly or at most 15, at least or exactly or at most 16, at least or exactly or at most 17 at least or exactly or at most 18, at least or exactly or at most 19, at least or exactly or at most 20, at least or exactly or at most 21, at least or exactly or at most 22, at least or exactly or at most 23, at least or exactly or at most 24, at least or exactly or at most 25, at least or exactly or at most 26, at least or exactly or at most 27, at least or exactly or at most 28, at least or exactly or at most 29, at feast or exactly or at most 30, at least or exactly or at most 31, at least or exactly or at most 32, at least or exactly or at most 33, at least or exactly or at most 34, at least or exactly or at most 35, at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55, at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94, at least or exactly or at most 95, at least or exactly or at most 96, at least or exactly or at most 97, at least or exactly or at most 98, at least or exactly or at most 99, at least or exactly or at most 100, at feast or exactly or at most 101, at least or exactly or at most 102, at least or exactly or at most 103, at least or exactly or at most 104, at least or exactly or at most 105, at least or exactly or at most 106, at least or exactly or at most 107, at least or exactly or at most 108, at least or exactly or at most 109, at least or exactly or at most 110, at least or exactly or at most 111, at least or exactly or at most 112, at least or exactly or at most 113, at least or exactly or at most 114, at least or exactly or at most 115, at least or exactly or at most 116, at least or exactly or at most 117, at least or exactly or at most 118, at least or exactly or at most 119, at least or exactly or at most 120, at least or exactly or at most 121, at least or exactly or at most 122, at least or exactly or at most 123, at least or exactly or at most 124, at least or exactly or at most 125, at least or exactly or at most 126, at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, at least or exactly or at most 139, at least or exactly or at most 140, at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, at least or exactly or at most 150, at least or exactly or at most 151, at least or exactly or at most 152, at least or exactly or at most 153, at least or exactly or at most 154, at least or exactly or at most 155, at least or exactly or at most 156, at least or exactly or at most 157, at least or exactly or at most 158, at least or exactly or at most 159, at least or exactly or at most 160, at least or exactly or at most 161, at least or exactly or at most 162, at least or exactly or at most 163, at least or exactly or at most 164, at least or exactly or at most 165, at least or exactly or at most 166, at least or exactly or at most 167, at least or exactly or at most 168, at least or exactly or at most 169, at least or exactly or at most 170, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182 and at least or exactly or at most 183 consecutive nucleotides in any one of SEQ ID NOs: 31-90. Longer fragments are contemplated, i.e. fragments having at least or exactly or at most 200, at least or exactly or at most 300 at least or exactly or at most 400, at least or exactly or at most 500, at least or exactly or at most 600, at least or exactly or at most 700, at least or exactly or at most 800, at least or exactly or at most 900, at least or exactly or at most 1000, at least or exactly or at most 1500, at least or exactly or at most 2000, at least or exactly or at most 2500, at least or exactly or at most 3000, at least or exactly or at most 3500, and at least or exactly or at most 4000 nucleotides from those of SEQ ID NOs: 31-90 that encompass fragments of such lengths. Particularly preferred nucleic acid fragments (DNA or RNA) are those fragments of any one of SEQ ID NOs 31-90, which encode a polypeptide of the present invention discussed supra.

The nucleic acid fragment of the invention discussed above typically has a sequence identity with the nucleotide sequence defined for i) or ii) above, which is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

The nucleic acid fragment of the invention discussed above may also have a sequence identity with the nucleotide sequence defined for iii) above, which is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

### The Vectors of the Invention

Vectors of the invention fall into several categories discussed infra. One preferred vector of the invention comprises in operable linkage and in the 5-3′ direction, an expression control region comprising an enhancer/promoter for driving expression of the nucleic acid fragment defined for option I) above, optionally a signal peptide coding sequence, a nucleotide sequence defined for option I), and optionally a terminator. Hence, such a vector constitutes an expression vector useful for effecting production in cells of the polypeptide of the invention. Since the polypeptides of the invention are bacterial of origin, recombinant production is conveniently effected in bacterial host cells, so here it is preferred that the expression control region drives expression in prokaryotic cell such as a bacterium, e.g. in F coll. However, if the vector is to drive expression in mammalian cell (as would be the case for a DNA vaccine vector), the expression control region should be adapted to this particular use.

At any rate, certain vectors of the invention are capable of autonomous replication.

Also, the vector of the invention may be one that is capable of being integrated into the genome of a host cell—this is particularly useful if the vector is use in the production of stably transformed cells, where the progeny will also include the genetic information introduced via the vector. Alternatively, vectors incapable of being integrated into the genome of a mammalian host cell are useful in e.g. DNA vaccination.

Typically, the vector of the invention is selected from the group consisting of a virus, such as a attenuated virus (which may in itself be useful as a vaccine agent), a bacteriophage, a plasmid, a minichromosome, and a cosmid.

A more detailed discussion of vectors of the invention is provided in the following:

Polypeptides of the invention may be encoded by a nucleic acid molecule comprised in a vector. A nucleic acid sequence can be “heterologous,” which means that it is in a context foreign to the cell in which the vector is being introduced, which includes a sequence homologous to a sequence in the cell but in a position within the host cell where it is ordinarily not found. Vectors include naked DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al, 2001; Ausubel et al, 1996, both incorporated herein by reference). In addition to encoding the polypeptides of this invention, a vector of the present invention may encode polypeptide sequences such as a tag or immunogenicity enhancing peptide (e.g. an immunogenic carrier or a fusion partner that stimulates the immune system, such as a cytokine or active fragment thereof). Useful vectors encoding such fusion proteins include pIN vectors (Inouye et al, 1985), vectors encoding a stretch of histidines, and pGEX vectors, for use in generating glutathione S-transferase (GST) soluble fusion proteins for later purification and separation or cleavage.

Vectors of the invention may be used in a host cell to produce a polypeptide of the invention that may subsequently be purified for administration to a subject or the vector may be purified for direct administration to a subject for expression of the protein in the subject (as is the case when administering a nucleic acid vaccine).

Expression vectors can contain a variety of “control sequences,” which refer to nucleic acid sequences necessary for the transcription and possibly translation of an operably linked coding sequence In a particular host organism. In addition to control sequences that govern transcription and translation, vectors and expression vectors may contain nucleic acid sequences that serve other functions as well and are described infra,

### 1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked;” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved In the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

Naturally, it may be important to employ a promoter and/or enhancer that effectively direct(s) the expression of the DNA segment in the cell type or organism chosen for expression. Those of skill in the art of molecular biology generally know the use of promoters, enhancers, and cell type combinations for protein expression (see Sambrook et al, 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Examples of inducible elements, which are regions of a nucleic acid sequence that can be activated in response to a specific stimulus, include but are not limited to immunoglobulin Heavy Chain (Banerji et al, 1983; Gilles et al, 1983; Grosschedi et at, 1985; Atchinson et al, 1986, 1987; toiler et al, 1987; Weinberger et al, 1984; Kiledjian et al, 1988; Porton et al; 1990), Immunoglobulin Light Chain (Queen et al, 1983; Picard et al, 1984), T Cell Receptor (Luria et al, 1987; Winoto et al, 1989; Redondo et al; 1990), HLA DQα and/or DQβ (Sullivan et al, 1987), β-Interferon (Goodbourn et al, 1986; Fujita et al, 1987; Goodbourn et al, 1988), Interleukin-2 (Greene et al, 1989), Interleukin-2 Receptor (Greene et al. 1989; Lin et al, 1990), MHC Class II 5 (Koch et al, 1989), MHC Class II HLA-DRα (Sherman et al, 1989), β-Actin (Kawamoto et al, 1988; Ng et al; 1989), Muscle Creatine Kinase (MCK) (Jaynes et al, 1988; Hortick et al, 1989; Johnson et al, 1989), Prealbumin (Transthyretin) (Costa et al, 1988), Elastase I (Omitz et al, 1987), Metallothionein (MTII) (Karin et al, 1987; Culotta et al, 1989), Collagenase (Pinkert et al, 1987; Angel et al, 1987), Albumin (Pinkert et al, 1987; Tranche et al, 1989, 1990), α-Fetoprotein (Godbout et al, 1988; Campere et al, 1989), γ-Globin (Bodine et al, 1987; Perez-Stable et al, 1990), β-Globin (Trudel et al, 1987), c-fos (Cohen et al, 1987), c-HA-ras (Triesman, 1986; Deschamps et al, 1985), Insulin (Edlund et al, 1985), Neural Cell Adhesion Molecule (NCAM) (Hirsh et al, 1990), αl-Antitrypain (Larimer et al, 1990). H2B (TH2B) Histone (Hwang et al, 1990), Mouse and/or Type I Collagen (Ripe et al, 1989), Glucose-Regulated Proteins (GRP94 and GRP78) (Chang et al, 1989), Rat Growth Hormone (Larsen et al, 1986), Human Serum Amyloid A (SAA) (Edbrooke et al, 1989), Troponin I (TN I) (Yutzey et at, 1989), Platelet-Derived Growth Factor (PDGF) (Pech et al, 1989), Duchenne Muscular Dystrophy (Klamut et al, 1990), SV40 (Banerji et al, 1981; Moreau et al, 1981; Sleigh et al, 1985; Firak et al, 1986; Herr et al, 1986; Imbra et al, 1986; Kadesch et al, 1986; Wang et al, 1986; Ondek et al, 1987; Kuhl et al, 1987; Schaffner et al, 1988), Polyoma (Swartzendruber et at, 1975; Vasseur et al, 1980; Katinka et al, 1980, 1981; Tyndell et al, 1981; Dandolo et al, 1983; de Villiers et al, 1984; Hen et al, 1986; Satake et al, 1988; Campbell et al, 1988), Retroviruses (Kriegler et al, 1982, 1983; Levinson et al, 1982; Kriegler et al, 1983, 1984a, b, 1988; Bosze et al, 1986; Miksicek et al, 1986; Colander et al, 1987; Thiesen et al, 1988; Celander et al, 1988; Choi et al, 1988; Reisman at al, 1989), Papilloma Virus (Campo et al, 1983; Lusky et al, 1983; Spandidos and Wilkie, 1983; Spalholz et al, 1985; Lusky et al, 1986; Cripe et al, 1987; Gloss et al, 1987; Hirochika et al, 1987; Stephens et al, 1987), Hepatitis 8 Virus (Bulla et al, 1986; Jameel et at, 1986; Shaul et al, 1987; Spandau et al, 1988; Vannice et al, 1988), Human Immunodeficiency Virus (Muesing et al, 1987; Hauber et al, 1988; Jakobovits et al, 1988; Feng et al, 1988; Takebe et al, 1988; Rosen et al, 1988; Berkhout et al, 1989; Laspia et al, 1989; Sharp et at, 1989; Braddock et al, 1989), Cytomegalovirus (CMV) IE (Weber et al, 1984; Boshart et al, 1985; Foecking et al, 1986), Gibbon Ape Leukemia Virus (Holbrook et at, 1987; Quinn et al, 1989),

Inducible Elements include, but are not limited to MT II-Phorbol Ester (TFA)/Heavy metals (Palmiter et al, 1982; Haslinger et al, 1985; Searle et al, 1985; Stuart et al, 1985; Imagawa et al, 1987, Karin et al, 1987; Angel et al, 1987b; McNeall et al, 1989); MMTV (mouse mammary tumor virus)—Glucocorticoids (Huang et al, 1981; Lee et al, 1981; Majors et al, 1983; Chandler et al, 1983; Lee et al, 1984; Ponta et al, 1985; Sakal et at, 1988); β-Interferon-poly(rl)x/poly(rc) (Tavernier et at, 1983); Adenovirus S E2-EIA (Imperiale et al, 1984); Collagenase-Phorbol Ester (TPA) (Angel et al, 1987a); Stromelysin-Phorbol Ester (TPA) (Angel et al, 1987b); SV40-Phorbol Ester (TPA) (Angel et al, 1987b); Murine MX Gene—Interferon, Newcastle Disease Virus (Hug et al, 1988); GRP78 Gene-A23187 (Resendez et al, 1988); α-2-Macroglobulin IL-6 (Kunz et al, 1989); Vimentin-Serum (Rittling et al, 1989); MHC Class I Gene H-2Kb-Interferon (Blanar et al, 1989); HSP70-E1A/SV40 Large T Antigen (Taylor et al, 1989, 1990a, 1990b); Proliferin-Phorbot Ester/TPA (Mordacq et al, 1989); Tumor Necrosis Factor-PMA (Hensel et al, 1989); and Thyroid Stimulating Hormonea Gene-Thyroid Hormone (Chatterjee et al, 1989).

Also contemplated as useful In the present invention are the dectin-1 and dectin-2 promoters. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

In various embodiments, the human cytomegalovirus (CMV) Immediate early gene promoter, the SV40 early promoter, and the Rous sarcoma virus long terminal repeat can be used to obtain high level expression of a related polynucleotide to this invention. The use of other viral or mammalian cellular or bacterial phage promoters, which are well known in the art, to achieve expression of polynucleotides is contemplated as well.

In embodiments in which a vector is administered to a subject for expression of the protein, it is contemplated that a desirable promoter for use with the vector is one that is not down-regulated by cytokines or one that is strong enough that even if down-regulated, it produces an effective amount of the protein/polypeptide of the current invention in a subject to elicit an immune response. Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells or macrophages. The mammalian MHC I and MHC II promoters are examples of such tissue-specific promoters. 2. Initiation Signals and internal Ribosome Binding Sites (IRES) A specific initiation signal also may be required for efficient translation of coding sequences.

These signals include the ATG initiation codon or adjacent sequences. Exogenous translational control signals, including the ATG initiation codon, may need to be provided. One of ordinary skill In the art would readily be capable of determining this and providing the necessary signals. It is well known that the initiation codon must be “In-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated cap dependent translation and begin translation at internal sites (Pelletier and Sonenberg, 1988). IRES elements from two members of the picornavirus family (pollo and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages, By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

### 2. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al, 1999, Levenson et al, 1998, and Cocea, 1997, Incorporated herein by reference.) Frequently, a vector is linearized or fragmented using a restriction enzyme that cuts within the MCS to enable exogenous sequences to be ligated to the vector. Techniques involving restriction enzymes and ligation reactions are well known to those of skill in the art of recombinant technology,

### 3. Splicing Sites

Most transcribed eukaryotic RNA molecules will undergo RNA splicing to remove introns from the primary transcripts. If relevant In the context of vectors of the present invention, vectors containing genomic eukaryotic sequences may require donor and/or acceptor splicing sites to ensure proper processing of the transcript for protein expression. (See Chandler et al, 1997, incorporated herein by reference.)

### 4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site.

This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and It is more preferred that the terminator signal promotes polyadenylation of the message.

Terminators contemplated for use In the invention include any known terminator of transcription described herein or known to one of ordinary skill In the art, including but not limited to, for example, the bovine growth hormone terminator or viral termination sequences, such as the SV40 terminator. In certain embodiments, the termination signal may be a lack of transcribable or translatable sequence, such as due to a sequence truncation.

### 5. Polyadenylation Signals

In expression, particularly eukaryotic expression (as is relevant in nucleic acid vaccination), one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

### 6. Origins of Replication

In order to propagate a vector in a host cell, it may contain one or more origins of replication sites (often termed “on”), which is a specific nucleic acid sequence at which replication is initiated. Alternatively an autonomously replicating sequence (ARS) can be employed if the host cell is yeast.

### 7. Selectable and Screenable Markers

In certain embodiments of the invention, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein of the invention. Further examples of selectable and screenable markers are well known to one of skill in the art.

### The Transformed Cells of the Invention

Transformed cells of the invention are useful as organisms for producing the polypeptide of the invention, but also as simple “containers” of nucleic adds and vectors of the invention.

Certain transformed cells of the invention are capable of replicating the nucleic acid fragment defined for option I) of the second aspect of the invention. Preferred transformed cells of the invention are capable of expressing the nucleic acid fragment defined for option i), For recombinant production it is convenient, but not a prerequisite that the transformed cell according is prokaryotic, such as a bacterium, but generally both prokaryotic cells and eukaryotic cells may be used.

Suitable prokaryotic cells are bacterial calls selected from the group consisting of Escherichia (such as E. coli.), Bacillus [e.g. Bacillus subtilis], Salmonella, and Mycobacterium [preferably non-pathogenic, e.g. M. bovis BCG].

Eukaryotic cells can be in the form of yeasts (such as Saccharomyces cerevisiae) and protozoans. Alternatively, the transformed eukaryotic cells are derived from a multicellular organism such as a fungus, an insect cell, a plant cell, or a mammalian cell.

For production purposes, it is advantageous that the transformed cell of the invention is stably transformed by having the nucleic acid defined above for option I) stably integrated into its genome, and in certain embodiments it is also preferred that the transformed cell secretes or carries on its surface the polypeptide of the invention, since this facilitates recovery of the polypeptides produced. A particular version of this embodiment is one where the transformed cell is a bacterium and secretion of the polypeptide of the invention is into the periplasmic space.

As noted above, stably transformed cells are preferred—these i.a. allows that cell lines comprised of transformed cells as defined herein may be established—such cell lines are particularly preferred aspects of the invention.

Further details on cells and cell lines are presented in the following:

Suitable cells for recombinant nucleic acid expression of the nucleic acid fragments of the present invention are prokaryotes and eukaryotes. Examples of prokaryotic cells include E. coli; members of the Staphylococcus genus, such as S. epidermidis; members of the Lactobacillus genus, such as L. pantarum; members of the Lactococcus genus, such as L. laetis; members of the Bacillus genus, such as B. subtills; members of the Corynebacterium genus such as C. glutamicum; and members of the Pseudomonas genus such as Ps. fluorescens. Examples of eukaryotic cells include mammalian cells; insect cells; yeast cells such as members of the Saccharomyces genus (e.g. S. cerevisiae), members of the Pichia genus (e.g. P. pastoris), members of the Hansenula genus (e.g. H. polymorpha), members of the Kluyveromyces genus (e.g. K. lactis or K. fragilis) and members of the Schizosaccharomyces genus (e.g. S. pombe).

Techniques for recombinant gene production, introduction into a cell, and recombinant gene expression are well known in the art. Examples of such techniques are provided In references such as Ausubel, Current Protocols in Molecular Biology, John Wiley, 1987-2002, and Sambrook et al., Molecular Cloning, A Laboratory Manual, 2 nd Edition, Cold Spring Harbor Laboratory Press, 1989.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org) or from other depository institutions such as Deutsche Sammiung vor Micrroorganismen und Zellkulturen (DSM). An appropriate host can be determined by one of skill in the art based on the vector backbone and the desired result. A plasmid or cosmid, for example, can be introduced into a prokaryote host cell for replication of many vectors or expression of encoded proteins. Bacterial cells used as host cells for vector replication and/or expression include Staphylococcus strains, DH5α, JMI 09, and KC8, as well as a number of commercially available bacterial hosts such as SURE® Competent Cells and SOLOP ACK™ Gold Cells (STRATAGENE®, La Jolla, CA). Alternatively, bacterial cells such as E. coli LE392 could be used as host cells for phage viruses. Appropriate yeast cells include Saccharomyces cerevisiae., Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic add segment, such as described in U.S. Pat. Nos. 5,871,986, 4,879,236, both herein incorporated by reference, and which can be bought, for example, under the name MAXBAC® 2.0 from INVITROGEN® and BACPACK™ Baculovirus expression system from CLONTECH®

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### Amplification of Nucleic Acids

Nucleic acids used as a template for amplification may be isolated from cells, tissues or other samples according to standard methodologies (Sambrook et al, 2001). In certain embodiments, analysis is performed on whole cell or tissue homogenates or biological fluid samples without substantial purification of the template nucleic add. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to sequences of genes identified herein are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/VS89/01025, each of which is incorporated herein by reference in its entirety.

### Methods of Gene Transfer

Suitable methods for nucleic acid delivery to effect expression of compositions of the present invention are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, Incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al, 1987); by liposome mediated transfection (Nicolau and Sene, 1902; Fraley et al, 1979; Nicolau et al, 1987; Wong et al, 1980; Kaneda et al, 1989; Kato et al, 1991); by microprojectile bombardment (PCT Application Nos, WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al, 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al, 1993; U.S. Pat. Nos. 4,684,611 and 4,952,500, each incorporated herein by reference); by desiccation/inhibition mediated DNA uptake (Potrykus et al, 1985), Through the application of techniques such as these, organelle(s), cell(s), tissue(s) or organism(s) may be stably or transiently transformed.

The Antibodies of the invention—and their production/Isolation

Antibodies directed against the proteins of the invention are useful for affinity chromatography, immunoassays, and for distinguishing/identifying staphylococcus proteins as well as for passive immunisation and therapy.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antiserum is obtained by bleeding the immunized animal into a glass or plastic container, incubating the blood at 25 C for one hour, followed by incubating at 4 C for 2-18 hours. The serum is recovered by centrifugation (eg. 1,000 g for 10 minutes). About 20-50 ml per bleed may be obtained from rabbits.

Monoclonal antibodies are prepared using the standard method of Kohler & Milstein [Nature (1975) 256: 495-96], or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting 8-cells, or all dissociated spleen cells, are then induced to fuse with myeloma cells to form hybridomas, and are cultured in a selective I aedium (elg. hypexanthine, aminopterin, thymidine medium, “HAT”). The resulting hybridomas are plated by limiting dilution, and are assayed for production of antibodies, which bind specifically to the immunizing antigen (and which do not bind to unrelated antigens). The selected MAb-secreting hybridomas are then cultured either in vitro (eg. in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly 32p and 1251), electron-dense reagents, enzymes, and ligands having specific binding partners. Enzymes are typically detected by their activity. For example, horseradish peroxidase is usually detected by its ability to convert 3,3′, 5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, 1151 may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with, l25I, or with an anti-biotin MAb labeled with HRP. Other permutations and possibilities will be readily apparent to those of ordinary skill In the art, and are considered as equivalents within the scope of the instant invention.

According to the invention, the isolated monoclonal antibody or antibody analogue is preferably a monoclonal antibody selected from a multi-domain antibody such as a murine antibody, a chimeric antibody such as a humanized antibody, a fully human antibody, and single-domain antibody of a llama or a camel, or which is an antibody analogue selected from a fragment of an antibody such as an Fab or an F(ab′)₂, an scFV; cf. also the definition of the term “antibody” presented above.

Compositions of the invention: Vaccines

Pharmaceutical compositions, in particular vaccines, according to the invention may either be prophylactic (ie. to prevent infection) or therapeutic (ie, to treat disease after infection).

Such vaccines comprise immunising antigen(s), immunogen(s), polypeptide(s), protein(s) or nucleic acid(s), usually in combination with “pharmaceutically acceptable carriers”, which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc, pathogen, cf. the description of immunogenic carriers supra.

The pharmaceutical compositions of the invention thus typically contain an immunological adjuvant, which is commonly an aluminium based adjuvant or one of the other adjuvants described in the following: Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (WO 90/14837; Chapter 10 in vaccine design: the subunit and adjuvant approach, eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, MA), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi adjuvant system (RAS), (Ribi Immunochem, Hamilton, MT) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™); (3) saponin adjuvants such as Stimulon™ (Cambridge Bioscience, Worcester, MA) may be used or particles generated therefrom such as ISCOMs (immunostimulating complexes); (4) Complete Freund's Adjuvant (CFA) and incomplete Freund's Adjuvant (IFA); (5) cytokines, such as interleukins (eg. IL-1, IL-2, IL-4, IL-5, IL-6, IL-7, IL-12, etc.), interferons (eg. gamma interferon), macrophage colony stimulating factor (M-CSF), tumor necrosis factor (TNF), etc.; and (6) other substances that act as immunostimulating agents to enhance the effectiveness of the composition. Alum and MF59™ adjuvants are preferred.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

The immunogenic compositions (eg. the immunising antigen or immunogen or polypeptide or protein or nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (eg. nonhuma primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount will fall In a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, eg, by injection, either subcutaneously, intramuscularly, or transdermally/transcutaneously (eg. WO 98/20734). Additional formulations suitable for other modes of administration include oral and pulmonary formulations, suppositories, and transdermal applications. In the case of nucleic acid vaccination, also the intravenous or intraarterial routes may be applicable.

Dosage treatment may be a single dose schedule or a multiple dose schedule. The vaccine may be administered in conjunction with other immunoregulatory agents.

As an alternative to protein-based vaccines, DNA vaccination (also termed nucleic acid vaccination or gene vaccination) may be used [eq. Robinson & Torres (1997) Seminars in Imillunol 9: 271-283; Donnelly et al. (1997) Avnu Rev innnunol 1: 617-648; later herein].

A further aspect of the invention is the recognition that combination vaccines can be provided, wherein 2 or more antigens disclosed herein are combined to enhance the immune response by the vaccinated animal, including to optimize initial immune response and duration of immunity. For the purposes of this aspect of the invention, multiple antigenic fragments derived from the same, longer protein can also be used, such as the use a combination of different lengths of polypeptide sequence fragments from one protein.

Thus, embodiments of the invention relate to a composition (or the use as a vaccine of) comprising 2 distinct (i.e. non-identical) proteinaceous immunogens disclosed herein wherein the first of said immunogens is SEQ ID NO: 1 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 2 or a variant or fragment thereof disclosed herein in combination with a proteinaceous immunogen selected from any one of SEQ 10 NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NQ: 3 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 4 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 5 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 6 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 7 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30,

Thus, embodiments of the invention relate to a composition (or the use as a vaccine of) comprising 2 distinct (i.e. non-identical) proteinaceous immunogens disclosed herein wherein the first of said immunogens is SEQ ID NO: 8 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 9 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 10 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 11 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 12 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 13 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 14 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 15 or a variant or fragment thereof disclosed herein In combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 16 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (I.e. non-identical) proteinaceous immunogens disclosed herein wherein the first of said immunogens is SEQ ID NO: 17 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 18 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 19 or a variant or fragment thereof disclosed herein In combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 20 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 21 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 22 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 1.7, 18, 19, 20, 21, 22, 23, 24, 25, 26, 27, 20, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, Thus, embodiments of the invention relate to a composition (or the use as a vaccine of) comprising 2 distinct (i.e. non-identical) proteinaceous immunogens disclosed herein wherein the first of said immunogens is SEQ ID NO: 23 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24. 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 24 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 25 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 26 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 27 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 28 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, Thus, embodiments of the invention relate to a composition (or the use as a vaccine of) comprising 2 distinct (i.e. non-identical) proteinaceous immunogens disclosed herein wherein the first of said immunogens is SEQ ID NO: 29 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Thus, embodiments of the invention relate to a composition (or the use as a vaccine of) comprising 2 distinct (i.e. non-identical) proteinaceous immunogens disclosed herein wherein the first of said immunogens is SEQ ID NO: 30 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, or a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30.

Treatment Methods of the invention

The method of the sixth aspect of the invention generally relates to induction of immunity and as such also entails method that relate to treatment, prophylaxis and amelioration of disease.

When immunization methods entail that a polypeptide of the invention or a composition comprising such a polypeptide is administered the animal (e.g. the human) typically receives between 0.5 and 5,000 μg of the polypeptide of the invention per administration.

In preferred embodiments of the sixth aspect, the immuniation scheme includes that the animal (e.g. the human) receives a priming administration and one or more booster administrations.

Preferred embodiments of the 6^{th} aspect of the invention comprise that the administration is for the purpose of inducing protective immunity against A. baumannii. In this embodiment it is particularly preferred that the protective immunity is effective in reducing the risk of attracting infection with A. baumannii or is effective in treating or ameliorating infection with A. baumannii.

As mentioned herein, the preferred vaccines of the invention induce humoral immunity, so it is preferred that the administration is for the purpose of inducing antibodies specific for A. baumannii and wherein said antibodies or B-lymphocytes producing said antibodies are subsequently recovered from the animal, But, as also mentioned the method of the 6^{th} aspect may also be useful in antibody production, so in other embodiments the administration is for the purpose of inducing antibodies specific for A. baumannii and wherein B-lymphocytes producing said antibodies are subsequently recovered from the animal and used for preparation of monoclonal antibodies.

Pharmaceutical compositions can as mentioned above comprise polypeptides, antibodies, or nucleic acids of the invention. The pharmaceutical compositions will comprise a therapeutically effective amount thereof.

The term “therapeutically effective amount” or “prophylactically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. Reference is however made to the ranges for dosages of immunologically effective amounts of polypeptides, cf. above.

However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA or RNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity, suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N. J. 1991).

Pharmaceutically acceptable carriers in therapeutic compositions may contain liquids such as water, saline, glycerol and ethanol, Additionally, auxiliary substances, such as wetting or emulsifying agents, pH buffering substances, and the like, may be present in such vehicles.

Typically, the therapeutic compositions are prepared as injectables, either as liquid solutions or suspensions; solid forms suitable for solution In. or suspension in, liquid vehicles prior to injection may also be prepared. Liposomes are included within the definition of a pharmaceutically acceptable carrier.

As is apparent from the claims, the invention also relates to related embodiments to the treatment and prophylaxis disclosed herein: the invention also includes embodiments where
the polypeptide of the invention is for use as a pharmaceutical, in particular for use as a pharmaceutical in the treatment, prophylaxis or amelioration of infection with A. baumannii;
the nucleic acid fragment of the invention or the vector of the invention is for use as a pharmaceutical, in particular for use as a pharmaceutical in the treatment, prophylaxis or amelioration of infection with A. baumanii;
the transformed cell of the invention is for use as a pharmaceutical, in particular for use as a pharmaceutical in the treatment, prophylaxis or amelioration of infection with A. baumannii.
the antibody, antibody fragment or antibody analogue of the invention is for use as a pharmaceutical, in particular for use as a pharmaceutical in the treatment, prophylaxis or amelioration of infection with A. baumannii.

### Example 1

immunization study

### Experimental

16 individual proteins derived from A. baumanii (where 1 protein is a positive control) were tested. 16 mice were included in each group. The study was divided into 4 separate experiments comprising testing of 5 groups, that is 4 protein groups and 1 control group (adjuvant only), The 4 experiments are run in parallel, that is staggered with 7-14 days between each immunization. Hence the studies had a duration of 70-98 days (˜2.3-3.3 months):
Challenge strain: A. baumanii AB5075.
Mouse strain; C57BL/6 (inbred).
Dose: 25 μg protein in each immunization.
Immunization route and interval: 3×subcutaneous with 14 days intervals,
Inoculation route (challenge): intranasal.
End point: Lethal challenge.
Adjuvant: Priming immunization: Alum+Incomplete Freund's adjuvant (IFA). 1st and second boost: Alum.
Bled and ELISA: Mice were bled 4 days prior to challenge and ELISAs were run.
Mouse Tag: Each mouse was tagged, hence making it possible to track the mouse-specific antibody titer with survival.
Trial type: Double blinded.
Number of mice per group: 16 mice.
Monitoring period: 7-10 days.

### Experiment 1

Group 1 was immunized with the protein AB57_3582-22-253, i.e. SEQ ID NO: 14, amino acid residues 22-253.

Study Group 3 was immunized with the protein AB57_1088-22-159, i.e. SEQ ID NO: 9, amino acid residues 22-159.

Group 5 (the negative control) received phosphate buffered saline (PBS).

Groups 2 and 4 were immunized with proteins irrelevant for the present invention and did not provide protection. Data are hence not shown.

### Experiment 2

Group 1 was immunized with a protein consisting of amino acids 2-346 of the protein having ATCC accession number 17978.

Group 2 was immunized with a cocktail of AB57.2465-45-550, AB57_2465-551-906, AB57_1136-35-420, and AB57_1136-421-1071, i.e. 2 fragments of SEQ ID NO: 24 (amino acid residues 45 to 550 and 551 to 906, respectively) and 2 fragments of SEQ 10 NO: 25 (amino acid residues 35 to 420 and 421 to 1071, respectively).

Group 3 was immunized with a cocktail of the proteins AB57_1893-26-711, AB57_1893-48-176, AB57.1893-478-711, and AB57_1893-26-477, i.e. 4 fragments of SEQ ID NO: 20 (amino acid residues 26-711, 48-176, 478-711, and 26-477, respectively).

Group 4 was immunized with the protein AB57_2233-22-162, i.e. a fragment of SEQ ID NO: (amino acids 22.162).

Group 5 (the negative control) received phosphate buffered saline (PBS).

### Experiment 3

Group 1 was immunized with the protein (AB57.3370-27-356), i.e. a fragment of SEQ ID NO: 16 (amino acid residues 27-356)

Group 2 was immunized with a cocktail of the proteins AB57..1059-1-754+AB57_1059-25-754+AB57_1059-25-466+AB57_1059-58-177, I.e. the complete protein having SEQ ID NO: 23, as well a 3 fragments thereof (amino acid residues 25-754, 25-466, and 58-177, respectively).

Group 3 was immunized with the protein AB57_0.121-1-367, I.e. SEQ ID NO: 17.

Group 4 was immunized with a cocktail of the proteins AB57..3396-23-691+AB57_3396-306-691+AB57_3396-23-305, i.e. 3 fragments of a positive control (the protein having accession number ACJ 41966).

Group 5 (the negative control) received phosphate buffered saline (PBS).

### Experiment 4

Group 1 was immunized with a cocktail of the 4 proteins AB57_0478-1-550, AB57_0478-551-1110, AB57.2309-22-294, and AB57.2309-1-294, i.e. 2 fragments of SEQ ID NO: 27 (amino acids 1-550 and 551-1110, respectively) and a fragment (amino acids 22-294) and the full-length version of SEQ ID NO: 15.

Group 2 was immunized with a cocktail of the 4 proteins AB57_0053-19-108, AB57_1336-19-114. AB57_0830-1-166, and AB57_3389-22-126, i.e. amino acids 19-108 of SEQ ID NO: 5, amino acids 19-11.4 of SEQ ID NO: 6, the entire sequence SEQ ID NO: 11, and amino acids 22-126 of SEQ ID NO: 7.

Group 3 was immunized with a cocktail of the 3 proteins AB57_3081-2651-3047, AB57_0596-29-580, and AB57_0596-581-727, i.e. amino acids 2651-3047 of SEQ ID NO: 30, and 2 fragments of SEQ ID NO: 21 (amino acids 29-580 and 581-727, respectively).

Group 4 was immunized with a cocktail of the 2 proteins AB57_3778-23-230 and AB57_1830-35-238, i.e. amino acids 22-230 of SEQ ID NO: 12 and amino acids 35-238 of SEQ ID NO: 13.

Group 5 (the negative control) received phosphate buffered saline (PBS).

### Results

Experiment 1

| Group | Survival | Day 0 | Day 1 | Day 2 | Day 3 | Day 4 | Day 5 | Day 6 | Day 7 |
|---|---|---|---|---|---|---|---|---|---|
| 1 | # Mice | 13 | 13 | 2 | 2 | 2 | 2 | 2 | 2 |
| | alive | | | | | | | | |
| | % | N/A | 100.0 | 15.4 | 15.4 | 15.4 | 15.4 | 15.4 | 15.4 |
| | Survival | | | | | | | | |
| 3 | # Mice | 15 | 15 | 0 | 0 | 0 | 0 | 0 | 0 |
| | alive | | | | | | | | |
| | % | N/A | 100 | 0 | 0 | 0 | 0 | 0 | 0 |
| | Survival | | | | | | | | |
| 5 | # Mice | 12 | 12 | 0 | 0 | 0 | 0 | 0 | 0 |
| | alive | | | | | | | | |
| | % | N/A | 100.0 | 0 | 0 | 0 | 0 | 0 | 0 |
| | Survival | | | | | | | | |

Experiment 2

| Group | Survival | Day 0 | Day 1 | Day 2 | Day 3 | Day 4 | Day 5 | Day 6 | Day 7 |
|---|---|---|---|---|---|---|---|---|---|
| 1 | # Mice | 12 | 12 | 3 | 2 | 2 | 2 | 2 | 2 |
| | alive | | | | | | | | |
| | % | N/A | 100.0 | 25.0 | 16.7 | 16.7 | 16.7 | 16.7 | 16.7 |
| | Survival | | | | | | | | |
| 2 | # Mice | 10 | 10 | 5 | 4 | 4 | 4 | 4 | 3 |
| | alive | | | | | | | | |
| | % | N/A | 100 | 50 | 40 | 40 | 40 | 40 | 30 |
| | Survival | | | | | | | | |
| 3 | # Mice | 13 | 13 | 0 | 0 | 0 | 0 | 0 | 0 |
| | alive | | | | | | | | |
| | % | N/A | 100 | 0 | 0 | 0 | 0 | 0 | 0 |
| | Survival | | | | | | | | |
| 4 | # Mice | 9 | 9 | 0 | 0 | 0 | 0 | 0 | 0 |
| | alive | | | | | | | | |
| | % | N/A | 100 | 0 | 0 | 0 | 0 | 0 | 0 |
| | Survival | | | | | | | | |
| 5 | # Mice | 13 | 13 | 2 | 1 | 1 | 1 | 1 | 1 |
| | alive | | | | | | | | |
| | % | | 100.0 | 15.4 | 7.7 | 7.7 | 7.7 | 7.7 | 7.7 |
| | Survival | | | | | | | | |

Experiment 3

| Group | Survival | Day 0 | Day 1 | Day 2 | Day 3 | Day 4 | Day 5 | Day 6 | Day 7 |
|---|---|---|---|---|---|---|---|---|---|
| 1 | # Mice | 15 | 11 | 3 | 3 | 3 | 3 | 3 | 3 |
| | alive | | | | | | | | |
| | % | N/A | 73.3 | 20.0 | 20.0 | 20.0 | 20.0 | 20.0 | 20.0 |
| | Survival | | | | | | | | |
| 2 | # Mice | 16 | 15 | 8 | 8 | 7 | 7 | 7 | 7 |
| | alive | | | | | | | | |
| | % | N/A | 93.8 | 50.0 | 50.0 | 43.8 | 43.8 | 43.8 | 43.8 |
| | Survival | | | | | | | | |
| 3 | # Mice | 16 | 13 | 0 | | | | | |
| | alive | | | | | | | | |
| | % | N/A | 81.3 | 0.0 | | | | | |
| | Survival | | | | | | | | |
| 4 | # Mice | 16 | 15 | 6 | 6 | 6 | 6 | 6 | 6 |
| | alive | | | | | | | | |
| | % | N/A | 93.8 | 37.5 | 37.5 | 37.5 | 37.5 | 37.5 | 37.5 |
| | Survival | | | | | | | | |
| 5 | # Mice | 15 | 13 | 1 | 0 | | | | |
| | alive | | | | | | | | |
| | % | | 86.7 | 6.7 | 0.0 | | | | |
| | Survival | | | | | | | | |

Experiment 4

| Group | Survival | Day 0 | Day 1 | Day 2 | Day 3 | Day 4 | Day 5 | Day 6 | Day 7 |
|---|---|---|---|---|---|---|---|---|---|
| 1 | # Mice | 12 | 12 | 9 | 7 | 6 | 6 | 6 | 6 |
| | alive | | | | | | | | |
| | % | N/A | 100 | 75 | 58 | 50 | 50 | 50 | 50 |
| | Survival | | | | | | | | |
| 2 | # Mice | 13 | 13 | 5 | 2 | 2 | 2 | 2 | 2 |
| | alive | | | | | | | | |
| | % | N/A | 100 | 38 | 15 | 15 | 15 | 15 | 15 |
| | Survival | | | | | | | | |
| 3 | # Mice | 10 | 10 | 5 | 2 | 2 | 2 | 2 | 2 |
| | alive | | | | | | | | |
| | % | N/A | 100 | 50 | 20 | 20 | 20 | 20 | 20 |
| | Survival | | | | | | | | |
| 4 | # Mice | 13 | 13 | 4 | 0 | 0 | 0 | 0 | 0 |
| | alive | | | | | | | | |
| | % | N/A | 100 | 31 | 0 | 0 | 0 | 0 | 0 |
| | Survival | | | | | | | | |
| 5 | # Mice | 13 | 13 | 1 | 1 | 1 | 1 | 1 | 1 |
| | alive | | | | | | | | |
| | % | | 100.0 | 7.7 | 7.7 | 7.7 | 7.7 | 7.7 | 7.7 |
| | Survival | | | | | | | | |

The above observations has let the inventors to the following conclusions:

Proteins tested that provide significant protection against challenge:
1: AB57_1059-1-754, AB57_1059-25-754, AB57-1059-25-466, and AB57_1059-58-177;
2: AB57_3396-23-691 (positive control), AB57.3396-306-691 (positive controls, and AB_3396-23-305 (positive control);
3: AB57_0478-1-550 and AB57_0478-551-1110;
4: AB57_2309-22-294 and AB57_2309-22-294.

A number of proteins tested are found to possibly provide protection against challenge infection:
1: AB57_3582-22-253;
2: AB57_2465-45-550 and AB57_2465-551-906;
3: AB57_1136-35-420 and AB57_1136-421-1071;
4. AB57_3370-27-356,
   Amino Acid Sequences of the proteins of the invention;

In the present specification and claims, the amino acid sequences of the proteins are identified via the SEQ ID NOs. set forth in the sequence listing and the table below. However, alternative designations are used in the examples, according to the following table:

| Designation: | SEQ ID NO: |
|---|---|
| AB57_1759 | 1 |
| AB57_1009 | 2 |
| AB57_1902 | 3 |
| AB57_1614 | 4 |
| AB57_0053 | 5 |
| AB57_1336 | 6 |
| AB57_3389 | 7 |
| AB57_3578 | 8 |
| AB57_1088 | 9 |
| AB57_2233 | 10 |
| AB57_0830 | 11 |
| AB57_3778 | 12 |
| AB57_1830 | 13 |
| AB57_3582 | 14 |
| AB57_2309 | 15 |
| AB57_3370 | 16 |
| AB57_1621 | 17 |
| AB57_0504 | 18 |
| AB57_0619 | 19 |
| AB57_1893 | 20 |
| AB57_0596 | 21 |
| AB57_2870 | 22 |
| AB57_1059 | 23 |
| AB57_2465 | 24 |
| AB57_0791 | 25 |
| AB57_1136 | 26 |
| AB57_0478 | 27 |
| AB57_1048 | 28 |
| AB57_3621 | 29 |
| AB57_3081 | 30 |

When designating a fragment of one of these proteins, this is done using the nomenclature AB57_XXXX-A-p1-p2, where XXXX is any of the 4 digit numbers following “AB57” in the table above, and p1 and p2 are the start and end amino acids relative to the entire sequence of the protein. For instance AB57_3081-50-200 is the fragment of AB57_3081 that has the amino acid sequence defined by residues 50 to 200 of AB57_3081.

SEQ ID NO: 1
MTQLINKGGFRERANRSRKYQQSENKQVALPSKKYQPQTKLQDNQSEMIQAKAGTAETSD
SEQ ID NO: 2
MKLAKTLLATTLALTAASTFAASKHDQAHNTAGEEKVVVSTQEQANTANAASDAVGSASEAAPATR
SEQ ID NO: 3
MIDEEKPLNFEDDDEPLDFEDERFIDDKKEDEMYNSTTKDGSSVDPADDGTRHIRPEDGDPIEIDE
SEQ ID NO: 4
MSTTNNQANQRNNQQQQQQQNDNRNQQQHGNQQQNDQQQQNNQQQQQNDNRGQQQGSNQKDSGQQNSNNNQQR
SEQ ID NO: 5
MSAKLVVTLLATSLLTVGCVAYTDDPYYRGGYGYHDHDDDRYDRNDGRRYSEWERKRWEERKRLYEQQRKDIREQQKDR
REWEKRHREWEKKRLEDRDHDHRDYRHDD
SEQ ID NO: 6
MNKLLVALGLAATVALVGCNKDKAPETGATTGEHLENAAQQATADIKSAGDQAASDIATATDNASAKIDAAADHAADAT
AKAAAETEATARKATADTAQAVENAAADVKKDAQH
SEQ ID NO: 7
MKMTAKIALFSAAIVTMGSLAACQSTTQPPKPEHGMMQDGPRDGHHHRMKHREFTPEQKAAWEQHEAERMARFEQIQKA
CEGKVVGQTVNVQVGDKTLEGTCNLRFEPKRPQPPVNAPAPVATQAK
SEQ ID NO: 8
MKAIKILCITSSILVSSSLFAETPQPQQVNEATSKTMPYGDNPSLGRVLLYKTGKGIQNLGDSIQGASEKTSNKISEKW
KDTKEFTAEKAEVVQQKADTAKVFTEQKIEQAKQNITSSRNGENIPIEQGELSKSSTTAN
SEQ ID NO: 9
MKKSLLAIALMSTLLVACNKHENKTETTSDASTPVQTAQSNNNEAVDTAHTAENSLDWDGKYKGILPCADCEGIKTELE
LKDDKTYELTETYLGKGDANPFETHGKFTPDKDNTSVITLDDKAQNRKPPIGENTATALDMEGKKVEGSLAEHYVLKKE
D
SEQ ID NO: 10
MANKKLLICAAIAAGLLLTACVKKETPKEEEQDKVETAVSEPQPQKPAKFESLESVDTQEAQVQEQPQVEVHREETANT
TTEIRRETRPARSDESSQTQVAEQPKSETPKVEPKPEKKPEPKAEPKPEKAQSKPAAKATEPANTEDDAVAAAIAAATP
ALKN
SEQ ID NO: 11
MTTENKLDELKANAADAKVQGEKALDDLKENVKEKQTAGKEAIADKVDELKTKAADAKVQGEKALEDLKENVKEKQAAA
KEAVEDKASDLKGKLDDAQHSLQDKFDHLRTEAAHKLDDAKAKAAELKEEAATKFDELKTQATAKFDELKKTATEKLNK
LKNHDSAE
SEQ ID NO: 12
MHTRRILLAFSLAASAASVAFADYQNINQSTDSDRLEQLSKTLSQGSYTHPDDLDLPASAKVSVTLREKTVELNNDSLA
KKYGTTTAKNSFKTSSSNPYSWLVSHPLPDTVRVSSNFGGRTMGGRAEHHGGLDMAAPSGTPIYATGPGIVTKSGWGTG
YGQYVEINHGNGYLTRYAHASRLMVRVGDQVSAGDHIANVGCTGRCTGPHLHYEVVKDGQRKNPSTYLAMLP
SEQ ID NO: 13
MGMTFTDIENKSAKRLIGIAAVIFLHLLVAYILMSGLANNIQKPAEKPVELQIIQDIKPPPPPKPEEPKPKEKPPEPPK
MVEKVAKVPEPPKEVEKVATPVQKTTPVAQTTKVATPAPAAPSTPSPSPVAAPAPVAAAAPALKPAGVTRGVSEGSAGC
E
SEQ ID NO: 14
MKMMKTAIVTASVLASASTFAQSAGVNAGASAQVNVQPGGLVSGVANTVKNTAHTVGNTAKHAGHVAADTTVKATKKTT
GKVTELSSKAATGTKHVASRAVTGTKHFATEAATGTKNLATKAATGTKNLAVEAKADTKAHLDAVKTKVAEKQADQKEF
TAEKQADAQARVDAVKARVAQNQAEQKEFVADTKADAQAKLNTAQPAHGVNAQTGVNVGVNVAGINANANVNAGAQAST
QKGEKKSFILGLFGTN
SEQ ID NO: 15
MQNKKHSLLFIALMSTTSLVANIPIESRGLSQNDGSASNTSSSNISVPTNLNWEIMQKNQQLENDIRTLRGQLEEQAND
IEQLKKDLANRYTDLDQRLELLHQKVDPDSATQDDSSNATSDNTTPASAPAPQTTESNKVAAVPATQTSEQQPSAPTTT
TQPAPAAAQNQSNSLELEKAAYTVALDAYKQGGAKKATAPMQNFIKNHPNSIYTGNAYPWLAEPHLATDPVNYNEAKKN
YNVVANQYPNSSKAPRALYQLYSIAKGVLKNTVSANQYKNKLLSQYPKSEEAKFFNK
SEQ ID NO: 16
MSMNNKQRWMGGVVLLGGGVLLAALLLKGNEEIKQVDVQPQTSTSPKLQAKPKQSAQEGQMVQLQPLAVDVETEKRLLE
EQRRSREKAVAEQEARAAEFLAMQQQAEADAARKAAAEYAAINARRAAAQESSDNIPPEVAGSENKAKGQQTDTKKSVD
LAKADADKKAAEAKRLAEADKKAAEAKRQAEADKKAAEAKRQAEADKKAAEAKRQAEADKKAAEAKRQAEADKKAAEAK
RQAEADKKAAKAKRKAEAEKKAEAEKARELLENGDKKWMVQVALAANQANADAVVSKLRAKGYKVTTSPTSKGIRIMVG
PAKDRDTADTTRKKITSDASLNMKSAWVIDWVPLDQRKSD
SEQ ID NO: 17
MANTRYEDDNNSSGTSNRGFASMDPERVREIASKGGRAAHASGNAHEFTSEEAREAGRAAHASGNAHEFTSEEAREAGA
LSHKNDDRNGRGRSRYDDDEDDDRGRSSGRGRGRSRYDDDDEDDDRGRSGGRGRGRGRDDDDEDDDRGRSGGRGRGRSR
DDDDEDDDRGRSGGRGRGRSRRDDDDEDDDRGRSGGRGRGRSRRDDDDEDDDRGRSGGRGRGRSRYDDDDEDDDRGRSG
GRGRGRSRRDDDDEDDDRGRSGGRGRGRSRYDDDDEDDDRGRSGGRGRGRSRRDDDDEDDDRGRSGGRGRGRSRYDDDD
EDDDRGRSGGRGRGRSRRDDDDDDDDRRGRSDGRGQNSRNQKRDAYGRFTS
SEQ ID NO: 18
MLYVIPFIILLVVAVILKKRENSQKQEATSPKNINRKSGKKASAKSSKSSREKIKAKVIEENIPAIPQSNPVPEALRHN
IQQLIQEKQFSAAEAQVNQALKKDNTQHELYLLLLEIHIAQKDEFAIQQLISHIRSLGLNEIAAQAETRQKEYESSSQP
DAIDFPQAQTYEEPKNTDTTAGFDELTTSSSEASFDDLQKDYPPVKQEPAIEIEPLEFNPSFEQNSATENTNQPAQQPE
LSSTQETNELADLEPSFDLAPLHETEEKSQAVEVKADQENSINALDFNPDLNPSSSETKSVQQAPSLDEIKLIEQAPLE
ATSIAPLEFSLDEPALVPAPELETQNHIDVVNEAATQTQIEDPLLEAPPELKQINENELDLKLAEQYIKFGANQAARNL
LQGDEQKFNTEQQQHAKNLLNRIAS
SEQ ID NO: 19
MPKIKPIKLVIIVVCIAIIAVLAWKFLKPKQQQPQKITAEVTRGDIENNVLATGTLDATKLISVGAQVSGQVKKMYVQL
GDQVKQGQLIAQIDSTTQENSLKTSDANIKNLEAQRLQQIAELNEKQLEYRRQQQMYAQDATPRADLESAEAAYKTAQA
QVKALDAQIESAKITRSTAQTNIGYTRIVAPTDGTVVAIVTEEGQTVNANQSAPTIVKIAKLQNMTIKAQVSEADIMKV
EKGQQVYFTTLGDETKRYATLRQIEPAPDSISSESNSTTSSTTSSAVYYNALFDVPNTDGLKRIDMTAQVYIVLNSAKN
ALLVPSSALSSKQFSGQRKQGQSADKASSTPSAERKHQGNGVRLERLNLTPEQKQLIEQGKATLSVVRVLQADGTTKPT
QILVGINNRVNAQVLAGLKQGDQVVIADSSENSAASAMSGNNRRRGPMGM
SEQ ID NO: 20
MNIPPRPPKLSVIACAICYANLTYAQDAQVQALQTIQVKASNAEQSSEQTKAYNVKNSSSATKLNIEAKETPQTINVVT
RQQIEDFGLTSTRDVLRNTPGVTVSNQETERTTYMARGFEISNILTDGVGPPLSGYNYNNTNPDTYFYDRVEVVKGADS
LTNAFGDPSATINNIRKRPTQEFQASGGVSYGSWDTQRYEADVSGSILPSGKVRGRIMGYEQTGDSYLDRYSAEKNGFA
GIVEADLTDSTLLTAGYSQEQNKPNANNWGALPLLDANGKQISYDRSYNPNPDWAHWDNETQNAFVELKQKLNDQWNAK
LTYNYLDTKHNSRLLYYYGYPKADGSGVSLTPWGGQEHQEKHAVDFNLEGTYKLFNREHEATLGYSYVRNHQQDKQSTG
TINDSNVIKSTTTDWASWTPQSITWSDFTEAANYKQNINSIYAATRLHLNEDLKLLLGANYVQAESKGESYSSPMSYSE
SKVSPYVGLTYNFTPEYTGYMSYTSIFRPQTGIDKDTNQALKPIEGKSYEMGVKSSWLDDRLTGTLSVFKTEQNNYPLR
NSDGNPLNRKVPTSDLESQGVEVGLSGQITDNVNLSFGYAQFSIKDTKNGGEARTYNPNQTLNLLTTYTPPVLPKLKVG
AGLQWQDGIKLYDSNVNGTIKQDAYALVNLMASYEVNDHITLQANGNNIFDKKYLNSFPDGQAFYGAPANYTVAVKFKY
SEQ ID NO: 21
MKLQTIACAVAIATGGLFFSHTMNEARAATNTAAVSQSIQPTQEQALVARQLATLVDRQHYLNMRLDANTSNRILDMYL
DSLDPDHSLFLDAEVQNYKKLYGSNFGASLKAGNLTGPFAIHQQYRERLKQFYEFMLAELKKPQNLKQPNTFIEVDREK
APYFKTSAEQQNHWRKMLVSQLINLTISREEEQAKQKALKENPSLADGQDLTGPEDLTPAQTLTKRYTRQLERISRVKS
DDVLDKTLNAMLATYDPHSNYYPPIDAIELNRQTTLQLEGIGVSIRPERGNEDYTKIETIVEGGPASKSGQVKSGDRIV
GVAQEGGKMIDVVGWSSSEIVGLIRGKRGTKVTLKLLGAGASMSQARNVTLVRDVIQEEDAGVRSRTVEVTRDGKKHLL
GVIEIPSFYFDYRSRRAGQQYRSVSEDTANAFEALKAKKVEGIIIDLRNDPGGSLEEVARMLGQVIKSGPVVQIRDGNG
NVSVPEDNDGGQQIYTGPLAVLVNLASASASEIYSAAIQDYERGIIIGSTTTGKGTAQVQLDTLAYGQATLTQRKFYRV
TGGSTQNKGVVPDIKLVDIYNEEFGERKSKNALKWDTIPTAPFKREGSVQPYVAKLSQLSEQRVAVDPQFKYLNKRTAI
AKVTSDQKQVVLDIDKRRAELLSLEKQTLDAENERRIATGQKPFPNWESYQASLDALAESRAKMKANQRPALPEEETFV
NEAANVLMDYAKLQNR
SEQ ID NO: 22
MTRIIVASKEGLDVLQDGQLNKVVLNQPTIIQIGVSQKDIASMEKQGGSLVIHLKNGETIVLENFFNEATNTTEHSLVF
PTEQGKFVEAQFDAQGKVIDYRGLNHVTDLAYTSTSPSAATMAVDNDPSFSMGNVLKAGLAVLAAEGLYLWAFDKDDKD
DSPSTPDLIAPAAPTATLADDTVTVTGKTEANAKIYIKDAAGNTVASGVADASGNYTIKLDKPLVNGDKLNVIAQDAAG
NNSKVTVVTGTKDTIAPDVPQAQLSDDGSLLTGKAEANAKITVYDATGKVLGTVFANKDGIYSLKLTPPLTSEAGGKVV
AEDAAGNKSEEVKIIAGKDTIPPASPFVEVNKEGSVIHGKTEANAKVQIKDADGKVIGSGTADAQGEFQITLSPALKEA
QKGTVVVEDAAGNVSKPVEITPGFDSIAPDKPTVQINTDGTSVTGTAEANAKIEIKDTTGKVIGSGTADANGKFTISIS
PALTDNKHASVSAIDNAGNKSEVVDIVGTKDTTPPAKPILNSVDDDVGAVKGAITAGSETDDARPKLTGSGEANATLTI
YDNGVAIGVVTVTSGRSWSFTPDKDLALGKHTITLTQTDAAGLTSEASSPFTFYVVAPKAASLSETSVDILSTEGPSLA
DSVGLHTLKVAQNTTTETNNPQKSVPLDDLLKSSTASESDPIAKLLSSTALKTTQASEPIEVNASVGQTTSNPNHPLPD
TTSSVLQNLLDQTYPVV
SEQ ID NO: 23
MSKRIIQSVLSVSVLASMMSMAFAAQNEQEQAEQTLEKPAEPVKLETIFVTAEEQVKQSLGVSVITKEDLEKLPVRNDI
SDYVRRMPGVNLTGNSATGQRGNNRQIDIRGMGPENTLILVDGKPINSRNSVRYGWKGERDTRGDSNWVPAEAIESIEV
LRGPAAARYGSGAAGGVVNIITKKVTNETHGSVEFYTSQPEDSKEGSSNRVGFNVSGPLIKDVLSYRLYGNYNKTEADD
VDINKSIGSTAAGREGVKNKDISGRLAWQATDQQTVLLDISSSKQGNIYSGDSQLNANAEADAILSQLIGKETNTMYRD
SYALTHEGDWSWGKSKLVAQYDKTHNKRLPEGLAGSVEGKINNLDDKATSRLETLRFNGEANIPPEYYLPQVLTVGTEW
VEDRFKDNVSTTQGKDSSGSGYGDQLAKGDRSKMESRIASAYIEDNLKVTDSTDVVLGLRFDDHSKSGSNWSPSLNITQ
KLNDNFTLKGGVAKAYKAPNMYQNAEGYLLSTNGNGCPANIESRCLLQGNGDLKPETSVNKELGIQFQRDIVNASLTWF
RNDYKDKIVAGTHVVGTVDGSSTNANTGAVTNTKWNILRWENTPKALIQGFEGSLGLDFGDIRWTNNFTYMMDSKDKQT
GNPLSLVPIYTINSIFDYDITDQLDVNFVFTQYGRQKSRQFAENRLESGIGSGGANSALKPSTVKSYSTAGINVGYKFS
DQISTRVGVSNLFDKQILRDSNSISQTYNEPGRAYYASLKYSF
SEQ ID NO: 24
MPSKIKFKQSTLSHSMHLILKMQSIPKLICSSLLLSLCVTPCYAQSSAETVIPEANQTVTDSLVQQTNTNNPSDVPITD
VATLVTQAQQQQDSLAILQQQEQFPNQIEEFKPITLDNLEDLPVMPVDQNMANEIYRVAEEAKNEAQNFQNGTQKQPEM
VVSDADQAELHEINQAPVNIDQLMHEIQSDSKIVVEANETGKTLPELTAAVEEPPEEKGFPRRIFNKIRPPRVIPMEQI
PRITAEVTGAPDDLAKNIKGKLSTPTQESPEDFNAALPQLRSLSNQAAQAVGYYNAEFRFEKLSASRVRVNVTPNEPVR
INEQNIEFTGAGAKQPQFQVIRLVPDQDVGDIFNHGLYETTKSRIVDAASDNGYFDAYWRLHDVKVSQPENKADINLKY
ETGERYKLGKVEFRMSDPSKPLPLNMNILESMAPWKEGDDYAFWRVNVLANNLTNSRYFNYTLVDSIKPDPIEKPLELP
PDLQALVDQQNVDIDESKLLPLEQQQLAKARQLASSSKEVTQNVVDEKQFAGTESVQAAPASLKAATVQHEEQESEQDR
LQAQAREEKRIPVIVTLNADKLNSLETGIGYGTDTGARLRSQYRRSIVNKYGHSFDANLELSQIRQSIDGRYSIPYKHP
LNDYFNIVGGYERETRDDIGPDVSLLTESAVLGGERVIKKPLGNWQHTIVGRYRLDRLTQKGNVDISELPDAFKTAASE
QEALLPSYETSKTSSNTRLNPTKAFKQTYKLELGSESLLSDANMAIATAGWRFIYSLGENDLHQPVGRSDFSYIFTDEF
DKVPYNLRFFTGGDQTIRGFDYKSLSPEDNGYKIGGQALAVGSLEYNYQPKEGWRAAVFSDFGNAYDKSFSNPTAYSVG
VGIRWKSPIGPIRLDVASGISDDNHPIRLHPFIGPQL
SEQ ID NO: 25
MPIKSILSSITSIIPLPENSNTSSNLGNGSGDGLLNGISSGNGEHNYGIGNGIADDASITAPITIPLNLSGNSITLIGN
SGSSSVNSSPTTTSNNVNDNDVTNNGNGSTIGSGTGNGSGDGLLNGAASGNGEHNYGIGNGIADDASITAPLSIPINLA
GNSITLIGDSSSSSVNNSATNTSNTVNDNDTTYNGNGSGGGNGSGDGLLNGIGSGNGEQNYGIGNGIADDASITAPITL
PINLSGNSITLIGNSSASSVNSSPTTTSNTVNDNDTTYNGNGTGDSGVSALGGSGNGSGDGAGNGIASGNGEHNYGIGN
GNGDDVDITAPITGVLNISGNSFTLIGNSSSSSVNTAPTTTSNTVNDNDTIDNGNSGGTGSGSGNGSGDGLLNGAASGN
GEHNYGIGNGNGDDVDITAPITGVFNFSGNSFSIIGNSSSSSINTAPTTTTNTVNDNDVTDNGNDGGGLVGGSSGNGSG
DGLLNGAASGNGEHNYGIGNGNGDDADFTFPLTGVLNFSGNSLSGFGSSSSDSVNVAPTTATNTVNDNDTIDNANTGGL
GDGSGNGSGDGLLNGAASGNGEHNYGIGNGNGDDADFTLPFTGGLNILGNALSGIGGSSTDSINISPTTTSNTVNDNDT
TNNGNTSGGVIGSGDSGNGSGDGLLNGISSGNGEHNYGIGNGNGDDVDVVAPITTPLNVLGNSFSFIGGEGTGDILGPI
TGIIGGIGGDGDILSPITGIIGGIGGDGDILSPITGIIGSIGGIGGDLGDNPLTGIIQSGIDVLQNLESLKTGLINTGI
DTIAGTIIGVFPDAEHPVGDFADLGKLIFETSRDSVNGTLEAISDLAGADLEGASGSITGVIDTLITNGSTASTIIQHI
VGDDLVTENGGLLGSITTIIGGVDSGDGGLLGGLDGLISINYGDSDNSNSIDVEDILGNILGSVGSNQGIAVGEPDPTG
GSLIHTISLNTVNQLTDQLLHALPTV
SEQ ID NO: 26
MYKPTTFVWQPSAASLFKITVLSSALAALGITTGCSSTPQSAKTSKTKQVSGAGYLDASSLDSLEDLLSATDMRAVEGD
RLLILKHGDVWKRMAVGFKMDLNHWDPRIEAQRSWFISRQPYLDRLSARASRYLYHTVKEAERRGLPTELALLPVIESS
YDPAATSGAAAAGLWQFIPSTGRIYGLQQTGMYDGRRDVVESTRAAYEFLGSLYNQFGSWELALAAYNAGPGRIQQAIN
RNQAAGLPTDYWSLKLPQETMNYVPRFLAVAQIIKNPRAYGVSLPPIANRPHFREVTLSAPLSLNEIASVTGLSRAELY
ALNPGYRGETVDPASPMRILIPADISPSVDNKLKGMKAGGSSGWWASVTSPSKPTTTTSTSVTVRTTPSTPAQPVRPST
PAKTSSSSVTVKTTTPRGSDALAAFAASADVPSAPRIPVAVTPAANIKPVRTEPPISATEREKILAAVRAEGEKETVDQ
ALEPQATQAEKDQVVAELKALAPQGTEIVDPYDGKIKLTAIQTSQSVAEQQGKEVSKGFAYPKTLAEDATLANSEDAQR
NKDKPYIKTDTDVVVVQPKGKRSTYTVQPGDTLAVIAMKNGVNWRDVAKWNQIDPEKTLPVGTSLYLYDAKPQEAETTA
KSAAKPDVYVVQANDSLTGVANQFNLSVKQLAEYNDLSVTDGLFVGQKLQLKEPKGNRAAKVEPKAIQASTRRIATKSY
TVKRGEYLKLIADRYALSNQELADLTPGLSAGSNLIVGQKINVPAKEITVDEVDDSKASGKYEKLAAGPSYKTESYKVQ
RGDTLSSIATKSKISLAELAELNNLKANSHVQLGQTLKVPAGASVPDQYVVQSGDSLNAIAAKYNLQTSYLADLNGLSR
TAGLPAGQRLKLTGEVETTSKVSAKNTKEETPETYTVKSGDSLGNIANRYHLQLDYLAALNGLSRNSNVRVGQRLKLTG
DLPTVETAKTDTAKSSPKAVVAGKNTEKYTVKAGESLNAIASRAGISVRELAEMNALKANANLQRGQNIVIPKTVVEYK
VKRGDTLIGLASKYGLETTLLAELNNLTPSTQLRIGDIIKVPNL
SEQ ID NO: 27
MKRMLINATHAEEVRVALITGNRLYDFDLENRTREQKKSNIYKGHVTRVEPSLEAVFVEYGAGRQGFLSMREIANSYFQ
ADPRQTSNIRELITEGTELLVQVEKEERGNKGAALSTFISLAGRYLVLMPNNPKGGGISRQISGSVREELKEILASLNV
PRGMSVIVRTAGIGRTQEELQLDLQHLLDLWAQIQGTASSGPSPMLVHQEAGVVTRAIRDYLRDDVAEILIDSEQAYNE
AYNFVKAVMPRQLDKLKTYTLNEPLFAHFGIESQIQTAYEREVKLPSGGSIVIDQTEALVSIDINSAKSTRGHDVEETA
LNTNLEAAEEIARQLRLRDIGGLVVIDFIDMTKERNQRMVEAKLREATQSDRARIQFGQLSRPGLMENSRQRLRPSLEE
ATGYVCPRCHGTGMVRDLRSLSLSIMRKVEEIALRERHGEVQVEVPVEIAAFLLNEKRHSLVYLEQTSGVRVTVLPHPH
LETPHYEIAYNPDGFAPSSYERTEATRSSEKELGYESSEWHLEEADHGHAHVTATASTHAAAQKKANHATQPVAQPSAQ
KAASPCAWLENLFVQKQAQTVDQSRSAQNAAAAIEQMVNTGAVSRGQPGQVAVPAVAEVAPVQSNNAYISQSPVKQDVR
EHVEKDDKSQQQRQNNKKRKHKEQREQHHQSHEQQHQVHEEVVQLSRQEQRELKRQQKRQQQQDQQHQNNDVQHTENAV
PRRDRNNQQRPNRPNRHRDPSVLNENQNTLVVVDEKQIKVDVIDAPKHDVMNTALIINVDQGQSEIVALTPERRHVERV
ETTSTEVAQEPTPAPVVAEKAAVVETKEEAQPSQEAAQPQIKRASNDPRMRRRQQREAKHAKAATPSIAPSQIPTLAQH
TIGSLIRHVYGEDCTVLIEQFGLVPTFNRALQKFAEQYASTLVVEVTAETEEKKPVTRDAELPSHKPAEEAEPAPVLPL
TPPQAPAPKVANDPRERRRLAKLAAEQAFEQVKQQHSAQEEVATPAPVAEETVAAPTAETQATVEPAQQPLELNQSTEV
VQPEAAPAEEKATEETVAEAPAAKEPAPSKAASKAKAAAEETVAPTEATTDAESEDVKADKDKPSRPRRPRGRPPKKAN
PVAE
SEQ ID NO: 28
KSTLATLKALLAKRILIIDGAMGTMIQRHKLEEADYRGERFADWAHDLKGNNDLLVLTQPQIIQGIHEAYLDAGADIIE
TNSFNGTRVSMSDYRMEDLVPEINREAARLAKAACEKYSTPDKPRPVAGVLGPTSRTCSISPDVNNPAFRNISFDELKE
NYIEATHALIEGGADIILIETVFDTLNCKAAIFAVKEVPKQIGRELPIMISGTITDASGRTLTGQTAEAFWNSVRHGDL
LSIGFNCALGADAMRPHVKTISDVADTFVSAHPHAGLPNAFGEYDETPEQTAAFLKEFASSGLINITGGCCGTTPDHIR
AIANAVKDIAPRQVPETVPACRLSGLEPFNIYDDSLFVNVGERTNVTGSKKFLRLIREENFAEALEVAQQQVEAGAQII
DINMDEGMLDSQNAMVHFLNLVASEPDISRVPIMIDSSKWEIIEAGLKCVQGKPVVNSISLKEGYDEFVEKARLCRQYG
AAIIVMAFDEVGQADTAERKREICKRSYDILVNEVGFPAEDIIFDPNVFAVATGIEEHNNYAVDFIEATGWIKQNLPHA
MISGGVSNVSFSFRGNEPVREAIHSVFLYHAIKQGMTMGIVNAGQMAIYDDIPTELKEAVEDVILNQNQGESGQAATEK
LLEVAEKYRGQGGATKEAENLEWRNESVEKRLEYALVKGITTYIDQDTEEARLKSKRPLDVIEGPLMDGMNVVGDLFGS
GKMFLPQVVKSARVMKQAVAWLNPYIEAEKTEGQSKGKVLMATVKGDVHDIGKNIVGVVLGCNGYDIVDLGVMVPCEKI
LQTAIDEKCDIIGLSGLITPSLDEMVFVAKEMQRKGFNIPLLIGGATTSKAHTAVKIDPQYQNDAVIYYADASRAVGVA
TTLLSKEMRGAFIEEHRAEYAKIRERLANKQPKAAKLTYKESVENGFKIDESYVPPKPNLLGTQVLKNYPLATLVDYFD
WTPFFISWSLTGKFPKILEDEVVGEAATDLYNQAQAMLKDIIDNNRFDARAVFGMFPAQRTDADTVSVFDEAGQNVTHT
FEHLRQQSDKVTGKPNLSLADYIRADREQQDYLGGFTVSIFGAEELANEYKAKGDDYSAILVQSLADRFAEAFAEHLHE
RIRKEFWGYKADEQLSNEELIKEKYVGIRPAPGYPACPEHSEKAVLFDWLGSTDKIGTKLTEHFAMMPPSSVSGFYYSH
PQSEYFNVGKISQDQLEDYAKRKGWTLDEAKRWLAPNLDDSIV
SEQ ID NO: 29
MKLKLKNFKPNNLWYAVCSSSMIFTWLMTSSVVQASDLQIYASPTAGKKTIVMMLDTSGSMTNNSYGENRLAMLKNGMN
AFLASNNPVLNDTRVGLGNFSANGDSRSGQILVAAAPLGDASTLNTVGSQRYKLKQAVANLTAGGSTPSAHAYAEAAAY
LMGTTTYSETNYAIRKDSYIKRVRRSDNRTEYSYCTNYRDSQIDTANLWQPCRSNSYWSSWSTNNPGVDNATAYDTSSD
WTYYYTYYYTTFNYAVANADSGIPKSKSNDTASNPNIVVDRNATNSNAVYQSPLPAVANRQSCDGQGIYFLSDGEPNNT
TNTRSASVMSTALGSTFGADFNCSGGLSNTTADSGWACMGEFAKRLFDKTKNPAGVSIQTAFVGFGSDFSSLNSSDVKN
ACRLSSRTQSDRKGDDACSPNQSTNAVAAPGYGNGGFFPTQSSQGVTDSVIAFINNLDKVPLEPLTTGAISVPYDALNP
KNLQEYGYLRAFEPNPANTYLTWFGNLKKYHVVLSGANAGAFEANSGGLVYNASGAFRTGTKDYWNSSTYTDGGKVFLG
GSYANVPLPIAGQPETRDASGNITKYYYAVQSKIRNLFTDVSAVAADGSLTKISTSGTNLLKIPAAPPEETNPFDTVAN
TASYVLGKFDPSTGQNILKAFPISLKLKILNYLGYSTDINATTLPSSLVTSNEPYLSMGGSIHSLPVQLTYNGTLDDNG
NLTSAREQSILYGTMEGGLHIVDASSGIEQMVFVPADILNDSVASKALVVGQSDASAPAHGMDGAWVSDPAYNITTVGS
GSSAVSKVTAKQMNIYGGMRMGGSSYYGLDVLSPTSPKLLFRIGADQNDYSRMGQSWSKPVLANIRYNGSIRRVLIVGG
GYDQCYEKPWITLTDACFTNGKAKGNAVYIIDAKTGQRLWWTSDTGSNTDNANMKHSIVSRISTLDRDADGLVDHLYFG
DLGGQIFRVDLNNNQTKTNSTYSSFGVRVVRLANLATNDSTYDGTNDYTGGNAPRFYEPPTVTIHDYGIHTFITVGIAS
GDRSTPLDVYPLTGREGMTPASALSGRPVNNVYGIIDEDFVKKNLMSLTDNQLETKDITRTGLRKNPQILRTGETRVAQ
IFFPTTGVGKGGWYRSLSSTSDGTEKANNMSFRIKGGLKAPEEPMAITGNLIILVYDPQGTGIAADPCLPRVVGETDRQ
TYCLPFGACLNSDGSIDQNKENHSGFETQTGTNCPVGASECNKNVIGSGIRSVTFVPTEDNPPTTNSCGKLKLSGNEQG
TGQWQCTSHLVPTRWYERYR
SEQ ID NO: 30
MTDAAGNTSEQAVQKVVVDTTAPQAGELTLSDLSDTGISATDQITQDKNFNLKLEGQESGSRVTYLVSTDEGKTWQETT
IAQKDLTDGVYQYKAVVTDAAGNTSETAVQKVVVDTTTPQAGELTLSDLNDTGVSVTDQITQDKNFNLKLEGQETGSRV
TYLVSTDEGKTWQETTIAQKDLADGVYKYKAVVTDAAGNTSETAVQKVVVDTTAPQAGKLTLSDLNDTGVSATDQITQD
NSFTLKLAQPIVIGEQAALLDHYEVSKDEGKTWQETTADQKDLADGIYQYKAIVTDLAGNISESAIQKVVVDNSLNVES
TTVIVKPITEDNTISLVEKDQVISIRLEIANLPTDLNSSLTSVNTTLGNVTYNFHFDEVTQEWVTEIPAEFLWSVEPQT
NISIEISLTDQAGNTAIIKHTQNYNVDHTPNSPTLDSLTFNNIDGAIISGSAYKGSKVDIYNKNGDWLASTITNEEGKF
TLQDLSINSNQEVYAVATYNGYSSENSSIGLVTEVPAISITRISPEGVISGYATEGSHFIVKDQNGNILQEFNSNVFDS
SGITPFSVMALGEVRPPILSLDQPLEEGAQIIISIDKDNISGHPQYITADYTPAVFLETPQFDISGETLSVHVNEPHSF
IRAFSGEGNLIATGFTDEQGFASLQVFQFLKEGETVSVQVVDKNQNTSETLIEVPNFAYIPHVERITQEGLISGVAEDN
STVIVRDADGNELGKVTLGDDNSWSDFSHFSLSVNRPLIDGEKISVQIIDNKGLMSPEQNIIVDLTPPPAPTELNFNDA
GDLVYGHAEPFSEILVKDGQGNILNKWFWNNWTDESGSFSIELGTFLTNAETVYVTATDVNGNVSLAAQIQAPNYAFAP
YVDSFTSDGVISGQAENNSTLVVKDAKGDVVAEIKVGEDNGWNGSSYFKLQLDRPLVDGEQFFLSIKDARGQVSADTVI
TADTVAPTPASNLVFSEDGSYLTGVAELNTTIQVFDHNGQLVNIWNNTINSDGTFTIYLGSNNLHQEAFTVTVKDQAGN
VSEAISINAPLDDIAPNPIKNILLDANGQNFTAQAEANSQIEVFDSLGNQTGWGSTDSAGNVSGSFNQTYLGHEELTFV
VIDRAGNRSIEFKQNALIDTIAPNPIANIIFNEDGQSFTAQAEAGSSIDVLDQTGNKIGFGYTDSSGNVSGYFQQVYLH
GEELTFVVIDRAGNRSEAVKQSALNDDVVPNPIENIVLDLNGQNFTAQAEANSQIEIKNNNGDVVGYGSADSAGNVSGY
LYQVHLHGEELTFIVVDRAGNRSTEVKQNALIDDIAPNPIENIVLDINGQNFTAQAEANTQIEVKNAVGEIVGLGYVDG
AGNVSGYLYQVYLHGEELTFVVVDRAGNRSTEVKQNALIDDIAPNPIENIVLDINGQNFTAQAEANTQIEVKNAVGEIV
GLGYVDGAGNVSGYLYQVYLHGEELTFVVVDRAGNRSTEVKQNALIDDIAPNPIENILLDANGQNFTAQAEANTQIEVK
NTAGEVIGSGSTDSMGNVSGYFYQVYLHGEELTFVVVDRAGNRSTEVKQNALIDDIAPNAIENIIFNENGQNFTAQAEA
NSKVEVKNAAGEVVGSGYVDSVGNVSGYLNQVYLKGEELTFVVIDQAGNRSIEVKQTAFLDNTAPENATNLVFSEDGSY
LSGMAEPNATIQIPDQYGQLLNQWNNNVNWDGTFNIYLNSNYMHGEVFKVVVVDHAGNLSGEVTVKAPLDDIAPVAASD
LVFNEDGSSLSGVAEPNTFIQIFDQNGQQMNTWSQSVNADGTFTIFFGTYNLHGEEFTVIVKDLAGNVSEAVSVKAPLD
DIAPKPIKNIVFDANGQSFTAQAEANSQIEIFDSFGSQIGWGSTDSTGSVTGYFYQVYLHGEELTFVVIDRVGNRSDEM
KLNALMDTIAPKPIENIIFNENGQNFTAQAEANSFISVKNAAGEFVGYGYVDSTGNVSGHFNQVYLKGEELTFIVIDKA
GNQSIEYKQNALTDDIAPNPIENIVLNKNGQNFTAQAEADSQIEVKNTAGEVVGSGYVDSIGNVSGSFNQVYLHGEELT
FVVVDPAGNRSTEVKQNALIDDIAPNQIENIVFDVNGQYFTGHAEADTRIEVLDQFGNRAGWGYVDSQGNVIGYFNQVY
LHGEELTFIVVDIAGNRSVEVKQNALIDNVAPPAAANITLTSDGLLFGEAEPNSTVEIIDQYGAVITTTYVWYDGTFNQ
WINLSQYQTQNLSIVVKDQAGNRSEVVHELVPVFTNSPIAATELKLDIDGHILTGKATVGMSVVVTSTDGQTINGGWNN
AVNEDGSFAIQLNDYYLQGQTLQVRVYDQNTNQYSLISEIIAPLDNIAFVINEVVINNDGYGITGQTDSKAIIQVMDAD
GDLRAEFQADETGYFNASIYPPILREGQLFITAIDLAKNISKPFNITFNADTNAPPSAEHIVVSENGFFIEGTAVAIST
VHIFDVHSNHVATNVADEAGNFNIQLYPPLASGQILRIVVEYNGYQSAYTEITAPIDTVAPNAATQLLLEDGNVLSGQA
EAYSIVNIFDANNNLVGQTNVGSDGAFLTHLWYEYWHGETLTVKVVDANQNVSVGTTIVAINDTVVPDVVTQLAIDEWG
SLTGRVESYATVELTYHFTDQPLSVTSTTALANGMFFIYLDRNATSLDLTVIDRAGNRSETISQIISDLPTVIIDHFKG
DATDNTYNIDTIDDFVQEYIVEPYAIYKDVWIDNSYMYSDWVIEGHYEQIWPVDGYYESQWATSGYSTVQNIYQNQNGI
TYIDNGTADSDYSRYEQQYYDFVNGQWQEGYELTYIRSEEGWVDTSHYEDVYIDTSHYEEVWVDTSHYQDIWVENSYWE
SQLVESGRRDVDLGGHDKIISSVNYSLVGLYQTVNDPTTVDSFLESGRYVEDLELVGSAHLNATGNALDNLLTGNSGNN
VLNGREGNDTYITNEGTDTIVFQLLNSQDATGGNGHDTVLDFTLGDIRTNLQADKIDLSELLIDYSKDVSALAKFITVE
QDAGNTTISLDRDGEGTMFNSVSLLTLNQVNTTLDELLNNQQIIV

1. A pharmaceutical composition comprising a polypeptide, said polypeptide comprising
a) an amino acid sequence selected from the group consisting of SEQ ID NO: 18 and a fragment of SEQ ID NO: 18 comprising at least 35 contiguous amino acids of SEQ ID NO: 18, or
c) an amino add sequence having a sequence identity of at least 60% with the amino add sequence of a),
in admixture with an immunological adjuvant and a pharmaceutically acceptable carrier, vehicle or diluent,
said polypeptide being antigenic in a mammal.

2. The polypeptide according to claim 1, wherein the at least 35 contiguous amino acids are at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55 and at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94, at least or exactly or at most 95, at least or exactly or at most 96, at least or exactly or at most 97, at least or exactly or at most 98, at least or exactly or at most 99, at least or exactly or at most 100, at least or exactly or at most 101, at least or exactly or at most 102, at least or exactly or at most 103, at least or exactly or at most 104, at least or exactly or at most 105, at least or exactly or at most 106, at least or exactly or at most 107, at least or exactly or at most 108, at least or exactly or at most 109, at least or exactly or at most 110, at least or exactly or at most 111, at least or exactly or at most 112, at least or exactly or at most 113, at least or exactly or at most 114, at least or exactly or at most 115, at least or exactly or at most 116, at least or exactly or at most 117, at least or exactly or at most 118, at least or exactly or at most 119, at least or exactly or at most 120, at least or exactly or at most 121, at least or exactly or at most 122, at least or exactly or at most 123, at least or exactly or at most 124, at least or exactly or at most 125, at least or exactly or at most 126, at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, at least or exactly or at most 139, at least or exactly or at most 140, at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, at least or exactly or at most 150, at least or exactly or at most 151, at least or exactly or at most 152, at least or exactly or at most 153, at least or exactly or at most 154, at least or exactly or at most 155, at least or exactly or at most 156, at least or exactly or at most 157, at least or exactly or at most 158, at least or exactly or at most 159, at least or exactly or at most 160, at least or exactly or at most 161, at least or exactly or at most 162, at least or exactly or at most 163, at least or exactly or at most 164, at least or exactly or at most 165, at least or exactly or at most 166, at least or exactly or at most 167, at least or exactly or at most 168, at least or exactly or at most 169, at least or exactly or at most 170, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182, at least or exactly or at most 183, at least or exactly or at most 184, at least or exactly or at most 185, at least or exactly or at most 186, at least or exactly or at most 187, at least or exactly or at most 188, at least or exactly or at most 189, at least or exactly or at most 190, at least or exactly or at most 191, at least or exactly or at most 192, at least or exactly or at most 193, at least or exactly or at most 194, at least or exactly or at most 195, at least or exactly or at most 196, at least or exactly or at most 197, at least or exactly or at most 198, at least or exactly or at most 199, at least or exactly or at most 200, at least or exactly or at most 201, at least or exactly or at most 202, at least or exactly or at most 203, at least or exactly or at most 204, at least or exactly or at most 205, at least or exactly or at most 206, at least or exactly or at most 207, at least or exactly or at most 208, at least or exactly or at most 209, at least or exactly or at most 210, at least or exactly or at most 211, at least or exactly or at most 212, at least or exactly or at most 213, at least or exactly or at most 214, at least or exactly or at most 215, at least or exactly or at most 216, at least or exactly or at most 217, at least or exactly or at most 218, at least or exactly or at most 219, at least or exactly or at most 220, at least or exactly or at most 221, at least or exactly or at most 222, at least or exactly or at most 223, at least or exactly or at most 224, at least or exactly or at most 225, at least or exactly or at most 226, at least or exactly or at most 227, at least or exactly or at most 228, at least or exactly or at most 229, at least or exactly or at most 230, at least or exactly or at most 231, at least or exactly or at most 232, at least or exactly or at most 233, at least or exactly or at most 234, at least or exactly or at most 235, at least or exactly or at most 236, at least or exactly or at most 237, at least or exactly or at most 238, at least or exactly or at most 239, at least or exactly or at most 240, at least or exactly or at most 241, at least or exactly or at most 242, at least or exactly or at most 243, at least or exactly or at most 244, at least or exactly or at most 245, at least or exactly or at most 246, at least or exactly or at most 247, at least or exactly or at most 248, at least or exactly or at most 249, at least or exactly or at most 250, at least or exactly or at most 251, at least or exactly or at most 252, at least or exactly or at most 253, at least or exactly or at most 254, at least or exactly or at most 255, at least or exactly or at most 256, at least or exactly or at most 257, at least or exactly or at most 258, at least or exactly or at most 259, at least or exactly or at most 260, at least or exactly or at most 261, at least or exactly or at most 262, at least or exactly or at most 263, at least or exactly or at most 264, at least or exactly or at most 265, at least or exactly or at most 266, at least or exactly or at most 267, at least or exactly or at most 268, at least or exactly or at most 269, at least or exactly or at most 270, at least or exactly or at most 271, at least or exactly or at most 272, at least or exactly or at most 273, at least or exactly or at most 274, at least or exactly or at most 275, at least or exactly or at most 276, at least or exactly or at most 277, at least or exactly or at most 278, at least or exactly or at most 279, at least or exactly or at most 280, at least or exactly or at most 281, at least or exactly or at most 282, at least or exactly or at most 283, at least or exactly or at most 284, at least or exactly or at most 285, at least or exactly or at most 286, at least or exactly or at most 287, at least or exactly or at most 288, at least or exactly or at most 289, at least or exactly or at most 290, at least or exactly or at most 291, at least or exactly or at most 292, at least or exactly or at most 293, at least or exactly or at most 294, at least or exactly or at most 295, at least or exactly or at most 296, at least or exactly or at most 297, at least or exactly or at most 298, at least or exactly or at most 299, at least or exactly or at most 300, at least or exactly or at most 301, at least or exactly or at most 302, at least or exactly or at most 303, at least or exactly or at most 304, at least or exactly or at most 305, at least or exactly or at most 306, at least or exactly or at most 307, at least or exactly or at most 308, at least or exactly or at most 309, at least or exactly or at most 310, at least or exactly or at most 311, at least or exactly or at most 312, at least or exactly or at most 313, at least or exactly or at most 314, at least or exactly or at most 315, at least or exactly or at most 316, at least or exactly or at most 317, at least or exactly or at most 318, at least or exactly or at most 319, at least or exactly or at most 320, at least or exactly or at most 321, at least or exactly or at most 322, at least or exactly or at most 323, at least or exactly or at most 324, at least or exactly or at most 325, at least or exactly or at most 326, at least or exactly or at most 327, at least or exactly or at most 328, at least or exactly or at most 329, at least or exactly or at most 330, at least or exactly or at most 331, at least or exactly or at most 332, at least or exactly or at most 333, at least or exactly or at most 334, at least or exactly or at most 335, at least or exactly or at most 336, at least or exactly or at most 337, at least or exactly or at most 338, at least or exactly or at most 339, at least or exactly or at most 340, at least or exactly or at most 341, at least or exactly or at most 342, at least or exactly or at most 343, at least or exactly or at most 344, at least or exactly or at most 345, at least or exactly or at most 346, at least or exactly or at most 347, at least or exactly or at most 348, at least or exactly or at most 349, at least or exactly or at most 350, at least or exactly or at most 351, at least or exactly or at most 352, at least or exactly or at most 353, at least or exactly or at most 354, at least or exactly or at most 355, at least or exactly or at most 356, at least or exactly or at most 357, at least or exactly or at most 358, at least or exactly or at most 359, at least or exactly or at most 360, at least or exactly or at most 361, at least or exactly or at most 362, at least or exactly or at most 363, at least or exactly or at most 364, at least or exactly or at most 365, at least or exactly or at most 366, at least or exactly or at most 367, at least or exactly or at most 368, at least or exactly or at most 369, at least or exactly or at most 370, at least or exactly or at most 371, at least or exactly or at most 372, at least or exactly or at most 373, at least or exactly or at most 374, at least or exactly or at most 375, at least or exactly or at most 376, at least or exactly or at most 377, at least or exactly or at most 378, at least or exactly or at most 379, at least or exact or at most 380, at least or exactly or at most 381, at least or exactly or at most 382, at least or exactly or at most 383, at least or exactly or at most 384, at least or exactly or at most 385, at least or exactly or at most 386, at least or exactly or at most 387, at least or exactly or at most 388, at least or exactly or at most 389, at least or exactly or at most 390, at least or exactly or at most 391, at least or exactly or at most 392, at least or exactly or at most 393, at least or exactly or at most 394, at least or exactly or at most 395, at least or exactly or at most 396, at least or exactly or at most 397, at least or exactly or at most 398, at least or exactly or at most 399, at least or exactly or at most 400, at least or exactly or at most 401, at least or exactly or at most 402, at least or exactly or at most 403, at least or exactly or at most 404, at least or exactly or at most 405, at least or exactly or at most 406, at least or exactly or at most 407, at least or exactly or at most 408, at least or exactly or at most 409, at least or exactly or at most 410, at least or exactly or at most 411, at least or exactly or at most 412, at least or exactly or at most 413, at least or exactly or at most 414, at least or exactly or at most 415, at least or exactly or at most 416, at least or exactly or at most 417, at least or exactly or at most 418, at least or exactly or at most 419, or exactly or at most 420 contiguous amino acid residues.

3. The pharmaceutical composition according to claim 1, wherein said sequence identity is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

4. The polypeptide according to claim 1, wherein the at least 35 contiguous amino acid residues has an N-terminal amino acid residue corresponding to any one of amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, and 386 in any one of amino add residues In SEQ ID NO: 18.

5. The pharmaceutical composition according to claim 1, wherein said polypeptide is fused or conjugated to an immunogenic carrier molecule.

6. The pharmaceutical composition according to claim 5, wherein the immunogenic carrier molecule is a polypeptide that induces T-helper lymphocyte responses In a majority of humans, such as immunogenic carrier proteins selected from the group consisting of keyhole limpet hemocyanin or a fragment thereof, tetanus toxoid or a fragment thereof, diphtheria toxoid or a fragment thereof.

7. The pharmaceutical composition according to claim 1, wherein said polypeptide is capable of inducing an adaptive immune response against the polypeptide in a mammal, in particular in a human being.

8. The pharmaceutical composition according to claim 7, wherein the polypeptide is capable of inducing, in the mammal, a protective adaptive immune response against infection with A. baumannii.

9. The pharmaceutical composition according to claim 7, wherein said polypeptide induces a humoral and/or a cellular immune response.

10. A nucleic acid fragment, which comprises a nucleotide sequence encoding a polypeptide as defined in claim 1 in admixture with a pharmaceutically acceptable carrier, vehicle or diluent, wherein said nucleic acid fragment, when being DNA, comprises a polyadenylation signal and wherein said nucleic acid fragment, when being RNA, comprises a polyA tail.

11. The nucleic acid fragment according to claim 10, which is selected from a DNA fragment and an RNA fragment.

12. An expression vector comprising the nucleic acid according to claim 10.

13. The expression vector according to claim 12, which comprises in operable linkage and in the 5′-3′ direction, an expression control region comprising an enhancer/promoter for driving expression of the nucleic acid fragment 10, optionally a signal peptide coding sequence, a nucleotide sequence defined in claim 10, and optionally a terminator.

14. The expression vector according to claim 13, wherein the expression control region drives expression in prokaryotic cell such as a bacterium, e.g. In E coli.

15. The vector according to claim 12, which is capable of autonomous replication.

16. The vector according to claim 12, which is incapable of being integrated into the genome of a mammalian host cell.

17. The vector according to claim 12, which is selected from the group consisting of a virus, such as a attenuated virus, a bacteriophage, a plasmid, a minichromosome, and a cosmid.

18. A pharmaceutical composition comprising the nucleic acid fragment according to claim 10 or the expression vector according to claim 12, and a pharmaceutically acceptable carrier, vehicle or diluent.

19. The pharmaceutical composition according to claim 18, which further comprises an immunological adjuvant.

20. The pharmaceutical composition according to claim 1, wherein the adjuvant is an aluminium based adjuvant.

21. The pharmaceutical composition according to claim 19, wherein the adjuvant is an aluminium based adjuvant.

22. A method for inducing Immunity In an animal by administering at least once an immunogenically effective amount of a polypeptide defined in claim 1 or a pharmaceutical composition according to claim 1 so as to induce adaptive immunity against A. baumannii In the animal.

23. The method according to claim 22, wherein the animal receives between 0.5 and 5,000 μg of the polypeptide defined In claim 1 per administration.

24. The method according to claim 22, wherein the animal receives a priming administration and one or more booster administrations.

25. The method according to claim 22, wherein the animal is a human being.

26. A method for inducing Immunity In an animal by administering at least once an immunogenically effective amount of a nucleic acid fragment according to claim 10 or an expression vector according to claim 12 so as to induce adaptive immunity against A. baumannii in the animal.
